(19) United States
(12) Patent Application Publication
(10) Pub. No.: US 2012/0088814 A1
(43) Pub. Date: Apr. 12, 2012

(54) METHODS OF MODULATING AN IMMUNE RESPONSE TO A VIRAL INFECTION

(75) Inventor: Susan GREGORY, San Diego, CA (US)

(73) Assignee: Isis Pharmaceuticals, Inc., Carlsbad, CA (US)

(21) Appl. No.: 13/262,218

(22) PCT Filed: Mar. 31, 2010
(86) PCT No.: PCT/US10/29408

§ 371 Date: Dec. 15, 2011

Related U.S. Application Data
(60) Provisional application No. 61/165,384, filed on Mar. 31, 2009.
(60) Provisional application No. 61/242,186, filed on Sep. 14, 2009.
(60) Provisional application No. 61/289,942, filed on Dec. 23, 2009.

(51) Int. Cl. A61K 31/7088; A61P 31/12; A61P 37/02

(52) U.S. Cl. 514/44 A

(57) ABSTRACT

Disclosed herein are methods for treating respiratory disorders via administration of antisense compounds targeting IL-4Rα. Provided herein, for example, are compositions and methods of modulating immune responses to a viral infection in a subject. Also provided, for example, are compositions and methods for managing, treating, ameliorating, preventing and/or delaying the onset of pulmonary inflammation, airway hyperreactivity and/or loss of lung function, or a symptom thereof in a subject during the course of or resulting from a viral infection. Further provided, for example, are compositions and methods of inducing or augmenting hypo-responsiveness, non-responsiveness or tolerance to a virus in a subject. Also provided, for example, are compositions and methods of enhancing the efficacy of a viral vaccine in a subject. In certain embodiments, the compositions and methods provided herein utilize an antisense compound 12 to 35 nucleobases in length targeted to a nucleic acid molecule encoding human IL-4 receptor alpha IL-4Rα, wherein said antisense compound inhibits expression of human IL-4Rα protein and/or expression of functional IL-4 and IL-13 receptors.

### FIELD

Provided herein, for example, are compositions and methods of modulating immune responses to a viral infection in a subject. Also provided, for example, are compositions and methods for managing, treating, ameliorating, preventing and/or delaying the onset of pulmonary inflammation, airway hyperreactivity and/or loss of lung function, or a symptom thereof in a subject during the course of or resulting from a viral infection. Further provided, for example, are compositions and methods of inducing or augmenting hypo-responsiveness, non-responsiveness or tolerance to a virus in a subject. Also provided, for example, are compositions and methods of enhancing the efficacy of a viral vaccine in a subject. In certain embodiments, the compositions and methods provided herein utilize an antisense compound 12 to 35 nucleobases in length targeted to a nucleic acid molecule encoding human IL-4 receptor alpha (IL-4Rα) (SEQ ID NO:1), wherein said antisense compound inhibits expression of human IL-4Rα protein and/or cellular responses to IL-4 and IL-13.

### BACKGROUND

Allergic rhinitis and asthma are widespread conditions with complex and multifactoral etiologies. The severity of the conditions vary widely between individuals, and within individuals, dependent on factors such as genetics, environmental conditions, and cumulative respiratory pathology associated with duration and severity of disease. Both diseases are a result of immune system hyper-responsiveness to innocuous environmental antigens, with asthma typically including an atopic (i.e., allergic) component. Both are Th2-mediated respiratory disorders.

In asthma, the pathology manifests as inflammation, mucus overproduction and reversible airway obstruction, which can result in scarring, airway hyper-responsiveness and changes in airway structure, referred to as airway remodelling, including thickening of the epithelial reticular basement membrane, goblet cell hyperplasia, increased airway smooth muscle, recruitment and activation of myofibroblasts and new blood vessel formation (Murray, (2008) Curr Opin Allergy Clin Immunol 8:77-81), as well as clinical symptoms including chest tightening, wheezing, coughing, shortness of breath, night time awakenings and the need for bronchodilator therapy. Some patients with mild asthma can achieve good control with current therapeutic interventions, including short-acting beta-agonists (SABA) and low dose inhaled corticosteroids (ICS) or cromolyn. A substantial portion of the mild and moderate asthma population, however, can not achieve good control despite compliance with an ICS or an ICS in combination with a long acting beta agonist (LABA) (Bateman, E. D., et al. 2004 Am J Resp Crit. Care Med 170:836-844). Moderate and severe asthma are associated with frequent or chronic symptoms, reduced lung function and exacerbations that require emergency care or intermittent oral corticosteroid treatment. Patients with severe asthma often experience daily symptoms, night time awakenings, limitations on activities, and require periodic emergency care or hospitalization. Serious asthma exacerbations are often associated with increased tissue lymphocytes and airway eosinophils and neutrophils, which can be recruited to the lung and airways by leukotrines and chemokines such as the eotaxins and IL-8, which are in turn produced by epithelial cells and inflammatory cells directly or indirectly in response to the Th2 cytokines IL-4 and IL-13. Despite chronic treatment with combinations of control medications, e.g., high dose ICS supplemented with a leukotriene inhibitor or anti-IgE antibody, and a bronchodilator to achieve control of asthma symptoms and normal lung function, many patients with moderate or severe asthma fail to achieve good control and normal lung function and remain at risk for serious exacerbations. Chronic corticosteroid therapy has a number of unwanted side effects in adults, including oral thrush, and also in children (e.g., damage to bones resulting in decreased growth and risk of fracture).

Allergic rhinitis is inflammation of the nasal passages, and is typically associated with increased eosinophils in the upper airways and nasal tissues, watery nasal discharge, sneezing, congestion and itching of the nose and eyes. It is frequently caused by exposure to irritants, particularly allergens. Allergic rhinitis affects about 20% of the American population and ranks as one of the most common illnesses in the US. Most suffer from seasonal symptoms due to exposure to allergens, such as pollen, that are produced during the natural plant growth season(s). A smaller proportion of patients experience persistent symptoms associated with exposure to allergens that are produced throughout the year, such as house dust mites or animal dander. A number of over-the-counter treatments are available for the treatment of allergic rhinitis, including oral and nasal antihistamines and decongestants. Antihistamines are utilized to suppress itching and sneezing, and many of these drugs are associated with side effects, such as sedation and performance impairment at high doses. Decongestants are often ineffective therapies and frequently cause insomnia, tremor, tachycardia, and hypertension. Intranasal corticosteroids and leukotriene receptor antagonists are also utilized in rhinitis but offer a limited activity profile, e.g., fail to relieve nasal congestion. Allergen immunotherapy is expensive and time consuming and carries a low risk of anaphylaxis.

Persistent nasal polyposis results from chronic eosinophilic inflammation of the nasal and sinus mucous membranes. Chronic inflammation causes a reactive hyperplasia of the intranasal mucosal membrane, which results in the formation of polyps. Nasal polyps are associated with nasal airway obstruction, postnasal drainage, dull headaches, snoring, anosmia, and rhinorrhea. Medical therapies include treatment for underlying chronic allergic rhinitis using antihistamines and topical nasal corticosteroid sprays. Although nasal polyps can be treated pharmacologically, many of the therapeutics have undesirable side effects. Moreover, polyps tend to be recurrent, eventually requiring surgical intervention. Compositions and methods to inhibit post-surgical recurrence of nasal polyps are not presently available.

Other diseases characterized by similar inflammatory sequellae and mediated by Th2 cell responses include, but are not limited to, chronic bronchitis, pneumonia, pulmonary fibrosis (Jakubzick, C., et al. 2003 J Immunol 171:1684), emphysema, chronic obstructive pulmonary disease (COPD), idiopathic pulmonary fibrosis (IPF), and infection with respiratory viruses, including respiratory syncytial virus (RSV), coronavirus, rhinovirus (Message, S. D. et al. 2008 PNAS105:13562), or influenza (Moran T. M., et al. 1996 J Virol 70:5230), including type A/H1N1 influenza virus (Bot, A., et al. 2000 Virology 269:66). In addition, bronchiectasis is a chronic supportive lung disease of diverse etiology characterised by irreversible dilatation of the bronchi and persistent purulent sputum production with increased T cells, activated eosinophils, macrophages and IL-8-expressing cells in the bronchial mucosa (Gaga, M., et al. 1998 Thorax 53:685).

Interleukin-4 Receptor Alpha and Inflammatory Signaling Pathways

It is generally acknowledged that allergy and asthma are a result of the dysregulation of the Th2 cytokine response. Of the Th2 cytokines, IL-4 and IL-13 are most strongly linked to asthma pathogenesis. IL-4 mediates afferent immunity, including Th2 cell maturation and differentiation, IgE production, lung eosinophilia, and vascular endothelial adhesion molecule expression. IL-13 operates in concert with IL-4 and other Th2 cytokines in the generation of immune responses to, for example, inhaled allergens, viruses and noxious particulates. IL-13 also regulates epithelial cell activation and goblet cell maturation. IL-13 is also intimately involved in the manifestation of AHR, lung neutrophilia, lung remodeling, and development of the secretory phenotype in the inflamed airway epithelium (Chatila, T. A., et al. 2004 Trends in Mol Med 10:493).

The IL-4 and IL-13 receptors share a common signaling chain, IL-4Rα. The IL-4Rα gene was cloned independently by two groups (Galizzi, et al. 1990 Int. Immunol. 2:669-675; Idzerda, et al. 1990 J. Exp. Med. 171:861-873), and expression of the IL-4Rα protein indicates that it is a required receptor protein for cellular responses to the Th2 cytokines IL-4 and IL-13 (Nelms, K., et al. 1999 Ann Rev Immunol 17:701-38). IL-4Rα is expressed at low levels ubiquitously and is up-regulated on cells of hematopoietic and non-hematopoietic origin during inflammation. In asthma, critical cell types expressing IL-4Rα include lymphocytes, upper and lower respiratory tract epithelial cells, and antigen-presenting cells such as dendritic cells, alveolar macrophages and eosinophils.

Antisense Oligonucleotides and Compositions Comprising the Same

Compositions and methods for formulation of antisense oligonucleotides (ASOs) and devices for delivery to the lung and nose are well known. ASOs are soluble in aqueous solution and can be delivered using standard nebulizer (Nyce, Exp. Opin. Invest. Drugs, 1997, 6:1149-1156; Gavreau et al. 2008 Am. J. Respir. Crit. Care Med. 177: 952), intranasal spray devices or intranasal gel formulations. Formulations and methods for modulating the size of droplets using, for example, nebulizer or nasal spray devices to target specific portions of the respiratory tract and lungs are also known to those skilled in the art. Oligonucleotides can also be delivered using other devices such as dry powder inhalers, metered dose inhalers and others provided herein.

ASOs targeted to a number of mRNAs or pre-RNAs including, but not limited to those encoding IL-4Rα (U.S. Pat. No. 7,507,810, U.S. Publ. Nos. 20070161549 and 20080103106, and U.S. Ser. No. 11/816,705), Ikappa B Kinase beta (IKKβ; U.S. Pat. Nos. 5,962,673; 5,977,341 and 6,395,545), Stat6, p38 alpha MAP kinase (U.S. Publ. No. 20040171566); the CD28 receptor ligands B7-1 and B7-2 (U.S. Publ. No. 20040235164); intracellular adhesion molecule (ICAM) (WO 2004108945); adenosine A1 receptor (Nyce and Metzger, Nature, 1997, 385:721-725); CCR3 and the βchain subunit of IL-3, IL-5, and GM-CSF receptors (Gavreau et al. 2008 Am. J. Respir. Crit. Care Med. 177: 952) have been tested for their ability to inhibit pulmonary inflammation and airway hyper-responsiveness in humans as well as in mouse, rabbit, and/or monkey models of asthma when delivered by inhalation. Various endpoints were analyzed in each case, and a portion of the results are presented herein. Oligonucleotides are effectively delivered by inhalation to cells within the lungs of multiple species, including a non-human primate, and are effective at reducing allergen-induced changes in lung function, airway hyper-responsiveness and/or pulmonary inflammation.

A number of ASOs and siRNAs designed to target IL-4Rα have been reported for use as research or diagnostic tools, or as pharmaceuticals for the treatment of respiratory disease (Hershey et al., 1997 NEJM337:1720-1725; Rosa-Rosa, et al., 1999 J. Allergy Clin. Immunol 104:1008-1014; Kruse et al. 1999 Immunol. 96, 365-371; WO 2000034789; WO 2002085309; WO 2004011613; WO 2004045543; and U.S. Publ. Nos. 20030104410, US 20040049022, and US 20050143333). However, none of these reports includes a demonstration of the efficacy of the compounds in vivo for the prevention, amelioration, and/or treatment of any disease or disorder.

The methods, compositions and kits provided herein can be used, among other things, to overcome one or more of the problems discussed above.

### SUMMARY

In a first aspect, provided herein is a method for modulating an immune response to a viral infection in a child or adult subject, comprising administering to the subject an effective amount of an antisense compound 12 to 35 nucleobases in length targeted to a nucleic acid molecule encoding an IL-4Rα, wherein said antisense compound inhibits expression of the IL-4Rα protein and/or expression of functional IL-4 and IL-13 receptors. In some embodiments, the viral infection is a primary viral infection. In other embodiments, the viral infection is a secondary viral infection.

In a second aspect provided herein is a method of managing, treating and/or ameliorating pulmonary inflammation, airway hyperreactivity and/or loss of lung function, or a symptom thereof, in a child or adult subject during the course of or resulting from a primary viral infection, comprising administering to a subject an effective amount of an antisense compound 12 to 35 nucleobases in length targeted to a nucleic acid molecule encoding an IL-4Rα, wherein said antisense compound inhibits expression of the IL-4Rα protein and/or expression of functional IL-4 and IL-13 receptors.

In a third aspect, provided herein is a method of managing, treating and/or ameliorating pulmonary inflammation, airway hyperreactivity and/or loss of lung function, or a symptom thereof, in a child or adult subject during the course of or resulting from a secondary viral infection, comprising administering to a subject an effective amount of an antisense compound 12 to 35 nucleobases in length targeted to a nucleic acid molecule encoding an IL-4Rα, wherein said antisense compound inhibits expression of the IL-4Rα protein and/or expression of functional IL-4 and IL-13 receptors.

In a fourth aspect, provided herein is a method of managing, treating and/or ameliorating pulmonary inflammation, airway hyperreactivity and/or loss of lung function, or a symptom thereof, in a child or adult subject during the course of or resulting from exposure to a non-viral environmental irritant, wherein the subject had a primary viral infection as an infant, said method comprising administering to a subject an effective amount of an antisense compound 12 to 35 nucleobases in length targeted to a nucleic acid molecule encoding an IL-4Rα, wherein said antisense compound inhibits expression of the IL-4Rα protein and/or expression of functional IL-4 and IL-13 receptors. In certain embodiments, the non-viral environmental irritant is cigarette smoke, bacteria, mold, dust mites, acarids, pollen, insects, animals (e.g., cats, dogs, rabbits, mice, rats, hamsters, guinea pigs, and birds) and animal dander, fungi, exercise, air pollutants (e.g., tobacco smoke), irritant gases, aerosols, vapors, fumes, chemicals, or cold air.

In a fifth aspect, provided herein is a method of preventing or delaying the onset of pulmonary inflammation, airway hyperreactivity and/or loss of lung function, or a symptom thereof, in a child or adult subject during the course of or resulting from a primary viral infection, comprising administering to a subject an effective amount of an antisense compound 12 to 35 nucleobases in length targeted to a nucleic acid molecule encoding an IL-4Rα, wherein said antisense compound inhibits expression of the IL-4Rα protein and/or expression of functional IL-4 and IL-13 receptors.

In a sixth aspect, provided herein is a method of preventing or delaying the onset of pulmonary inflammation, airway hyperreactivity and/or loss of lung function, or a symptom thereof, in a child or adult subject during the course of or resulting from a secondary viral infection, comprising administering to a subject an effective amount of an antisense compound 12 to 35 nucleobases in length targeted to a nucleic acid molecule encoding an IL-4Rα, wherein said antisense compound inhibits expression of the IL-4Rα protein and/or expression of functional IL-4 and IL-13 receptors.

In a seventh aspect, provided herein is a method of preventing or delaying the onset of pulmonary inflammation, airway hyperreactivity and/or loss of lung function, or a symptom thereof, in a child or adult subject during the course of or resulting from exposure to a non-viral environmental irritant, wherein the subject had a primary viral infection as an infant, said method comprising administering to a subject an effective amount of an antisense compound 12 to 35 nucleobases in length targeted to a nucleic acid molecule encoding an IL-4Rα, wherein said antisense compound inhibits expression of the IL-4Rα protein and/or expression of functional IL-4 and IL-13 receptors. In some embodiments, the non-viral environmental irritant is an allergen. In certain embodiments, the non-viral environmental irritant is cigarette smoke, bacteria, mold, dust mites, acarids, pollen, insects, animals (e.g., cats, dogs, rabbits, mice, rats, hamsters, guinea pigs, and birds) and animal dander, fungi, exercise, air pollutants (e.g., tobacco smoke), irritant gases, aerosols, vapors, fumes, chemicals, or cold air.

In certain embodiments of the methods presented herein, a method comprises decreasing a Th2 response in the subject, increasing a Th1 response in the subject, or a combination thereof. In some embodiments, the pulmonary inflammation, airway hyperreactivity and/or loss of lung function is associated with or caused by an atopic disease. In other embodiments, the pulmonary inflammation, airway hyperreactivity and/or loss of lung function is associated with or caused by a non-atopic disease. In some embodiments, the atopic or non-atopic disease is an allergy, asthma or rhinitis (e.g., allergic rhinitis). In some embodiments, the symptom is bronchoconstriction (i.e., wheezing) or coughing, shortness of breath, coughing, or chest tightening, and objective test findings include increased sputum in the lungs, eosinophilic inflammation, neutrophilic inflammation, elevated level of mucus or mucin protein, subepithelial fibrosis, elevated IgE levels, or elevated level of exhaled nitric oxide, which is associated with or leads to a need for additional immunosuppressive or anti-inflammatory therapies, a need for bronchodilators, a need for corticosteroids, a need for leukotriene inhibitors, a need for anti-IgE antibody therapy, a need for hospitalization, or a combination thereof. In certain embodiments, the nucleic acid molecule encodes human IL-4Rα (SEQ ID NO:1) and the antisense compound inhibits expression of human IL-4Rα protein and/or expression of functional IL-4 and IL-13 receptors. In some embodiments, the subject is a human child. In other embodiments, the subject is a human adult, such as an elderly adult. In some embodiments, the subject is immunocompromised and/or immunosuppressed (e.g., receiving an immunosuppressive therapy).

In an eighth aspect, provided herein is a method of inducing or augmenting hypo-responsiveness, non-responsiveness or tolerance to a virus in a child or adult subject, comprising administering to the subject (i) an antigen of the virus, and (ii) an effective amount of an antisense compound 12 to 35 nucleobases in length targeted to a nucleic acid molecule encoding an IL-4Rα, wherein said antisense compound inhibits expression of the IL-4Rα protein and/or expression of functional IL-4 and IL-13 receptors. In some embodiments, the subject was previously exposed to the virus, for example, as an infant. In other embodiments, the subject has not been previously exposed to the virus. In one embodiment, the method comprises decreasing a Th2 response in the subject, increasing a Th1 response in the subject, or a combination thereof. In certain embodiments, the nucleic acid molecule encodes human IL-4Rα (SEQ ID NO:1) and the antisense compound inhibits expression of human IL-4Rα protein and/or expression of functional IL-4 and IL-13 receptors. In some embodiments, the subject is a human child. In other embodiments, the subject is a human adult, such as an elderly adult. In some embodiments, the subject is immunocompromised and/or immunosuppressed. In certain embodiments, the virus is a respiratory virus infection, such as a rhinovirus, influenza virus (e.g., an influenza A-type virus subtype H1N1 swine flu virus), coronavirus (e.g., SARS virus), RSV infection, or a combination thereof. In some embodiments, the virus infection is not a RSV infection.

In a ninth aspect, provided herein is a method of enhancing the efficacy of a viral vaccine in a child or adult subject, comprising administering to the subject (i) the viral vaccine, and (ii) an effective amount of an antisense compound 12 to 35 nucleobases in length targeted to a nucleic acid molecule encoding an IL-4Rα, wherein said antisense compound inhibits expression of the IL-4Rα protein and/or expression of functional IL-4 and IL-13 receptors. In certain embodiments, the nucleic acid molecule encodes human IL-4Rα (SEQ ID NO:1) and the antisense compound inhibits expression of human IL-4Rα protein and/or expression of functional IL-4 and IL-13 receptors. In some embodiments, the subject is a human child. In other embodiments, the subject is a human adult, such as an elderly adult. In some embodiments, the subject is immunocompromised and/or immunosuppressed. In certain embodiments, the viral vaccine is a respiratory virus vaccine, such as a rhinovirus, influenza virus (e.g., an influenza A-type virus subtype H1N1 swine flu virus), coronavirus (e.g., SARS virus), RSV vaccine, or a combination thereof. In another embodiment, the vaccine is not directed to a respiratory syncytial virus or is not a RSV vaccine. In some embodiments, the vaccine is an attenuated vaccine, inactivated vaccine, toxoid vaccine, subunit vaccine, conjugated vaccine, DNA vaccine, monovalent vaccine, multivalent vaccine, or a combination thereof. In other embodiments, the vaccine is the method of administering the composition sequentially or concurrently in combination with an attenuated vaccine, inactivated vaccine, toxoid vaccine, subunit vaccine, conjugated vaccine, DNA vaccine, monovalent vaccine, multivalent vaccine, or a combination thereof. In certain embodiments, the composition further comprises an adjuvant, which can be sequentially or concurrently administered in combination with the vaccine. In some embodiments, the method comprises decreasing a Th2 response in the subject, increasing a Th1 response in the subject, or a combination thereof.

In a tenth aspect, provided herein is a method for modulating an immune response to a virus (e.g., a rhinovirus, influenza virus or coronavirus) infection in an infant subject, comprising administering to the subject an effective amount of an antisense compound 12 to 35 nucleobases in length targeted to a nucleic acid molecule encoding an IL-4Rα, wherein said antisense compound inhibits expression of the IL-4Rα protein and/or expression of functional IL-4 and IL-13 receptors. In some embodiments, the viral infection is a primary viral infection. In other embodiments, the viral infection is a secondary viral infection.

In an eleventh aspect, provided herein is a method of managing, treating and/or ameliorating pulmonary inflammation, airway hyperreactivity and/or loss of lung function, or a symptom thereof, in an infant subject during the course of or resulting from a primary virus (e.g., rhinovirus, influenza virus or coronavirus) infection, comprising administering to a subject an effective amount of an antisense compound 12 to 35 nucleobases in length targeted to a nucleic acid molecule encoding an IL-4Rα, wherein said antisense compound inhibits expression of the IL-4Rα protein and/or expression of functional IL-4 and IL-13 receptors.

In a twelfth aspect, provided herein is a method of managing, treating and/or ameliorating pulmonary inflammation, airway hyperreactivity and/or loss of lung function, or a symptom thereof, in an infant subject during the course of or resulting from a secondary virus (e.g., rhinovirus, influenza virus or coronavirus) infection, comprising administering to a subject an effective amount of an antisense compound 12 to 35 nucleobases in length targeted to a nucleic acid molecule encoding an IL-4Rα, wherein said antisense compound inhibits expression of the IL-4Rα protein and/or expression of functional IL-4 and IL-13 receptors.

In a thirteenth aspect, provided herein is a method of managing, treating and/or ameliorating pulmonary inflammation, airway hyperreactivity and/or loss of lung function, or a symptom thereof, in an infant subject during the course of or resulting from exposure to a non-viral environmental irritant, wherein the subject previously had a primary (e.g., rhinovirus, influenza virus or coronavirus) infection, said method comprising administering to a subject an effective amount of an antisense compound 12 to 35 nucleobases in length targeted to a nucleic acid molecule encoding an IL-4Rα, wherein said antisense compound inhibits expression of the IL-4Rα protein and/or expression of functional IL-4 and IL-13 receptors. In certain embodiments, the non-viral environmental irritant is cigarette smoke, bacteria, mold, dust mites, acarids, pollen, insects, animals (e.g., cats, dogs, rabbits, mice, rats, hamsters, guinea pigs, and birds) and animal dander, fungi, exercise, air pollutants (e.g., tobacco smoke), irritant gases, aerosols, vapors, fumes, chemicals, or cold air.

In a fourteenth aspect, provided herein is a method of preventing or delaying the onset of pulmonary inflammation, airway hyperreactivity and/or loss of lung function, or a symptom thereof, in an infant subject during the course of or resulting from a primary virus (e.g., rhinovirus, influenza virus or coronavirus) infection, comprising administering to a subject an effective amount of an antisense compound 12 to 35 nucleobases in length targeted to a nucleic acid molecule encoding an IL-4Rα, wherein said antisense compound inhibits expression of the IL-4Rα protein and/or expression of functional IL-4 and IL-13 receptors.

In a fifteenth aspect, provided herein is a method of preventing or delaying the onset of pulmonary inflammation, airway hyperreactivity and/or loss of lung function, or a symptom thereof, in an infant subject during the course of or resulting from a secondary virus (e.g., rhinovirus, influenza virus or coronavirus) infection, comprising administering to a subject an effective amount of an antisense compound 12 to 35 nucleobases in length targeted to a nucleic acid molecule encoding an IL-4Rα, wherein said antisense compound inhibits expression of the IL-4Rα protein and/or expression of functional IL-4 and IL-13 receptors.

In a sixteenth aspect, provided herein is a method of preventing or delaying the onset of pulmonary inflammation, airway hyperreactivity and/or loss of lung function, or a symptom thereof, in an infant subject during the course of or resulting from exposure to a non-viral environmental irritant, wherein the subject previously had a primary virus (e.g., rhinovirus, influenza virus or coronavirus) infection, said method comprising administering to a subject an effective amount of an antisense compound 12 to 35 nucleobases in length targeted to a nucleic acid molecule encoding human IL-4Rα (SEQ ID NO:1), wherein said antisense compound inhibits expression of human IL-4Rα protein and/or expression of functional IL-4 and IL-13 receptors. In certain embodiments, the non-viral environmental irritant is cigarette smoke, bacteria, mold, dust mites, acarids, pollen, insects, animals (e.g., cats, dogs, rabbits, mice, rats, hamsters, guinea pigs, and birds) and animal dander, fungi, exercise, air pollutants (e.g., tobacco smoke), irritant gases, aerosols, vapors, fumes, chemicals, or cold air.

In certain embodiments of the methods provided herein, the viral infection is a respiratory virus infection, such as a rhinovirus, influenza virus (e.g., an influenza A-type virus subtype H1N1 swine flu virus), coronavirus (e.g., SARS virus), RSV infection, or a combination thereof. In some embodiments, the virus infection is not a respiratory syncytial virus infection. In certain embodiments, the method comprises decreasing a Th2 response in the subject, increasing a Th1 response in the subject, or a combination thereof. In some embodiments, the pulmonary inflammation, airway hyperreactivity and/or loss of lung function is associated with or caused by an atopic disease. In other embodiments, the pulmonary inflammation, airway hyperreactivity and/or loss of lung function is associated with or caused by a non-atopic disease. In some embodiments, the atopic or non-atopic disease is an allergy, asthma or rhinitis (e.g., allergic rhinitis). In some embodiments, the symptom is bronchoconstriction (i.e., wheezing, shortness of breath, cough or chest tightness, night time awakenings), or objective test measures including but not limited to increased sputum or sputum proteins in the airways, eosinophilic and/or eosinophilic inflammation in sputum, bronchialalveolar lavage fluid (BALF), nasal or lung tissue biopsy samples, increased mucus or mucin proteins in similar samples, histological, radiological or biochemical evidence of subepithelial fibrosis, collagen deposition or airway basement membrane thickening, elevated IgE levels in serum, sputum or BALF, a need for additional immunosuppressive or anti-inflammatory therapies, a need for bronchodilators, a need for oral or inhaled or intranasal corticosteroids or higher doses of corticosteroids, a need for leukotriene inhibitors, a need for anti-IgE antibody therapy, a need for emergency room treatment or hospitalization, a need for bronchodilators or higher doses of bronchodilators or more frequent use of short-acting bronchodilators, or a combination thereof. In certain embodiments, the nucleic acid molecule encodes human IL-4Rα (SEQ ID NO:1) and the antisense compound inhibits expression of human IL-4Rα protein and/or expression of functional IL-4 and IL-13 receptors. In some embodiments, the subject is a human infant, such as pre-term infant. In some embodiments, the infant is immunocompromised and/or immunosuppressed.

In a seventeenth aspect, provided herein is a method of inducing or augmenting hypo-responsiveness, non-responsiveness or tolerance to a virus (e.g., rhinovirus, influenza virus or coronavirus) in an infant subject, comprising administering to the subject (i) an antigen of the virus, and (ii) an effective amount of an antisense compound 12 to 35 nucleobases in length targeted to a nucleic acid molecule encoding an IL-4Rα, wherein said antisense compound inhibits expression of human IL-4Rα protein and/or expression of functional IL-4 and IL-13 receptors. In some embodiments, the subject was previously exposed to the virus. In other embodiments, the subject has not been previously exposed to the virus. In one embodiment, the method comprises decreasing a Th2 response in the subject, increasing a Th1 response in the subject, or a combination thereof. In certain embodiments, the nucleic acid molecule encodes human IL-4Rα (SEQ ID NO:1) and the antisense compound inhibits expression of human IL-4Rα protein and/or expression of functional IL-4 and IL-13 receptors. In some embodiments, the subject is a human infant, such as pre-term infant. In some embodiments, the infant is immunocompromised and/or immunosuppressed. In certain embodiments, the virus is a respiratory virus infection, such as a rhinovirus, influenza virus (e.g., an influenza A-type virus subtype H1N1 swine flu virus), coronavirus (e.g., SARS virus), RSV infection, or a combination thereof. In some embodiments, the virus infection is not a RSV infection.

In an eighteenth aspect, provided herein is a method of enhancing the efficacy of a viral (e.g., rhinovirus, influenza virus or coronavirus) vaccine in an infant subject, comprising administering to the subject (i) the vaccine, and (ii) an effective amount of an antisense compound 12 to 35 nucleobases in length targeted to a nucleic acid molecule encoding an IL-4Rα, wherein said antisense compound inhibits expression of human IL-4Rα protein and/or expression of functional IL-4 and IL-13 receptors. In certain embodiments, the nucleic acid molecule encodes human IL-4Rα (SEQ ID NO:1) and the antisense compound inhibits expression of human IL-4Rα protein and/or expression of functional IL-4 and IL-13 receptors. In some embodiments, the subject is a human infant, such as pre-term infant. In some embodiments, the infant is immunocompromised and/or immunosuppressed. In certain embodiments, the viral vaccine is a respiratory virus vaccine, such as a rhinovirus, influenza virus (e.g., an influenza A-type virus subtype H1N1 swine flu virus), coronavirus (e.g., SARS virus), RSV vaccine, or a combination thereof. In another embodiment, the vaccine is not directed to a respiratory syncytial virus or is not a RSV vaccine. In some embodiments, the vaccine is an attenuated vaccine, inactivated vaccine, toxoid vaccine, subunit vaccine, conjugated vaccine, DNA vaccine, monovalent vaccine, multivalent vaccine, or a combination thereof. In other embodiments, the vaccine is the method of administering the composition sequentially or concurrently in combination with an attenuated vaccine, inactivated vaccine, toxoid vaccine, subunit vaccine, conjugated vaccine, DNA vaccine, monovalent vaccine, multivalent vaccine, or a combination thereof. In certain embodiments, the composition further comprises an adjuvant, which can be sequentially or concurrently administered in combination with the vaccine. In some embodiments, the method comprises decreasing a Th2 response in the subject, increasing a Th1 response in the subject, or a combination thereof.

In a nineteenth aspect, provided herein is a composition comprising (i) an antigen, and (ii) an antisense compound 12 to 35 nucleobases in length targeted to a nucleic acid molecule encoding an IL-4Rα, wherein said antisense compound inhibits expression of human IL-4Rα protein and/or expression of functional IL-4 and IL-13 receptors. In certain embodiments, the nucleic acid molecule encodes human IL-4Rα (SEQ ID NO:1) and the antisense compound inhibits expression of human IL-4Rα.

In a twentieth aspect, provided herein is a composition comprising (i) a virus or viral antigen thereof, and (ii) an antisense compound 12 to 35 nucleobases in length targeted to a nucleic acid molecule encoding an IL-4Rα, wherein said antisense compound inhibits expression of human IL-4Rα protein and/or expression of functional IL-4 and IL-13 receptors. In certain embodiments, the nucleic acid molecule encodes human IL-4Rα (SEQ ID NO:1) and the antisense compound inhibits expression of human IL-4Rα.

In a twenty-first aspect, provided herein is a composition comprising (i) a vaccine, and (ii) an antisense compound 12 to 35 nucleobases in length targeted to a nucleic acid molecule encoding an IL-4Rα, wherein said antisense compound inhibits expression of human IL-4Rα protein and/or expression of functional IL-4 and IL-13 receptors. In certain embodiments, the nucleic acid molecule encodes human IL-4Rα (SEQ ID NO:1) and the antisense compound inhibits expression of human IL-4Rα protein and/or expression of functional IL-4 and IL-13 receptors.

In a twenty-second aspect, provided herein is a kit comprising in one or more containers (i) a virus or viral antigen thereof, and (ii) an antisense compound 12 to 35 nucleobases in length targeted to a nucleic acid molecule encoding an IL-4Rα, wherein said antisense compound inhibits expression of human IL-4Rα protein and/or expression of functional IL-4 and IL-13 receptors. In certain embodiments, the nucleic acid molecule encodes human IL-4Rα (SEQ ID NO:1) and the antisense compound inhibits expression of human IL-4Rα protein and/or expression of functional IL-4 and IL-13 receptors.

In a twenty-third aspect, provided herein is a kit comprising in one or more containers (i) a vaccine, and (ii) an antisense compound 12 to 35 nucleobases in length targeted to a nucleic acid molecule encoding an IL-4Rα, wherein said antisense compound inhibits expression of human IL-4Rα protein and/or expression of functional IL-4 and IL-13 receptors. In certain embodiments, the nucleic acid molecule encodes human IL-4Rα (SEQ ID NO:1) and the antisense compound inhibits expression of human IL-4Rα protein and/or expression of functional IL-4 and IL-13 receptors.

In a twenty-fourth aspect, provided herein is a method of treating a respiratory disorder in a subject comprising administering (e.g., topically) to the subject (e.g., no more frequently than about once per week) a composition comprising an antisense compound 12 to 35 nucleobases in length targeted to a nucleic acid molecule encoding an IL-4Rα, wherein said compound inhibits expression of human IL-4Rα protein and/or expression of functional IL-4 and IL-13 receptors. In specific embodiments, the respiratory disorder occurs during the course of or results from a viral infection, such as a primary or secondary viral infection. In certain embodiments, the virus is a respiratory virus infection, such as a rhinovirus, influenza virus (e.g., an influenza A-type virus subtype H1N1 swine flu virus), coronavirus (e.g., SARS virus), RSV infection, or a combination thereof. In some embodiments, the virus infection is not a RSV infection.

In a twenty-fifth aspect, provided herein are methods of treating a respiratory disorder in a subject, wherein said subject has at least one of the surrogates of airway or pulmonary inflammation or atopy, consisting of, but not limited to, measurable serum IgE, sputum eosinophilia, sputum neutrophilia, sputum 15-hydroxyeicosatetraenoic acid (15-HETE, the predominant oxidative metabolite of arachidonic acid in human lung), or sputum IL-4Rα mRNA, comprising administering (e.g., topically) to the subject a composition comprising an antisense compound 12 to 35 nucleobases in length targeted to a nucleic acid molecule encoding an IL-4Rα, wherein said compound inhibits expression of human IL-4Rα protein and/or expression of functional IL-4 and IL-13 receptors. In certain embodiments, the nucleic acid molecule encodes human IL-4Rα (SEQ ID NO:1) and the antisense compound inhibits expression of human IL-4Rα protein and/or expression of functional IL-4 and IL-13 receptors. In specific embodiments, the respiratory disorder occurs during the course of or results from a viral infection, such as a primary or secondary viral infection. In certain embodiments, the virus is a respiratory virus infection, such as a rhinovirus, influenza virus (e.g., an influenza A-type virus subtype H1N1 swine flu virus), coronavirus (e.g., SARS virus), RSV infection, or a combination thereof. In some embodiments, the virus infection is not a RSV infection. In a further embodiment, the subject has inflammatory respiratory disease.

In one embodiment, the respiratory disorder is Th2-mediated or associated with Th2 immunity. In another embodiment, the respiratory disorder is selected from the group consisting of allergic and non-allergic asthma, COPD, IPF, cystic fibrosis, chronic bronchitis, rhinitis (e.g., allergic rhinitis), nasal polyposis, and respiratory inflammatory conditions associated with or resulting from a viral infection (e.g., RSV, rhinovirus, influenza virus and coronavirus), chronic pneumonia, pulmonary inflammation, and airway hyper-responsiveness. With respect to the virus infection, treatment according to methods provided herein can, in certain embodiments, change the host immune response to the viral infection. The disorder can, in yet another embodiment, be a viral infection. Such viral infection can, for example, be a RSV, rhinovirus, influenza virus or coronavirus infection. In still another embodiment, the methods provided herein comprise obtaining the antisense compound or composition comprising the same.

Also provided herein are other prophylactic and therapeutic uses of an antisense compound to a nucleic acid molecule encoding an IL-4Rα, such as human IL-4Rα (SEQ ID NO:1), wherein said antisense compound inhibits expression of the IL-4Rα protein and/or expression of functional IL-4 and IL-13 receptors. In specific embodiments of the compositions, kits and methods provided herein, the antisense compound is AIR645.

In some embodiments, the antisense compound is administered to an infant (e.g., a premature infant), a child, an adult (e.g., an elderly adult), or an immunocompromised and/or immunosuppressed individual of any age. In certain embodiments, the antisense compound is administered as (i) as a vaccine, e.g., for prevention of a RSV, rhinovirus, influenza or other viral infection (e.g., a respiratory virus infection) and/or its ensuing complications, such as uncontrolled pulmonary inflammation or asthma, allergy or rhinitis, (ii) as supportive treatment of an identified infection, or (iii) a combination thereof.

In one embodiment of the methods provided herein, the administration (e.g., topical administration) is to a respiratory tract of the subject. In another embodiment, the administration comprises aerosol administration. In certain embodiments, the portion of the respiratory tract selected as target of administration of a composition comprising an antisense compound as described herein is dependent upon the location of the inflammation. For example, in the case of asthma, the compound can be delivered predominantly to the lung. In the case of rhinitis (e.g., allergic rhinitis), the compound can be delivered predominantly to the nasal cavity and/or sinus. The compound can be delivered using any of a number of standard delivery devices and methods well known to those skilled in the art, including, but not limited to nebulizers, nasal and pulmonary inhalers, dry powder inhalers, and metered dose inhalers.

In a further embodiment of the compositions and methods provided herein, the antisense compound 12 to 35 nucleobases in length is targeted (e.g., coding/translated region, 5′ untranslated region, 3′ untranslated region or a combination thereof, including regions spanning the translated and untranslated regions) to a nucleic acid molecule (e.g., pre-RNA or mRNA) encoding an IL-4Rα protein, wherein said antisense compound inhibits expression of the IL-4Rα protein and/or expression of functional IL-4 and IL-13 receptors. In specific embodiments, the compound targets a human IL-4Rα. In some embodiments, the compound is targeted to nucleotides 167-265, 487-525, 2056-2101, 2524-2598, 2731-2791, 3053-3072, or 3168-3187 of SEQ ID NO:1.

In certain embodiments of the methods provided herein, the antisense compound targets an 8, 9, 10, 11, 12, 13, 14, 15, 16, 17, 18, 19, 20, 21, 22, 23, 24, 25, 26, 27, 28, 29, 30, 31, 32, 33, 34 or 35 nucleobase region of human IL-4Rα (SEQ ID NO:1), wherein the target nucleobase region starts at position 21, 49, 78, 101, 167, 173, 176, 193, 194, 196, 197, 199, 200, 201, 202, 203, 205, 206, 207, 208, 209, 210, 211, 212, 213, 215, 217, 219, 220, 221, 222, 223, 224, 225, 226, 227, 228, 229, 233, 234, 244, 246, 284, 287, 317, 330, 340, 353, 355, 388, 428, 429, 430, 431, 438, 443, 487, 494, 496, 497, 499, 500, 501, 502, 503, 504, 506, 509, 510, 530, 531, 611, 619, 620, 621, 624, 642, 645, 647, 649, 716, 721, 726, 735, 736, 737, 741, 758, 777, 891, 900, 917, 918, 931, 936, 998, 999, 1000, 1001, 1003, 1004, 1005, 1006, 1014, 1053, 1077, 1078, 1079, 1080, 1082, 1083, 1085, 1087, 1088, 1090, 1092, 1093, 1094, 1095, 1096, 1098, 1100, 1104, 1114, 1121, 1126, 1131, 1136, 1160, 1175, 1182, 1221, 1223, 1224, 1225, 1227, 1364, 1385, 1390, 1395, 1397, 1398, 1399, 1400, 1401, 1424, 1430, 1435, 1440, 1492, 1499, 1505, 1506, 1507, 1508, 1509, 1510, 1515, 1575, 1608, 1670, 1671, 1673, 1674, 1676, 1700, 1701, 1703, 1705, 1706, 1708, 1716, 1777, 1779, 1780, 1781, 1782, 1834, 1839, 1844, 1845, 1880, 1960, 1976, 1991, 1996, 1997, 2000, 2001, 2006, 2011, 2038, 2043, 2056, 2057, 2058, 2059, 2060, 2062, 2064, 2065, 2066, 2067, 2068, 2079, 2082, 2084, 2087, 2126, 2128, 2130, 2131, 2166, 2204, 2230, 2301, 2315, 2390, 2403, 2437, 2442, 2467, 2469, 2497, 2502, 2506, 2507, 2524, 2526, 2528, 2529, 2530, 2531, 2532, 2541, 2548, 2569, 2578, 2579, 2626, 2643, 2674, 2719, 2731, 2743, 2751, 2763, 2772, 2788, 2793, 2804, 2813, 2827, 2836, 2856, 2859, 2869, 2909, 2915, 2952, 3048, 3053, 3103, 3169, 3198, 3235, 3290, 3297, 3303, 3327, 3340, 3345, 3350, 3350, 3355, 3420, 3432, 3477, 3572, 3578, 3559, 3671 or 3678 of SEQ ID NO:1, and extends in the 3′ direction thereof. In other embodiments of the methods and compositions (e.g., pharmaceutical compositions or formulations) provided herein, the antisense compound targets an 8, 9, 10, 11, 12, 13, 14, 15, 16, 17, 18, 19, 20, 21, 22, 23, 24, 25, 26, 27, 28, 29, 30, 31, 32, 33, 34 or 35 nucleobase region of human IL-4Rα (SEQ ID NO:1), wherein the target nucleobase region starts at position 40, 68, 97, 120, 186, 192, 195, 112, 113, 115, 116, 118, 119, 220, 221, 222, 224, 225, 226, 227, 228, 229, 230, 231, 232, 234, 236, 238, 239, 240, 241, 242, 243, 244, 245, 246, 247, 248, 252, 253, 263, 265, 303, 306, 336, 349, 359, 372, 374, 407, 447, 448, 449, 450, 457, 462, 506, 513, 515, 516, 518, 519, 520, 521, 522, 523, 525, 528, 529, 549, 550, 630, 638, 639, 640, 643, 661, 664, 666, 668, 735, 740, 745, 754, 755, 756, 760, 777, 796, 910, 919, 936, 937, 950, 955, 1017, 1018, 1019, 1020, 1022, 1023, 1024, 1025, 1033, 1072, 1096, 1097, 1098, 1099, 1101, 1102, 1204, 1106, 1107, 1109, 1111, 1112, 1113, 1114, 1115, 1117, 1119, 1123, 1133, 1140, 1145, 1150, 1155, 1179, 1194, 1201, 1240, 1242, 1243, 1244, 1246, 1383, 1404, 1409, 1414, 1416, 1417, 1418, 1419, 1420, 1443, 1449, 1454, 1459, 1511, 1518, 1524, 1525, 1526, 1527, 1528, 1529, 1534, 1594, 1627, 1689, 1690, 1692, 1693, 1695, 1719, 1720, 1722, 1724, 1725, 1727, 1735, 1796, 1798, 1799, 1800, 1801, 1853, 1858, 1863, 1864, 1899, 1979, 1995, 2010, 2015, 2016, 2019, 2020, 2025, 2030, 2057, 2062, 2075, 2076, 2077, 2078, 2079, 2081, 2083, 2084, 2085, 2086, 2087, 2098, 2101, 2103, 2106, 2145, 2147, 2149, 2150, 2185, 2223, 2249, 2320, 2334, 2409, 2422, 2456, 2461, 2486, 2488, 2516, 2521, 2525, 2526, 2543, 2545, 2547, 2548, 2549, 2550, 2551, 2570, 2567, 2588, 2597, 2598, 2645, 2662, 2693, 2738, 2750, 2762, 2770, 2782, 2791, 2807, 2812, 2823, 2832, 2846, 2855, 2875, 2878, 2888, 2928, 2934, 2971, 3067, 3072, 3122, 3188, 3217, 3254, 3309, 3316, 3322, 3346, 3359, 3364, 3369, 3369, 3374, 3439, 3451, 3496, 3591, 3597, 3578, 3690 or 3697 of SEQ ID NO:1, and extends in the 5′ direction thereof. In other embodiments, the antisense compound targets an 8, 9, 10, 11, 12, 13, 14, 15, 16, 17, 18, 19, 20, 21, 22, 23, 24, 25, 26, 27, 28, 29, 30, 31, 32, 33, 34 or 35 nucleobase region of human IL-4Rα (SEQ ID NO:1) spanning positions 1 and 3697 of SEQ ID NO:1, such as from or between positions 21, 49, 78, 101, 167, 173, 176, 193, 194, 196, 197, 199, 200, 201, 202, 203, 205, 206, 207, 208, 209, 210, 211, 212, 213, 215, 217, 219, 220, 221, 222, 223, 224, 225, 226, 227, 228, 229, 233, 234, 244, 246, 284, 287, 317, 330, 340, 353, 355, 388, 428, 429, 430, 431, 438, 443, 487, 494, 496, 497, 499, 500, 501, 502, 503, 504, 506, 509, 510, 530, 531, 611, 619, 620, 621, 624, 642, 645, 647, 649, 716, 721, 726, 735, 736, 737, 741, 758, 777, 891, 900, 917, 918, 931, 936, 998, 999, 1000, 1001, 1003, 1004, 1005, 1006, 1014, 1053, 1077, 1078, 1079, 1080, 1082, 1083, 1085, 1087, 1088, 1090, 1092, 1093, 1094, 1095, 1096, 1098, 1100, 1104, 1114, 1121, 1126, 1131, 1136, 1160, 1175, 1182, 1221, 1223, 1224, 1225, 1227, 1364, 1385, 1390, 1395, 1397, 1398, 1399, 1400, 1401, 1424, 1430, 1435, 1440, 1492, 1499, 1505, 1506, 1507, 1508, 1509, 1510, 1515, 1575, 1608, 1670, 1671, 1673, 1674, 1676, 1700, 1701, 1703, 1705, 1706, 1708, 1716, 1777, 1779, 1780, 1781, 1782, 1834, 1839, 1844, 1845, 1880, 1960, 1976, 1991, 1996, 1997, 2000, 2001, 2006, 2011, 2038, 2043, 2056, 2057, 2058, 2059, 2060, 2062, 2064, 2065, 2066, 2067, 2068, 2079, 2082, 2084, 2087, 2126, 2128, 2130, 2131, 2166, 2204, 2230, 2301, 2315, 2390, 2403, 2437, 2442, 2467, 2469, 2497, 2502, 2506, 2507, 2524, 2526, 2528, 2529, 2530, 2531, 2532, 2541, 2548, 2569, 2578, 2579, 2626, 2643, 2674, 2719, 2731, 2743, 2751, 2763, 2772, 2788, 2793, 2804, 2813, 2827, 2836, 2856, 2859, 2869, 2909, 2915, 2952, 3048, 3053, 3103, 3169, 3198, 3235, 3290, 3297, 3303, 3327, 3340, 3345, 3350, 3350, 3355, 3420, 3432, 3477, 3572, 3578, 3559, 3671 and/or 3678 of SEQ ID NO:1, or any region thereof. In certain embodiments, the antisense compound targets a 19 or 20 nucleobase region of human IL-4Rα (SEQ ID NO:1) spanning positions 2056 to 2079 of SEQ ID NO:1. In specific embodiments, the antisense compound does not target a nucleobase region consisting of an 8, 9, 10, 11, 12, 13, 14, 15, 16, 17 or 18 nucleobase region of human IL-4Rα (SEQ ID NO:1) spanning positions 2056 to 2079 of SEQ ID NO:1. In specific embodiments, the antisense compound does not target a nucleobase region of human IL-4Rα (SEQ ID NO:1) comprising position 2051, 2052, 2053, 2054, 2055, 2080, 2081, 2082, 2083, and/or 2084 of SEQ ID NO:1. In some embodiments, the antisense compound does not target a nucleobase region of human IL-4Rα (SEQ ID NO:1) consisting of positions 2055 to 2073 of SEQ ID NO:1 In other embodiments, the antisense compound does not target a nucleobase region of human IL-4Rα (SEQ ID NO:1) consisting of or comprising the region spanning 2258 to 2282 of SEQ ID NO:1.

In another embodiment of the methods provided herein, the compound is at least about 80% identical to the complement of a 20-nucleobase portion of nucleotides 167-265, 487-525, 2056-2101, 2524-2598, 2731-2791, 3053-3072, or 3168-3187 of SEQ ID NO:1. In still another embodiment, the compound is at least about 80% identical to the complement of a 20-nucleobase portion of nucleotides 2056-2087 of SEQ ID NO:1. In some embodiments, the compound comprises a nucleobase portion that is at least about 80% identical to SEQ ID NO:137, SEQ ID NO:155, SEQ ID NO:196, SEQ ID NO:276, SEQ ID NO:279, SEQ ID NO:280, SEQ ID NO:292, SEQ ID NO:298, SEQ ID NO:302 or SEQ ID NO:303. In a further embodiment, the compound is at least about 80% identical to SEQ ID NO:137, SEQ ID NO:155, SEQ ID NO:196, SEQ ID NO:276, SEQ ID NO:279, SEQ ID NO:280, SEQ ID NO:292, SEQ ID NO:298, SEQ ID NO:302 or SEQ ID NO:303. In another embodiment, the compound comprises SEQ ID NO:137, SEQ ID NO:155, SEQ ID NO:196, SEQ ID NO:276, SEQ ID NO:279, SEQ ID NO:280, SEQ ID NO:292, SEQ ID NO:298, SEQ ID NO:302 or SEQ ID NO:303. In yet another embodiment, the compound consists of SEQ ID NO:137, SEQ ID NO:155, SEQ ID NO:196, SEQ ID NO:276, SEQ ID NO:279, SEQ ID NO:280, SEQ ID NO:292, SEQ ID NO:298, SEQ ID NO:302 or SEQ ID NO:303.

In further embodiments of the compositions and methods provided herein, the compound is a single-stranded compound. In one embodiment, the compound comprises a chimeric oligonucleotide. In another embodiment, the compound comprises at least one modified internucleoside linkage, sugar moiety, or nucleobase. In additional embodiments, the modified internucleoside linkage is a phosphorothioate linkage, the modified sugar moiety is a 2′-MOE modification, and the modified nucleobase is a 5-methylcytosine.

The antisense compounds (also included under oligomeric compounds, especially nucleic acid and nucleic acid-like oligomers) described herein are targeted to a nucleic acid (e.g., pre-RNA or mRNA) encoding an IL-4Rα (e.g., coding region, 5′ untranslated region, 3′ untranslated region, region spanning the coding and an untranslated region, or a combination thereof). In certain embodiments, the antisense compounds are antisense oligonucleotides targeted to an IL-4Rα, particularly a human IL-4Rα (GenBank Accession No. X52425.1, entered 26 May 1992 (SEQ ID NO. 1); GenBank Accession No. BM738518.1, entered 1 Mar. 2002; nucleotides 18636000 to 18689000 of GenBank Accession No. NT 010393.14 entered 19 Feb. 2004, each of which is incorporated by reference), that modulate the expression of IL-4Rα. The compounds can comprise at least a 12-nucleobase portion, such as at least a 17-nucleobase portion, of the sequences listed in Table 3, 4 or 5, or are at least 90% identical to validated target segments, or the sequences listed in Table 3, 4, or 5.

Other aspects and embodiments of the invention are described in or are obvious from the following disclosure and are within the ambit of the invention. The following detailed description is given by way of figures and examples, but is not intended to limit the invention to specific embodiments described.

### BRIEF DESCRIPTION OF THE DRAWINGS

FIG. 1 shows a bar graph depicting the tissue exposure after 13-week recovery following the last inhalation dose (15 mg/kg/wk) with AIR645 compared to end of treatment (6 nebulizations over 28 days) in monkeys.

FIG. 2 shows, in bar graph form, AIR645 sputum drug concentration for various single doses administered via nebulization to healthy adult volunteers.

FIGS. 3A-3B graphically depict AIR645 sputum drug concentrations over the period of repeat dose administration (6 nebulizations over 22 days) and at the end of 14-day recovery following the last inhalation dose in (A) healthy volunteers and (B) adults with well controlled asthma (20 mg AIR645 dose).

FIG. 4 schematically depicts the schedule of AIR645 or saline (placebo) administration by inhalation in healthy adult volunteers (Cohorts 6-9 in AIR645-CS1) or in adults with well controlled asthma (Cohort 10 of AIR645-CS1).

FIG. 5 shows a flow chart of induced sputum sample handling for AIR645-CS1 Cohort 10.

FIG. 6 graphically depicts the total serum IgE levels determined during AIR645 repeat-dose treatment and 14-day follow-up period in subjects with well controlled asthma (Cohort 10).

FIG. 7 shows, in bar graph form, the percent sputum eosinophils determined during AIR645 repeat-dose treatment and at the end of the 14-day follow-up period in subjects with well controlled asthma (Cohort 10). Dashes indicate inadequate sputum sample to provide data. PBO, placebo.

FIG. 8 shows, in bar graph form, the absolute numbers of sputum cells determined during AIR645 treatment and follow-up periods from subject 10-007 in Cohort 10.

FIG. 9 shows, in bar graph form, the sputum solute 15-HETE levels determined during AIR645 treatment and follow-up periods in samples collected from each of the 8 subjects of Cohort 10. Dashes indicate inadequate sputum sample to provide data. PBO, placebo.

FIGS. 10A-10B show, in bar graph form, (A) the level of IL-4Rα mRNA in sputum and (B) the relative level compared with the house-keeping gene, glucuronidase beta (GUS B), respectively, as determined by RT-PCR analysis of sputum samples collected during AIR645 repeat-dose treatment and at the end of the 14-day follow-up period in subjects with well controlled asthma (Cohort 10). PBO, placebo.

FIG. 11 shows the schedule and route of ovalbumin (OVA) and IL-4Rα ASO administration in mice. IL-4Rα antisense was administered by intranasal instillation.

FIGS. 12A-12B show suppression of OVA-induced nasal eosinophilia in a mouse model of allergic rhinitis following intranasal administration of IL-4Rα ASO, including (A) percentages of macrophages, lymphocytes, eosinophils, and neutrophils in nasal lavage fluid, and (B) numbers of eosinophils per square millimeter of nasal tissue. *Values are mean±standard deviation; p<0.05 vehicle control (Veh); NA=naive; Mac=macrophage; Lym=lymphocyte; Eos=eosinophil; Neu=neutrophil.

FIGS. 13A-13B shows reductions in behaviors associated with rhinitis symptoms in OVA-sensitized and -challenged mice following intranasal administration of IL-4Rα ASO, including (A) frequency of nasal rubbing and (B) frequency of sneezing. *Values are mean±standard deviation; p<0.05 vehicle control (Veh); NA=naive.

FIGS. 14A-14D depicts the levels of various endpoints in sham treated mice or mice treated with an IL-4Rα antisense compound, including (A) weight loss, (B) illness score, (C) RSV viral titers and (D) numbers of macrophages, lymphocytes, neutrophils, eosinophil and total cell counts in the bronchoalveolar lavage (BAL).

### TERMINOLOGY

Unless defined otherwise, all technical and scientific terms used herein have the same meaning as is commonly understood by one of ordinary skill in the art. All patents, applications, published applications and other publications are incorporated herein by reference in their entirety. In the event that there is a plurality of definitions for a term herein, those in this section prevail unless stated otherwise.

The term “about” or “approximately” means within 20%, such as within 10%, within 5%, or within 1% or less of a given value or range.

As used herein, “administer” or “administration” refers to the act of injecting or otherwise physically delivering a substance as it exists outside the body (e.g., an antisense compound provided herein) into a patient, such as by, but not limited to, pulmonary (e.g., inhalation), mucosal (e.g., intranasal), intradermal, intravenous, intramuscular delivery and/or any other method of physical delivery described herein or known in the art. When a disease, or a symptom thereof, is being managed or treated, administration of the substance typically occurs after the onset of the disease or symptoms thereof. When a disease, or symptom thereof, is being prevented, administration of the substance typically occurs before the onset of the disease or symptoms thereof and may be continued chronically to defer or reduce the appearance or magnitude of disease-associated symptoms, e.g., damage to the involved tissues and airways.

As used herein, the term “adult” subject refers, in certain embodiments, to a human subject that is sixteen years of age or older.

As used herein, the term “alternating motif” refers to an oligomeric compound comprising a contiguous sequence of nucleosides comprising two differentially sugar modified nucleosides that alternate for essentially the entire sequence of the oligomeric compound, or for essentially the entire sequence of a region of an oligomeric compound. The pattern of alternation can be described by the formula: 5′-A(-L-B-L-A)n(-L-B)nn-3′ where A and B are nucleosides differentiated by having at least different sugar groups, each L is an internucleoside linking group, nn can be 0 or 1 and n can be from about 5 to about 11; however, the number can be larger than about 11. This formula also allows for even and odd lengths for alternating oligomeric compounds wherein the 3′ and 5′-terminal nucleosides are the same (odd) or different (even).

The terms “antisense compound” or “antisense oligomeric compound,” as used herein, refer to an oligomeric compound that is at least partially complementary to the region of a target nucleic acid molecule to which it hybridizes and which modulates (increases or decreases) its expression.

An “antisense oligonucleotide” as used herein is an antisense compound that is a nucleic acid-based oligomer. An antisense oligonucleotide can, in some cases, include one or more chemical modifications to the sugar, base, and/or internucleoside linkages.

As used herein, “auto-catalytic” means a compound has the ability to promote cleavage of the target RNA in the absence of accessory factors, e.g., proteins.

As used herein, the term “blockmer motif” refers to a sequence of nucleosides that have uniform sugars (identical sugars, modified or unmodified) that is internally interrupted by a block of sugar modified nucleosides that are uniformly modified and wherein the modification is different from the other nucleosides. In certain embodiments, oligomeric compounds having a blockmer motif comprise a sequence of β-D-deoxyribonucleosides having one internal block of from 2 to 6 sugar modified nucleosides. The internal block region can be at any position within the oligomeric compound as long as it is not at one of the termini which would then make it a hemimer.

As used herein, the term “child” subject refers, in certain embodiments, to a human subject that is older than two years of age, but younger than sixteen years of age (e.g., younger than 5 years of age or younger than 10 years of age).

As used herein, the term “chimeric oligomeric compound” refers to an oligomeric compound having at least one sugar, nucleobase and/or internucleoside linkage that is differentially modified as compared to the other sugars, nucleobases and internucleoside linkages within the same oligomeric compound. The remainder of the sugars, nucleobases and internucleoside linkages can be independently modified or unmodified provided that they are distinguishable from the differentially modified moiety or moieties. In general a chimeric oligomeric compound will have modified nucleosides that can be in isolated positions or grouped together in regions that will define a particular motif. Any combination of modifications and/or mimetic groups can comprise a chimeric oligomeric compound.

As used herein, the term “composition” is intended to encompass a product containing the specified ingredients (e.g., an antisense compound provided herein) in, optionally, the specified amounts, as well as any product which results, directly or indirectly, from combination of the specified ingredients in, optionally, the specified amounts.

The terms “comprises”, “comprising”, are intended to have the broad meaning ascribed to them in U.S. Patent Law and can mean “includes”, “including” and the like.

As used herein, the term “elderly” subject refers, in certain embodiments, to a human subject that is older than sixty-five years of age.

The term “effective amount” as used herein refers to the amount of a therapy (e.g., an antisense compound or pharmaceutical composition provided herein) which is sufficient to reduce and/or ameliorate the severity and/or duration of a given disease and/or a symptom related thereto. This term also encompasses an amount necessary for the reduction or amelioration of the advancement or progression of a given disease, reduction or amelioration of the recurrence, development or onset of a given disease, and/or to improve or enhance the prophylactic or therapeutic effect(s) of another therapy (e.g., a therapy other than an antisense compound provided herein). In some embodiments, the effective amount of an antisense compound provided herein is from about 0.1 mg/kg (mg of antisense compound per kg weight of the subject) to about 100 mg/kg. In certain embodiments, an effective amount is about 0.001 μg/kg (μg of antisense compound per kg weight of the subject), about 0.01 μg/kg, about 0.1 μg/kg, about 0.001 mg/kg, about 0.01 mg/kg, about 0.1 mg/kg, about 0.5 mg/kg, about 1 mg/kg, 3 mg/kg, 5 mg/kg, about 8 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg about 90 mg/kg or about 100 mg/kg (or a range therein). The effective amounts provided herein may be administered one time as a single dose or as fractionated doses over a period of time (e.g., over the course of days, weeks, months, years of the lifetime of the subject). For example, in certain embodiments, the effective amount is administered once per week (one dose of 3.5 mg/kg; total dose of 3.5 mg/kg per week), but can also be fractionated for more frequent administration, such as once per day for one week (seven doses of 0.5 mg/kg; total dose of 3.5 mg/kg/week).

In some embodiments, “effective amount” as used herein also refers to the amount of an antisense compound provided herein to achieve a specified result(e.g., decreasing a Th2 immune response, increasing a Th1 immune response, decreasing airway hyperreactivity, decreasing pulmonary inflammation, maintaining or increasing lung function, maintaining or decreasing airway resistance, maintaining or increasing airway compliance, or a combination thereof). In certain embodiments, the term “effective amount” or “therapeutically effective amount” as used herein refers to an amount sufficient to produce a beneficial or desired clinical result upon treatment. In some embodiments, the term “effective amount” or “therapeutically effective amount” means an amount of an antisense compound as described herein sufficient to measurably (i) reduce or inhibit the expression of IL-4Rα mRNA or protein in a fluid or tissue (e.g., sputum or sputum cells, bronchoalveolar cells, nasal lavage cells, lung biopsy or nasal tissue biopsy) as determined in a relevant in vitro assay or (ii) cause a measurable improvement in an animal model of, for example, asthma or allergy as determined by measurements of clinical symptoms or immune or inflammatory response measurements. Alternatively, an “effective amount” or “therapeutically effective amount” is, in some embodiments, an amount of an antisense compound as described herein sufficient to confer a therapeutic or prophylactic effect on the treated subject against a respiratory disorder, in particular, a Th2-mediated disorder. A (therapeutically or prophylactically) effective amount will vary, as recognized by those skilled in the art, depending on the specific disorder treated, the route of administration, the administration regimen and duration, the excipient(s), delivery device selected, and the possibility of combination therapy, such as the administration of other effective therapies (e.g., therapeutic medications).

As used herein, the term “fully modified motif” refers to an oligomeric compound comprising a contiguous sequence of nucleosides wherein essentially each nucleoside is a sugar modified nucleoside having uniform modification.

As used herein, the term “gapped motif” refers to an oligomeric compound comprising a contiguous sequence of nucleosides that is divided into three regions, an internal region (gap) flanked by two external regions (wings). The regions are differentiated from each other at least by having differentially modified sugar groups that comprise the nucleosides. In some embodiments, each modified region is uniformly modified (e.g., the modified sugar groups in a given region are identical); however, other motifs can be applied to regions. For example, the wings in a gapmer could have an alternating motif. The internal region or the gap can, in some instances, comprise uniform unmodified β-D-ribonucleosides or β-D-deoxyribonucleosides or can be a sequence of nucleosides having uniformly modified sugars. The nucleosides located in the gap of a gapped oligomeric compound have sugar moieties that are different than the modified sugar moieties in each of the wings.

As used herein, the term “hemimer motif” refers to a sequence of nucleosides that have uniform sugar moieties (identical sugars, modified or unmodified) and wherein one of the 5′-end or the 3′-end has a sequence of from 2 to 12 nucleosides that are sugar modified nucleosides that are different from the other nucleosides in the hemimer modified oligomeric compound. An example of a typical hemimer is an oligomeric compound comprising β-D-deoxyribonucleosides having a contiguous sequence of sugar modified nucleosides at one of the termini.

The terms “hypo-responsiveness” as used herein refers to a reduction of immune reactivity to a specific antigen or group of antigens to which a person is normally responsive, such as upon primary or secondary (e.g., re-stimulation) exposure to the antigen, wherein the immune response is a less-than-predicted response based on normal population studies, or, for example compared to a subject receiving no antisense compound or the same subject prior to receiving antisense compound therapy. The reduction may be in the form of reducing an immune response already in progress, or may involve reducing the induction of an immune response, which can result in the reduction or elimination of a discernable symptom resulting from the antigenic stimulation (e.g., a viral infection or non-viral environmental irritant).

As used herein, the term “in combination” in the context of the administration of other therapies refers to the use of more than one therapy. The use of the term “in combination” does not restrict the order in which therapies are administered to a subject. A first therapy can be administered before (e.g., 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks), concurrently, or after (e.g., 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks) the administration of a second therapy to a subject. Any additional therapy can be administered in any order and/or by any route with the other additional therapies. In certain embodiments, the antisense compounds provided herein can be administered in combination with one or more therapies (e.g., one or more additional different antisense compound(s) and/or one or more additional therapies that are not an antisense compound). Non-limiting examples of therapies that can be administered in combination with an antisense compound provided herein of the invention include analgesic agents, anesthetic agents, antibiotics, or immunomodulatory agents or any other agent listed in the U.S. Pharmacopoeia—National Formulary (2009) U.S. Pharmacopoeia, including revisions, and/or Physician's Desk Reference (2009) 63^{rd} ed., Thomson Reuters.

As used herein, the term “infant” subject refers, in certain embodiments, to a human subject that is two years of age or younger.

As used herein, the terms “infection” and “viral infection” refers to all stages of a virus life cycle in a host subject (including, but not limited to the invasion by and replication of the virus in a cell or body tissue), as well as the pathological state resulting from the invasion by and replication of a virus. The invasion by and multiplication of a virus can include, but is not limited to, the following steps: the docking of the virus particle to a cell, fusion of the virus with a cell membrane, the introduction of viral genetic information into a cell, the expression of virus proteins, the production of new virus particles and the release of virus particles from a cell. In certain embodiments, a subject can be clinically diagnosed with a viral infection, e.g., by medical personnel, for example, following a diagnostic test, such an ELISA or PCR. In other embodiments, a viral infection can be diagnosed by virtue of the subject having one or more clinical manifestations or symptoms of the virus infection, such as a fever, wheezing, coughing, shortness of breath or other symptom described herein. In certain embodiments, the viral infection requires the subject to obtain medical intervention, such as hospitalization, administration of oxygen, intubation and/or ventilation.

The term “lower respiratory” tract refers to the major passages and structures of the lower respiratory tract including the windpipe (trachea) and the lungs, including the bronchi, bronchioles, and alveoli of the lungs.

As used herein, the terms “manage,” “managing,” and “management” refer to the beneficial effects that a subject derives from a therapy (e.g., a prophylactic or therapeutic agent), which does not result in a cure of the disease or symptom thereof. In certain embodiments, a subject is administered one or more therapies (e.g., prophylactic or therapeutic agents, such as an antisense compound provided herein) to “manage” or otherwise control pulmonary inflammation, airway hyperreactivity and/or loss of lung function, and/or one or more symptoms thereof, so as to prevent the progression or worsening of the disease or symptom(s), reduce the number or severity of disease exacerbations, or reduce the requirement for or dose of other effective therapies, such as therapeutic medications.

As used herein the term “mimetic” refers to groups that are substituted for a sugar, a nucleobase, and/or internucleoside linkage. Mimetics are typically groups that are structurally quite different (not simply a modification) but functionally similar to the linked nucleosides of oligonucleotides.

As used herein, the term “motif” refers to the orientation of modified sugar moieties and/or sugar mimetic groups in an oligomeric compound relative to like or differentially modified or unmodified nucleosides. As used herein, the terms “sugars,” “sugar moieties” and “sugar mimetic groups” are used interchangeably. Such motifs include, but are not limited to, gapped motifs, alternating motifs, fully modified motifs, hemimer motifs, blockmer motifs, and positionally modified motifs. The sequence and the structure of the nucleobases and type of internucleoside linkage is not a factor in determining the motif of an oligomeric compound.

The term “non-responsiveness” as used herein refers to the amelioration or elimination of immune reactivity to a specific antigen or group of antigens to which a person is normally responsive, such as upon primary or secondary (e.g., re-stimulation) exposure to the antigen, wherein the immune response is not detectable, for example, compared to a subject receiving no antisense compound or the same subject prior to receiving antisense compound therapy. The elimination may be in the form of amelioration of an immune response already in progress, or may involve eliminating the induction of an immune response, which can result in the elimination of a discernable symptom resulting from the antigenic stimulation (e.g., a virus, viral infection or non-viral environmental irritant).

As used herein, a “non-viral environmental irritant” refers to an allergen, bacteria, fungus, prion or other non-viral agent that causes or is associated with pulmonary inflammation, airway hyperreactivity and/or loss of lung function and/or a symptom related thereto. In certain embodiments, the non-viral environmental irritant is cigarette smoke, bacteria, mold, dust mites, acarids, pollen, insects, animals (e.g., cats, dogs, rabbits, mice, rats, hamsters, guinea pigs, and birds) and animal dander, fungi, exercise, air pollutants (e.g., tobacco smoke), irritant gases, aerosols, vapors, fumes, chemicals, or cold air.

The term “nucleobase” or “heterocyclic base moiety” as used herein, refers to the heterocyclic base portion of a nucleoside. In general, a nucleobase is any group that contains one or more atom or groups of atoms capable of hydrogen bonding to a base of another nucleic acid. In addition to “unmodified” or “natural” nucleobases such as the purine nucleobases adenine (A) and guanine (G), and the pyrimidine nucleobases thymine (T), cytosine (C) and uracil (U), many modified nucleobases or nucleobase mimetics known to the art skilled and can be used in the compounds provided herein. The terms modified nucleobase and nucleobase mimetic can overlap but generally a modified nucleobase refers to a nucleobase that is fairly similar in structure to the parent nucleobase such as for example a 7-deaza purine or a 5-methyl cytosine whereas a nucleobase mimetic would include more complicated structures such as for example a tricyclic phenoxazine nucleobase mimetic. Methods for preparation of the above-noted modified nucleobases are well known to those skilled in the art.

As used herein the term “nucleoside” includes nucleosides, abasic nucleosides, modified nucleosides, and nucleosides having mimetic bases and/or sugar groups.

As used herein the term “nucleoside mimetic” is intended to include those structures used to replace the sugar or the sugar and the base and not necessarily the linkage at one or more positions of an oligomeric compound such as for example nucleoside mimetics having morpholino, cyclohexenyl, cyclohexyl, tetrahydropyranyl, bicyclo or tricyclo sugar mimetics, e.g., non furanose sugar units.

The term “nucleotide mimetic” is intended to include those structures used to replace the nucleoside and the linkage at one or more positions of an oligomeric compound such as for example peptide nucleic acids or morpholinos (morpholinos linked by —N(H)—C(═O)—O— or other non-phosphodiester linkage).

The term “obtaining” as in “obtaining the compound” is intended to include purchasing, synthesizing or otherwise acquiring the compound (or indicated substance or material).

The term “oligomeric compound” as used herein refers to a polymeric structure capable of hybridizing to a region of a nucleic acid molecule. This term includes oligonucleotides, oligonucleosides, oligonucleotide analogs, oligonucleotide mimetics, and chimeric combinations of these.

As used herein, the term “oligonucleotide” refers to an oligomeric compound which is an oligomer or polymer of ribonucleic acid (RNA) or deoxyribonucleic acid (DNA). This term includes oligonucleotides composed of naturally- and non-naturally-occurring nucleobases, sugars and covalent internucleoside linkages, possibly further including non-nucleic acid conjugates.

The term “pharmaceutically acceptable” as used herein means being approved by a regulatory agency of the Federal or a state government, or listed in the U.S. Pharmacopeia, European Pharmacopeia or other generally recognized Pharmacopeia for use in animals, and more particularly in humans.

The term “pharmaceutically acceptable salts” refers to physiologically and pharmaceutically acceptable salts of the antisense compounds described herein: i.e., salts that retain the desired biological activity of the parent compound and do not impart undesired toxicological effects thereto. Sodium salts of antisense oligonucleotides are useful and are well accepted for therapeutic administration to humans. In another embodiment, sodium salts of dsRNA compounds are also provided.

A “pharmaceutical carrier” or “excipient” can be a pharmaceutically acceptable solvent, suspending agent, or any other pharmacologically inert vehicle for delivering one or more nucleic acids to an animal and are known in the art. The excipient can be liquid or solid and is selected, with the planned manner of administration in mind, so as to provide for the desired bulk, consistency, etc., when combined with a nucleic acid and the other components of a given pharmaceutical composition. The term “excipients” as used herein refers to inert substances which are commonly used as a diluent, vehicle, preservatives, binders, or stabilizing agent for drugs and includes, but not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, lactose, maltose, trehalose, etc.) and polyols (e.g., mannitol, sorbitol, etc.). See, also, Remington's Pharmaceutical Sciences (1990) Mack Publishing Co., Easton, Pa., Remington: The Science and Practice of Pharmacy (2000; 20^{th} Ed.) Lippencott Williams and Wilkins, Philadelphia, Pa., each of which is hereby incorporated by reference in its entirety.

As used herein, the term “polynucleotide,” “nucleotide,” nucleic acid” “nucleic acid molecule” and other similar terms are used interchangeable and include DNA, RNA, mRNA and the like.

As used herein, the term “positionally modified motif” comprises all other motifs. Methods of preparation of positionally modified oligonucleotide compounds are well known to those skilled in the art.

The term “preterm infant” subject refers, in certain embodiments, to a human subject born at less than 38 weeks of gestational age, such as less than 35 weeks gestational age, wherein the infant is less than 2 years old, less than 12 months old, such as less than 6 months old, less than 3 months old, less than 2 months old, or less than 1 month old.

As used herein, the terms “prevent,” “preventing,” and “prevention” refer to the total or partial inhibition of the development, recurrence, onset or spread of a disease and/or symptom related thereto, resulting from the administration of a therapy (e.g., an antisense compound provided herein) or combination of therapies provided herein (e.g., a combination of prophylactic or therapeutic agents, such as an antisense compound provided herein).

As used herein, the term “primary” viral infection refers to a first or original infection, for example, following a first exposure to a virus.

As used herein, the term “prodrug” refers to a therapeutic agent that is prepared in an inactive or less active form that is converted to an active form (i.e., drug) within the body or cells thereof by the action of endogenous enzymes, chemicals, and/or conditions.

As used herein, the term “prophylactic agent” refers to any agent that can totally or partially inhibit the development, recurrence, onset or spread of a disease and/or symptom related thereto in a subject. In some embodiments, the prophylactic agent is used to prevent or delay the onset of pulmonary inflammation, airway hyperreactivity and/or loss of lung function, which can, in certain instances, be the direct or indirect result of a viral infection. In certain embodiments, the term “prophylactic agent” refers to an antisense compound provided herein. In certain other embodiments, the term “prophylactic agent” refers to an agent other than an antisense compound. In certain embodiments, a prophylactic agent is an agent which is known to be useful to or has been or is currently being used to prevent pulmonary inflammation, airway hyperreactivity and/or loss of lung function and/or a symptom related thereto or impede the onset, development, progression and/or severity of pulmonary inflammation, airway hyperreactivity and/or loss of lung function and/or a symptom related thereto. In specific embodiments, the prophylactic agent is an antisense compound, such as AIR645.

The term “respiratory tract” as used herein refers to the part of a subject's anatomy that has to do with the process of respiration. The respiratory tract is divided into 3 segments: the upper respiratory tract (nose and nasal passages, paranasal sinuses, and throat or pharynx), the respiratory airways (voice box or larynx, trachea, bronchi, and bronchioles), and the lungs (respiratory bronchioles, alveolar ducts, alveolar sacs, and alveoli).

As used herein, the term “secondary” viral infection refers to a second (or third, fourth or subsequent) infection with a virus, for example, following a secondary exposure to the same or different virus as in a primary viral infection.

As used herein, the term “side effects” encompasses unwanted and adverse effects of a therapy (e.g., a prophylactic or therapeutic agent). Unwanted effects are not necessarily adverse. An adverse effect from a therapy (e.g., a prophylactic or therapeutic agent) might be harmful or uncomfortable or risky. Examples of side effects include, but are not limited to, rhinitis symptoms, asthma symptoms, congestion, cough, headache, diarrhea, gastroenteritis, nausea, vomiting, anorexia, abdominal cramping, fever, pain, loss of body weight, dehydration, alopecia, dyspenea, insomnia, dizziness, mucositis, nerve and muscle effects, fatigue, dry mouth, and loss of appetite, rashes or swellings at the site of administration, flu-like symptoms such as fever, chills and fatigue, digestive tract problems and allergic reactions. Additional undesired effects experienced by patients are numerous and known in the art. Many are described, for example, in the Physician's Desk Reference (2009) 63^{rd} ed., Thomson Reuters.

As used herein, the term “siRNA” refers to a double-stranded compound having a first and second strand, each strand having a central portion and two independent terminal portions. The central portion of the first strand is complementary to the central portion of the second strand, allowing hybridization of the strands. The terminal portions are independently, optionally complementary to the corresponding terminal portion of the complementary strand.

As used herein, the terms “subject” and “patient” are used interchangeably. As used herein, a subject is can be a mammal, such as a non-primate (e.g., cows, pigs, horses, cats, dogs, rats, etc.) or a primate (e.g., monkey and human). In certain embodiments, the term “subject”, as used herein, refers to a vertebrate, such as a mammal. Mammals include, without limitation, humans, primates, wild animals, feral animals, farm animals, sports animals, and pets. In one embodiment, the subject is a mammal, such as a human, having a viral infection and/or exhibiting pulmonary inflammation, airway hyperreactivity and/or loss of lung function. In another embodiment, the subject is a mammal, such as a human, that is at risk for developing a viral infection, pulmonary inflammation, airway hyperreactivity and/or loss of lung function. In certain embodiments, the subject is a human subject, such as an infant (e.g., a pre-term infant), child or adult subject. In some embodiments, the subject is immunocompromised and/or immunosuppressed. In certain embodiments, the subject is not an infant.

The term “sugar surrogate” overlaps with the slightly broader term “nucleoside mimetic” but is intended to indicate replacement of the sugar unit (furanose ring) only. The tetrahydropyranyl rings provided herein are illustrative of an example of a sugar surrogate wherein the furanose sugar group has been replaced with a tetrahydropyranyl ring system.

The term “synergistic” as used herein refers to a combination of therapies (e.g., use of prophylactic or therapeutic agents) which is more effective than the additive effects of any two or more single therapy. For example, a synergistic effect of a combination of prophylactic or therapeutic agents permits the use of lower dosages of one or more of the agents and/or less frequent administration of said agents to a subject. The ability to utilize lower dosages of prophylactic or therapeutic therapies and/or to administer said therapies less frequently can reduce the toxicity associated with the administration of said therapies to a subject without reducing the efficacy of said therapies in the prevention, management, treatment or amelioration of a viral infection, and/or pulmonary inflammation, airway hyperreactivity and/or loss of lung function, or a symptom thereof. In addition, a synergistic effect can result in improved efficacy of therapies in the prevention, management, treatment or amelioration of a viral infection, and/or pulmonary inflammation, airway hyperreactivity and/or loss of lung function, or a symptom thereof. Finally, synergistic effect of a combination of therapies (e.g., prophylactic or therapeutic agents) may avoid or reduce adverse or unwanted side effects associated with the use of any single therapy.

As used herein, the term “therapeutic agent” refers to any agent that can be used in the treatment, management or amelioration of a disease and/or a symptom related thereto. In some embodiments, the therapeutic agent is used in the treatment, management or amelioration of pulmonary inflammation, airway hyperreactivity and/or loss of lung function, which can, in certain instances, be the direct or indirect result of a viral infection and/or a non-viral environmental irritant. In certain embodiments, the term “therapeutic agent” refers to an antisense compound provided herein. In certain other embodiments, the term “therapeutic agent” refers to an agent other than an antisense compound provided herein. In one embodiment, a therapeutic agent is an agent which is known to be useful for, or has been or is currently being used for the treatment, management or amelioration pulmonary inflammation, airway hyperreactivity and/or loss of lung function and/or a symptom related thereto. In specific embodiments, the prophylactic agent is an antisense compound, such as AIR645.

As used herein, the term “therapy” refers to any protocol, method and/or agent that can be used in the prevention, management, treatment and/or amelioration of a disease or a symptom related thereto, such as pulmonary inflammation, airway hyperreactivity and/or loss of lung function. In certain embodiments, the term “therapy” refers to any protocol, method and/or agent that can be used in the modulation of an immune response to an infection in a subject or a symptom related thereto. In certain embodiments, the terms “therapies” and “therapy” refer to a biological therapy, supportive therapy, and/or other therapies useful in the prevention, management, treatment and/or amelioration of a disease or a symptom related thereto, such as pulmonary inflammation, airway hyperreactivity and/or loss of lung function or respiratory disease associated therewith known to one of skill in the art such as medical personnel. In other embodiments, the terms “therapies” and “therapy” refer to a biological therapy, supportive therapy, and/or other therapies useful in the modulation of an immune response to an infection in a subject or a symptom related thereto known to one of skill in the art such as medical personnel.

The term “tolerance” or “immune tolerance” as used herein refers to a state of unresponsiveness to a specific antigen or group of antigens to which a person is normally responsive, for a period of at least one year. Producing immune tolerance can involve inducing or eliciting nonresponsiveness or anergy in T cells and can be distinguished from immunosuppression in that it is generally antigen-specific and persists after exposure to the tolerogen has ceased. Tolerance can be demonstrated, for example, by the lack of a T cell or B cell response upon reexposure to specific antigen in the absence of the tolerogen.

The term “topical administration”, as used herein, refers to administration to the skin or mucous membrane. In the context of administration to the respiratory tract of a subject, topical administration refers to administration to the respiratory mucosa—the mucous membrane lining the respiratory tract (including, for example, the nasal cavity, the larynx, the trachea, the bronchi tree, and the alveoli).

As used herein, the terms “treat,” “treatment” and “treating” refer to the reduction or amelioration of the progression, severity, and/or duration of a disease or a symptom related thereto, such as pulmonary inflammation, airway hyperreactivity and/or loss of lung function, resulting from the administration of one or more therapies (including, but not limited to, the administration of one or more prophylactic or therapeutic agents, such as an antisense compound provided herein). The term “treating,” as used herein, can also refer to altering the disease course of the subject being treated. Therapeutic effects of treatment include, without limitation, preventing occurrence or recurrence of disease, alleviation of symptom(s), diminishment of direct or indirect pathological consequences of the disease, decreasing the rate of disease progression, amelioration or palliation of the disease state, and remission or improved prognosis. In one embodiment, “treatment” or “treating” refers to an amelioration of a respiratory disorder, in particular, a Th2-mediated respiratory disorder, or at least one discernable symptom thereof. In another embodiment, “treatment” or “treating” refers to an amelioration of at least one measurable physical parameter, not necessarily discernible by the patient. In yet another embodiment, “treatment” or “treating” refers to inhibiting the progression of a respiratory disorder, in particular, a Th2-mediated respiratory disorder, either physically, e.g., stabilization of a discernible symptom, physiologically, e.g., stabilization of a physical parameter, or both. In yet another embodiment, “treatment” or “treating” refers to delaying the onset of a respiratory disorder, in particular, a Th2-mediated respiratory disorder, or symptoms thereof. With respect to viral infections, “treatment” additionally refers to inhibition of the local and systemic host response to the virus or the acute or chronic host inflammatory response induced by virus infection.

The term “upper respiratory” tract refers to the major passages and structures of the upper respiratory tract including the nose or nostrils, nasal cavity, mouth, throat (pharynx), and voice box (larynx).

### DETAILED DESCRIPTION

Respiratory disorders, Th2-mediated respiratory disorders in particular, such as asthma, allergy, and a number of other diseases or conditions related to pulmonary inflammation, airway hyperreactivity (AHR) and/or loss of lung function share common inflammatory mediators, including IL-4Rα, the common subunit of the IL-4R and IL-13R. Therapeutic interventions for these diseases or conditions are not completely satisfactory due to lack of efficacy and/or unwanted side effects of the compounds. Provided herein are compositions and methods for preventing, managing or treating such disorders using, in certain embodiments, oligomeric compounds, such as antisense compounds, including ASO. In some embodiments, the compounds are administered no more frequently than about once per week.

The compositions and methods provided herein may employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described in the references cited herein and are fully explained in the literature. See, e.g., Maniatis et al. (1982) Molecular Cloning: A Laboratory Manual, Cold Spring Harbor Laboratory Press; Sambrook et al. (1989), Molecular Cloning: A Laboratory Manual, Second Edition, Cold Spring Harbor Laboratory Press; Sambrook et al. (2001) Molecular Cloning: A Laboratory Manual, Cold Spring Harbor Laboratory Press, Cold Spring Harbor, N.Y.; Ausubel et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren et al. (eds.) (1999) Genome Analysis: A Laboratory Manual, Cold Spring Harbor Laboratory Press.

Compounds

Oligomeric compounds, including antisense oligonucleotides and other antisense compounds for use in modulating the expression of nucleic acid molecules encoding IL-4Rα are employed in the methods disclosed herein. The oligomeric compounds hybridize with one or more target nucleic acid molecules encoding IL-4Rα. As used herein, the terms “target nucleic acid” and “nucleic acid molecule encoding IL-4Rα” have been used for convenience to encompass DNA encoding IL-4Rα, RNA (including pre-mRNA and mRNA or portions thereof) transcribed from such DNA, and also cDNA derived from such RNA. In one embodiment, the target nucleic acid is an mRNA encoding IL-4Rα, such as human IL-4Rα (SEQ ID NO:1).

Antisense compounds hybridize to a target nucleic acid, modulating gene expression activities such as transcription or translation. This sequence specificity makes antisense compounds extremely attractive as tools for target validation and gene functionalization, as well as therapeutics to selectively modulate the expression of genes involved in disease. Although not limited by mechanism of action, the compounds provided herein are proposed to work by an antisense, non-autocatalytic mechanism.

In some embodiments, the compounds provided herein are oligomeric compounds, which are polymeric structures capable of hybridizing to a region of a nucleic acid molecule. This term includes oligonucleotides, oligonucleosides, oligonucleotide analogs, oligonucleotide mimetics, and chimeric combinations thereof. Generally, oligomeric compounds comprise a plurality of monomeric subunits linked together by internucleoside linking groups and/or internucleoside linkage mimetics. Each of the monomeric subunits comprises a sugar, abasic sugar, modified sugar, or a sugar mimetic, and except for the abasic sugar includes a nucleobase, modified nucleobase or a nucleobase mimetic. Monomeric subunits can comprise nucleosides and modified nucleosides. Oligomeric compounds are routinely prepared linearly but can be joined or otherwise prepared to be circular. Moreover, branched structures are known in the art.

In specific embodiments, the oligomeric compound is an antisense compound (or antisense oligomeric compound), which is at least partially complementary to the region of a target nucleic acid molecule to which it hybridizes and which modulates (e.g., increases or decreases) its expression. Consequently, while all antisense compounds can be said to be oligomeric compounds, not all oligomeric compounds are antisense compounds. In certain embodiments, the antisense compound is an antisense oligonucleotide. An antisense oligonucleotide can, in some cases, include one or more chemical modifications to the sugar, base, and/or internucleoside linkages. Non-limiting examples of oligomeric compounds include primers, probes, antisense compounds, antisense oligonucleotides, external guide sequence (EGS) oligonucleotides, alternate splicers, and siRNAs. As such, these compounds can be introduced in the form of single-stranded, double-stranded, circular, branched or hairpins and can contain structural elements such as internal or terminal bulges or loops. Oligomeric double-stranded compounds can be two strands hybridized to form double-stranded compounds or a single strand with sufficient self complementarity to allow for hybridization and formation of a fully or partially double-stranded compound. In specific embodiments, the compounds of the compositions or that are administered according to the methods provided herein are not auto-catalytic.

In one embodiment of the methods provided herein, the oligomeric compound is an antisense compound comprising a single stranded oligonucleotide. In additional embodiments, the antisense compound contains chemical modifications. The antisense compound can, for example, be a single stranded, chimeric oligonucleotide wherein the modifications of sugars, bases, and internucleoside linkages are independently selected.

The oligomeric compounds provided herein can comprise an oligomeric compound from about 12 to about 35 nucleobases (i.e., from about 12 to about 35 linked nucleosides). In certain embodiments, a single-stranded compound comprises from about 12 to about 35 nucleobases, and a double-stranded antisense compound (such as a siRNA, for example) comprises two strands, each of which is from about 12 to about 35 nucleobases. In specific embodiments, the antisense compound (e.g., antisense oligonucleotide) is 12, 13, 14, 15, 16, 17, 18, 19, 20, 21, 22, 23, 24, 25, 26, 27, 28, 29, 30, 31, 32, 33, 34, or 35 nucleobases in length. Contained within certain oligomeric compounds provided herein (whether single or double stranded and on at least one strand) are antisense portions. The “antisense portion” is that part of the oligomeric compound that is designed to work by one of the aforementioned antisense mechanisms. One of ordinary skill in the art will appreciate that this comprises antisense portions of 12, 13, 14, 15, 16, 17, 18, 19, 20, 21, 22, 23, 24, 25, 26, 27, 28, 29, 30, 31, 32, 33, 34, or 35 nucleobases.

In specific embodiments, the antisense portion is the same length as the antisense compound (e.g., antisense oligonucleotide). For example, in certain embodiments, an antisense compound (e.g., antisense oligonucleotide) is 20 nucleobases in length and the antisense portion spans the entire 20 nucleobase length of the compound. In other embodiments, the antisense portion is contained within a longer antisense compound. For example, in some embodiments, the antisense compound (e.g., antisense oligonucleotide) is 22 nucleobases in length, and the antisense portion is only 20 nucleobases in length, wherein the antisense portion comprises 20 consecutive nucleobases, and wherein two nucleobases at the 5′ end, two nucleobases at the 3′ end, or one nucleobase at each of the 5′ and 3′ ends of the molecule are not antisense portions.

In one embodiment, the antisense compounds have antisense portions of 12 to 35 nucleobases. It is understood that the antisense portion can be about 12, 13, 14, 15, 16, 17, 18, 19, 20, 21, 22, 23, 24, 25, 26, 27, 28, 29, 30, 31, 32, 33, 34, or 35 nucleobases in length. Antisense compounds 12 to 35 nucleobases in length comprising a stretch of at least the 8, at least the 9, at least the 10, at least the 11, at least the 12, at least the 13, at least the 14, at least the 15, at least the 16, at least the 17, at least the 18, at least the 19 or at least the 20, at least the 21, at least the 22, at least the 23, at least the 24, at least the 25, at least the 26, at least the 27, at least the 28, at least the 29, at least the 30, at least the 31, at least the 32, at least the 33, at least the 34, or at least the 35 consecutive nucleobases selected from within the illustrative antisense compounds are considered to be suitable antisense compounds, as well.

Compounds provided and administered via the methods provided herein can include oligonucleotide sequences that comprise at least the 8, at least the 9, at least the 10, at least the 11, at least the 12, at least the 13, at least the 14, at least the 15, at least the 16, at least the 17, at least the 18, at least the 19 or at least the 20, at least the 21, at least the 22, at least the 23, at least the 24, at least the 25, at least the 26, at least the 27, at least the 28, at least the 29, at least the 30, at least the 31, at least the 32, at least the 33, at least the 34, or at least the 35 consecutive nucleobases from the 5′-terminus of one of the illustrative antisense compounds, with the remaining nucleobases being a consecutive stretch of the same oligonucleotide beginning immediately upstream of the 5′-terminus of the antisense compound which is specifically hybridizable to the target nucleic acid and continuing until the oligonucleotide contains about 12 to 35 nucleobases. Other compounds are represented by oligonucleotide sequences that comprise at least the 8, at least the 9, at least the 10, at least the 11, at least the 12, at least the 13, at least the 14, at least the 15, at least the 16, at least the 17, at least the 18, at least the 19 or at least the 20, at least the 21, at least the 22, at least the 23, at least the 24, at least the 25, at least the 26, at least the 27, at least the 28, at least the 29, at least the 30, at least the 31, at least the 32, at least the 33, at least the 34, or at least the 35 consecutive nucleobases from the 3′-terminus of one of the illustrative antisense compounds, with the remaining nucleobases being a consecutive stretch of the same oligonucleotide beginning immediately downstream of the 3′-terminus of the antisense compound which is specifically hybridizable to the target nucleic acid and continuing until the oligonucleotide contains about 12 to about 35 nucleobases. It is also understood that compounds can be represented by oligonucleotide sequences that comprise at least the 8, at least the 9, at least the 10, at least the 11, at least the 12, at least the 13, at least the 14, at least the 15, at least the 16, at least the 17, at least the 18, at least the 19 or at least the 20, at least the 21, at least the 22, at least the 23, at least the 24, at least the 25, at least the 26, at least the 27, at least the 28, at least the 29, at least the 30, at least the 31, at least the 32, at least the 33, at least the 34, or at least the 35 consecutive nucleobases from an internal portion of the sequence of an illustrative compound, and can extend in either or both directions until the oligonucleotide contains about 12 to about 35 nucleobases.

Modifications can be made to the compounds provided herein and can include conjugate groups attached to one of the termini, selected nucleobase positions, sugar positions or to one of the internucleoside linkages. Possible modifications include, but are not limited to, 2′-F and 2′-O-methyl sugar modifications, inverted abasic caps, deoxynucleobases, and nucleobase analogs such as locked nucleic acids (LNA).

In one embodiment, double-stranded antisense compounds encompass short interfering RNAs (siRNAs). The ends of the strands can be modified by the addition of one or more natural or modified nucleobases to form an overhang. In one non-limiting example, the first strand of the siRNA is antisense to the target nucleic acid, while the second strand is complementary to the first strand. Once the antisense strand is designed to target a particular nucleic acid target, the sense strand of the siRNA can then be designed and synthesized as the complement of the antisense strand and either strand can contain modifications or additions to either terminus. For example, in one embodiment, both strands of the siRNA duplex would be complementary over the central nucleobases, each having overhangs at one or both termini. It is possible for one end of a duplex to be blunt and the other to have overhanging nucleobases. In one embodiment, the number of overhanging nucleobases is from 1 to 6 on the 3′ end of each strand of the duplex. In another embodiment, the number of overhanging nucleobases is from 1 to 6 on the 3′ end of only one strand of the duplex. In a further embodiment, the number of overhanging nucleobases is from 1 to 6 on one or both 5′ ends of the duplexed strands. In another embodiment, the number of overhanging nucleobases is zero. In one embodiment, each of the strands is 19 nucleobases in length, fully hybridizable with the complementary strand, and includes no overhangs.

Each strand of the siRNA duplex can be from about 12 to about 35 nucleobases. In one embodiment, each strand of the siRNA duplex is about 17 to about 25 nucleobases, such as about 17, 18, 19, 20, 21, 22, 23, 24 or 25 nucleobases. The central complementary portion can be from about 12 to about 35 nucleobases in length, such as about 12, 13, 14, 15, 16, 17, 18, 19, 20, 21, 22, 23, 24, 25, 26, 27, 28, 29, 30, 31, 32, 33, 34, or 35 nucleobases in length. In another embodiment, the central complimentary portion is about 17 to about 25 nucleobases in length, such as about 17, 18, 19, 20, 21, 22, 23, 24, 25, 26, 27, 28, 29, 30, 31, 32, 33, 34, or 35 nucleobases in length. It is understood that each the strand of the siRNA duplex and the central complementary portion can be about 12, 13, 14, 15, 16, 17, 18, 19, 20, 21, 22, 23, 24, 25, 26, 27, 28, 29, 30, 31, 32, 33, 34, or 35 nucleobases in length. The terminal portions can be from 1 to 6 nucleobases. It is understood that the terminal portions can be about 1, 2, 3, 4, 5, or 6 nucleobases in length. The siRNAs can also have no terminal portions. The two strands of a siRNA can be linked internally leaving free 3′ or 5′ termini, or can be linked to form a continuous hairpin structure or loop. The hairpin structure can contain an overhang on either the 5′ or 3′ terminus producing an extension of single-stranded character.

Double-stranded compounds can be made to include chemical modifications as discussed herein.

AIR645 (ISIS369645, SEQ ID NO:280) is a chimeric 20-oligonucleotide molecule composed of a 2′-deoxyphosphorothioate decanucleotide flanked at each end by 2′-O-(2-methoxy)-ethyl (2′-MOE) substituted phosphorothioate pentanucleotides. As such, it is a second-generation antisense phosphorothioate oligonucleotide (also called a 2′-MOE gapmer). The 2′-MOE substitution of second-generation molecules increases the binding affinity for target mRNAs and increases resistance to nuclease-mediated metabolism relative to first-generation antisense phosphorothioate oligodeoxynucleotides and to unmodified DNA. These increases in affinity and stability result in improved antisense potency both in vitro and in vivo, as well as increased tissue half-life and duration of activity (Dean, N. M. 2001 Antisense Technology: Principles, Strategies and Applications. NY:Marcel Dekker, Inc. 319-338). This design enables the RNase H recognition and cleavage mechanism that has demonstrated reductions in target protein and function in recent clinical studies (Kastelein, J. J., et al. 2006 Circulation 114:1729). In addition, the second-generation 2′-MOE modified phosphorothioate oligonucleotides have an improved safety and tolerability profile in rodents and primates, including humans, relative to the first-generation phosphorothioate oligodeoxynucleotides. Non-specific pro-inflammatory effects displayed by phosphorothioate oligodeoxynucleotides are reduced or eliminated with 2′-MOE chemistry (Henry, S., et al. 2000 J Pharm Exp Ther 292:468).

Antisense Oligonucleotides and Pulmonary Disease

Antisense oligonucleotides are being pursued as therapeutics for pulmonary inflammation, airway hyper-responsiveness, and/or asthma. Lung provides an ideal tissue for aerosolized ASOs for several reasons (Nyce and Metzger, Nature, 1997:385:721-725, Gavreau et al. 2008 Am. J. Respir. Crit. Care Med. 177: 952, each of which is incorporated herein by reference); the lung can be targeted non-invasively and specifically, it has a large absorption surface; and it is lined with surfactant that can facilitate distribution and uptake of ASOs. Delivery of ASOs to the lung by aerosol results in excellent distribution throughout the lung in both mice and primates.

Immunohistochemical staining of inhaled ASOs in normalized and inflamed mouse lung tissue shows heavy staining in alveolar macrophages, eosinophils, and epithelium, moderate staining in blood vessels endothelium, and weak staining in bronchiolar epithelium. ASO-mediated target protein reduction is observed in dendritic cells, macrophages, eosinophils, and epithelial cells recovered from lung tissue after aerosol administration via nebulization, intratracheal instillation or intranasal instillation of the ASO.

The estimated lung half-life of a 2′-O-methoxyethoxy (2′-MOE) modified oligonucleotide delivered by aerosol administration to mouse or monkey is about 9 and 14 days, respectively. The half-life of a 2′-MOE modified oligonucleotide in human induced sputum following aerosol administration is about 5 days. Oligonucleotides have relatively predictable toxicities and pharmacokinetics based on backbone and nucleotide chemistry. Pulmonary administration of ASOs results in minimal systemic exposure, potentially increasing the safety of such compounds as compared to other classes of drugs.

Chemical Modifications

As is known in the art, a nucleoside is a base-sugar combination. The base portion of the nucleoside is normally a heterocyclic base (sometimes referred to as a “nucleobase” or simply a “base”). The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound. Within oligonucleotides, the phosphate groups are commonly referred to as forming the internucleoside backbone of the oligonucleotide. The normal linkage or backbone of RNA and DNA is a 3′ to 5′ phosphodiester linkage. Chemical modifications in oligonucleotides can also be used to alter their activity. Chemical modifications can alter oligonucleotide activity by, for example: increasing affinity of an antisense oligonucleotide for its target RNA, increasing nuclease resistance, and/or altering the pharmacokinetics of the oligonucleotide. The use of chemistries that increase the affinity of an oligonucleotide for its target can allow for the use of shorter oligonucleotide compounds.

Antisense and other oligomeric compounds can optionally contain one or more nucleosides wherein the sugar group has been modified. Such sugar modified nucleosides can impart enhanced nuclease stability, increased binding affinity or some other beneficial biological property to the antisense compounds. The furanosyl sugar ring of a nucleoside can be modified in a number of ways including, but not limited to, addition of a substituent group, bridging of two non-geminal ring atoms to form a bicyclic nucleic acid (BNA) and substitution of an atom or group such as —S—, —N(R) or —C(R₁)(R₂) for the ring oxygen at the 4′-position. Modified sugar moieties are well known and can be used to alter, typically increase, the affinity of the oligomeric compound for its target and/or increase nuclease resistance. A representative list of modified sugars includes but is not limited to bicyclic modified sugars (BNA's), including LNA and ENA (4′-(CH₂)₂—O-2′ bridge); and substituted sugars, especially 2′-substituted sugars having a 2′-F, 2′-OCH₂ or a 2′-O(CH₂)₂—OCH₃ substituent group. Sugars can also be replaced with sugar mimetic groups among others. Methods for the preparations of modified sugars are well known to those skilled in the art.

In certain embodiments, nucleosides comprise a chemically modified ribofuranose ring moieties. Examples of chemically modified ribofuranose rings include without limitation, addition of substituent groups (including 5′ and 2′ substituent groups, bridging of non-geminal ring atoms to form bicyclic nucleic acids (BNA), replacement of the ribosyl ring oxygen atom with S, N(R), or C(R1)(R)₂ (R═H, C1-C12 alkyl or a protecting group) and combinations thereof. Examples of chemically modified sugars include 2′-F-5′-methyl substituted nucleoside (see PCT International Application WO 2008/101157 for other disclosed 5′,2′-bis substituted nucleosides) or replacement of the ribosyl ring oxygen atom with S with further substitution at the 2′-position (see U.S. Publ. No. US2005-0130923) or alternatively 5′-substitution of a BNA (see WO 2007/134181, wherein LNA is substituted with for example a 5′-methyl or a 5′-vinyl group).

Examples of nucleosides having modified sugar moieties include without limitation nucleosides comprising 5′-vinyl, 5′-methyl (R or S), 4′-S, 2′-F, 2′-OCH₃ and 2′-O(CH₂)₂OCH₃ substituent groups. The substituent at the 2′ position can also be selected from allyl, amino, azido, thio, O-allyl, O—C1-C10 alkyl, OCF₃, O(CH₂)₂SCH₃, O(CH₂)₂—O—N(Rm)(Rn), and O—CH₂—C(═O)—N(Rn)(Rn), where each Rm and Rn is, independently, H or substituted or unsubstituted C1-C10 alkyl.

Examples of bicyclic nucleic acids (BNAs) include without limitation nucleosides comprising a bridge between the 4′ and the 2′ ribosyl ring atoms. In certain embodiments, antisense compounds provided herein include one or more BNA nucleosides wherein the bridge comprises one of the formulas: 4′-(CH₂)—O-2′ (LNA); 4′-(CH₂)—S-2′; 4′-(CH₂)₂—O-2′ (ENA); 4′-C(CH₃)₂—O-2′ (see PCT/US2008/068922); 4′-CH(CH₃)—O-2′ and 4′-C—H(CH₂OCH₃)—O-2′ (see U.S. Pat. No. 7,399,845); 4′-CH₂—N(OCH₃)-2′ (see PCT/US2008/064591); 4′-CH₂—O—N(CH₃)-2′ (see U.S. Publ. No. US2004-0171570); 4′-CH₂—N(R)—O-2′ (see U.S. Pat. No. 7,427,672); 4′-CH₂—C(CH₃)-2′ and 4′-CH₂—C(═CH₂)-2′ (see PCT/US2008/066154); and wherein R is, independently, H, C₁-C₁₂ alkyl, or a protecting group. Each of the foregoing BNAs include various stereochemical sugar configurations including for example α-L-ribofuranose and β-D-ribofuranose (see PCT international application PCT/DK98/00393, published on Mar. 25, 1999 as WO 99/14226).

In certain embodiments, nucleosides are modified by replacement of the ribosyl ring with a sugar surrogate. Such modification includes without limitation, replacement of the ribosyl ring with a surrogate ring system (sometimes referred to as DNA analogs) such as a morpholino ring, a cyclohexenyl ring, a cyclohexyl ring or a tetrahydropyranyl ring such as one having one of the formula:

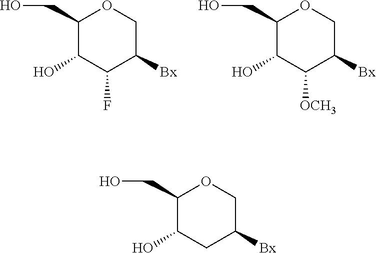

Many other bicyclo and tricyclo sugar surrogate ring systems are also know in the art that can be used to modify nucleosides for incorporation into antisense compounds (see for example review article: Leumann, Christian J., Bioorganic & Medicinal Chemistry, 2002, 10, 841-854). Such ring systems can undergo various additional substitutions to enhance activity.

Additionally contemplated are internucleoside linking groups that link the nucleosides or otherwise modified monomer units together thereby forming an oligomeric compound. The two main classes of internucleoside linking groups are defined by the presence or absence of a phosphorus atom. Representative phosphorus containing internucleoside linkages include, but are not limited to, phosphodiesters, phosphotriesters, methylphosphonates, phosphoramidate, and phosphorothioates. Representative non-phosphorus containing internucleoside linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Oligomeric compounds having non-phosphorus internucleoside linking groups are referred to as oligonucleosides. Modified internucleoside linkages, compared to natural phosphodiester linkages, can be used to alter, typically increase, nuclease resistance of the oligomeric compound. Internucleoside linkages having a chiral atom can be prepared racemic, chiral, or as a mixture. Representative chiral internucleoside linkages include, but are not limited to, alkylphosphonates and phosphorothioates. Methods of preparation of phosphorous-containing and non-phosphorous-containing linkages are well known to those skilled in the art.

In certain embodiments, a sugar, a nucleobase, and/or internucleoside linkage is substituted by a mimetic. Generally, a mimetic is used in place of the sugar or sugar-internucleoside linkage combination, and the nucleobase is maintained for hybridization to a selected target. Representative examples of a sugar mimetic include, but are not limited to, cyclohexenyl or morpholino. Representative examples of a mimetic for a sugar-internucleoside linkage combination include, but are not limited to, peptide nucleic acids (PNA) and morpholino groups linked by uncharged achiral linkages. In some instances a mimetic is used in place of the nucleobase. Representative nucleobase mimetics are well known in the art and include, but are not limited to, tricyclic phenoxazine analogs and universal bases (Berger, et al., 2000 Nuc Acid Res 28:2911-14, incorporated herein by reference). Methods of synthesis of sugar, nucleoside and nucleobase mimetics are well known to those skilled in the art.

In certain embodiments, the oligomeric compound is an oligonucleotide, which comprises naturally- and normaturally-occurring nucleobases, sugars and covalent internucleoside linkages, and possibly further include non-nucleic acid conjugates.

Further disclosed herein are compounds having reactive phosphorus groups useful for forming internucleoside linkages including for example phosphodiester and phosphorothioate internucleoside linkages. Methods of preparation and/or purification of precursors or oligomeric compounds are not a limitation of the compositions or methods provided herein. Methods for synthesis and purification of DNA, RNA, and the oligomeric compounds provided herein are well known to those skilled in the art.

In some embodiments, the antisense compound is a chimeric oligomeric compound, which said compound has at least one sugar, nucleobase and/or internucleoside linkage that is differentially modified as compared to the other sugars, nucleobases and internucleoside linkages within the same oligomeric compound. The remainder of the sugars, nucleobases and internucleoside linkages can be independently modified or unmodified provided that they are distinguishable from the differentially modified moiety or moieties. In general a chimeric oligomeric compound will have modified nucleosides that can be in isolated positions or grouped together in regions that will define a particular motif. Any combination of modifications and/or mimetic groups can comprise a chimeric oligomeric compound.

Chimeric oligomeric compounds typically contain at least one region modified so as to confer increased resistance to nuclease degradation, increased cellular uptake, and/or increased binding affinity for the target nucleic acid. An additional region of the oligomeric compound can serve as a substrate for enzymes capable of cleaving RNA:DNA or RNA:RNA hybrids. By way of example, RNase H is a cellular endonuclease that cleaves the RNA strand of an RNA:DNA duplex. Activation of RNase H, therefore, results in cleavage of the RNA target, thereby greatly enhancing the efficiency of inhibition of gene expression. Consequently, comparable results can often be obtained with shorter oligomeric compounds when chimeras are used, compared to for example phosphorothioate deoxyoligonucleotides hybridizing to the same target region. Cleavage of the RNA target can be routinely detected by gel electrophoresis and, if necessary, associated nucleic acid hybridization techniques known in the art.

Certain chimeric as well as non-chimeric oligomeric compounds can be further described as having a particular motif. Such motifs include, but are not limited to, gapped motifs, alternating motifs, fully modified motifs, hemimer motifs, blockmer motifs, and positionally modified motifs. The sequence and the structure of the nucleobases and type of internucleoside linkage is not a factor in determining the motif of an oligomeric compound. In some embodiments, the antisense compounds provided herein comprise one or more gapped motifs, alternating motifs, fully modified motifs, hemimer motifs, blockmer motifs, or positionally modified motifs. Methods for preparation of chimeric oligonucleotide compounds are well known to those skilled in the art.

The compounds described herein contain one or more asymmetric centers and thus give rise to enantiomers, diastereomers, and other stereoisomeric configurations that can be defined, in terms of absolute stereochemistry, as (R) or (S), α or β, or as (D) or (L) such as for amino acids. All such possible isomers, as well as their racemic and optically pure forms, are contemplated.

Also described herein are oligomeric compounds modified by covalent attachment of one or more conjugate groups. Conjugate groups can be attached by reversible or irreversible attachments. Conjugate groups can be attached directly to oligomeric compounds or by use of a linker. Linkers can be mono- or bifunctional linkers. Such attachment methods and linkers are well known to those skilled in the art. In general, conjugate groups are attached to oligomeric compounds to modify one or more properties. Such considerations are well known to those skilled in the art.

Oligomer Synthesis

Oligomerization of modified and unmodified nucleosides can be routinely performed according to literature procedures for DNA (Protocols for Oligonucleotides and Analogs, Ed. Agrawal (1993), Humana Press) and/or RNA (Scaringe, Methods (2001), 23, 206-217. Gait et al., Applications of Chemically synthesized RNA in RNA: Protein Interactions, Ed. Smith (1998), 1-36. Gallo et al., Tetrahedron (2001), 57, 5707-5713).

Oligomeric compounds can be conveniently and routinely made through the well-known technique of solid phase synthesis. Equipment for such synthesis is sold by several vendors including, for example, Applied Biosystems (Foster City, Calif.). Any other means for such synthesis known in the art can additionally or alternatively be employed. It is well known to use similar techniques to prepare oligonucleotides such as the phosphorothioates and alkylated derivatives. The compositions and methods provided herein are not limited by the method of oligomer synthesis.

Oligomer Purification and Analysis

Methods of oligonucleotide purification and analysis are known to those skilled in the art. Analysis methods include capillary electrophoresis (CE) and electrospray-mass spectroscopy. Such synthesis and analysis methods can be performed in multi-well plates. The compositions and methods provided herein are not limited by the method of oligomer purification.

Hybridization

“Hybridization” means the pairing of complementary strands of oligomeric compounds. While not limited to a particular mechanism, the most common mechanism of pairing involves hydrogen bonding, which can be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances.

An oligomeric compound is specifically hybridizable when there is a sufficient degree of complementarity to avoid non-specific binding of the oligomeric compound to non-target nucleic acid sequences under conditions in which specific binding is desired, i.e., under physiological conditions in the case of in vivo assays or therapeutic treatment, and under conditions in which assays are performed in the case of in vitro assays.

“Stringent hybridization conditions” or “stringent conditions” refer to conditions under which an oligomeric compound will hybridize to its target sequence, but to a minimal number of other sequences. Stringent conditions are sequence-dependent and will be different in different circumstances, and “stringent conditions” under which oligomeric compounds hybridize to a target sequence are determined by the nature and composition of the oligomeric compounds and the assays in which they are being investigated.

“Complementarity,” as used herein, refers to the capacity for precise pairing between two nucleobases on one or two oligomeric compound strands. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be a complementary position. The oligomeric compound and the further DNA or RNA are complementary to each other when a sufficient number of complementary positions in each molecule are occupied by nucleobases which can hydrogen bond with each other. Thus, “specifically hybridizable” and “complementary” are terms which are used to indicate a sufficient degree of precise pairing or complementarity over a sufficient number of nucleobases such that stable and specific binding occurs between the oligomeric compound and a target nucleic acid.

Identity

To determine the percent identity of two nucleic acid sequences, the sequences are aligned for optimal comparison purposes (e.g., gaps can be introduced in the sequence of a first nucleic acid sequence for optimal alignment with a second nucleic acid sequence). The nucleotides at corresponding nucleotide positions are then compared. When a position in the first sequence is occupied by the same nucleotide as the corresponding position in the second sequence, then the molecules are identical at that position. The percent identity between the two sequences is an expression of the number of identical positions shared by the sequences (i.e., % identity=number of identical overlapping positions/total number of positions×100%). In one embodiment, the two sequences are the same length.

The determination of percent identity between two sequences (e.g., nucleic acid sequences) can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules provided herein. To obtain gapped alignments for comparison purposes, Gapped BLAST can be utilized as described in Altschul et al., 1997, Nucleic Acids Res. 25:3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih gov). Another non limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM 120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

Oligomeric compounds, or a portion thereof, can have a defined percent identity to a SEQ ID NO, or a compound having a specific ISIS-designated number. As used herein, a sequence is identical to the sequence disclosed herein if it has the same nucleobase pairing ability. For example, an RNA which contains uracil in place of thymidine in the disclosed sequences would be considered identical as they both pair with adenine. Similarly, a G-clamp modified heterocyclic base would be considered identical to a cytosine or a 5-Me cytosine in the sequences of the instant application as it pairs with a guanine. This identity can be over the entire length of the oligomeric compound, or in a portion of the oligomeric compound (e.g., nucleobases 1-20 of a 27-mer can be compared to a 20-mer to determine percent identity of the oligomeric compound to the SEQ ID NO.) It is understood by those skilled in the art that an oligonucleotide need not have an identical sequence to those described herein to function similarly to the oligonucleotides described herein. Shortened (i.e., deleted, and therefore non-identical) versions of oligonucleotides taught herein, or non-identical (e.g., one base replaced with another with non-identical nucleobase pairing, or abasic site) versions of the oligonucleotides taught herein fall can be used in the compositions and methods provided herein. Percent identity is calculated according to the number of bases that have identical base pairing corresponding to the SEQ ID NO or compound to which it is being compared. The non-identical bases can be adjacent to each other, dispersed throughout the oligonucleotide, or both.

For example, a 16-mer having the same sequence as nucleobases 2-17 of a 20-mer is 80% identical to the 20-mer. Alternatively, a 20-mer containing four nucleobases not identical to the 20-mer is also 80% identical to the 20-mer. A 14-mer having the same sequence as nucleobases 1-14 of an 18-mer is 78% identical to the 18-mer. Such calculations are well within the ability of those skilled in the art.

The percent identity is based on the percent of nucleobases in the original sequence present in a portion of the modified sequence. Therefore, a 30 nucleobase oligonucleotide comprising the full sequence of a 20 nucleobase SEQ ID NO would have a portion of 100% identity with the 20 nucleobase SEQ ID NO while further comprising an additional 10 nucleobase portion. In certain embodiments, the full length of the modified sequence constitutes a single portion. In a one embodiment, the oligonucleotides are at least about 80%, at least 80%, at least about 85%, at least 85%, at least about 90%, at least 90%, at least about 95%, at least 95%, at least about 99%, at least 99% or 100% identical to the active target segments and/or oligonucleotides presented herein.

It is well known by those skilled in the art that it is possible to increase or decrease the length of an antisense oligonucleotide and/or introduce mismatch bases without eliminating activity. For example, in Woolf et al. (Proc. Natl. Acad. Sci. USA 89:7305-7309, 1992, incorporated herein by reference), a series of oligomers 13-25 nucleobases in length were tested for their ability to induce cleavage of a target RNA in an oocyte injection model. Oligonucleotides 25 nucleobases in length with 8 or 11 mismatch bases near the ends of the oligonucleotide were able to direct specific cleavage of the target mRNA, albeit to a lesser extent than the oligonucleotide that contained no mismatches. Similarly, target specific cleavage was achieved using a 13 nucleobase oligomer, including those with 1 or 3 mismatches. Maher and Dolnick (Nuc. Acid. Res. 16:3341-3358.1988, incorporated herein by reference) tested a series of tandem 14 nucleobase oligonucleotides, and a 28 and 42 nucleobase oligonucleotide comprised of the sequence of two or three of the tandem oligonucleotides, respectively, for their ability to arrest translation of human DHFR in a rabbit reticulocyte assay. Each of the three 14 nucleobase oligonucleotides alone was able to inhibit translation, albeit at a more modest level, than the 28 or 42 nucleobase oligonucleotide.

Target Nucleic Acids

“Targeting” an oligomeric compound to a particular target nucleic acid molecule can be a multistep process. The process usually begins with the identification of a target nucleic acid whose expression is to be modulated. For example, the target nucleic acid can be a cellular gene (or mRNA transcribed from the gene) whose expression is associated with a particular disorder or disease state, or a nucleic acid molecule from an infectious agent. As disclosed herein, the target nucleic acid encodes IL-4Rα, for example a coding/translated region, 5′ untranslated region, 3′ untranslated region or a combination thereof, including regions spanning the translated and untranslated regions. In certain embodiments, the target nucleic acid is a pre-RNA. In other embodiments, the target nucleic acid is an mRNA.

Target Regions, Segments, and Sites

The targeting process usually also includes determination of at least one target region, segment, or site within the target nucleic acid for the antisense interaction to occur such that the desired effect, e.g., modulation of expression, will result. “Region” is defined as a portion of the target nucleic acid having at least one identifiable structure, function, or characteristic. Target regions include, but are not limited to translation initiation and termination regions, coding regions, open reading frames, introns, exons, 3′-untranslated regions (3′-UTR), 5′-untranslated regions (5′-UTR), splice sites, and 5′ CAPs. Within regions of target nucleic acids are segments. “Segments” are defined as smaller or sub-portions of regions within a target nucleic acid such as stop codons and start codons. “Sites,” as used herein, are defined as unique nucleobase positions within a target nucleic acid such as splice junctions. Such regions, segments, and sites are well known to those skilled in the art.

Variants

It is also known in the art that alternative RNA transcripts can be produced from the same genomic region of DNA. These alternative transcripts are generally known as “variants.” More specifically, “pre-mRNA variants” are transcripts produced from the same genomic DNA that differ from other transcripts produced from the same genomic DNA in either their start or stop position and contain both intronic and exonic sequence. Variants can result in mRNA variants including, but not limited to, those with alternate splice junctions, or alternate initiation and termination codons. Variants in genomic and mRNA sequences can result in disease. Oligonucleotides to such variants can be used in the compositions and methods provided herein.

Target Names, Synonyms, Features

Compositions and methods are provided herein for modulating the expression of IL-4Rα (also known as interleukin 4 alpha receptor alpha chain; CD 124; IL-4Rα). Table 1 lists the GenBank accession numbers of sequences corresponding to nucleic acid molecules encoding IL-4Rα (nt=nucleotide), the date the version of the sequence was entered in GenBank, and the corresponding SEQ ID NO in the instant application, when assigned, each of which is incorporated herein by reference.

TABLE 1
Gene Targets

| Species | Genbank # | Genbank Date | SEQ ID NO |
|---|---|---|---|
| Human | BM738518.1 | 1 Mar. 2002 | |
| Human | nt 18636000 to 18689000 | 19 Feb. 2004 | |
| | of NT_010393.14 | | |
| Human | X52425.1 | 26 May 1992 | 1 |
| Mouse | AF000304.1 | 1 Dec. 1997 | |
| Mouse | assembled from M64868.1 | Both 6 May 1996 | |
| | and M64879.1 | | |
| Mouse | BB867141.1 | 9 Jul. 2003 | |
| Mouse | BC012309.1 | 3 Jan. 2005 | |
| Mouse | M27959.1 | 16 Sep. 1994 | |
| Mouse | M27960.1 | 12 Jun. 1993 | 2 |
| Mouse | M29854.1 | 12 Jun. 1993 | |

Modulation of Target Expression

Modulation of expression of a target nucleic acid can be achieved through alteration of any number of nucleic acid (DNA or RNA) functions. “Modulation” in the context of target expression means a perturbation of function, for example, either an increase (stimulation or induction) or a decrease (inhibition or reduction) in expression. As another example, modulation of expression can include perturbing splice site selection of pre-mRNA processing. “Expression” includes all the functions by which a gene's coded information is converted into structures present and operating in a cell. These structures include the products of transcription and translation. “Modulation of expression” means the perturbation of such functions. The functions of RNA to be modulated can include translocation functions, which include, but are not limited to, translocation of the RNA to a site of protein translation, translocation of the RNA to sites within the cell which are distant from the site of RNA synthesis, and translation of protein from the RNA. RNA processing functions that can be modulated include, but are not limited to, splicing of the RNA to yield one or more RNA species, capping of the RNA, 3′ maturation of the RNA and catalytic activity or complex formation involving the RNA which can be engaged in or facilitated by the RNA. Modulation of expression can result in the increased level of one or more nucleic acid species or the decreased level of one or more nucleic acid species, either temporally or by net steady state level. One result of such interference with target nucleic acid function is modulation of the expression of IL-4Rα. Thus, in one embodiment modulation of expression can mean increase or decrease in target RNA or protein levels. In another embodiment modulation of expression can mean an increase or decrease of one or more RNA splice products, or a change in the ratio of two or more splice products.

The effect of oligomeric compounds on target nucleic acid expression can be tested in any of a variety of cell types provided that the target nucleic acid is present at measurable levels. The effect of oligomeric compounds on target nucleic acid expression can be routinely determined using, for example, PCR or Northern blot analysis. Cell lines are derived from both normal tissues and cell types and from cells associated with various disorders (e.g., hyperproliferative disorders). Cell lines derived from multiple tissues and species can be obtained from American Type Culture Collection (ATCC, Manassas, Va.) and are well known to those skilled in the art. Primary cells, or those cells which are isolated from an animal and not subjected to continuous culture, can be prepared according to methods known in the art or obtained from various commercial suppliers. Additionally, primary cells include those obtained from donor human subjects in a clinical setting (i.e., blood donors, surgical patients). Primary cells prepared by methods known in the art.

Assaying Modulation of Expression

Modulation of IL-4Rα expression can be assayed in a variety of ways known in the art. IL-4Rα mRNA levels can be quantified by, e.g., Northern blot analysis, competitive polymerase chain reaction (PCR), or real-time PCR. RNA analysis can be performed on total cellular RNA or poly(A)+ mRNA by methods known in the art. Methods of RNA isolation are taught in, for example, Ausubel, F. M. et al., Current Protocols in Molecular Biology, Volume 1, pp. 4.1.1-4.2.9 and 4.5.1-4.5.3, John Wiley & Sons, Inc., 1993.

Northern blot analysis is routine in the art and is taught in, for example, Ausubel, F. M. et al., Current Protocols in Molecular Biology, Volume 1, pp. 4.2.1-4.2.9, John Wiley & Sons, Inc., 1996. Real-time quantitative (PCR) can be conveniently accomplished using the commercially available ABI PRISM™ 7700 Sequence Detection System, available from PE-Applied Biosystems, Foster City, Calif. and used according to manufacturer's instructions. The method of analysis of modulation of RNA levels is not a limitation of the methods provided herein.

Levels of a protein encoded by IL-4Rα can be quantified in a variety of ways well known in the art, such as immunoprecipitation, Western blot analysis (immunoblotting), ELISA or fluorescence-activated cell sorting (FACS). Antibodies directed to a protein encoded by IL-4Rα can be identified and obtained from a variety of sources, such as the MSRS catalog of antibodies (Aerie Corporation, Birmingham, Mich.), or can be prepared via conventional antibody generation methods. Methods for preparation of polyclonal antisera are taught in, for example, Ausubel, F. M. et al., Current Protocols in Molecular Biology, Volume 2, pp. 11.12.1-11.12.9, John Wiley & Sons, Inc., 1997. Preparation of monoclonal antibodies is taught in, for example, Ausubel, F. M. et al., Current Protocols in Molecular Biology, Volume 2, pp. 11.4.1-11.11.5, John Wiley & Sons, Inc., 1997.

Immunoprecipitation methods are standard in the art and can be found at, for example, Ausubel, F. M. et al., Current Protocols in Molecular Biology, Volume 2, pp. 10.16.1-10.16.11, John Wiley & Sons, Inc., 1998. Western blot (immunoblot) analysis is standard in the art and can be found at, for example, Ausubel, F. M. et al., Current Protocols in Molecular Biology, Volume 2, pp. 10.8.1-10.8.21, John Wiley & Sons, Inc., 1997. Enzyme-linked immunosorbent assays (ELISA) are standard in the art and can be found at, for example, Ausubel, F. M. et al., Current Protocols in Molecular Biology, Volume 2, pp. 11.2.1-11.2.22, John Wiley & Sons, Inc., 1991.

Validated Target Segments

The locations on the target nucleic acid to which active oligomeric compounds hybridize are herein below referred to as “validated target segments.” As used herein the term “validated target segment” is defined as at least an 8 nucleobase portion of a target region, such as at least a 9, 10, 11, 12, 13, 14, 15, 16, 17, 18, 19, 20, 21, 22, 23, 24, 25, 26, 27, 28, 29, 30, 31, 32, 33, 34 or 35 nucleobase portion of a target region, to which an active oligomeric compound is targeted. In certain embodiments, the validated target segment is a 20 nucleobase portion of a target region, to which an active oligomeric compound is targeted. While not wishing to be bound by theory, it is presently believed that these target segments represent portions of the target nucleic acid which are accessible for hybridization.

Target segments can include DNA or RNA sequences that comprise at least the 8, at least the 9, at least the 10, at least the 11, at least the 12, at least the 13, at least the 14, at least the 15, at least the 16, at least the 17, at least the 18, at least the 19 or at least the 20, at least the 21, at least the 22, at least the 23, at least the 24, at least the 25, at least the 26, at least the 27, at least the 28, at least the 29, at least the 30, at least the 31, at least the 32, at least the 33, at least the 34, or at least the 35 consecutive nucleobases from the 5′-terminus of a validated target segment (the remaining nucleobases being a consecutive stretch of the same DNA or RNA beginning immediately upstream of the 5′-terminus of the target segment and continuing until the DNA or RNA contains about 12 to about 35 nucleobases). Similarly validated target segments are represented by DNA or RNA sequences that comprise at least the 8, at least the 9, at least the 10, at least the 11, at least the 12, at least the 13, at least the 14, at least the 15, at least the 16, at least the 17, at least the 18, at least the 19 or at least the 20, at least the 21, at least the 22, at least the 23, at least the 24, at least the 25, at least the 26, at least the 27, at least the 28, at least the 29, at least the 30, at least the 31, at least the 32, at least the 33, at least the 34, or at least the 35 consecutive nucleobases from the 3′-terminus of a validated target segment (the remaining nucleobases being a consecutive stretch of the same DNA or RNA beginning immediately downstream of the 3′-terminus of the target segment and continuing until the DNA or RNA contains about 12 to about 35 nucleobases). It is also understood that a validated oligomeric target segment can be represented by DNA or RNA sequences that comprise at least the 8, at least the 9, at least the 10, at least the 11, at least the 12, at least the 13, at least the 14, at least the 15, at least the 16, at least the 17, at least the 18, at least the 19 or at least the 20, at least the 21, at least the 22, at least the 23, at least the 24, at least the 25, at least the 26, at least the 27, at least the 28, at least the 29, at least the 30, at least the 31, at least the 32, at least the 33, at least the 34, or at least the 35 consecutive nucleobases from an internal portion of the sequence of a validated target segment, and can extend in either or both directions until the oligonucleotide contains about 12 to about 35 nucleobases.

Screening for Modulator Oligomeric Compounds

In another embodiment, the validated target segments identified herein can be employed in a screen for additional compounds that modulate the expression of IL-4Rα. “Modulators” are those compounds that modulate the expression of IL-4Rα and which comprise at least an 8-nucleobase portion which is complementary to a validated target segment. In certain embodiments, the modulators comprise at least an 8, 9, 10 11, 12, 13, 14, 15, 16, 17, 18, 19, 20, 21, 22, 23, 24, 25, 26, 27, 28, 29, 30, 31, 32, 33, 34 or 35 nucleobase portion which is complementary to a validated target segment. In a specific embodiment, the modulator comprises at least a 20 nucleobase portion which is complementary to a validated target segment.

The screening method comprises the steps of contacting a validated target segment of a nucleic acid molecule encoding IL-4Rα with one or more candidate modulators, and selecting for one or more candidate modulators which perturb the expression of a nucleic acid molecule encoding IL-4Rα. Once it is shown that the candidate modulator or modulators are capable of modulating the expression of a nucleic acid molecule encoding IL-4Rα, the modulator can then be employed in further investigative studies of the function of IL-4Rα, or for use as a research, diagnostic, or therapeutic agent.

Modulator compounds of IL-4Rα can also be identified or further investigated using one or more phenotypic assays, each having measurable endpoints predictive of efficacy in the treatment of a particular disease state or condition. Phenotypic assays, kits and reagents for their use are well known to those skilled in the art.

Methods of Using Antisense Compounds

In a first aspect, provided herein is a method for modulating an immune response to a viral infection (e.g., a rhinovirus, influenza virus or coronavirus) in a child or adult subject, comprising administering to the subject an effective amount of an antisense compound 12 to 35 nucleobases in length targeted to a nucleic acid molecule encoding an IL-4Rα (e.g., a human IL-4Rα (SEQ ID NO:1)), wherein said antisense compound inhibits expression of the IL-4Rα protein and/or expression of functional IL-4 and IL-13 receptors. In some embodiments, the viral infection is a primary viral infection. In other embodiments, the viral infection is a secondary viral infection. In certain embodiments, the method comprises decreasing a Th2 response in the subject, increasing a Th1 response in the subject, or a combination thereof. In some embodiments, the modulating comprises decreasing CD4+Th2+ T cell (e.g., CD4+IL-4+, CD4+IL-5+, CD4+IL-9+, CD4+IL-10+, or CD4+IL-13+ T cell, or a combination thereof) production in the subject, decreasing CD8+Th2+ T cell production in a subject, increasing CD4+Th1+ and CD8+Th1+ T cell production in a subject, increasing CD4+IFNγ+ T cell or CD8+IFNγ+ T cell production in the subject, decreasing CD8+IL-4+ T cell production in the subject, decreasing airway hyperreactivity in the subject, decreasing pulmonary inflammation in the subject, maintaining or improving lung function in the subject, decreasing airway resistance in the subject, maintaining or increasing airway compliance in the subject, decreasing absolute numbers of airway eosinophils and/or neutrophils in the subject, decreasing an airway Th2 cytokine (e.g., IL-4, IL-5, IL-9, IL-10, or IL-13, or a combination thereof in the subject), decreasing chemokines (e.g. eotaxin 1 or 2, RANTES, IL-8, MIP-1 alpha, MCP-1), increasing serum IgG2a levels in the subject, decreasing serum IgE levels in the subject, decreasing anti-viral antibody titers in the subject, or any combination thereof.

In a second aspect provided herein is a method of managing, treating and/or ameliorating pulmonary inflammation, airway hyperreactivity and/or loss of lung function, or a symptom thereof, in a child or adult subject during the course of or resulting from a primary viral infection, comprising administering to a subject an effective amount of an antisense compound 12 to 35 nucleobases in length targeted to a nucleic acid molecule encoding an IL-4Rα (e.g., a human IL-4Rα (SEQ ID NO:1)), wherein said antisense compound inhibits expression of the IL-4Rα protein and/or expression of functional IL-4 and IL-13 receptors.

In a third aspect, provided herein is a method of managing, treating and/or ameliorating pulmonary inflammation, airway hyperreactivity and/or loss of lung function, or a symptom thereof, in a child or adult subject during the course of or resulting from a secondary viral infection, comprising administering to a subject an effective amount of an antisense compound 12 to 35 nucleobases in length targeted to a nucleic acid molecule encoding an IL-4Rα (e.g., a human IL-4Rα (SEQ ID NO:1)), wherein said antisense compound inhibits expression of the IL-4Rα protein and/or expression of functional IL-4 and IL-13 receptors.

In a fourth aspect, provided herein is a method of managing, treating and/or ameliorating pulmonary inflammation, airway hyperreactivity and/or loss of lung function, or a symptom thereof, in a child or adult subject during the course of or resulting from exposure to a non-viral environmental irritant, wherein the subject had a primary viral infection as an infant, said method comprising administering to a subject an effective amount of an antisense compound 12 to 35 nucleobases in length targeted to a nucleic acid molecule encoding an IL-4Rα (e.g., a human IL-4Rα (SEQ ID NO:1)), wherein said antisense compound inhibits expression of the IL-4Rα protein and/or expression of functional IL-4 and IL-13 receptors.

In a fifth aspect, provided herein is a method of preventing or delaying the onset of pulmonary inflammation, airway hyperreactivity and/or loss of lung function, or a symptom thereof, in a child or adult subject during the course of or resulting from a primary viral infection, comprising administering to a subject an effective amount of an antisense compound 12 to 35 nucleobases in length targeted to a nucleic acid molecule encoding an IL-4Rα (e.g., a human IL-4Rα (SEQ ID NO:1)), wherein said antisense compound inhibits expression of the IL-4Rα protein and/or expression of functional IL-4 and IL-13 receptors.

In a sixth aspect, provided herein is a method of preventing or delaying the onset of pulmonary inflammation, airway hyperreactivity and/or loss of lung function, or a symptom thereof, in a child or adult subject during the course of or resulting from a secondary viral infection, comprising administering to a subject an effective amount of an antisense compound 12 to 35 nucleobases in length targeted to a nucleic acid molecule encoding an IL-4Rα (e.g., a human IL-4Rα (SEQ ID NO:1)), wherein said antisense compound inhibits expression of the IL-4Rα protein and/or expression of functional IL-4 and IL-13 receptors.

In a seventh aspect, provided herein is a method of preventing or delaying the onset of pulmonary inflammation, airway hyperreactivity and/or loss of lung function, or a symptom thereof, in a child or adult subject during the course of or resulting from exposure to a non-viral environmental irritant, wherein the subject had a primary viral infection as an infant, said method comprising administering to a subject an effective amount of an antisense compound 12 to 35 nucleobases in length targeted to a nucleic acid molecule encoding an IL-4Rα (e.g., a human IL-4Rα (SEQ ID NO:1)), wherein said antisense compound inhibits expression of the IL-4Rα protein and/or expression of functional IL-4 and IL-13 receptors. In some embodiments, the non-viral environmental irritant is an allergen. In certain embodiments, the non-viral environmental irritant is cigarette smoke, bacteria, fungus, mold, dust mites, animal dander, or pollen.

In certain embodiments of the methods presented herein, the viral infection is a respiratory virus infection, such as a rhinovirus, influenza virus (e.g., an influenza A-type virus subtype H1N1 swine flu virus), coronavirus (e.g., SARS virus), RSV infection, or a combination thereof. In some embodiments, the virus infection is not a RSV infection. In certain embodiments, the method comprises decreasing a Th2 response in the subject, increasing a Th1 response in the subject, or a combination thereof. In some embodiments, the pulmonary inflammation, airway hyperreactivity and/or loss of lung function is associated with or caused by an atopic disease. In other embodiments, the pulmonary inflammation, airway hyperreactivity and/or loss of lung function is associated with or caused by a non-atopic disease. In some embodiments, the atopic or non-atopic disease is an allergy, asthma or rhinitis (e.g., allergic rhinitis). In some embodiments, the symptom is bronchoconstriction (i.e., wheezing, shortness of breath, cough or chest tightness, night time awakenings), or objective test measures including but not limited to increased sputum or sputum proteins in the airways, eosinophilic and/or eosinophilic inflammation in sputum, bronchialalveolar lavage fluid (BALF), nasal or lung tissue biopsy samples, increased mucus or mucin proteins in similar samples, histological, radiological or biochemical evidence of subepithelial fibrosis, collagen deposition or airway basement membrane thickening, elevated IgE levels in serum, sputum or BALF, a need for additional immunosuppressive or anti-inflammatory therapies, a need for bronchodilators, a need for oral or inhaled or intranasal corticosteroids or higher doses of corticosteroids, a need for leukotriene inhibitors, a need for anti-IgE antibody therapy, a need for emergency room treatment or hospitalization, a need for bronchodilators or higher doses of bronchodilators or more frequent use of short-acting bronchodilators, or a combination thereof. In yet other embodiments, the subject is in need thereof. In some embodiments, the subject is a human infant, such as pre-term infant. In some embodiments, the subject is a human child. In other embodiments, the subject is a human adult, such as an elderly adult. In some embodiments, the subject is immunocompromised and/or immunosuppressed.

In certain embodiments of the various methods provided herein, a primary and/or secondary viral infection can be assessed using standard immunological techniques, e.g., to assess the immunological status of a subject (e.g., B cell or T cell responses) to the virus (e.g., by ELISA to detect virus-specific antibodies). For example, in some instances, a healthy subject following an initial exposure to a virus will develop relatively low virus-specific antibody titers (and other B cell and T cell immune responses), which develop over the course of several weeks (e.g., within 4-6 weeks), and upon subsequent exposure to the same or closely related virus, the subject will quickly (e.g., within 1-2 weeks) develop higher virus-specific antibody titers (and other B cell and T cell immune responses) than following the primary infection due to a memory immune response. Avidity of the virus-specific antibodies also tends to be higher following secondary viral infections.

In an eighth aspect, provided herein is a method of inducing or augmenting hypo-responsiveness, non-responsiveness or tolerance to a virus in a child or adult subject, comprising administering to the subject (i) an antigen of the virus, and (ii) an effective amount of an antisense compound 12 to 35 nucleobases in length targeted to a nucleic acid molecule encoding an IL-4Rα (e.g., a human IL-4Rα (SEQ ID NO:1)), wherein said antisense compound inhibits expression of the IL-4Rα protein and/or expression of functional IL-4 and IL-13 receptors. In some embodiments, the subject was previously exposed to the virus, for example, as an infant. In other embodiments, the subject has not been previously exposed to the virus. In some embodiments, the antigen is not an RSV antigen. In one embodiment, the method comprises decreasing a Th2 response in the subject, increasing a Th1 response in the subject, or a combination thereof. In yet other embodiments, the subject is in need thereof. In some embodiments, the subject is a human child. In other embodiments, the subject is a human adult, such as an elderly adult. In some embodiments, the subject is immunocompromised and/or immunosuppressed. In certain embodiments, the virus is a respiratory virus infection, such as a rhinovirus, influenza virus (e.g., an influenza A-type virus subtype H1N1 swine flu virus), coronavirus (e.g., SARS virus), RSV infection, or a combination thereof. In some embodiments, the virus infection is not a RSV infection. In certain embodiments, a composition (e.g., comprising the antigen and the antisense compound, together or separate) is formulated for use in a child or adult subject. For example, in some embodiments, the composition comprises (i) an effective amount of an IL-4Rα antisense compound (e.g., a MOE oligonucleotide inhibitor of IL-4Rα), (ii) an effective amount of antigen, or (iii) an effective amount of an antisense compound IL-4Rα (e.g., a MOE oligonucleotide inhibitor of IL-4Rα) and an effective amount of antigen, for use in a child or adult subject.

In a ninth aspect, provided herein is a method of enhancing the efficacy of a viral vaccine in a child or adult subject, comprising administering to the subject (i) the viral vaccine, and (ii) an effective amount of an antisense compound 12 to 35 nucleobases in length targeted to a nucleic acid molecule encoding an IL-4Rα (e.g., a human IL-4Rα (SEQ ID NO:1)), wherein said antisense compound inhibits expression of the IL-4Rα protein and/or expression of functional IL-4 and IL-13 receptors. In one embodiment, the subject is in need thereof. In some embodiments, the subject is a human child. In other embodiments, the subject is a human adult, such as an elderly adult. In some embodiments, the subject is immunocompromised and/or immunosuppressed. In certain embodiments, the viral vaccine is a respiratory virus vaccine, such as a rhinovirus, influenza virus (e.g., an influenza A-type virus subtype H1N1 swine flu virus), coronavirus (e.g., SARS virus), RSV vaccine, or a combination thereof. In another embodiment, the vaccine is not directed to a respiratory syncytial virus or is not a RSV vaccine. In some embodiments, the vaccine is an attenuated vaccine, inactivated vaccine, toxoid vaccine, subunit vaccine, conjugated vaccine, DNA vaccine, monovalent vaccine, multivalent vaccine, or a combination thereof. In other embodiments, the vaccine is the method of administering the composition sequentially or concurrently in combination with an attenuated vaccine, inactivated vaccine, toxoid vaccine, subunit vaccine, conjugated vaccine, DNA vaccine, monovalent vaccine, multivalent vaccine, or a combination thereof. In certain embodiments, the composition further comprises an adjuvant, which can be sequentially or concurrently administered in combination with the vaccine. In some embodiments, the method comprises decreasing a Th2 response in the subject, increasing a Th1 response in the subject, or a combination thereof. In certain embodiments, a composition (e.g., comprising the vaccine and the antisense compound, together or separate) is formulated for use in a child or adult subject. For example, in some embodiments, the composition comprises (i) an effective amount of an IL-4Rα antisense compound (e.g., a MOE oligonucleotide inhibitor of IL-4Rα), (ii) an effective amount of vaccine, immunogen or antigen, or (iii) an effective amount of an antisense compound IL-4Rα (e.g., a MOE oligonucleotide inhibitor of IL-4Rα) and an effective amount of vaccine, immunogen or antigen, for use in a child or adult subject.

In a tenth aspect, provided herein is a method for modulating an immune response to a virus (e.g., a rhinovirus, influenza virus or coronavirus) infection in an infant subject, comprising administering to the subject an effective amount of an antisense compound 12 to 35 nucleobases in length targeted to a nucleic acid molecule encoding an IL-4Rα (e.g., a human IL-4Rα (SEQ ID NO:1)), wherein said antisense compound inhibits expression of the IL-4Rα protein and/or expression of functional IL-4 and IL-13 receptors. In some embodiments, the viral infection is a primary viral infection. In other embodiments, the viral infection is a secondary viral infection. In certain embodiments, the method comprises decreasing a Th2 response in the subject, increasing a Th1 response in the subject, or a combination thereof. In some embodiments, the modulating comprises decreasing CD4+Th2+ T cell (e.g., CD4+IL-4+, CD4+IL-5+, CD4+IL-9+, CD4+IL-10+, or CD4+IL-13+ T cell, or a combination thereof) production in the subject, decreasing CD8+Th2+ T cell production in a subject, increasing CD4+Th1+ and CD8+Th1+ T cell production in a subject, increasing CD4+IFNγ+ T cell or CD8+IFNγ+ T cell production in the subject, decreasing CD8+IL-4+ T cell production in the subject, decreasing airway hyperreactivity in the subject, decreasing pulmonary inflammation in the subject, maintaining or improving lung function in the subject, decreasing airway resistance in the subject, maintaining or increasing airway compliance in the subject, decreasing absolute numbers of airway eosinophils and/or neutrophils in the subject, decreasing an airway Th2 cytokine (e.g., IL-4, IL-5, IL-9, IL-10, or IL-13, or a combination thereof in the subject), decreasing chemokines (e.g. eotaxin 1 or 2, RANTES, IL-8, MIP-1 alpha, MCP-1), increasing serum IgG2a levels in the subject, decreasing serum IgE levels in the subject, decreasing anti-viral antibody titers in the subject, or any combination thereof.

In an eleventh aspect, provided herein is a method of managing, treating and/or ameliorating pulmonary inflammation, airway hyperreactivity and/or loss of lung function, or a symptom thereof, in an infant subject during the course of or resulting from a primary virus (e.g., rhinovirus, influenza virus or coronavirus) infection, comprising administering to a subject an effective amount of an antisense compound 12 to 35 nucleobases in length targeted to a nucleic acid molecule encoding an IL-4Rα (e.g., a human IL-4Rα (SEQ ID NO:1)), wherein said antisense compound inhibits expression of the IL-4Rα protein and/or expression of functional IL-4 and IL-13 receptors.

In a twelfth aspect, provided herein is a method of managing, treating and/or ameliorating pulmonary inflammation, airway hyperreactivity and/or loss of lung function, or a symptom thereof, in an infant subject during the course of or resulting from a secondary virus (e.g., rhinovirus, influenza virus or coronavirus) infection, comprising administering to a subject an effective amount of an antisense compound 12 to 35 nucleobases in length targeted to a nucleic acid molecule encoding an IL-4Rα (e.g., a human IL-4Rα (SEQ ID NO:1)), wherein said antisense compound inhibits expression of the IL-4Rα protein and/or expression of functional IL-4 and IL-13 receptors.

In a thirteenth aspect, provided herein is a method of managing, treating and/or ameliorating pulmonary inflammation, airway hyperreactivity and/or loss of lung function, or a symptom thereof, in an infant subject during the course of or resulting from exposure to a non-viral environmental irritant, wherein the subject previously had a primary (e.g., rhinovirus, influenza virus or coronavirus) infection, said method comprising administering to a subject an effective amount of an antisense compound 12 to 35 nucleobases in length targeted to a nucleic acid molecule encoding an IL-4Rα (e.g., a human IL-4Rα (SEQ ID NO:1)), wherein said antisense compound inhibits expression of the IL-4Rα protein and/or expression of functional IL-4 and IL-13 receptors.

In a fourteenth aspect, provided herein is a method of preventing or delaying the onset of pulmonary inflammation, airway hyperreactivity and/or loss of lung function, or a symptom thereof, in an infant subject during the course of or resulting from a primary virus (e.g., rhinovirus, influenza virus or coronavirus) infection, comprising administering to a subject an effective amount of an antisense compound 12 to 35 nucleobases in length targeted to a nucleic acid molecule encoding an IL-4Rα (e.g., a human IL-4Rα (SEQ ID NO:1)), wherein said antisense compound inhibits expression of the IL-4Rα protein and/or expression of functional IL-4 and IL-13 receptors.

In a fifteenth aspect, provided herein is a method of preventing or delaying the onset of pulmonary inflammation, airway hyperreactivity and/or loss of lung function, or a symptom thereof, in an infant subject during the course of or resulting from a secondary virus (e.g., rhinovirus, influenza virus or coronavirus) infection, comprising administering to a subject an effective amount of an antisense compound 12 to 35 nucleobases in length targeted to a nucleic acid molecule encoding an IL-4Rα (e.g., a human IL-4Rα (SEQ ID NO:1)), wherein said antisense compound inhibits expression of the IL-4Rα protein and/or expression of functional IL-4 and IL-13 receptors.

In a sixteenth aspect, provided herein is a method of preventing or delaying the onset of pulmonary inflammation, airway hyperreactivity and/or loss of lung function, or a symptom thereof, in an infant subject during the course of or resulting from exposure to a non-viral environmental irritant, wherein the subject previously had a primary virus (e.g., rhinovirus, influenza virus or coronavirus) infection, said method comprising administering to a subject an effective amount of an antisense compound 12 to 35 nucleobases in length targeted to a nucleic acid molecule encoding an IL-4Rα (e.g., a human IL-4Rα (SEQ ID NO:1)), wherein said antisense compound inhibits expression of the IL-4Rα protein and/or expression of functional IL-4 and IL-13 receptors. In certain embodiments, the non-viral environmental irritant is cigarette smoke, bacteria, fungus, mold, dust mites, animal dander, or pollen.

In certain embodiments of the methods provided herein, the viral infection is a respiratory virus infection, such as a rhinovirus, influenza virus (e.g., an influenza A-type virus subtype H1N1 swine flu virus), coronavirus (e.g., SARS virus), RSV infection, or a combination thereof. In some embodiments, the virus infection is not a RSV infection. In certain embodiments, the method comprises decreasing a Th2 response in the subject, increasing a Th1 response in the subject, or a combination thereof. In some embodiments, the pulmonary inflammation, airway hyperreactivity and/or loss of lung function is associated with or caused by an atopic disease. In other embodiments, the pulmonary inflammation, airway hyperreactivity and/or loss of lung function is associated with or caused by a non-atopic disease. In some embodiments, the atopic or non-atopic disease is an allergy, asthma or rhinitis (e.g., allergic rhinitis). In some embodiments, the symptom is wheezing, dyspenea (i.e., shortness of breath), increased sputum in the lungs, eosinophilic inflammation, neutrophilic inflammation, mucus hyper-secretion, subepithelial fibrosis, elevated IgE levels, coughing, chest tightening, a need for additional immunosuppressive or anti-inflammatory therapies, a need for bronchodilators, a need for corticosteroids, a need for leukotriene inhibitors, a need for anti-IgE antibody therapy, a need for hospitalization, or a combination thereof. In yet other embodiments, the subject is in need thereof. In some embodiments, the subject is a human infant, such as pre-term infant. In some embodiments, the infant is immunocompromised and/or immunosuppressed.

In a seventeenth aspect, provided herein is a method of inducing or augmenting hypo-responsiveness, non-responsiveness or tolerance to a virus (e.g., rhinovirus, influenza virus or coronavirus) in an infant subject, comprising administering to the subject (i) an antigen of the virus, and (ii) an effective amount of an antisense compound 12 to 35 nucleobases in length targeted to a nucleic acid molecule encoding an IL-4Rα (e.g., a human IL-4Rα (SEQ ID NO:1)), wherein said antisense compound inhibits expression of the IL-4Rα protein and/or expression of functional IL-4 and IL-13 receptors. In some embodiments, the subject was previously exposed to the virus. In other embodiments, the subject has not been previously exposed to the virus. In one embodiment, the method comprises decreasing a Th2 response in the subject, increasing a Th1 response in the subject, or a combination thereof. In one embodiment, the subject is in need thereof. In some embodiments, the subject is a human infant, such as pre-term infant. In some embodiments, the infant is immunocompromised and/or immunosuppressed. In certain embodiments, the virus is a respiratory virus infection, such as a rhinovirus, influenza virus (e.g., an influenza A-type virus subtype H1N1 swine flu virus), coronavirus (e.g., SARS virus), RSV infection, or a combination thereof. In some embodiments, the virus infection is not a RSV infection.

In an eighteenth aspect, provided herein is a method of enhancing the efficacy of a viral (e.g., rhinovirus, influenza virus or coronavirus) vaccine in an infant subject, comprising administering to the subject (i) the vaccine, and (ii) an effective amount of an antisense compound 12 to 35 nucleobases in length targeted to a nucleic acid molecule encoding an IL-4Rα (e.g., a human IL-4Rα (SEQ ID NO:1)), wherein said antisense compound inhibits expression of the IL-4Rα protein and/or expression of functional IL-4 and IL-13 receptors. In one embodiment, the subject is in need thereof. In some embodiments, the subject is a human infant, such as pre-term infant. In some embodiments, the infant is immunocompromised and/or immunosuppressed. In certain embodiments, the viral vaccine is a respiratory virus vaccine, such as a rhinovirus, influenza virus (e.g., an influenza A-type virus subtype H1N1 swine flu virus), coronavirus (e.g., SARS virus), RSV vaccine, or a combination thereof. In another embodiment, the vaccine is not directed to a respiratory syncytial virus or is not a RSV vaccine. In some embodiments, the vaccine is an attenuated vaccine, inactivated vaccine, toxoid vaccine, subunit vaccine, conjugated vaccine, DNA vaccine, monovalent vaccine, multivalent vaccine, or a combination thereof. In other embodiments, the vaccine is the method of administering the composition sequentially or concurrently in combination with an attenuated vaccine, inactivated vaccine, toxoid vaccine, subunit vaccine, conjugated vaccine, DNA vaccine, monovalent vaccine, multivalent vaccine, or a combination thereof. In certain embodiments, the composition further comprises an adjuvant, which can be sequentially or concurrently administered in combination with the vaccine. In some embodiments, the method comprises decreasing a Th2 response in the subject, increasing a Th1 response in the subject, or a combination thereof. In certain embodiments, a composition (e.g., comprising the vaccine and the antisense compound, together or separate) is formulated for use in an infant subject. For example, in some embodiments, the composition comprises (i) an effective amount of an IL-4Rα antisense compound (e.g., a MOE oligonucleotide inhibitor of IL-4Rα), (ii) an effective amount of vaccine, immunogen or antigen, or (iii) an effective amount of an antisense compound IL-4Rα (e.g., a MOE oligonucleotide inhibitor of IL-4Rα) and an effective amount of vaccine, immunogen or antigen, for use in an infant subject.

In another aspect, provided herein are methods of treating an upper respiratory tract infection (e.g., a viral infection, such as a cold) or a symptom thereof, comprising administering to the subject a composition comprising an antisense compound 12 to 35 nucleobases in length targeted to a nucleic acid molecule encoding an IL-4Rα (e.g., a human IL-4Rα (SEQ ID NO:1)), wherein said compound inhibits expression of the IL-4Rα protein and/or expression of functional IL-4 and IL-13 receptors.

In yet another aspect, provided herein are methods of preventing or delaying the onset of an upper respiratory tract infection (e.g., a viral infection, such as a cold) or a symptom thereof, comprising administering to the subject a composition comprising an antisense compound 12 to 35 nucleobases in length targeted to a nucleic acid molecule encoding an IL-4Rα (e.g., a human IL-4Rα (SEQ ID NO:1)), wherein said compound inhibits expression of the IL-4Rα protein and/or expression of functional IL-4 and IL-13 receptors.

In another aspect, provided herein is a method of treating a respiratory disorder in a subject comprising administering (e.g., topically) to the subject a composition comprising an antisense compound 12 to 35 nucleobases in length targeted to a nucleic acid molecule encoding an IL-4Rα (e.g., a human IL-4Rα (SEQ ID NO:1)), wherein said compound inhibits expression of the IL-4Rα protein and/or expression of functional IL-4 and IL-13 receptors. In specific embodiments, the respiratory disorder occurs during the course of or results from a viral infection, such as a primary or secondary viral infection. In certain embodiments, the virus is a respiratory virus infection, such as a rhinovirus, influenza virus (e.g., an influenza A-type virus subtype H1N1 swine flu virus), coronavirus (e.g., SARS virus), RSV infection, or a combination thereof. In some embodiments, the virus infection is not a RSV infection. In certain embodiments, the compound is administered no more frequently than about once per week. In some embodiments, the disorder is associated with high baseline levels (e.g., above 2% or above 3%) of eosinophils or neutrophils in the sputum or BAL. In other embodiments, the disorder is Th2-mediated or associated with Th2 immunity, such as allergic and non-allergic asthma, COPD, IPF, cystic fibrosis, chronic bronchitis, rhinitis (e.g., allergic rhinitis), nasal polyposis, pulmonary inflammation, airway hyper-responsiveness, and a respiratory inflammatory condition associated with or resulting from a viral infection, such as a respiratory virus, such as a rhinovirus, influenza, coronavirus, parainfluenza, metapneumovirus, or RSV infection. In certain embodiments, the viral infection is not an RSV infection. In certain embodiments, the topical administration is to a respiratory tract of the subject. In one embodiment, the topical administration comprises aerosol administration.

In another aspect, provided herein is a method of treating a respiratory disorder in a subject, wherein said subject has at least one of the group consisting of measurable serum IgE, sputum or BAL eosinophilia, sputum or BAL neutrophilia, sputum 15-HETE, or sputum IL-4Rα mRNA or protein or exhaled nitric oxide level, comprising administering (e.g., topically) to the subject a composition comprising an antisense compound 12 to 35 nucleobases in length targeted to a nucleic acid molecule encoding an IL-4Rα (e.g., a human IL-4Rα (SEQ ID NO:1)), wherein said compound inhibits expression of the IL-4Rα protein and/or expression of functional IL-4 and IL-13 receptors. In some embodiments, the composition is administered to the subject no more frequently than about once per week. In certain embodiments, the disorder is Th2-mediated or associated with Th2 immunity, such as allergic and non-allergic asthma, COPD, IPF, CF, chronic bronchitis, rhinitis (e.g., allergic rhinitis), nasal polyposis, pulmonary inflammation, airway hyper-responsiveness, and a respiratory inflammatory condition associated with or resulting from a viral infection. In some embodiments, the viral infection is a rhinovirus, influenza virus, coronavirus or RSV infection. In some embodiments, the viral infection is not an RSV infection. In certain embodiments, the topical administration is to a respiratory tract of the subject. In one embodiment, the topical administration comprises aerosol administration.

In yet another aspect, provided herein is a method of enhancing the efficacy of a anti-viral therapy in a subject, comprising administering to the subject (i) the anti-viral therapy, and (ii) an effective amount of an antisense compound 12 to 35 nucleobases in length targeted to a nucleic acid molecule encoding an IL-4Rα (e.g., a human IL-4Rα (SEQ ID NO:1)), wherein said antisense compound inhibits expression of the IL-4Rα. In certain embodiments, the subject is in need thereof. In some embodiments, the subject is a human infant, such as pre-term infant. In some embodiments, the subject is a human child. In other embodiments, the subject is a human adult, such as an elderly adult. In some embodiments, the subject is immunocompromised and/or immunosuppressed. In certain embodiments, the anti-viral therapy is an anti-respiratory virus therapy, such as an anti-RSV, anti-rhinovirus, anti-influenza virus (e.g., influenza A-type virus subtype H1N1 swine flu virus), anti-coronavirus (e.g., SARS virus) therapy, or a combination thereof. In some embodiments, the anti-viral therapy is oseltamivir (Tamiflu®) or zanamivir (Relenza®), amantadine (Symmetrel®), rimantadine (Flumadine®), ribavirin (Copegus®, Rebetol®, Ribasphere®, Vilona® or Virazole®), RSV-IG (RespiGam®), palivizumab (Synagis®), or combination thereof. In certain embodiments, the anti-viral therapy is not an anti-RSV therapy (e.g., not an anti-RSV antibody, such as not RSV-IG and/or palivizumab).

In certain embodiments of the various methods provided herein, levels of serum IgE, exhaled nitric oxide, sputum or BAL eosinophilia, sputum or BAL neutrophilia, sputum 15-HETE, or sputum IL-4Rα mRNA or protein or exhaled nitric oxide level, or a combination thereof are decreased as compared to the levels in the absence of antisense compound administration.

In some embodiments of the methods provided herein, the subject is a human subject, such as an infant (e.g., a pre-term infant), child or adult subject. In certain embodiments, the subject is not an infant. In one embodiment, the methods provided herein comprise the administration of one or more antisense compounds provided herein to subjects that are immunocompromised and/or immunosuppressed, including human adult, child and infant subjects. For example, in certain embodiments, an antisense compound provided herein is administered to an immunocompromised and/or immunosuppressed human, for example, with a congenital immunodeficiency, acquired immunodeficiency, cystic fibrosis, bronchopulmonary dysplasia, congenital heart disease, a human who has a cancer or tumor, or to a human who has had a bone marrow transplant or is undergoing chemotherapy, steroid therapy or other immunosuppressive therapy. In certain embodiments, the subject (e.g., an infant, child or adult) has an immune deficiency (primary or secondary), and in certain cases does not limit virus replication and/or has increased and prolonged viral shedding and/or increased clinical illness from the infection. In some embodiments, the subject has been previously hospitalized for a viral infection or a symptom thereof. In other embodiments, the subject has or is at risk for pulmonary inflammation, airway hyperreactivity and/or loss of lung function (e.g., associated with or caused by an atopic or non-atopic disease, such as asthma, allergy, rhinitis or other respiratory disease or disorder, including chronic bronchitis, pneumonia, pulmonary fibrosis, emphysema, COPD, IPF, CF, nasal polyposis, or infection with RSV, rhinovirus, influenza virus, coronavirus or other respiratory virus), or a symptom thereof. In some embodiments, the subject has been previously hospitalized for pulmonary inflammation, airway hyperreactivity and/or loss of lung function (e.g., associated with or caused by an atopic or non-atopic disease, such as asthma, allergy, rhinitis or other respiratory disease or disorder, including chronic bronchitis, pneumonia, pulmonary fibrosis, emphysema, COPD, IPF, CF, nasal polyposis, or infection with RSV, rhinovirus, influenza virus, coronavirus or other respiratory virus), or a symptom thereof. In some embodiments, the subject has high baseline levels of serum IgE, exhaled nitric oxide, sputum or BAL eosinophilia, sputum or BAL neutrophilia, sputum 15-HETE, or sputum IL-4Rα mRNA or protein or exhaled nitric oxide level, for example, associated with or resulting from a respiratory disease or disorder, which can be treated or prevented using the compositions and methods provided herein.

In other embodiments of the various methods provided herein, an antisense compound provided herein is administered to a human infant, such as a pre-term infant, child or adult at risk of hospitalization for a viral infection or other respiratory disorder. In another embodiment, an antisense compound provided herein is administered to an adult, such as an elderly subject. In certain embodiments, the subject has or is at risk for developing pulmonary inflammation, airway hyperreactivity and/or loss of lung function (e.g., associated with or caused by an atopic or non-atopic disease, such as asthma, allergy, rhinitis or other respiratory disease or disorder, including chronic bronchitis, pneumonia, pulmonary fibrosis, emphysema, COPD, IPF, CF, nasal polyposis, or infection with RSV, rhinovirus, influenza virus, coronavirus or other respiratory virus), or a symptom thereof; has a family history of a respiratory disorder (e.g., is genetically pre-disposed) or has, for example a polymorphism in a gene such as surfactant associated proteins A and D. In certain embodiments, the subject is at exacerbation of pulmonary or nasal symptoms (e.g. congestion, wheezing, difficulty breathing, chest tightness, sinus pain, cough), pulmonary inflammation, airway hyperreactivity and/or loss of lung function (e.g., associated with or caused by an atopic or non-atopic disease, such as asthma, allergy, rhinitis or other respiratory disease or disorder, including chronic bronchitis, pneumonia, pulmonary fibrosis, emphysema, COPD, IPF, CF, nasal polyposis, or infection with RSV, rhinovirus, influenza virus, coronavirus or other respiratory virus), or a symptom thereof. In some embodiments, the subject has allergic bronchopulmonary aspergillosis (ABPA) or pseudomonas, and can further have another respiratory disease, such as asthma and/or CF. In some embodiments, the subject has high baseline levels of serum IgE, exhaled nitric oxide, sputum or BAL eosinophilia, sputum or BAL neutrophilia, sputum 15-HETE, or sputum IL-4Rα mRNA or protein or exhaled nitric oxide level, or other Th2 biomarker provided herein. In specific embodiments, the subject has high IgE levels and/or high baseline sputum eosinophils (e.g., greater than about 2% or greater than about 3%).

In some embodiments, the subject has a polymorphism or other dysfunction in one or more ligands or receptors associated with a Th2 immune response (e.g., IL-4, IL-13, an IL-4 receptor or an IL-13 receptor, or a combination thereof) and/or one or more components in the signaling pathway thereof (e.g., Stat6), for example, such that the subject has increased expression or responsiveness to one or more of the ligands, receptors or signaling pathway components. In certain embodiments, the subject has a polymorphism associated with the IL-4Rα gene, such that IL-4Rα is over-expressed or over-functioning or hypersensitive to stimulation by IL-4 and/or IL-13 in the patient, for example, in the lung tissue, airway or blood cells, BALF or sputum. In other embodiments, the subject has a polymorphism or other dysfunction in one or more ligands or receptors associated with a Th1 immune response (e.g., IFNγ, IL-12, IFNγ receptor, or a combination thereof) and/or one or more components in the signaling pathway thereof, for example, such that the subject has decreased expression or responsiveness to one or more of the 11-4 or IL-13 ligands, receptors or signaling pathway components. Such subjects can be genetically predisposed to developing, for example, high IgE levels, asthma, airway hyperreactivity, and atopy. Examples of such polymorphisms associated with a Th2 immune response include, for example, those associated with the 5q31-5q33 region, which affect IL-4 and IL-13 (see, e.g., Szalai et al. (2008) Brit. J. Pharmacol. 153:1602; Kabesch et al. (2003) J. Allergy Clin. Immunol. 112:893; Chen et al. (2004) J. Allergy Clin. Immunol. 114:553, each of which is incorporated herein by reference in its entirety). Examples of populations known to comprise these chromosome 5 genetic polymorphisms include Amish, German, USA Caucasian, USA Hispanic, Chinese and Koreans. Other examples of such polymorphisms include those associated with the 16p21 region, which affects IL-4Rα (see, e.g., Szalai et al. (2008) Brit. J. Pharmacol. 153:1602; Hytonen et al. (2004) Clin. Exp. Allergy 34:1570, each of which is incorporated herein by reference in its entirety). Examples of populations known to comprise such chromosome 16 mutations include Chinese, German and Spanish. Similarly, subjects can comprise one or more single nucleotide polymorphisms (SNPs) associated with IL-13 pathway genes (e.g., IL-4, IL-13, IL-4Rα, IL-13Rα1, IL-13Rα2Jak1, Jak3 and Stat6), which can contribute to atopy and asthma, such as rs2243250 (IL-4), rs1881457, rs1800925, rs2066960, rs20541, rs1295685 (IL-13); rs1805010, rs1805015, rs1801275 (IL-4Rα); rs2250747 (IL-13Rα1), rs5946040 (IL-13Rα2), and rs324011 (Stat6) (see, e.g., Beghe et al. (2009 Oct. 1) Allergy; ePub ahead of print, which is incorporated herein by reference in its entirety). In certain embodiments, the subject comprises one or more exemplary genetic polymorphisms and variants associated with IL-4 and IL-4Rα genes provided in, e.g., Beghe et al. (2003) Clin. Exp. Allergy 33:1111; Hershey et al. (1997) N. Engl. J. Med. 337:1720; Howard et al. (2002) Am. J. Hum. Genet. 70:230; Ober et al. (2000) Am. J. Hum. Genet. 55:517, each of which is incorporated herein by reference in its entirety. In other embodiments, the subject has an I75V, N98Tm E400A, C431R, S503P or Q576R SNP in IL-4Rα (see, e.g., Knutsen et al. (2006) Clin. Mol. Allergy. 4:3, which is incorporated herein by reference in its entirety). In certain embodiments, the subject has a Q576R polymorphism in the human IL-4Rα chain gene (see, e.g., Hershey et al. (1997) N. Engl. J. Med. 337:1720; Rosa-Rosa (1999) J. Allergy Clin. Immunol. 104:1008; Sandford et al. (2000) J. Allergy Clin. Immunol. 106:135; Ober et al. (2000) Am. J. Hum. Genet. 55:517; Wenzel et al. (2007) Am. J. Respir. Crit. Care Med. 175:570; each of which is incorporated herein by reference in its entirety), which is overrepresented in the African-American population (70% allelic frequency in African-Americans versus 20% in Caucasians, giving rise to 50% and 4% homozygosity, respectively (Tachdjian et al. (2009) J. Exp. Med. 206:2191, which is incorporated herein by reference in its entirety)). Mouse models corresponding to the Q576R polymorphism exist (Id.) and can be similarly used in the exemplary experiments provided elsewhere herein.

In some embodiments of the methods provided herein, one or more antisense compounds and optionally one or more additional therapeutic agents are administered to a subject suffering from or expected to suffer from (e.g., patients with a genetic predisposition for or patients that have previously suffered from) a viral infection, pulmonary inflammation, airway hyperreactivity and/or loss of lung function (e.g., associated with or caused by an atopic or non-atopic disease, such as asthma, allergy, rhinitis or other respiratory disease, including chronic bronchitis, pneumonia, pulmonary fibrosis, emphysema, COPD, IPF, CF, nasal polyposis, or infection with RSV, rhinovirus, influenza virus, coronavirus or other respiratory virus), or a symptom thereof. Such patients may have been previously treated or are currently being treated for the pulmonary inflammation, airway hyperreactivity and/or loss of lung function, or symptom thereof, e.g., with a therapy other than an antisense compound provided herein.

In other embodiments of the methods provided herein, an antisense compound provided herein can be used as any line of therapy, including, but not limited to, a first, second, third, fourth and/or fifth line of therapy. In yet other embodiments, antisense compounds provided herein can be used before or after any adverse effects or intolerance of other therapies occurs.

In some embodiments of the methods provided herein, the antisense compound is formulated to be administered by systemic administration. In other embodiments, the antisense compound is formulated to be administered by local administration. In some embodiments of the methods provided herein, the antisense compound is administered to the upper respiratory tract (e.g., nose, ears, sinuses, and throat) and/or lower respiratory tract (e.g., trachea, bronchial tubes, and lungs) of the subject. In some embodiments, the antisense compound is formulated to be administered by intranasal, intratracheal, sublingual, aerosol and/or respiratory administration. In certain embodiments, the antisense compounds are formulated as a particle size of from about 0.2 μm to about 10 μm, such as about a mean aerodynamic diameter of about 1.0 μm to about 5.0 μm, such as about 0.5 μm to about 0.8 μm, for administration (e.g., aerosol or respiratory administration). In other embodiments, the antisense compound is formulated to be administered by insufflation or as a nasal spray or nasal gel. In yet other embodiments, the antisense compound is formulated to be administered using a nebulizer, nasal inhaler, metered dose inhaler, dry powder inhaler, pulmonary inhaler, or a combination thereof.

In any of the various methods provided herein, the antisense compound can be administered intermittently or chronically. In one embodiment, the antisense compound is administered no more frequently than two times per day, for example, two times per one day or one time per one day, two days, three days, four days, five days or six days. In other embodiments, the antisense compound is administered no more frequently than one time per week, for example, one time per one week, two weeks or three weeks. In some embodiments, the antisense compound is administered no more frequently than one time per month, for example, one time per one month, two months, three months, four months, five months, six months, seven months, eight months, nine months, ten months, eleven months. In some embodiments, the antisense compound is administered no more frequently than one time per year, for example, one time per one year, two years, three years, four years, five years, ten years, fifteen years, twenty years or longer. In certain embodiments, the antisense compound is administered at a given frequency over the course of, for example, one week, two weeks, three weeks, one month, two months, three months, four months, five months, six months, seven months, eight months, nine months, ten months, eleven months, one year, two years, three years, four years, five years, ten years, fifteen years, twenty years or the lifetime of the subject. Various combinations of the above dosing frequencies and schedules are also contemplated (e.g., one time per day for one week, then one time weekly or one time monthly thereafter).

In some embodiments, the antisense compound is administered seasonally, such as, for example, one, two, three, four or five times per season. In other embodiments, the antisense compound is administered prior to the start of a season, such as a viral season. In other embodiments, the antisense compound is administered over the course of a season, such as a viral season. In one embodiment, the season is spring, summer, winter or autumn. In some embodiments, the season is an allergy season (e.g., a ragweed season or a pollen season, the timing of which can be dependent on geographical area).

In certain embodiments of the various methods provided herein, the antisense compound is administered at a total dose and/or effective amount of from about 0.01 mg, about 0.02 mg, about 0.04 mg, about 0.06 mg, about 0.08 mg, about 0.1 mg to about 100 mg, such as from about 0.25 mg to about 30 mg, or about 2 mg to about 20 mg (or a range therein). In some embodiments, the antisense compound is administered at a total and/or effective amount of about 0.1 mg, about 0.2 mg, about 0.25 mg, about 0.3 mg, about 0.4 mg, about 0.5 mg, about 0.75 mg, about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, about 200 mg, about 250 mg, about 300 mg, about 350 mg, about 400 mg, about 450 mg, or about 500 mg (or any range therein).

The total doses and/or or effective amounts provided herein may be administered one time, or as fractionated doses over a period of time (e.g., over the course of days, weeks, months, years of the lifetime of the subject). For example, in certain embodiments, the total dose and/or effective amount is administered once per week (one dose of 3.5 mg; total dose of 3.5 mg/week), but can also be fractionated for more frequent administration, such as once per day for one week (seven doses of 0.01 mg (i.e., 0.01 mg/day); total dose of 0.07 mg/week).

In some embodiments of the various methods provided herein, the total dose and/or effective amount of the antisense compound administered is in a range of from about 0.001 μg/kg (μg of antisense compound per kg weight of the subject) to about 0.01 μg/kg, about 0.1 μg/kg, about 0.001 mg/kg, about 0.01 mg/kg, about 0.1 mg/kg, about 1 mg/kg, about 10 mg/kg or about 100 mg/kg. In other embodiments, the total dose and/or effective amount of the antisense compound administered is in a range of from about 0.01 μg/kg to about 0.1 μg/kg, about 0.001 mg/kg, about 0.01 mg/kg, about 0.1 mg/kg, about 1 mg/kg, about 10 mg/kg or about 100 mg/kg. In one embodiment, the total dose and/or effective amount of the antisense compound administered is in a range of from about 0.1 μg/kg to about 0.001 mg/kg, about 0.01 mg/kg, about 0.1 mg/kg, about 1 mg/kg, about 10 mg/kg or about 100 mg/kg. In another embodiment, the total dose and/or effective amount of the antisense compound administered is in a range of from about 0.001 mg/kg to about 0.01 mg/kg, about 0.1 mg/kg, about 1 mg/kg, about 10 mg/kg or about 100 mg/kg. In other embodiments, the total dose and/or effective amount of the antisense compound administered is in a range of from about 0.01 mg/kg to about 0.1 mg/kg, about 1 mg/kg or about 10 mg/kg. In another embodiment, the total dose and/or effective amount of the antisense compound administered is in a range of from about 0.1 mg/kg to about 1 mg/kg, about 10 mg/kg or about 100 mg/kg. In yet other embodiments, the total dose and/or effective amount of the antisense compound administered is in a range of from about 1 mg/kg to about 10 mg/kg or about 100 mg/kg.

In specific embodiments, the total dose and/or effective amount of the antisense compound administered is in a range of from about 0.001 μg/kg to about 100 mg/kg, such as from about 0.01 μg/kg to about 100 mg/kg, from about 0.1 μg/kg to about 100 mg/kg, from about 0.001 mg/kg to about 100 mg/kg, from about 0.01 mg/kg to about 100 mg/kg; from about 0.1 mg/kg to about 100 mg/kg, from about 1 mg/kg to about 100 mg/kg or from about 10 mg/kg to about 100 mg/kg (or any range therein). In other embodiments, the total dose and/or effective amount of the antisense compound administered is in a range of from about 0.001 μg/kg to about 20 mg/kg, such as from about 0.01 μg/kg to about 20 mg/kg, from about 0.1 μg/kg to about 20 mg/kg, from about 0.001 mg/kg to about 20 mg/kg, from about 0.01 mg/kg to about 20 mg/kg; from about 0.1 mg/kg to about 20 mg/kg, from about 1 mg/kg to about 100 mg/kg or from about 10 mg/kg to about 20 mg/kg (or any range therein).

In specific embodiments, the antisense compound is administered at a total dose and/or effective amount of from about 0.033 mg/kg and about 0.33 mg/kg. In certain embodiments, the antisense compound is administered at a total dose and/or an effective amount of about 0.001 μg/kg, about 0.01 μg/kg, about 0.1 μg/kg, about 0.001 mg/kg, about 0.01 mg/kg, about 0.033 mg/kg, about 0.05 mg/kg, about 0.1 mg/kg, about 0.33 mg/kg, about 0.5 mg/kg, about 1 mg/kg, about 3 mg/kg, 5 mg/kg, about 8 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg about 90 mg/kg or about 100 mg/kg (or any range therein).

In specific embodiments of the various methods provided herein, the antisense compound is administered to the respiratory tract (e.g., by intranasal, intratracheal, aerosol and/or respiratory administration) and the total dose and/or effective amount is less than about 10 mg/kg, such as less than about 0.001 μg/kg, less than about 0.01 μg/kg, less than about 0.1 μg/kg, less than about 0.001 mg/kg, less than about 0.01 mg/kg, less than about 0.033 mg/kg, less than about 0.05 mg/kg, less than about 0.1 mg/kg, about 0.33 mg/kg, less than about 0.5 mg/kg, less than about 1 mg/kg, less than about 3 mg/kg, less than about 5 mg/kg, or less than about 8 mg/kg (or any range therein).

In certain embodiments, of the various methods provided herein, the total dose and/or effective amount of antisense compound is administered (e.g., by inhalation or other method described herein) to a subject, such that the concentration of antisense compound following administration in the subject's lung is greater than 10 ng/g lung tissue. In certain embodiments, of the various methods provided herein, the total dose and/or effective amount of antisense compound is administered (e.g., by inhalation or other method described herein) to a subject, such that the concentration of antisense compound following administration in the subject's lung is from about 0.01 μg/g and about 1 mg/g, such as about 0.01 μg/g, about 0.1 μg/g, about 1 μg/g, about 10 μg/g, about 100 μg/g or about 1 mg/g (or any range therein).

In some embodiments, the total dose and/or effective amount of the antisense compound is administered as a single dose. In other embodiments, the total dose and/or effective amount of the antisense compound is divided into multiple doses, which can be administered at a desired frequency over a desired period of time, and can include any of the various frequencies, dosing schedules and/or routes of administration provided herein.

For example, in certain embodiments, the antisense compound is administered three times over the course of one week at a dose of 0.1 mg/kg (total dose of 0.3 mg/kg/week), for example by aerosol or respiratory administration (e.g., using a nebulizer or inhaler). In another embodiment, the antisense compound is administered three times over the course of one week at a dose of 0.01 mg/kg (total dose of 0.03 mg/kg/week), for example by aerosol or respiratory administration (e.g., using a nebulizer or inhaler). In other embodiments, the antisense compound is administered three times over the course of one week at a dose of 0.033 mg/kg (total dose of 0.1 mg/kg/week), for example by aerosol or respiratory administration (e.g., using a nebulizer or inhaler). In yet other embodiments, the antisense compound is administered three to seven times over the course of one week at a dose of 1 μg/kg day (total dose of 3 μg/kg/week to 7 μg/kg/week), for example by aerosol or respiratory administration (e.g., using a nebulizer or inhaler).

In some embodiments of the methods provided herein, the antisense compound is present in the subject (e.g., in the lung or sputum of the subject) for a period of time (days, weeks, months or years) after the administration of the first dose of antisense compound and prior to the optional administration of a subsequent dose, wherein said certain period of time is from about 3 days to about 365 days or longer, such as from 3 days to 21 days, and in certain embodiments is at least 3 days, at least 7 days, at least 10 days, at least 14 days, at least 21 days, at least 28 days, at least 45 days, at least 60 days, at least 90 days, at least 180 days, or at least 365 days or longer. In some embodiments, said certain period of time is about 3 days, about 5 days, about 7 days, about 10 days, about 14 days, about 21 days, about 28 days, about 2 months, about 3 months, about 4 months, about 5 months, about 6 months, about 7 months, about 8 months, about 9 months, about 10 months, about 11 months, about 12 months, about 18 months or about 24 months or longer.

In other embodiments of the methods provided herein, one or more biomarkers of a decrease in a Th2 immune response and/or of an increase in a Th1 response associated with or caused by administration of an antisense compound provided herein is present for a certain period of time (days, weeks, months or years) after the administration of the first dose of antisense compound and prior to the optional administration of a subsequent dose, wherein said certain period of time is from about 3 days to about 365 days or longer, such as from 3 days to 21 days, and in certain embodiments is at least 3 days, at least 7 days, at least 10 days, at least 14 days, at least 21 days, at least 28 days, at least 45 days at least 60 days, at least 90 days, at least 180 days, or at least 365 days or longer. In some embodiments, said certain period of time is about 3 days, about 5 days, about 7 days, about 10 days, about 14 days, about 21 days, about 28 days, about 2 months, about 3 months, about 4 months, about 5 months, about 6 months, about 7 months, about 8 months, about 9 months, about 10 months, about 11 months, about 12 months, about 18 months or about 24 months or longer.

Any of the compounds, dosing amounts, routes of administration, and/or frequencies, durations or other dosing schedules provided herein may be used alone or in any combination for use with any of the various methods provided herein.

In a further embodiment of the methods provided herein, the antisense compound 12 to 35 nucleobases in length is targeted (e.g., coding/translated region, 5′ untranslated region, 3′ untranslated region or a combination thereof, including regions spanning the translated and untranslated regions) to a nucleic acid molecule encoding an IL-4Rα, wherein said antisense compound inhibits expression of the IL-4Rα protein and/or expression of functional IL-4 and IL-13 receptors. In specific embodiments, the compound targets a human IL-4Rα.

In some embodiments of the methods provided herein, the antisense compound is targeted to nucleotides 167-265, 487-525, 2056-2101, 2524-2598, 2731-2791, 3053-3072, or 3168-3187 of SEQ ID NO:1. In another embodiment, the compound is at least about 80%, at least 80%, at least about 85%, at least 85%, at least about 90%, at least 90%, at least about 95%, at least 95%, at least about 99% or at least 99% identical to the complement of a 20-nucleobase portion of nucleotides 167-265, 487-525, 2056-2101, 2524-2598, 2731-2791, 3053-3072, or 3168-3187 of SEQ ID NO:1. In still another embodiment, the compound is at least about 80%, at least 80%, at least about 85%, at least 85%, at least about 90%, at least 90%, at least about 95%, at least 95%, at least about 99% or at least 99% identical to the complement of a 20-nucleobase portion of nucleotides 2056-2087 of SEQ ID NO:1. In some embodiments, the compound comprises a nucleobase portion that is at least about 80%, at least 80%, at least about 85%, at least 85%, at least about 90%, at least 90%, at least about 95%, at least 95%, at least about 99% or at least 99% identical to SEQ ID NO:137, SEQ ID NO:155, SEQ ID NO:196, SEQ ID NO:276, SEQ ID NO:279, SEQ ID NO:280, SEQ ID NO:292, SEQ ID NO:298, SEQ ID NO:302 or SEQ ID NO:303. In a further embodiment, the compound is at least about 80%, at least 80%, at least about 85%, at least 85%, at least about 90%, at least 90%, at least about 95%, at least 95%, at least about 99% or at least 99% identical to SEQ ID NO:137, SEQ ID NO:155, SEQ ID NO:196, SEQ ID NO:276, SEQ ID NO:279, SEQ ID NO:280, SEQ ID NO:292, SEQ ID NO:298, SEQ ID NO:302 or SEQ ID NO:303. In another embodiment, the compound comprises SEQ ID NO:137, SEQ ID NO:155, SEQ ID NO:196, SEQ ID NO:276, SEQ ID NO:279, SEQ ID NO:280, SEQ ID NO:292, SEQ ID NO:298, SEQ ID NO:302 or SEQ ID NO:303. In yet another embodiment, the compound consists of SEQ ID NO:137, SEQ ID NO:155, SEQ ID NO:196, SEQ ID NO:276, SEQ ID NO:279, SEQ ID NO:280, SEQ ID NO:292, SEQ ID NO:298, SEQ ID NO:302 or SEQ ID NO:303. In other embodiments, the compound comprises a nucleobase portion that is at least about 50%, at least 50%, at least about 60%, at least 60%, at least about 70%, at least 70%, at least about 75%, at least 75%, at least about 80%, at least 80%, at least about 85%, at least 85%, at least about 90%, at least 90%, at least about 95%, at least 95%, at least about 99% or at least 99% to any one of SEQ ID NOS:9-306. In a further embodiment, the compound is at least about 50%, at least 50%, at least about 60%, at least 60%, at least about 70%, at least 70%, at least about 75%, at least 75%, at least about 80%, at least 80%, at least about 85%, at least 85%, at least about 90%, at least 90%, at least about 95%, at least 95%, at least about 99% or at least 99% identical to any one of SEQ ID NOS:9-306. In another embodiment, the compound comprises any one of SEQ ID NOS:9-306. In yet another embodiment, the compound consists of any one of SEQ ID NOS:9-306. While certain SEQ ID NOS are recited above, any of the antisense compounds provided herein can be suitable for the methods provided herein, including those provided in Tables 3, 4, and 5.

In some embodiments of the methods provided herein, the antisense compound is an antisense oligonucleotide. In another embodiment, the antisense compound is a siRNA. In other embodiments, the antisense compound is not a siRNA. In certain embodiments, the antisense compound is a single-stranded compound. In specific embodiments, the antisense compound comprises DNA (e.g., does not comprise the nucleotide uracil). In some embodiments, the antisense compound comprises at least one modified internucleoside linkage, sugar moiety, or nucleobase. In one embodiment, the modified internucleoside linkage is a phosphorothioate, phosphodiester, phosphotriester, methylphosphonate, phosphoramidate, methylenemethylimino, thiodiester, thionocarbamate; siloxane, N,N′-dimethylhydrazine linkage, or a combination thereof. In some embodiments, the at least one modified sugar is a bicyclic sugar, such as a bicyclic sugar comprising a 4′-CH(CH3)-O-2′ bridge. In certain embodiments, the modified sugar moiety is a 2′-O-(2-methoxyethyl) (2′-MOE) or 2′-F-(2-methoxyethyl) modification, or a combination thereof. In some embodiments, the modified nucleobase is a locked nucleic acid (LNA). In other embodiments, the modified nucleobase is a 5-methylcytosine. In specific embodiments, the antisense compound is a single stranded compound and comprises a central region of ten 2′-deoxynucleotides flanked on each side by five 2′-MOE nucleotides and phosphorothioate internucleoside linkages at each position, and optionally further comprises a 5-methylcytidine at each cytidine residue. In some embodiments, the antisense compound comprises at least one tetrahydropyran modified nucleoside, wherein a tetrahydropyran ring replaces a furanose ring. In one embodiment, each of the at least one tetra-hydropyran modified nucleoside has the structure:

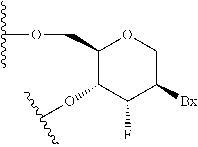

wherein Bx is an optionally protected heterocyclic base moiety. In some embodiments the antisense compound comprises a chimeric oligonucleotide. In certain embodiments, the chimeric oligonucleotide comprises a gapped motif, alternating motif, fully modified motif, hemimer motif, blockmer motif, or positionally modified motif. In one embodiment, the antisense compound is a 2′-MOE gapmer.

In certain embodiments of the methods provided herein, the antisense compound is formulated in a pharmaceutical composition. For example, in certain embodiments, the pharmaceutical composition comprises a biocompatible carrier and optionally further comprises a pharmaceutically acceptable penetration enhancer, carrier, and/or diluent.

In other embodiments of the methods provided herein, administration of more than one antisense compound is contemplated. In specific embodiments, the more than one antisense compounds are different. In certain embodiments, the more than one antisense compounds are individually selected from the group consisting of SEQ ID NO:137, SEQ ID NO:155, SEQ ID NO:196, SEQ ID NO:276, SEQ ID NO:279, SEQ ID NO:280, SEQ ID NO:292, SEQ ID NO:298, SEQ ID NO:302 or SEQ ID NO:303, but may also include any other antisense compound provided herein (e.g., in Tables 3, 4 or 5). In certain embodiments, the more than one antisense compounds are administered sequentially. A period of time, e.g., minutes, hours, days or weeks can separate the sequential administration of the more than one antisense compounds. In other embodiments, the more than one antisense compounds are administered simultaneously. In other embodiments, one or more of the antisense compounds, either alone or in combination with each other, and optionally in combination with an additional therapeutic agent, are concurrently or sequentially administered to the subject more than one time (e.g., several minutes, hours, days or months apart).

Antisense compounds described herein can be used to modulate the expression of IL-4Rα in a subject such as an animal, for example, a human. In one non-limiting embodiment, a method provided herein comprises administering (e.g., topically) to a respiratory tract of said subject an effective amount of an antisense compound that inhibits expression of IL-4Rα protein and/or expression of functional IL-4 and IL-13 receptors. In certain embodiments, the antisense compound is administered no more frequently than about once per day, such as no more frequently than about once per week. In another embodiment, the antisense compounds described herein effectively inhibit the levels or function of IL-4Rα RNA. Because reduction in IL-4Rα mRNA levels can lead to alteration in IL-4Rα protein products of expression as well, such resultant alterations can be measured based on protein level of function of the IL-4Rα protein complex in cells or in tissues of the upper or lower respiratory tract. Antisense compounds described herein that effectively inhibit the level or function of IL-4Rα RNA or protein products of expression are considered active antisense compounds.

In one embodiment of the methods, compositions and kits provided herein, the antisense compounds described herein inhibit the expression of IL-4Rα causing a reduction of IL-4Rα RNA by at least 10%, by at least 20%, by at least 25%, by at least 30%, by at least 40%, by at least 50%, by at least 60%, by at least 70%, by at least 75%, by at least 80%, by at least 85%, by at least 90%, by at least 95%, by at least 98%, by at least 99%, or by 100%. For example, the reduction of the expression of IL-4Rα can be measured in a bodily fluid, tissue, or organ of the animal. Methods of obtaining samples for analysis, such as body fluids (e.g., sputum, nasal lavage fluid, broncheoalveolar lavage fluid, urine, blood serum or plasma), tissues (e.g., biopsy), or organs, and methods of preparation of the samples to allow for analysis are well known to those skilled in the art. Methods for analysis of RNA and protein levels are discussed above and are well known to those skilled in the art. The effects of treatment can be assessed by measuring biomarkers associated with the target gene expression in the aforementioned fluids, tissues or organs, collected from an animal contacted with one or more compounds provided herein, by routine clinical methods known in the art. These biomarkers include but are not limited to: target mRNA or protein (e.g., in sputum, BAL cells, fluid or biopsy, white blood cells, lung structural cells, eosinophils, or a combination thereof), interleukins, tumor necrosis factors, intracellular adhesion molecules, C-reactive protein, inflammatory cell counts (either as absolute cell number or as a percentage of total cells, for example, eosinophil count), immunoglobulins (especially IgE), chemokines, including IL-8, MCP-1, TARC, MDC and eotaxins 1, 2 and 3 and chemokine receptors, arachidonic acid pathway components (for example, 15-HETE, leukotrienes, prostaglandins or other eicosanoids), myeloperoxidase, mast cell factors, including chymase and tryptase, eosinophil granule proteins, including eosinophil cationic protein, and eosinophil peroxidase, exhaled nitric oxide and markers of increased vascular permeability, such as alpha2-macroglobulin (albumin), and other markers of inflammation.

The antisense compounds described herein can be utilized in pharmaceutical compositions by adding an effective amount of a compound to a suitable pharmaceutically acceptable diluent or carrier. Acceptable carriers and diluents are well known to those skilled in the art. Selection of a diluent or carrier is based on a number of factors, including, but not limited to, the solubility of the compound, desired pH, and the route of administration. Such considerations are well understood by those skilled in the art. In one aspect, the antisense compounds described herein inhibit the expression of IL-4Rα. The antisense compounds described herein can also be used in the manufacture of a medicament for the treatment of diseases and disorders related to IL-4Rα expression described herein, including respiratory disorders.

Mild asthma refers to asthma that does not drastically affect the quality of the afflicted subject's life. The symptoms—including coughing, wheezing, shortness of breath, and tightness in the chest cavity—are those of chronic asthma, but they are less severe. The most common causes of asthma exacerbations are intermittent or chronic exposures to, for example, inhaled antigens or particulate materials in susceptible individuals. Upon exposure to these environmental stimuli, the airways become constricted and obstructed by inflammatory cells and mucus, which affects breathing. Mild asthma can be referred to herein as mild, controlled asthma, and can be a limited and typically controllable disease.

Treatment can require administration of multiple doses at regular intervals, or prior to exposure to an agent (e.g., an allergen) to alter the course of the condition or disease. Moreover, a single agent can be used in a single individual for each treatment of a condition or disease sequentially, or concurrently. In another embodiment, the ASOs are delivered by aerosol for topical delivery to the respiratory tract, thereby limiting systemic exposure and reducing potential side effects.

Combination Therapy

In certain embodiments, a combination of therapies (e.g., use of prophylactic or therapeutic agents) can be more effective than the additive effects of any two or more single therapy. For example, a synergistic effect of a combination of prophylactic and/or therapeutic agents permits the use of lower dosages of one or more of the agents and/or less frequent administration of said agents to a subject. The ability to utilize lower dosages of prophylactic or therapeutic therapies and/or to administer said therapies less frequently reduces the toxicity associated with the administration of said therapies to a subject without reducing the efficacy of said therapies in the prevention, management, treatment or amelioration of a disease or a symptom related thereto, such as pulmonary inflammation, airway hyperreactivity and/or loss of lung function. In addition, a synergistic effect can result in improved efficacy of therapies in the prevention, or in the management, treatment or amelioration of a disease or a symptom related thereto, such as pulmonary inflammation, airway hyperreactivity and/or loss of lung function. Finally, synergistic effect of a combination of therapies (e.g., prophylactic or therapeutic agents) may avoid or reduce adverse or unwanted side effects associated with the use of any single therapy.

In certain embodiments of the various methods provided herein, the method further comprises administering one or more additional therapeutic agents. In a specific embodiment, an effective amount of an antisense compound provided herein and an effective amount of another therapeutic agent is used. The antisense compounds provided herein can be administered alone or in combination with other types of therapies (e.g., hormonal therapy, immunotherapy, and anti-inflammatory agents). In a specific embodiment, the other therapy is one or more of a corticosteroid (e.g., an inhaled corticosteroid (ICS)), long-acting beta-2 agonist (LABA) or leukotriene antagonist (e.g., montelukast, zafirlukast or zileuton). In some embodiments, the antisense compounds provided herein act synergistically with the other therapies.

In one embodiment of the methods, compositions and kits provided herein, the therapeutic agent is a Th2 antagonist (e.g., an IL-4 antagonist (e.g., an IL-4 antibody, IL-4 receptor fusion or an IL-4 mutein), IL-5 antagonist, IL-10 antagonist, IL-9 antagonist, IL-13 antagonist, IL-4R antagonist, IL-5R antagonist, IL-10R antagonist, IL-9R antagonist and/or IL-13R antagonist), a Th1 agonist (e.g., an IFNγ agonist and/or IL-12 agonist), or a combination thereof. In some embodiments, the therapeutic agent is a cytokine, cytokine antagonist, cytokine receptor antagonist, growth factor antagonist, growth factor receptor antagonist, immunosuppressant, anti-inflammatory agent, metabolic inhibitor, enzyme inhibitor, cytostatic or cytotoxic agent (e.g., paclitaxol, cytochalasin B, gramicidin D, ethidium bromide, emetine, mitomycin, etoposide, tenoposide, anti-mitotic agent (e.g., vincristine and vinblastine), antimetabolite (e.g., methotrexate, 6-mercaptopurine, 6-thioguanine, cytarabine, 5-fluorouracil decarbazine), colchicin, anthracycline (e.g., daunorubicin (formerly daunomycin) and doxorubicin), dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof), soluble receptor, soluble Th2 cytokine receptor, peptide inhibitor, small molecule, adhesion molecule variant, antibody or fragment thereof, steroid (e.g., inhaled steroids; corticosteroids; prednisone), beta agonists (e.g., a short-acting or long-acting beta-agonist), leukotriene antagonist, leukotriene receptor antagonist, IgE antagonist, anti-IgE antibody (e.g., XOLAIR®), fluticasone and salmeterol (ADVAIR®); phosphodiesterase inhibitor (e.g., PDE4 inhibitor), eotaxin/CCR3 inhibitor; xanthine, anticholinergic agent (e.g., atropine and ipratorpium bromide), mast stabilizing agent (e.g., cromolyn), anti-histamine, hormone, vitamin, peptide, peptide analog, enzyme, narcotic, sedative, short-acting or long-acting bronchodilator, β2-agonist, theophylline, ipratorpium bromide, pirbuterol, salbutamol, levosalbutamol, levosalbutamol, clenbuterol, local anesthetic agent, general anesthetic agent, β-adrenergic drug (e.g., epinephrine and isoproterenol), theophylline, anticholinergic drug, nedocromil, TNF antagonist (e.g., a soluble fragment of a TNF receptor (e.g., p55 or p75 human TNF receptor or derivatives thereof (e.g., 75 kD TNFR-IgG fusion protein, ENBREL®)), TNF enzyme antagonist (e.g., TNFα), converting enzyme (TACE) inhibitors, muscarinic receptor antagonists, TGF-theta antagonist, perfenidone, chemotherapeutic agent (e.g., methotrexate, leflunomide, or a sirolimus (rapamycin) or an analog thereof (e.g., CCl-779; COX2 and cPLA2 inhibitor), NSAID; immunomodulator, p38 inhibitor, TPL-2, Mk-2, NF-kB inhibitor, budesonide, testosterone, progesterone, beclomethasone, momethasone, estrogen, dexamethasone, hydrocortisone, triamcinolone, flunisolide, methylprednisolone, hydrocortisone, glucocorticoid steroid, budesonide, testosterone, progesterone, estrogen, flunisolide, triamcinolone, beclomethasone, betamethasone, dexamethasone, methylprednisolone, hydrocortisone, mometasone, alkylating agent (e.g., mechlorethamine, thioepa chlorambucil, melphalan, carmustine (BSNU), lomustine (CCNU), cyclothosphamide, busulfan, dibromomannitol, streptozotocin, mitomycin C, or cisdichlorodiamine platinum (II) (DDP) cisplatin), antibiotic (e.g., dactinomycin (formerly actinomycin), bleomycin, mithramycin, and anthramycin (AMC)), drug moiety (e.g., abrin, ricin A, pseudomonas exotoxin, diphtheria toxin), protein (e.g., tumor necrosis factor, interferon-α, interferon-β, nerve growth factor (NGF), platelet derived growth factor (PDGF), tissue plasminogen activator (TPA)), apoptotic agent (e.g., TNF-α, TNF-β, AIM I (see, e.g., PCT Publication No. WO 97/33899), AIM II (see, e.g., PCT Publication No. WO 97/34911), Fas Ligand (see, e.g., Takahashi et al., J. Immunol., 6:1567-1574, 1994) or VEGF (see, e.g., PCT Publication No. WO 99/23105)), thrombotic agent, anti-angiogenic agent (e.g., angiostatin or endostatin), biological response modifier (e.g., a lymphokine (e.g., IL-1, IL-2, IL-6, or GM-CSF), G-CSF or growth factor (e.g., growth hormone), including analogues thereof or any combination thereof.

In certain embodiments of the methods, compositions and kits provided herein, the therapeutic agent is an anti-allergenic agent, anti-asthma agent, anti-viral agent, anti-bacterial agent, anti-fungal agent, anti-protozoan agent, or a combination thereof. In one embodiment, the therapeutic agent is an anti-respiratory virus therapy, such as an anti-RSV, anti-rhinovirus, anti-influenza virus (e.g., influenza A-type virus subtype H1N1 swine flu virus), anti-coronavirus (e.g., SARS virus) therapy, or a combination thereof. In some embodiments, the anti-viral agent is oseltamivir (Tamiflu®) or zanamivir (Relenza®), amantadine (Symmetrel®), rimantadine (Flumadine®), ribavirin (Copegus®, Rebetol®, Ribasphere®, Vilona® or Virazole®), RSV-IG (RespiGam®), palivizumab (Synagis®), or combination thereof. In certain embodiments, the anti-viral agent is not an anti-RSV agent (e.g., an anti-RSV antibody, such as RSV-IG or palivizumab).

Any combination of the therapeutic agents provided herein can be use in the compositions and methods provided herein.

The herein-described methods for treating a respiratory disorder, such as, a Th2-mediated respiratory disorder, in a subject can, in certain embodiments, further comprise administering to the subject being administered a composition (e.g., a pharmaceutical composition or formulation) provided herein, e.g., an antisense compound, an effective amount of one or more other therapeutic agents. In one embodiment, wherein another therapeutic agent is administered to a subject, the effective amount of the composition is less than its effective amount would be where the other therapeutic agent is not administered. In another embodiment, the effective amount of the other therapeutic agent is less than its effective amount would be where the composition is not administered.

A combination therapy can include administering an agent that reduces the side effects of other therapies, should they exist. A combinational therapy can also include administering an agent that reduces the frequency of administration of other therapies.

Useful combination therapies will be understood and appreciated by those of skill in the art. Potential advantages of such combination therapies include the ability to use less of each of the individual active ingredients (IL-4Rα ASO and other therapeutic agent or vaccine) to minimize toxic side effects, synergistic improvements in efficacy (to achieve the same effect using less of one or of each agent in combination, or administering at a decreased dose frequency either or both agents, than would be used/administered individually to achieve such effect), improved ease of administration or use, and/or reduced overall expense of compound preparation or formulation. Additivity is also contemplated, e.g., addition of AIR645 or some other IL-4Rα ASO as described herein on top of the optimal dose of standard of care therapy or therapies (e.g., addition to ICS or beta-agonist at their optimal dose).

As a result, combination therapy can achieve a reduction in the effective dose, and, thereby, the adverse effects, of an inhaled or intranasal corticosteroid, anti-histamine, leukotriene inhibitor, monoclonal antibody therapy, or bronchodilator via the addition of AIR645 or another ASO as described herein. A combination therapy can also include administering an agent that enhances the effectiveness of another therapeutic agent or vaccine, resulting in a reduction in the effective dose or dose frequency required for the other agent.

Compositions and formulations provided herein can comprise two or more oligomeric compounds. In another embodiment, compositions can contain one or more antisense compounds, particularly oligonucleotides, targeted to a first nucleic acid and one or more additional antisense compounds targeted to a second nucleic acid target. Alternatively, compositions can comprise two or more antisense compounds targeted to different regions of the same nucleic acid target. Two or more combined compounds can be used together, such as simultaneously or sequentially. Compositions can also be combined with other non-oligomeric compound therapeutic agents.

Salts, Prodrugs and Bioequivalents

The oligomeric compounds described herein can, in certain embodiments, comprise any pharmaceutically acceptable salts, esters, or salts of such esters, or any other functional chemical equivalent which, upon administration to an animal including a human, is capable of providing (directly or indirectly) the biologically active metabolite or residue thereof. Accordingly, for example, the compositions and methods provided herein can comprise prodrugs and pharmaceutically acceptable salts of the oligomeric compounds, pharmaceutically acceptable salts of such prodrugs, and other bioequivalents.

In certain embodiments prodrug versions of the oligonucleotides can be prepared as SATE ((S-acetyl-2-thioethyl) phosphate) derivatives according to the methods disclosed in WO 93/24510 or WO 94/26764. Prodrugs can also include oligomeric compounds wherein one or both ends comprise nucleobases that are cleaved (e.g., phosphodiester backbone linkages) to produce the active compound.

The pharmaceutically acceptable salts can be physiologically and pharmaceutically acceptable salts of the antisense compounds described herein: i.e., salts that retain the desired biological activity of the parent compound and do not impart undesired toxicological effects thereto. Sodium salts of antisense oligonucleotides are useful and are well accepted for therapeutic administration to humans. In another embodiment, sodium salts of dsRNA compounds are also provided.

Formulations

Provided herein are pharmaceutical compositions and formulations which include the antisense compounds described herein. For example, the antisense compounds described herein can be admixed, encapsulated, conjugated or otherwise associated with other molecules, molecule structures or mixtures of compounds. The pharmaceutical compositions can be administered in a number of ways depending on, for example, whether local or systemic treatment is desired and/or the area to be treated. In one embodiment, administration is topical to the surface of the respiratory tract, particularly nasal and pulmonary, e.g., by nebulization, inhalation, or insufflation of powders, solutions, gels, or aerosols (e.g., drops or sprays), by mouth and/or nose. For example, in additional embodiments, a once-daily inhaler or a once-weekly nebulized formulation can be used.

The pharmaceutical compositions and formulations provided herein can, in certain embodiments, be conveniently presented in unit dosage form and can be prepared according to conventional techniques well known in the pharmaceutical industry. Such techniques can include bringing into association the active ingredients with the pharmaceutical carrier(s) or excipient(s). In general, the formulations can be prepared by uniformly and intimately bringing into association the active ingredients with liquid carriers, finely divided solid carriers, or both, and then, if necessary, shaping the product (e.g., into a specific particle size for delivery). In a one embodiment, the pharmaceutical formulations are prepared for pulmonary administration in an appropriate solvent, e.g., water or normal saline, and optionally in a sterile formulation with carriers or other agents to allow for the formation of droplets of the desired diameter for delivery using inhalers, nasal delivery devices, nebulizers, and other devices for pulmonary delivery. Alternatively, the pharmaceutical formulations can be formulated as dry powders for use in dry powder inhalers.

In one aspect, provided herein is a composition (e.g., a pharmaceutical composition or formulation) comprising (i) an antigen, and (ii) an antisense compound 12 to 35 nucleobases in length targeted to a nucleic acid molecule encoding an IL-4Rα, wherein said antisense compound inhibits expression of the IL-4Rα protein and/or expression of functional IL-4 and IL-13 receptors. In some embodiments, the antigen is an antigen of a virus (viral antigen) thereof. In certain embodiments, the virus is a respiratory virus, such as a rhinovirus, influenza virus (e.g., an influenza A-type virus subtype H1N1 swine flu virus), coronavirus (e.g., SARS virus), RSV, or a combination thereof. In some embodiments, the virus is not RSV. In other embodiments, the antigen is a non-viral environmental irritant. In one embodiment, the antigen is an allergen. In other embodiments, the antigen is a bacteria, fungus, mold, dust mite, animal dander or pollen antigen, or a combination thereof. In some embodiments, the antigen is not a RSV antigen. In certain embodiments, the composition (e.g., comprising the antigen and the antisense compound, together or separate) is formulated for use in a child or adult subject. For example, in some embodiments, the composition comprises (i) an effective amount of an IL-4Rα antisense compound (e.g., a MOE oligonucleotide inhibitor of IL-4Rα), (ii) an effective amount of antigen, or (iii) an effective amount of an antisense compound IL-4Rα (e.g., a MOE oligonucleotide inhibitor of IL-4Rα) and an effective amount of antigen, for use in a child or adult subject.

In another aspect, provided herein is a composition (e.g., a pharmaceutical composition or formulation) comprising (i) a vaccine, and (ii) an antisense compound 12 to 35 nucleobases in length targeted to a nucleic acid molecule encoding an IL-4Rα, wherein said antisense compound inhibits expression of the IL-4Rα protein and/or expression of functional IL-4 and IL-13 receptors. In certain embodiments, the vaccine is a viral vaccine. In some embodiments, the vaccine is a respiratory virus vaccine, such as a rhinovirus, influenza virus (e.g., an influenza A-type virus subtype H1N1 swine flu virus), coronavirus (e.g., SARS virus), RSV vaccine, or a combination thereof. In another embodiment, the vaccine is not directed to a respiratory syncytial virus or is not a RSV vaccine. In some embodiments, the vaccine is an attenuated vaccine, inactivated vaccine, toxoid vaccine, subunit vaccine, conjugated vaccine, DNA vaccine, monovalent vaccine, multivalent vaccine, or a combination thereof. In other embodiments, the vaccine is the method of administering the composition sequentially or concurrently in combination with an attenuated vaccine, inactivated vaccine, toxoid vaccine, subunit vaccine, conjugated vaccine, DNA vaccine, monovalent vaccine, multivalent vaccine, or a combination thereof. In certain embodiments, the composition further comprises an adjuvant, which can be sequentially or concurrently administered in combination with the vaccine. In certain embodiments, a composition (e.g., comprising the vaccine and the antisense compound, together or separate) is formulated for use in a child or adult subject. For example, in some embodiments, the composition comprises (i) an effective amount of an IL-4Rα antisense compound (e.g., a MOE oligonucleotide inhibitor of IL-4Rα), (ii) an effective amount of vaccine, or (iii) an effective amount of an antisense compound IL-4Rα (e.g., a MOE oligonucleotide inhibitor of IL-4Rα) and an effective amount of vaccine, immunogen or antigen, for use in a child or adult subject.

In another aspect, provided herein is a composition (e.g., a pharmaceutical composition or formulation) provided herein comprising (i) an anti-viral therapy, and (ii) an antisense compound 12 to 35 nucleobases in length targeted to a nucleic acid molecule encoding an IL-4Rα, including chronic bronchitis, pneumonia, pulmonary fibrosis, emphysema, COPD, IPF, CF, nasal polyposis, or infection with RSV rhinovirus, influenza virus or coronavirus, wherein said antisense compound inhibits expression of the IL-4Rα protein and/or expression of functional IL-4 and IL-13 receptors. In certain embodiments, the anti-viral therapy is an anti-respiratory virus therapy, such as an anti-RSV, anti-rhinovirus, anti-influenza virus (e.g., influenza A-type virus subtype H1N1 swine flu virus), anti-coronavirus (e.g., SARS virus) therapy, or a combination thereof. In some embodiments, the anti-viral therapy is oseltamivir (Tamiflu®) or zanamivir (Relenza®), amantadine (Symmetrel®), rimantadine (Flumadine®), ribavirin (Copegus®, Rebetol®, Ribasphere®, Vilona® or Virazole®), RSV-IG (RespiGam®), palivizumab (Synagis®), or combination thereof. In certain embodiments, the anti-viral therapy is not an anti-RSV therapy (e.g., not an anti-RSV antibody, such as not RSV-IG or palivizumab).

In some embodiments of the compositions (e.g., pharmaceutical compositions or formulations) provided herein, the antisense compound is formulated to be administered by systemic administration. In other embodiments, the antisense compound is formulated to be administered by local administration. In some embodiments, the antisense compound is formulated to be administered by intranasal, intratracheal, sublingual, aerosol and/or respiratory administration. In other embodiments, the antisense compound is administered by insufflation or as a nasal spray or nasal gel. In yet other embodiments, the antisense compound is formulated to be administered using a nebulizer, nasal inhaler, metered dose inhaler, dry powder inhaler, pulmonary inhaler, or a combination thereof.

In a further embodiment of the compositions (e.g., pharmaceutical compositions or formulations) provided herein, the antisense compound 12 to 35 nucleobases in length is targeted (e.g., coding/translated region, 5′ untranslated region, 3′ untranslated region or a combination thereof, including regions spanning the translated and untranslated regions) to a nucleic acid molecule encoding an IL-4Rα, wherein said antisense compound inhibits expression of the IL-4Rα protein and/or expression of functional IL-4 and IL-13 receptors. In specific embodiments, the compound targets a human IL-4Rα.

In certain embodiments of the compositions (e.g., pharmaceutical compositions or formulations) provided herein, the antisense compound targets an 8, 9, 10, 11, 12, 13, 14, 15, 16, 17, 18, 19, 20, 21, 22, 23, 24, 25, 26, 27, 28, 29, 30, 31, 32, 33, 34 or 35 nucleobase region of human IL-4Rα (SEQ ID NO:1), wherein the target nucleobase region starts at position 21, 49, 78, 101, 167, 173, 176, 193, 194, 196, 197, 199, 200, 201, 202, 203, 205, 206, 207, 208, 209, 210, 211, 212, 213, 215, 217, 219, 220, 221, 222, 223, 224, 225, 226, 227, 228, 229, 233, 234, 244, 246, 284, 287, 317, 330, 340, 353, 355, 388, 428, 429, 430, 431, 438, 443, 487, 494, 496, 497, 499, 500, 501, 502, 503, 504, 506, 509, 510, 530, 531, 611, 619, 620, 621, 624, 642, 645, 647, 649, 716, 721, 726, 735, 736, 737, 741, 758, 777, 891, 900, 917, 918, 931, 936, 998, 999, 1000, 1001, 1003, 1004, 1005, 1006, 1014, 1053, 1077, 1078, 1079, 1080, 1082, 1083, 1085, 1087, 1088, 1090, 1092, 1093, 1094, 1095, 1096, 1098, 1100, 1104, 1114, 1121, 1126, 1131, 1136, 1160, 1175, 1182, 1221, 1223, 1224, 1225, 1227, 1364, 1385, 1390, 1395, 1397, 1398, 1399, 1400, 1401, 1424, 1430, 1435, 1440, 1492, 1499, 1505, 1506, 1507, 1508, 1509, 1510, 1515, 1575, 1608, 1670, 1671, 1673, 1674, 1676, 1700, 1701, 1703, 1705, 1706, 1708, 1716, 1777, 1779, 1780, 1781, 1782, 1834, 1839, 1844, 1845, 1880, 1960, 1976, 1991, 1996, 1997, 2000, 2001, 2006, 2011, 2038, 2043, 2056, 2057, 2058, 2059, 2060, 2062, 2064, 2065, 2066, 2067, 2068, 2079, 2082, 2084, 2087, 2126, 2128, 2130, 2131, 2166, 2204, 2230, 2301, 2315, 2390, 2403, 2437, 2442, 2467, 2469, 2497, 2502, 2506, 2507, 2524, 2526, 2528, 2529, 2530, 2531, 2532, 2541, 2548, 2569, 2578, 2579, 2626, 2643, 2674, 2719, 2731, 2743, 2751, 2763, 2772, 2788, 2793, 2804, 2813, 2827, 2836, 2856, 2859, 2869, 2909, 2915, 2952, 3048, 3053, 3103, 3169, 3198, 3235, 3290, 3297, 3303, 3327, 3340, 3345, 3350, 3350, 3355, 3420, 3432, 3477, 3572, 3578, 3559, 3671 or 3678 of SEQ ID NO:1, and extends in the 3′ direction thereof.

In other embodiments of the methods and compositions (e.g., pharmaceutical compositions or formulations) provided herein, the antisense compound targets an 8, 9, 10, 11, 12, 13, 14, 15, 16, 17, 18, 19, 20, 21, 22, 23, 24, 25, 26, 27, 28, 29, 30, 31, 32, 33, 34 or 35 nucleobase region of human IL-4Rα (SEQ ID NO:1), wherein the target nucleobase region starts at position 40, 68, 97, 120, 186, 192, 195, 112, 113, 115, 116, 118, 119, 220, 221, 222, 224, 225, 226, 227, 228, 229, 230, 231, 232, 234, 236, 238, 239, 240, 241, 242, 243, 244, 245, 246, 247, 248, 252, 253, 263, 265, 303, 306, 336, 349, 359, 372, 374, 407, 447, 448, 449, 450, 457, 462, 506, 513, 515, 516, 518, 519, 520, 521, 522, 523, 525, 528, 529, 549, 550, 630, 638, 639, 640, 643, 661, 664, 666, 668, 735, 740, 745, 754, 755, 756, 760, 777, 796, 910, 919, 936, 937, 950, 955, 1017, 1018, 1019, 1020, 1022, 1023, 1024, 1025, 1033, 1072, 1096, 1097, 1098, 1099, 1101, 1102, 1204, 1106, 1107, 1109, 1111, 1112, 1113, 1114, 1115, 1117, 1119, 1123, 1133, 1140, 1145, 1150, 1155, 1179, 1194, 1201, 1240, 1242, 1243, 1244, 1246, 1383, 1404, 1409, 1414, 1416, 1417, 1418, 1419, 1420, 1443, 1449, 1454, 1459, 1511, 1518, 1524, 1525, 1526, 1527, 1528, 1529, 1534, 1594, 1627, 1689, 1690, 1692, 1693, 1695, 1719, 1720, 1722, 1724, 1725, 1727, 1735, 1796, 1798, 1799, 1800, 1801, 1853, 1858, 1863, 1864, 1899, 1979, 1995, 2010, 2015, 2016, 2019, 2020, 2025, 2030, 2057, 2062, 2075, 2076, 2077, 2078, 2079, 2081, 2083, 2084, 2085, 2086, 2087, 2098, 2101, 2103, 2106, 2145, 2147, 2149, 2150, 2185, 2223, 2249, 2320, 2334, 2409, 2422, 2456, 2461, 2486, 2488, 2516, 2521, 2525, 2526, 2543, 2545, 2547, 2548, 2549, 2550, 2551, 2570, 2567, 2588, 2597, 2598, 2645, 2662, 2693, 2738, 2750, 2762, 2770, 2782, 2791, 2807, 2812, 2823, 2832, 2846, 2855, 2875, 2878, 2888, 2928, 2934, 2971, 3067, 3072, 3122, 3188, 3217, 3254, 3309, 3316, 3322, 3346, 3359, 3364, 3369, 3369, 3374, 3439, 3451, 3496, 3591, 3597, 3578, 3690 or 3697 of SEQ ID NO:1, and extends in the 5′ direction thereof.

In other embodiments of the methods and compositions (e.g., pharmaceutical compositions or formulations) provided herein, the antisense compound targets an 8, 9, 10, 11, 12, 13, 14, 15, 16, 17, 18, 19, 20, 21, 22, 23, 24, 25, 26, 27, 28, 29, 30, 31, 32, 33, 34 or 35 nucleobase region of human IL-4Rα (SEQ ID NO:1), wherein the target nucleobase region comprises a region that (i) starts at position 21, 49, 78, 101, 167, 173, 176, 193, 194, 196, 197, 199, 200, 201, 202, 203, 205, 206, 207, 208, 209, 210, 211, 212, 213, 215, 217, 219, 220, 221, 222, 223, 224, 225, 226, 227, 228, 229, 233, 234, 244, 246, 284, 287, 317, 330, 340, 353, 355, 388, 428, 429, 430, 431, 438, 443, 487, 494, 496, 497, 499, 500, 501, 502, 503, 504, 506, 509, 510, 530, 531, 611, 619, 620, 621, 624, 642, 645, 647, 649, 716, 721, 726, 735, 736, 737, 741, 758, 777, 891, 900, 917, 918, 931, 936, 998, 999, 1000, 1001, 1003, 1004, 1005, 1006, 1014, 1053, 1077, 1078, 1079, 1080, 1082, 1083, 1085, 1087, 1088, 1090, 1092, 1093, 1094, 1095, 1096, 1098, 1100, 1104, 1114, 1121, 1126, 1131, 1136, 1160, 1175, 1182, 1221, 1223, 1224, 1225, 1227, 1364, 1385, 1390, 1395, 1397, 1398, 1399, 1400, 1401, 1424, 1430, 1435, 1440, 1492, 1499, 1505, 1506, 1507, 1508, 1509, 1510, 1515, 1575, 1608, 1670, 1671, 1673, 1674, 1676, 1700, 1701, 1703, 1705, 1706, 1708, 1716, 1777, 1779, 1780, 1781, 1782, 1834, 1839, 1844, 1845, 1880, 1960, 1976, 1991, 1996, 1997, 2000, 2001, 2006, 2011, 2038, 2043, 2056, 2057, 2058, 2059, 2060, 2062, 2064, 2065, 2066, 2067, 2068, 2079, 2082, 2084, 2087, 2126, 2128, 2130, 2131, 2166, 2204, 2230, 2301, 2315, 2390, 2403, 2437, 2442, 2467, 2469, 2497, 2502, 2506, 2507, 2524, 2526, 2528, 2529, 2530, 2531, 2532, 2541, 2548, 2569, 2578, 2579, 2626, 2643, 2674, 2719, 2731, 2743, 2751, 2763, 2772, 2788, 2793, 2804, 2813, 2827, 2836, 2856, 2859, 2869, 2909, 2915, 2952, 3048, 3053, 3103, 3169, 3198, 3235, 3290, 3297, 3303, 3327, 3340, 3345, 3350, 3350, 3355, 3420, 3432, 3477, 3572, 3578, 3559, 3671 or 3678 of SEQ ID NO:1, and extends in the 3′ direction thereof, and (ii) starts at position 40, 68, 97, 120, 186, 192, 195, 112, 113, 115, 116, 118, 119, 220, 221, 222, 224, 225, 226, 227, 228, 229, 230, 231, 232, 234, 236, 238, 239, 240, 241, 242, 243, 244, 245, 246, 247, 248, 252, 253, 263, 265, 303, 306, 336, 349, 359, 372, 374, 407, 447, 448, 449, 450, 457, 462, 506, 513, 515, 516, 518, 519, 520, 521, 522, 523, 525, 528, 529, 549, 550, 630, 638, 639, 640, 643, 661, 664, 666, 668, 735, 740, 745, 754, 755, 756, 760, 777, 796, 910, 919, 936, 937, 950, 955, 1017, 1018, 1019, 1020, 1022, 1023, 1024, 1025, 1033, 1072, 1096, 1097, 1098, 1099, 1101, 1102, 1204, 1106, 1107, 1109, 1111, 1112, 1113, 1114, 1115, 1117, 1119, 1123, 1133, 1140, 1145, 1150, 1155, 1179, 1194, 1201, 1240, 1242, 1243, 1244, 1246, 1383, 1404, 1409, 1414, 1416, 1417, 1418, 1419, 1420, 1443, 1449, 1454, 1459, 1511, 1518, 1524, 1525, 1526, 1527, 1528, 1529, 1534, 1594, 1627, 1689, 1690, 1692, 1693, 1695, 1719, 1720, 1722, 1724, 1725, 1727, 1735, 1796, 1798, 1799, 1800, 1801, 1853, 1858, 1863, 1864, 1899, 1979, 1995, 2010, 2015, 2016, 2019, 2020, 2025, 2030, 2057, 2062, 2075, 2076, 2077, 2078, 2079, 2081, 2083, 2084, 2085, 2086, 2087, 2098, 2101, 2103, 2106, 2145, 2147, 2149, 2150, 2185, 2223, 2249, 2320, 2334, 2409, 2422, 2456, 2461, 2486, 2488, 2516, 2521, 2525, 2526, 2543, 2545, 2547, 2548, 2549, 2550, 2551, 2570, 2567, 2588, 2597, 2598, 2645, 2662, 2693, 2738, 2750, 2762, 2770, 2782, 2791, 2807, 2812, 2823, 2832, 2846, 2855, 2875, 2878, 2888, 2928, 2934, 2971, 3067, 3072, 3122, 3188, 3217, 3254, 3309, 3316, 3322, 3346, 3359, 3364, 3369, 3369, 3374, 3439, 3451, 3496, 3591, 3597, 3578, 3690 or 3697 of SEQ ID NO:1, and extends in the 5′ direction thereof.

In yet other embodiments of the methods and compositions (e.g., pharmaceutical compositions or formulations) provided herein, the antisense compound targets an 8, 9, 10, 11, 12, 13, 14, 15, 16, 17, 18, 19, 20, 21, 22, 23, 24, 25, 26, 27, 28, 29, 30, 31, 32, 33, 34 or 35 nucleobase region of human IL-4Rα (SEQ ID NO:1) spanning positions 1 and 3697 of SEQ ID NO:1, such as from or between positions 21, 49, 78, 101, 167, 173, 176, 193, 194, 196, 197, 199, 200, 201, 202, 203, 205, 206, 207, 208, 209, 210, 211, 212, 213, 215, 217, 219, 220, 221, 222, 223, 224, 225, 226, 227, 228, 229, 233, 234, 244, 246, 284, 287, 317, 330, 340, 353, 355, 388, 428, 429, 430, 431, 438, 443, 487, 494, 496, 497, 499, 500, 501, 502, 503, 504, 506, 509, 510, 530, 531, 611, 619, 620, 621, 624, 642, 645, 647, 649, 716, 721, 726, 735, 736, 737, 741, 758, 777, 891, 900, 917, 918, 931, 936, 998, 999, 1000, 1001, 1003, 1004, 1005, 1006, 1014, 1053, 1077, 1078, 1079, 1080, 1082, 1083, 1085, 1087, 1088, 1090, 1092, 1093, 1094, 1095, 1096, 1098, 1100, 1104, 1114, 1121, 1126, 1131, 1136, 1160, 1175, 1182, 1221, 1223, 1224, 1225, 1227, 1364, 1385, 1390, 1395, 1397, 1398, 1399, 1400, 1401, 1424, 1430, 1435, 1440, 1492, 1499, 1505, 1506, 1507, 1508, 1509, 1510, 1515, 1575, 1608, 1670, 1671, 1673, 1674, 1676, 1700, 1701, 1703, 1705, 1706, 1708, 1716, 1777, 1779, 1780, 1781, 1782, 1834, 1839, 1844, 1845, 1880, 1960, 1976, 1991, 1996, 1997, 2000, 2001, 2006, 2011, 2038, 2043, 2056, 2057, 2058, 2059, 2060, 2062, 2064, 2065, 2066, 2067, 2068, 2079, 2082, 2084, 2087, 2126, 2128, 2130, 2131, 2166, 2204, 2230, 2301, 2315, 2390, 2403, 2437, 2442, 2467, 2469, 2497, 2502, 2506, 2507, 2524, 2526, 2528, 2529, 2530, 2531, 2532, 2541, 2548, 2569, 2578, 2579, 2626, 2643, 2674, 2719, 2731, 2743, 2751, 2763, 2772, 2788, 2793, 2804, 2813, 2827, 2836, 2856, 2859, 2869, 2909, 2915, 2952, 3048, 3053, 3103, 3169, 3198, 3235, 3290, 3297, 3303, 3327, 3340, 3345, 3350, 3350, 3355, 3420, 3432, 3477, 3572, 3578, 3559, 3671 and/or 3678 of SEQ ID NO:1, or any region thereof.

In certain embodiments, the antisense compound targets a 19 or 20 nucleobase region of human IL-4Rα (SEQ ID NO:1) spanning positions 2056 to 2079 of SEQ ID NO:1. In specific embodiments, the antisense compound does not target a nucleobase region consisting of an 8, 9, 10, 11, 12, 13, 14, 15, 16, 17 or 18 nucleobase region of human IL-4Rα (SEQ ID NO:1) spanning positions 2056 to 2079 of SEQ ID NO:1. In certain other embodiments, the antisense compound targets a 19 or 20 nucleobase region of human IL-4Rα (SEQ ID NO:1) spanning positions 2056 to 2079 of SEQ ID NO:1. In specific embodiments, the antisense compound does not target a nucleobase region of human IL-4Rα (SEQ ID NO:1) comprising position 2051, 2052, 2053, 2054, 2055, 2080, 2081, 2082, 2083, and/or 2084 of SEQ ID NO:1. In some embodiments, the antisense compound does not target a nucleobase region of human IL-4Rα (SEQ ID NO:1) consisting of positions 2055 to 2073 of SEQ ID NO:1 In other embodiments, the antisense compound does not target a nucleobase region of human IL-4Rα (SEQ ID NO:1) consisting of or comprising the region spanning 2258 to 2282 of SEQ ID NO:1.

In some embodiments, the antisense compound does not target a nucleobase region of human IL-4Rα (SEQ ID NO:1) comprising position 486, 487, 488, 489, 490, 491, 492, 493, 494 and/or 495 of SEQ ID NO:1. In other embodiments, the antisense compound does not target a nucleobase region of human IL-4Rα (SEQ ID NO:1) comprising position 2524, 2525, 2526, 2527, 2528, 2529, 2530 and/or 2531 of SEQ ID NO:1.

In some embodiments, the antisense compound comprises at least the 19 or at least the 20 consecutive nucleobases from the 5′-terminus of SEQ ID NO:280. In other embodiments, the antisense compound comprises at least the 18, at least the 19 or at least the 20 consecutive nucleobases from the 5′-terminus of SEQ ID NO:276. In other embodiments, the antisense compound comprises at least the 15, at least the 16, at least the 17, at least the 18, at least the 19 or at least the 20 consecutive nucleobases from the 5′-terminus of SEQ ID NO:279. In other embodiments, the antisense compound comprises at least the 16, at least the 17, at least the 18, at least the 19 or at least the 20 consecutive nucleobases from the 5′-terminus of SEQ ID NO:196. In some embodiments, the antisense compound comprises at least the 12, at least the 13, at least the 14, at least the 15, at least the 16, at least the 17, at least the 18, at least the 19 or at least the 20 consecutive nucleobases from the 5′-terminus of SEQ ID NO:298. In some embodiments, the antisense compound comprises at least the 15, at least the 16, at least the 17, at least the 18, at least the 19 or at least the 20 consecutive nucleobases consecutive nucleobases from the 3′-terminus of SEQ ID NO:280. In other embodiments, the antisense compound comprises at least the 19 or at least the 20 consecutive nucleobases from the 3′-terminus of SEQ ID NO:276. In other embodiments, the antisense compound comprises at least the 16, at least the 17, at least the 18, at least the 19 or at least the 20 consecutive nucleobases from the 3′-terminus of SEQ ID NO:279. In other embodiments, the antisense compound comprises at least the 8, at least the 9, at least the 10, at least the 11, at least the 12, at least the 13, at least the 14, at least the 15, at least the 16, at least the 17, at least the 18, at least the 19 or at least the 20 consecutive nucleobases from the 3′-terminus of SEQ ID NO:196. In some embodiments, the antisense compound comprises at least the 18, at least the 19 or at least the 20 consecutive nucleobases from the 3′-terminus of SEQ ID NO:298.

In some embodiments of the compositions (e.g., pharmaceutical compositions or formulations) provided herein, the antisense compound is targeted to nucleotides 167-265, 487-525, 2056-2101, 2524-2598, 2731-2791, 3053-3072, or 3168-3187 of SEQ ID NO:1. In another embodiment, the compound is at least about 80%, at least 80%, at least about 85%, at least 85%, at least about 90%, at least 90%, at least about 95%, at least 95%, at least about 99% or at least 99% identical to the complement of a 20-nucleobase portion of nucleotides 167-265, 487-525, 2056-2101, 2524-2598, 2731-2791, 3053-3072, or 3168-3187 of SEQ ID NO:1. In still another embodiment, the compound is at least about 80%, at least 80%, at least about 85%, at least 85%, at least about 90%, at least 90%, at least about 95%, at least 95%, at least about 99% or at least 99% identical to the complement of a 20-nucleobase portion of nucleotides 2056-2087 of SEQ ID NO:1. In some embodiments, the compound comprises a nucleobase portion that is at least about 80%, at least 80%, at least about 85%, at least 85%, at least about 90%, at least 90%, at least about 95%, at least 95%, at least about 99% or at least 99% identical to SEQ ID NO:137, SEQ ID NO:155, SEQ ID NO:196, SEQ ID NO:276, SEQ ID NO:279, SEQ ID NO:280, SEQ ID NO:292, SEQ ID NO:298, SEQ ID NO:302 or SEQ ID NO:303. In a further embodiment, the compound is at least about 80%, at least 80%, at least about 85%, at least 85%, at least about 90%, at least 90%, at least about 95%, at least 95%, at least about 99% or at least 99% identical to SEQ ID NO:137, SEQ ID NO:155, SEQ ID NO:196, SEQ ID NO:276, SEQ ID NO:279, SEQ ID NO:280, SEQ ID NO:292, SEQ ID NO:298, SEQ ID NO:302 or SEQ ID NO:303. In another embodiment, the compound comprises SEQ ID NO:137, SEQ ID NO:155, SEQ ID NO:196, SEQ ID NO:276, SEQ ID NO:279, SEQ ID NO:280, SEQ ID NO:292, SEQ ID NO:298, SEQ ID NO:302 or SEQ ID NO:303. In yet another embodiment, the compound consists of SEQ ID NO:137, SEQ ID NO:155, SEQ ID NO:196, SEQ ID NO:276, SEQ ID NO:279, SEQ ID NO:280, SEQ ID NO:292, SEQ ID NO:298, SEQ ID NO:302 or SEQ ID NO:303. In other embodiments, the compound comprises a nucleobase portion that is at least about 50%, at least 50%, at least about 60%, at least 60%, at least about 70%, at least 70%, at least about 75%, at least 75%, at least about 80%, at least 80%, at least about 85%, at least 85%, at least about 90%, at least 90%, at least about 95%, at least 95%, at least about 99% or at least 99% to any one of SEQ ID NOS:9-306. In a further embodiment, the compound is at least about 50%, at least 50%, at least about 60%, at least 60%, at least about 70%, at least 70%, at least about 75%, at least 75%, at least about 80%, at least 80%, at least about 85%, at least 85%, at least about 90%, at least 90%, at least about 95%, at least 95%, at least about 99% or at least 99% identical to any one of SEQ ID NOS:9-306. In another embodiment, the compound comprises any one of SEQ ID NOS:9-306. In yet another embodiment, the compound consists of any one of SEQ ID NOS:9-306. While certain SEQ ID NOS are recited above, any of the antisense compounds provided herein can be suitable for the methods provided herein, including those provided in Tables 3, 4, and 5.

In some embodiments of the compositions (e.g., pharmaceutical compositions or formulations) provided herein, the antisense compound is an antisense oligonucleotide. In another embodiment, the antisense compound is a siRNA. In other embodiments, the antisense compound is not a siRNA. In certain embodiments, the antisense compound is a single-stranded compound. In specific embodiments, the antisense compound comprises DNA (e.g., does not comprise the nucleotide uracil). In some embodiments, the antisense compound comprises at least one modified internucleoside linkage, sugar moiety, or nucleobase. In one embodiment, the modified internucleoside linkage is a phosphorothioate, phosphodiester, phosphotriester, methylphosphonate, phosphoramidate, methylenemethylimino, thiodiester, thionocarbamate; siloxane, N,N′-dimethylhydrazine linkage, or a combination thereof. In some embodiments, the at least one modified sugar is a bicyclic sugar, such as a bicyclic sugar comprising a 4′-CH(CH₃)—O-2′ bridge. In certain embodiments, the modified sugar moiety is a 2′-MOE or 2′-F-(2-methoxyethyl) modification, or a combination thereof. In some embodiments, the modified nucleobase is a locked nucleic acid (LNA). In other embodiments, the modified nucleobase is a 5-methylcytosine. In specific embodiments, the antisense compound is a single stranded compound and comprises a central region of ten 2′-deoxynucleotides flanked on each side by five 2′-MOE nucleotides and phosphorothioate internucleoside linkages at each position, and optionally further comprises a 5-methylcytidine at each cytidine residue. In some embodiments, the antisense compound comprises at least one tetrahydropyran modified nucleoside, wherein a tetrahydropyran ring replaces a furanose ring. In one embodiment, each of the at least one tetra-hydropyran modified nucleoside has the structure:

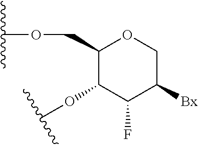

wherein Bx is an optionally protected heterocyclic base moiety. In some embodiments the antisense compound comprises a chimeric oligonucleotide. In certain embodiments, the chimeric oligonucleotide comprises a gapped motif, alternating motif, fully modified motif, hemimer motif, blockmer motif, or positionally modified motif. In one embodiment, the antisense compound is a 2′-MOE gapmer.

In certain embodiments, the compositions (e.g., pharmaceutical compositions or formulations) provided herein comprise a biocompatible carrier and optionally further comprises a pharmaceutically acceptable penetration enhancer, carrier, and/or diluent.

In other embodiments of the compositions (e.g., pharmaceutical compositions or formulations) provided herein, the composition comprises more than one antisense compound. In one embodiment, the more than one antisense compounds are different. In certain embodiments, the more than one antisense compounds are individually selected from the group consisting of SEQ ID NO:137, SEQ ID NO:155, SEQ ID NO:196, SEQ ID NO:276, SEQ ID NO:279, SEQ ID NO:280, SEQ ID NO:292, SEQ ID NO:298, SEQ ID NO:302 or SEQ ID NO:303, but may also include any other antisense compound provided herein (e.g., in Tables 3, 4 or 5).

Kits, Research Reagents, and Diagnostics

The oligomeric compounds described herein can, for example, be utilized for diagnostics, and as research reagents and kits. Furthermore, antisense compounds, which are able to inhibit gene expression with specificity, are often used by those of ordinary skill to elucidate the function of particular genes or to distinguish between functions of various members of a biological pathway.

For use in kits and diagnostics, the oligomeric compounds provided herein, either alone or in combination with other compounds or therapeutics, can be used as tools in differential and/or combinatorial analyses to elucidate expression patterns of a portion or the entire complement of genes expressed within cells and tissues. Methods of gene expression analysis are well known to those skilled in the art.

Also provided herein is a pharmaceutical pack or kit comprising one or more containers filled with one or more of the ingredients of the pharmaceutical compositions, such as one or more antisense compounds provided herein. Optionally associated with such container(s) can be a notice in the form prescribed by a governmental agency regulating the manufacture, use or sale of pharmaceuticals or biological products, which notice reflects approval by the agency of manufacture, use or sale for human administration. In certain embodiments, optionally associated with such container(s) are instructions for use.

Provided herein are kits that can be used in the methods provided herein. In one embodiment, a kit comprises one or more antisense compounds provided herein in one or more containers. In a specific embodiment, the kits of the present invention contain a control antisense compound (e.g., a mismatch control) that does not hybridize or interfere with the target sequence.

In one aspect, provided herein is a kit comprising in one or more containers (i) a viral antigen, and (ii) an antisense compound 12 to 35 nucleobases in length targeted to a nucleic acid molecule encoding an IL-4Rα, wherein said antisense compound inhibits expression of the IL-4Rα protein and/or expression of functional IL-4 and IL-13 receptors. In certain embodiments, the kit further comprises instructions for use. In some embodiments, the antigen and the antisense compound are in the same container. In other embodiments, the antigen and the antisense compound are in different containers In certain embodiments, the antigen is an antigen of a virus (viral antigen) thereof. In certain embodiments, the virus is a respiratory virus, such as a rhinovirus, influenza virus (e.g., an influenza A-type virus subtype H1N1 swine flu virus), coronavirus (e.g., SARS virus), RSV, or a combination thereof. In some embodiments, the virus is not RSV. In other embodiments, the antigen is a non-viral antigen. In one embodiment, the antigen is an allergen. In other embodiments, the antigen is a bacteria, fungus, mold, dust mite, animal dander or pollen antigen, or a combination thereof. In certain embodiments, a component of the kit is formulated for use in a child or adult subject. For example, in some embodiments, the kit comprises (i) an effective amount of an IL-4Rα antisense compound (e.g., a MOE oligonucleotide inhibitor of IL-4Rα), (ii) an effective amount of antigen, or (iii) an effective amount of an antisense compound IL-4Rα (e.g., a MOE oligonucleotide inhibitor of IL-4Rα) and an effective amount of antigen, for use in a child or adult subject.

In another aspect, provided herein is a kit comprising in one or more containers (i) a viral vaccine, and (ii) an antisense compound 12 to 35 nucleobases in length targeted to a nucleic acid molecule encoding an IL-4Rα, wherein said antisense compound inhibits expression of the IL-4Rα protein and/or expression of functional IL-4 and IL-13 receptors. In certain embodiments, the kit further comprises instructions for use. In some embodiments, the vaccine and the antisense compound are in the same container. In other embodiments, the vaccine and the antisense compound are in different containers. In certain embodiments, the antigen is an antigen of a virus (viral antigen) thereof. In certain embodiments, the virus is a respiratory virus, such as a rhinovirus, influenza virus (e.g., an influenza A-type virus subtype H1N1 swine flu virus), coronavirus (e.g., SARS virus), RSV, or a combination thereof. In some embodiments, the virus is not RSV. In other embodiments, the antigen is a non-viral antigen. In one embodiment, the antigen is an allergen. In other embodiments, the antigen is a bacteria, fungus, mold, dust mite, animal dander or pollen antigen, or a combination thereof. In some embodiments, the vaccine is an attenuated vaccine, inactivated vaccine, toxoid vaccine, subunit vaccine, conjugated vaccine, DNA vaccine, monovalent vaccine, multivalent vaccine, or a combination thereof. In other embodiments, the vaccine is the method of administering the composition sequentially or concurrently in combination with an attenuated vaccine, inactivated vaccine, toxoid vaccine, subunit vaccine, conjugated vaccine, DNA vaccine, monovalent vaccine, multivalent vaccine, or a combination thereof. In certain embodiments, the kit further comprises an adjuvant, which can be sequentially or concurrently administered in combination with the vaccine, and the adjuvant can be in the same or different container as one or more other component(s) of the kit. In certain embodiments, a component of the kit is formulated for use in a child or adult subject. For example, in some embodiments, the kit comprises (i) an effective amount of an IL-4Rα antisense compound (e.g., a MOE oligonucleotide inhibitor of IL-4Rα), (ii) an effective amount of vaccine, immunogen or antigen, or (iii) an effective amount of an antisense compound IL-4Rα (e.g., a MOE oligonucleotide inhibitor of IL-4Rα) and an effective amount of vaccine, immunogen or antigen, for use in a child or adult subject.

In another aspect, provided herein is a kit comprising in one or more containers (i) an anti-viral therapy, and (ii) an antisense compound 12 to 35 nucleobases in length targeted to a nucleic acid molecule encoding an IL-4Rα, wherein said antisense compound inhibits expression of the IL-4Rα, wherein said antisense compound inhibits expression of the IL-4Rα protein and/or expression of functional IL-4 and IL-13 receptors. In certain embodiments, the kit further comprises instructions for use. In some embodiments, the antigen and the anti-viral therapy are in the same container. In other embodiments, the antigen and the anti-viral therapy are in different containers. In certain embodiments, the anti-viral therapy is an anti-respiratory virus therapy, such as an anti-RSV, anti-rhinovirus, anti-influenza virus (e.g., influenza A-type virus subtype H1N1 swine flu virus), anti-coronavirus (e.g., SARS virus) therapy, or a combination thereof. In some embodiments, the anti-viral therapy is oseltamivir (Tamiflu®) or zanamivir (Relenza®), amantadine (Symmetrel®), rimantadine (Flumadine®), ribavirin (Copegus®, Rebetol®, Ribasphere®, Vilona® or Virazole®), RSV-IG (RespiGam®), palivizumab (Synagis®), or combination thereof. In certain embodiments, the anti-viral therapy is not an anti-RSV therapy (e.g., not an anti-RSV antibody, such as not RSV-IG or palivizumab).

In some embodiments of the kits provided herein, the antisense compound is formulated to be administered by systemic administration. In other embodiments, the antisense compound is formulated to be administered by local administration. In some embodiments, the antisense compound is formulated to be administered by intranasal, intratracheal, sublingual, aerosol and/or respiratory administration. In other embodiments, the antisense compound is administered by insufflation or as a nasal spray or nasal gel. In yet other embodiments, the antisense compound is formulated to be administered using a nebulizer, nasal inhaler, metered dose inhaler, dry powder inhaler, pulmonary inhaler, or a combination thereof.

In a further embodiment of the kits provided herein, the antisense compound 12 to 35 nucleobases in length is targeted (e.g., coding/translated region, 5′ untranslated region, 3′ untranslated region or a combination thereof, including regions spanning the translated and untranslated regions) to a nucleic acid molecule encoding an IL-4Rα, wherein said antisense compound inhibits expression of the IL-4Rα protein and/or expression of functional IL-4 and IL-13 receptors. In specific embodiments, the compound targets a human IL-4Rα.

In certain embodiments of the kits provided herein, the antisense compound targets an 8, 9, 10, 11, 12, 13, 14, 15, 16, 17, 18, 19, 20, 21, 22, 23, 24, 25, 26, 27, 28, 29, 30, 31, 32, 33, 34 or 35 nucleobase region of human IL-4Rα (SEQ ID NO:1), wherein the target nucleobase region starts at position 21, 49, 78, 101, 167, 173, 176, 193, 194, 196, 197, 199, 200, 201, 202, 203, 205, 206, 207, 208, 209, 210, 211, 212, 213, 215, 217, 219, 220, 221, 222, 223, 224, 225, 226, 227, 228, 229, 233, 234, 244, 246, 284, 287, 317, 330, 340, 353, 355, 388, 428, 429, 430, 431, 438, 443, 487, 494, 496, 497, 499, 500, 501, 502, 503, 504, 506, 509, 510, 530, 531, 611, 619, 620, 621, 624, 642, 645, 647, 649, 716, 721, 726, 735, 736, 737, 741, 758, 777, 891, 900, 917, 918, 931, 936, 998, 999, 1000, 1001, 1003, 1004, 1005, 1006, 1014, 1053, 1077, 1078, 1079, 1080, 1082, 1083, 1085, 1087, 1088, 1090, 1092, 1093, 1094, 1095, 1096, 1098, 1100, 1104, 1114, 1121, 1126, 1131, 1136, 1160, 1175, 1182, 1221, 1223, 1224, 1225, 1227, 1364, 1385, 1390, 1395, 1397, 1398, 1399, 1400, 1401, 1424, 1430, 1435, 1440, 1492, 1499, 1505, 1506, 1507, 1508, 1509, 1510, 1515, 1575, 1608, 1670, 1671, 1673, 1674, 1676, 1700, 1701, 1703, 1705, 1706, 1708, 1716, 1777, 1779, 1780, 1781, 1782, 1834, 1839, 1844, 1845, 1880, 1960, 1976, 1991, 1996, 1997, 2000, 2001, 2006, 2011, 2038, 2043, 2056, 2057, 2058, 2059, 2060, 2062, 2064, 2065, 2066, 2067, 2068, 2079, 2082, 2084, 2087, 2126, 2128, 2130, 2131, 2166, 2204, 2230, 2301, 2315, 2390, 2403, 2437, 2442, 2467, 2469, 2497, 2502, 2506, 2507, 2524, 2526, 2528, 2529, 2530, 2531, 2532, 2541, 2548, 2569, 2578, 2579, 2626, 2643, 2674, 2719, 2731, 2743, 2751, 2763, 2772, 2788, 2793, 2804, 2813, 2827, 2836, 2856, 2859, 2869, 2909, 2915, 2952, 3048, 3053, 3103, 3169, 3198, 3235, 3290, 3297, 3303, 3327, 3340, 3345, 3350, 3350, 3355, 3420, 3432, 3477, 3572, 3578, 3559, 3671 or 3678 of SEQ ID NO:1, and extends in the 3′ direction thereof.

In other embodiments of the kits provided herein, the antisense compound targets an 8, 9, 10, 11, 12, 13, 14, 15, 16, 17, 18, 19, 20, 21, 22, 23, 24, 25, 26, 27, 28, 29, 30, 31, 32, 33, 34 or 35 nucleobase region of human IL-4Rα (SEQ ID NO:1), wherein the target nucleobase region starts at position 40, 68, 97, 120, 186, 192, 195, 112, 113, 115, 116, 118, 119, 220, 221, 222, 224, 225, 226, 227, 228, 229, 230, 231, 232, 234, 236, 238, 239, 240, 241, 242, 243, 244, 245, 246, 247, 248, 252, 253, 263, 265, 303, 306, 336, 349, 359, 372, 374, 407, 447, 448, 449, 450, 457, 462, 506, 513, 515, 516, 518, 519, 520, 521, 522, 523, 525, 528, 529, 549, 550, 630, 638, 639, 640, 643, 661, 664, 666, 668, 735, 740, 745, 754, 755, 756, 760, 777, 796, 910, 919, 936, 937, 950, 955, 1017, 1018, 1019, 1020, 1022, 1023, 1024, 1025, 1033, 1072, 1096, 1097, 1098, 1099, 1101, 1102, 1204, 1106, 1107, 1109, 1111, 1112, 1113, 1114, 1115, 1117, 1119, 1123, 1133, 1140, 1145, 1150, 1155, 1179, 1194, 1201, 1240, 1242, 1243, 1244, 1246, 1383, 1404, 1409, 1414, 1416, 1417, 1418, 1419, 1420, 1443, 1449, 1454, 1459, 1511, 1518, 1524, 1525, 1526, 1527, 1528, 1529, 1534, 1594, 1627, 1689, 1690, 1692, 1693, 1695, 1719, 1720, 1722, 1724, 1725, 1727, 1735, 1796, 1798, 1799, 1800, 1801, 1853, 1858, 1863, 1864, 1899, 1979, 1995, 2010, 2015, 2016, 2019, 2020, 2025, 2030, 2057, 2062, 2075, 2076, 2077, 2078, 2079, 2081, 2083, 2084, 2085, 2086, 2087, 2098, 2101, 2103, 2106, 2145, 2147, 2149, 2150, 2185, 2223, 2249, 2320, 2334, 2409, 2422, 2456, 2461, 2486, 2488, 2516, 2521, 2525, 2526, 2543, 2545, 2547, 2548, 2549, 2550, 2551, 2570, 2567, 2588, 2597, 2598, 2645, 2662, 2693, 2738, 2750, 2762, 2770, 2782, 2791, 2807, 2812, 2823, 2832, 2846, 2855, 2875, 2878, 2888, 2928, 2934, 2971, 3067, 3072, 3122, 3188, 3217, 3254, 3309, 3316, 3322, 3346, 3359, 3364, 3369, 3369, 3374, 3439, 3451, 3496, 3591, 3597, 3578, 3690 or 3697 of SEQ ID NO:1, and extends in the 5′ direction thereof.

In other embodiments of the kits provided herein, the antisense compound targets an 8, 9, 10, 11, 12, 13, 14, 15, 16, 17, 18, 19, 20, 21, 22, 23, 24, 25, 26, 27, 28, 29, 30, 31, 32, 33, 34 or 35 nucleobase region of human IL-4Rα (SEQ ID NO:1) spanning positions 1 and 3697 of SEQ ID NO:1, such as from or between positions 21, 49, 78, 101, 167, 173, 176, 193, 194, 196, 197, 199, 200, 201, 202, 203, 205, 206, 207, 208, 209, 210, 211, 212, 213, 215, 217, 219, 220, 221, 222, 223, 224, 225, 226, 227, 228, 229, 233, 234, 244, 246, 284, 287, 317, 330, 340, 353, 355, 388, 428, 429, 430, 431, 438, 443, 487, 494, 496, 497, 499, 500, 501, 502, 503, 504, 506, 509, 510, 530, 531, 611, 619, 620, 621, 624, 642, 645, 647, 649, 716, 721, 726, 735, 736, 737, 741, 758, 777, 891, 900, 917, 918, 931, 936, 998, 999, 1000, 1001, 1003, 1004, 1005, 1006, 1014, 1053, 1077, 1078, 1079, 1080, 1082, 1083, 1085, 1087, 1088, 1090, 1092, 1093, 1094, 1095, 1096, 1098, 1100, 1104, 1114, 1121, 1126, 1131, 1136, 1160, 1175, 1182, 1221, 1223, 1224, 1225, 1227, 1364, 1385, 1390, 1395, 1397, 1398, 1399, 1400, 1401, 1424, 1430, 1435, 1440, 1492, 1499, 1505, 1506, 1507, 1508, 1509, 1510, 1515, 1575, 1608, 1670, 1671, 1673, 1674, 1676, 1700, 1701, 1703, 1705, 1706, 1708, 1716, 1777, 1779, 1780, 1781, 1782, 1834, 1839, 1844, 1845, 1880, 1960, 1976, 1991, 1996, 1997, 2000, 2001, 2006, 2011, 2038, 2043, 2056, 2057, 2058, 2059, 2060, 2062, 2064, 2065, 2066, 2067, 2068, 2079, 2082, 2084, 2087, 2126, 2128, 2130, 2131, 2166, 2204, 2230, 2301, 2315, 2390, 2403, 2437, 2442, 2467, 2469, 2497, 2502, 2506, 2507, 2524, 2526, 2528, 2529, 2530, 2531, 2532, 2541, 2548, 2569, 2578, 2579, 2626, 2643, 2674, 2719, 2731, 2743, 2751, 2763, 2772, 2788, 2793, 2804, 2813, 2827, 2836, 2856, 2859, 2869, 2909, 2915, 2952, 3048, 3053, 3103, 3169, 3198, 3235, 3290, 3297, 3303, 3327, 3340, 3345, 3350, 3350, 3355, 3420, 3432, 3477, 3572, 3578, 3559, 3671 and/or 3678 of SEQ ID NO:1, or any region thereof.

In certain embodiments, the antisense compound targets a 19 or 20 nucleobase region of human IL-4Rα (SEQ ID NO:1) spanning positions 2056 to 2079 of SEQ ID NO:1. In specific embodiments, the antisense compound does not target a nucleobase region consisting of an 8, 9, 10, 11, 12, 13, 14, 15, 16, 17 or 18 nucleobase region of human IL-4Rα (SEQ ID NO:1) spanning positions 2056 to 2079 of SEQ ID NO:1. In certain other embodiments, the antisense compound targets a 19 or 20 nucleobase region of human IL-4Rα (SEQ ID NO:1) spanning positions 2056 to 2079 of SEQ ID NO:1. In specific embodiments, the antisense compound does not target a nucleobase region of human IL-4Rα (SEQ ID NO:1) comprising position 2051, 2052, 2053, 2054, 2055, 2080, 2081, 2082, 2083, and/or 2084 of SEQ ID NO:1. In some embodiments, the antisense compound does not target a nucleobase region of human IL-4Rα (SEQ ID NO:1) consisting of positions 2055 to 2073 of SEQ ID NO:1 In other embodiments, the antisense compound does not target a nucleobase region of human IL-4Rα (SEQ ID NO:1) consisting of or comprising the region spanning 2258 to 2282 of SEQ ID NO:1.

In some embodiments, the antisense compound does not target a nucleobase region of human IL-4Rα (SEQ ID NO:1) comprising position 486, 487, 488, 489, 490, 491, 492, 493, 494 and/or 495 of SEQ ID NO:1. In other embodiments, the antisense compound does not target a nucleobase region of human IL-4Rα (SEQ ID NO:1) comprising position 2524, 2525, 2526, 2527, 2528, 2529, 2530 and/or 2531 of SEQ ID NO:1.

In some embodiments, the antisense compound comprises at least the 19 or at least the 20 consecutive nucleobases from the 5′-terminus of SEQ ID NO:280. In other embodiments, the antisense compound comprises at least the 18, at least the 19 or at least the 20 consecutive nucleobases from the 5′-terminus of SEQ ID NO:276. In other embodiments, the antisense compound comprises at least the 15, at least the 16, at least the 17, at least the 18, at least the 19 or at least the 20 consecutive nucleobases from the 5′-terminus of SEQ ID NO:279. In other embodiments, the antisense compound comprises at least the 16, at least the 17, at least the 18, at least the 19 or at least the 20 consecutive nucleobases from the 5′-terminus of SEQ ID NO:196. In some embodiments, the antisense compound comprises at least the 12, at least the 13, at least the 14, at least the 15, at least the 16, at least the 17, at least the 18, at least the 19 or at least the 20 consecutive nucleobases from the 5′-terminus of SEQ ID NO:298. In some embodiments, the antisense compound comprises at least the 15, at least the 16, at least the 17, at least the 18, at least the 19 or at least the 20 consecutive nucleobases consecutive nucleobases from the 3′-terminus of SEQ ID NO:280. In other embodiments, the antisense compound comprises at least the 19 or at least the 20 consecutive nucleobases from the 3′-terminus of SEQ ID NO:276. In other embodiments, the antisense compound comprises at least the 16, at least the 17, at least the 18, at least the 19 or at least the 20 consecutive nucleobases from the 3′-terminus of SEQ ID NO:279. In other embodiments, the antisense compound comprises at least the 8, at least the 9, at least the 10, at least the 11, at least the 12, at least the 13, at least the 14, at least the 15, at least the 16, at least the 17, at least the 18, at least the 19 or at least the 20 consecutive nucleobases from the 3′-terminus of SEQ ID NO:196. In some embodiments, the antisense compound comprises at least the 18, at least the 19 or at least the 20 consecutive nucleobases from the 3′-terminus of SEQ ID NO:298.

In some embodiments of the kits provided herein, the antisense compound is targeted to nucleotides 167-265, 487-525, 2056-2101, 2524-2598, 2731-2791, 3053-3072, or 3168-3187 of SEQ ID NO:1. In another embodiment, the compound is at least about 80%, at least 80%, at least about 85%, at least 85%, at least about 90%, at least 90%, at least about 95%, at least 95%, at least about 99% or at least 99% identical to the complement of a 20-nucleobase portion of nucleotides 167-265, 487-525, 2056-2101, 2524-2598, 2731-2791, 3053-3072, or 3168-3187 of SEQ ID NO:1. In still another embodiment, the compound is at least about 80%, at least 80%, at least about 85%, at least 85%, at least about 90%, at least 90%, at least about 95%, at least 95%, at least about 99% or at least 99% identical to the complement of a 20-nucleobase portion of nucleotides 2056-2087 of SEQ ID NO:1. In some embodiments, the compound comprises a nucleobase portion that is at least about 80%, at least 80%, at least about 85%, at least 85%, at least about 90%, at least 90%, at least about 95%, at least 95%, at least about 99% or at least 99% identical to SEQ ID NO:137, SEQ ID NO:155, SEQ ID NO:196, SEQ ID NO:276, SEQ ID NO:279, SEQ ID NO:280, SEQ ID NO:292, SEQ ID NO:298, SEQ ID NO:302 or SEQ ID NO:303. In a further embodiment, the compound is at least about 80%, at least 80%, at least about 85%, at least 85%, at least about 90%, at least 90%, at least about 95%, at least 95%, at least about 99% or at least 99% identical to SEQ ID NO:137, SEQ ID NO:155, SEQ ID NO:196, SEQ ID NO:276, SEQ ID NO:279, SEQ ID NO:280, SEQ ID NO:292, SEQ ID NO:298, SEQ ID NO:302 or SEQ ID NO:303. In another embodiment, the compound comprises SEQ ID NO:137, SEQ ID NO:155, SEQ ID NO:196, SEQ ID NO:276, SEQ ID NO:279, SEQ ID NO:280, SEQ ID NO:292, SEQ ID NO:298, SEQ ID NO:302 or SEQ ID NO:303. In yet another embodiment, the compound consists of SEQ ID NO:137, SEQ ID NO:155, SEQ ID NO:196, SEQ ID NO:276, SEQ ID NO:279, SEQ ID NO:280, SEQ ID NO:292, SEQ ID NO:298, SEQ ID NO:302 or SEQ ID NO:303. In other embodiments, the compound comprises a nucleobase portion that is at least about 50%, at least 50%, at least about 60%, at least 60%, at least about 70%, at least 70%, at least about 75%, at least 75%, at least about 80%, at least 80%, at least about 85%, at least 85%, at least about 90%, at least 90%, at least about 95%, at least 95%, at least about 99% or at least 99% to any one of SEQ ID NOS:9-306. In a further embodiment, the compound is at least about 50%, at least 50%, at least about 60%, at least 60%, at least about 70%, at least 70%, at least about 75%, at least 75%, at least about 80%, at least 80%, at least about 85%, at least 85%, at least about 90%, at least 90%, at least about 95%, at least 95%, at least about 99% or at least 99% identical to any one of SEQ ID NOS:9-306. In another embodiment, the compound comprises any one of SEQ ID NOS:9-306. In yet another embodiment, the compound consists of any one of SEQ ID NOS:9-306. While certain SEQ ID NOS are recited above, any of the antisense compounds provided herein can be suitable for the methods provided herein, including those provided in Tables 3, 4, and 5. In specific embodiments, a kit provided herein optionally further comprises a control antisense compound (e.g., a mismatch oligonucleotide) that does not target IL-4Rα.

In some embodiments of the kits provided herein, the antisense compound is an antisense oligonucleotide. In another embodiment, the antisense compound is a siRNA. In other embodiments, the antisense compound is not a siRNA. In certain embodiments, the antisense compound is a single-stranded compound. In specific embodiments, the antisense compound comprises DNA (e.g., does not comprise the nucleotide uracil). In some embodiments, the antisense compound comprises at least one modified internucleoside linkage, sugar moiety, or nucleobase. In one embodiment, the modified internucleoside linkage is a phosphorothioate, phosphodiester, phosphotriester, methylphosphonate, phosphoramidate, methylenemethylimino, thiodiester, thionocarbamate; siloxane, N,N′-dimethylhydrazine linkage, or a combination thereof. In some embodiments, the at least one modified sugar is a bicyclic sugar, such as a bicyclic sugar comprising a 4′-CH(CH₃)—O-2′ bridge. In certain embodiments, the modified sugar moiety is a 2′-O-(2-methoxyethyl) (2′-MOE) or 2′-F-(2-methoxyethyl) modification, or a combination thereof. In some embodiments, the modified nucleobase is a LNA. In other embodiments, the modified nucleobase is a 5-methylcytosine. In specific embodiments, the antisense compound is a single stranded compound and comprises a central region of ten 2′-deoxynucleotides flanked on each side by five 2′-MOE nucleotides and phosphorothioate internucleoside linkages at each position, and optionally further comprises a 5-methylcytidine at each cytidine residue. In some embodiments, the antisense compound comprises at least one tetrahydropyran modified nucleoside, wherein a tetrahydropyran ring replaces a furanose ring. In one embodiment, each of the at least one tetrahydropyran modified nucleoside has the structure:

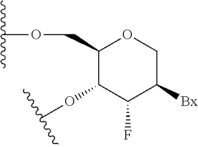

wherein Bx is an optionally protected heterocyclic base moiety. In some embodiments the antisense compound comprises a chimeric oligonucleotide. In certain embodiments, the chimeric oligonucleotide comprises a gapped motif, alternating motif, fully modified motif, hemimer motif, blockmer motif, or positionally modified motif. In one embodiment, the antisense compound is a 2′-MOE gapmer.

In certain embodiments of the kits provided herein, the antisense compound is formulated in a pharmaceutical composition. For example, in certain embodiments, the pharmaceutical composition comprises a biocompatible carrier and optionally further comprises a pharmaceutically acceptable penetration enhancer, carrier, and/or diluent.

In other embodiments of the kits provided herein, the kit comprises more than one antisense compound. In one embodiment, the more than one antisense compounds are different. In certain embodiments, the more than one antisense compounds are individually selected from the group consisting of SEQ ID NO:137, SEQ ID NO:155, SEQ ID NO:196, SEQ ID NO:276, SEQ ID NO:279, SEQ ID NO:280, SEQ ID NO:292, SEQ ID NO:298, SEQ ID NO:302 or SEQ ID NO:303, but may also include any other antisense compound provided herein (e.g., in Tables 3, 4 or 5).

Oligonucleotide Design and Screening

The effect of oligomeric compounds on target nucleic acid expression was tested in the following cell types (i) A549, and (ii) b.END.

The human lung carcinoma cell line A549 was obtained from the American Type Culture Collection (Manassas, Va.). A549 cells were routinely cultured in DMEM, high glucose (Invitrogen Life Technologies, Carlsbad, Calif.) supplemented with 10% fetal bovine serum, 100 units per ml penicillin, and 100 micrograms per ml streptomycin (Invitrogen Life Technologies, Carlsbad, Calif.). Cells were routinely passaged by trypsinization and dilution when they reached approximately 90% confluence. Cells were seeded into 96-well plates (Falcon-Primaria #3872) at a density of approximately 5000 cells/well for use in oligomeric compound transfection experiments.

The mouse brain endothelial cell line b.END was obtained from Dr. Werner Risau at the Max Plank Institute (Bad Nauheim, Germany). b.END cells were routinely cultured in DMEM, high glucose (Invitrogen Life Technologies, Carlsbad, Calif.) supplemented with 10% fetal bovine serum (Invitrogen Life Technologies, Carlsbad, Calif.). Cells were routinely passaged by trypsinization and dilution when they reached approximately 90% confluence. Cells were seeded into 96-well plates (Falcon-Primaria #353872, BD Biosciences, Bedford, Mass.) at a density of approximately 3000 cells/well for use in oligomeric compound transfection experiments.

Treatment with Oligomeric Compounds

When cells reach appropriate confluency, they are treated with oligonucleotide using a transfection lipid and method, such as Lipofectin™ essentially by the manufacturer's instructions, as described.

When cells reached 65-75% confluency, they were treated with oligonucleotide. Oligonucleotide was mixed with LIPOFECTIN™ Invitrogen Life Technologies, Carlsbad, Calif.) in Opti-MEM™-1 reduced serum medium (Invitrogen Life Technologies, Carlsbad, Calif.) to achieve the desired concentration of oligonucleotide and a LIPOFECTIN™ concentration of 2.5 or 3 μg/mL per 100 nM oligonucleotide. This transfection mixture was incubated at room temperature for approximately 0.5 hours. For cells grown in 96-well plates, wells were washed once with 100 μL OPTI-MEM™-1 and then treated with 130 μL of the transfection mixture. Cells grown in 24-well plates or other standard tissue culture plates are treated similarly, using appropriate volumes of medium and oligonucleotide. Cells are treated and data are obtained in duplicate or triplicate. After approximately 4-7 hours of treatment at 37° C., the medium containing the transfection mixture was replaced with fresh culture medium. Cells were harvested 16-24 hours after oligonucleotide treatment.

Other transfection reagents and methods (e.g., electroporation) for delivery of oligonucleotides to the cell are well known. The method of delivery of oligonucleotide to the cells is not a limitation of the methods provided herein.

Control Oligonucleotides

Control oligonucleotides are used to determine the optimal oligomeric compound concentration for a particular cell line. Furthermore, when oligomeric compounds are tested in oligomeric compound screening experiments or phenotypic assays, control oligonucleotides can be tested in parallel with the compounds.

The concentration of oligonucleotide used varies from cell line to cell line. To determine the optimal oligonucleotide concentration for a particular cell line, the cells are treated with a positive control oligonucleotide at a range of concentrations. The concentration of positive control oligonucleotide that results in 80% inhibition of the target mRNA is then utilized as the screening concentration for new oligonucleotides in subsequent experiments for that cell line. If 80% inhibition is not achieved, the lowest concentration of positive control oligonucleotide that results in 60% inhibition of the target mRNA is then utilized as the oligonucleotide screening concentration in subsequent experiments for that cell line. If 60% inhibition is not achieved, that particular cell line is deemed as unsuitable for oligonucleotide transfection experiments. The concentrations of antisense oligonucleotides used herein are from 50 nM to 300 nM when the antisense oligonucleotide is transfected using a liposome reagent and 1 μM to 40 μM when the antisense oligonucleotide is transfected by electroporation.

Real-time Quantitative PCR Analysis of IL-4Rα mRNA Levels

Quantitation of IL-4Rα mRNA levels was accomplished by real-time quantitative PCR using the ABI PRISM™ 7600, 7700, or 7900 Sequence Detection System (PE-Applied Biosystems, Foster City, Calif.) according to manufacturer's instructions.

Prior to quantitative PCR analysis, primer-probe sets specific to the target gene being measured were evaluated for their ability to be “multiplexed” with a GAPDH amplification reaction. After isolation the RNA is subjected to sequential reverse transcriptase (RT) reaction and real-time PCR, both of which are performed in the same well. RT and PCR reagents were obtained from Invitrogen Life Technologies (Carlsbad, Calif.). RT, real-time PCR was carried out in the same by adding 20 μL PCR cocktail (2.5×PCR buffer minus MgCl₂, 6.6 mM MgCl₂, 375 μM each of dATP, dCTP, dCTP and dGTP, 375 nM each forward primer and reverse primer, 125 nM of probe, 4 Units RNAse inhibitor, 1.25 Units PLATINUM® Taq, 5 Units MuLV reverse transcriptase, and 2.5×ROX dye) to 96-well plates containing 30 μL total RNA solution (20-200 ng). The RT reaction was carried out by incubation for 30 minutes at 48° C. Following a 10 minute incubation at 95° C. to activate the PLATINUM® Taq, 40 cycles of a two-step PCR protocol were carried out: 95° C. for 15 seconds (denaturation) followed by 60° C. for 1.5 minutes (annealing/extension).

Gene target quantities obtained by RT, real-time PCR were normalized using either the expression level of GAPDH, a gene whose expression is constant, or by quantifying total RNA using RiboGreen™ (Molecular Probes, Inc., Eugene, Oreg.). GAPDH expression was quantified by RT, real-time PCR, by being run simultaneously with the target, multiplexing, or separately. Total RNA was quantified using RiboGreen™ RNA quantification reagent (Molecular Probes, Inc., Eugene, Oreg.).

170 μL of RiboGreen™ working reagent (RiboGreen™ reagent diluted 1:350 in 10 mM Tris-HCl, 1 mM EDTA, pH 7.5) was pipetted into a 96-well plate containing 30 μL purified cellular RNA. The plate was read in a CytoFluor 4000 (PE Applied Biosystems) with excitation at 485 nm and emission at 530 nm.

The GAPDH PCR probes have JOE covalently linked to the 5′ end and TAMRA or MGB covalently linked to the 3′ end, where JOE is the fluorescent reporter dye and TAMRA or MGB is the quencher dye. In some cell types, primers and probe designed to a GAPDH sequence from a different species are used to measure GAPDH expression. For example, a human GAPDH primer and probe set is used to measure GAPDH expression in monkey-derived cells and cell lines.

Probes and primers for use in real-time PCR were designed to hybridize to target-specific sequences. The primers and probes and the target nucleic acid sequences to which they hybridize are presented in Table 2. The target-specific PCR probes have FAM covalently linked to the 5′ end and TAMRA or MGB covalently linked to the 3′ end, where FAM is the fluorescent dye and TAMRA or MGB is the quencher dye.

TABLE 2
Gene target-specific primers and probes for use in real-time PCR

| | | Target | | | SEQ |
|---|---|---|---|---|---|
| Target | | SEQ | Sequence | | ID |
| Name | Species | ID NO | Description | Sequence (5′ to 3′) | NO |
| IL-4R | Human | 1 | Fwd Primer | AATGGTCCCACCAATTGCA | 3 |
| alpha | | | | | |

| | | | | | |
|---|---|---|---|---|---|
| IL-4R | Human | 1 | Reverse Primer | CTCCGTTGTTCTCAGGGATACAC | 4 |
| alpha | | | | | |
| IL-4R | Human | 1 | Probe | TTTTTCTGCTCTCCGAAGCCC | 5 |
| alpha | | | | | |
| IL-4R | Mouse | 2 | Fwd Primer | TCCCATTTTGTCCACCGAATA | 6 |
| alpha | | | | | |
| IL-4R | Mouse | 2 | Reverse Primer | GTTTCTAGGCCCAGCTTCCA | 7 |
| alpha | | | | | |
| IL-4R | Mouse | 2 | Probe | TGTCACTCAAGGCTCTCAGCGGTCC | 8 |
| alpha | | | | | |

Antisense Inhibition of Mouse IL-4R Alpha by Oligomeric Compounds

A series of oligomeric compounds was designed to target different regions of mouse IL-4Rα RNA, using published sequences cited in Table 1. The compounds are shown in Table 3. All compounds in Table 3 are chimeric oligonucleotides (“gapmers”) 20 nucleotides in length, composed of a central “gap” region consisting of 10 2′-deoxynucleotides, which is flanked on both sides (5′ and 3′) by five-nucleotide “wings”. The wings are composed of 2′-β-(2-methoxyethyl) nucleotides, also known as 2′-MOE nucleotides. The internucleoside (backbone) linkages are phosphorothioate throughout the oligonucleotide. All cytidine residues are 5-methylcytidines. The compounds were analyzed for their effect on gene target mRNA levels by quantitative real-time PCR as described in other examples herein, using the target-specific primers and probes shown in Table 2. Data are averages from two experiments in which b.END cells were treated with 150 nM of the compounds in Table 3 using Lipofectin™. A reduction in expression is expressed as percent inhibition in Table 3. If the target expression level of oligomeric compound-treated cell was higher than control, percent inhibition is expressed as zero inhibition. The target regions to which these oligomeric compounds are inhibitory are herein referred to as “validated target segments.”

TABLE 3
Inhibition of mouse IL-4Rα mRNA levels by chimeric oligonucleotides
having 2′-MOE wings and deoxy gap

| | Target | | | | |
|---|---|---|---|---|---|
| | SEQ ID/ | Target | | % | SEQ |
| ISIS # | GenBank | Site | Sequence (5′ to 3′) | Inhib | ID NO |
| 231931 | Assm. fr. | 1364 | ACCCGCACAAGGTCCTGGGC | 20 | 9 |
| | M64868.1/ | | | | |
| | M64879.1 | | | | |
| 231932 | Assm. fr. | 2204 | CAGGTCTTACCATTACCACT | 33 | 10 |
| | M64868.1/ | | | | |
| | M64879.1 | | | | |
| 231933 | Assm. fr. | 2506 | GCCCACTCACTTCTGCAGGG | 50 | 11 |
| | M64868.1/ | | | | |
| | M64879.1 | | | | |
| 231934 | Assm. fr. | 2804 | CGGTTGTACCACGTGATGCT | 51 | 12 |
| | M64868.1/ | | | | |
| | M64879.1 | | | | |
| 231935 | Assm. fr. | 2813 | TGATACTCACGGTTGTACCA | 29 | 13 |
| | M64868.1/ | | | | |
| | M64879.1 | | | | |
| 231936 | Assm. fr. | 3327 | AGGAACTCACTTGGTAATGC | 9 | 14 |
| | M64868.1/ | | | | |
| | M64879.1 | | | | |
| 231937 | Assm. fr. | 3559 | TGTACCCTCTTACCTGTGCA | 30 | 15 |
| | M64868.1/ | | | | |
| | M64879.1 | | | | |
| 231929 | BB867141.1 | 49 | CAAAAGGTGCCTGCGAGTTC | 19 | 16 |
| 231930 | BC012309.1 | 101 | GGCTGGGTTACAGGAACAAG | 0 | 17 |
| 231928 | M27959.1 | 900 | AGCTGGAAGTGGTTGTACCA | 23 | 18 |
| 231860 | 2 | 78 | AATCAGAAGCCAGGTCCCTC | 66 | 19 |
| 231861 | 2 | 209 | CAAAAGGTGCCTGCACAAGG | 34 | 20 |
| 231862 | 2 | 233 | TGCAAAGCCGCCCCATTGGG | 66 | 21 |
| 231863 | 2 | 244 | CAGGAACTTGGTGCAAAGCC | 60 | 22 |
| 231864 | 2 | 330 | TAGTCAGAGAAGCAGGTGGG | 44 | 23 |
| 231865 | 2 | 340 | AGTGCGGATGTAGTCAGAGA | 39 | 24 |
| 231866 | 2 | 388 | CTGAGAACTGCAGTCCACAG | 58 | 25 |
| 231867 | 2 | 438 | GTGAGGTTTTCAGAGAACTC | 28 | 26 |
| 231868 | 2 | 443 | TGCATGTGAGGTTTTCAGAG | 48 | 27 |
| 231869 | 2 | 611 | GTGTGAGGTTGTCTGGAGCT | 63 | 28 |
| 231870 | 2 | 624 | ACATTGGTGTGGAGTGTGAG | 38 | 29 |
| 231871 | 2 | 716 | CTCTGGAGATGTTGACCATG | 48 | 30 |
| 231872 | 2 | 721 | GTCCTCTCTGGAGATGTTGA | 43 | 31 |
| 231873 | 2 | 726 | GGGTTGTCCTCTCTGGAGAT | 32 | 32 |
| 231874 | 2 | 758 | TGTAGGTCACATTATAGACT | 66 | 33 |
| 231875 | 2 | 891 | GGGTTGTACCACGTGATGCT | 27 | 34 |
| 231876 | 2 | 918 | TCACTCAGTCACAGATTTTC | 70 | 35 |
| 231877 | 2 | 1014 | AGCTGGAAGTCCATCTCCTG | 23 | 36 |
| 231878 | 2 | 1114 | CTTCTTAATCTTGGTAATGC | 40 | 37 |
| 231879 | 2 | 1121 | ACCATATCTTCTTAATCTTG | 25 | 38 |
| 231880 | 2 | 1126 | GTCCCACCATATCTTCTTAA | 11 | 39 |
| 231881 | 2 | 1131 | ATCTGGTCCCACCATATCTT | 36 | 40 |
| 231882 | 2 | 1136 | TGGGAATCTGGTCCCACCAT | 43 | 41 |
| 231883 | 2 | 1225 | GGTTGACTCCTGGCTTCGGG | 7 | 42 |
| 231884 | 2 | 1385 | GGACGGTCCTGCTGACCTCC | 65 | 43 |
| 231885 | 2 | 1390 | CCAGAGGACGGTCCTGCTGA | 55 | 44 |
| 231886 | 2 | 1395 | TCTGGCCAGAGGACGGTCCT | 65 | 45 |
| 231887 | 2 | 1424 | TACAGCGCACCACACTGACA | 80 | 46 |
| 231888 | 2 | 1430 | GCTCCATACAGCGCACCACA | 72 | 47 |
| 231889 | 2 | 1435 | AAACAGCTCCATACAGCGCA | 77 | 48 |
| 231890 | 2 | 1440 | GCCTCAAACAGCTCCATACA | 58 | 49 |
| 231891 | 2 | 1460 | CCTCCACATTCTGTACTGGG | 75 | 50 |
| 231892 | 2 | 1505 | CAGGTGACATGCTCAGGTCC | 63 | 51 |
| 231893 | 2 | 1510 | GTTCTCAGGTGACATGCTCA | 68 | 52 |
| 231894 | 2 | 1515 | CCGCTGTTCTCAGGTGACAT | 88 | 53 |
| 231895 | 2 | 1575 | AACAGGTTCTCAGTGAGCCG | 62 | 54 |
| 231896 | 2 | 1834 | CCGGTAGGCAGGATTGTCTG | 62 | 55 |
| 231897 | 2 | 1839 | AAACTCCGGTAGGCAGGATT | 68 | 56 |
| 231898 | 2 | 1844 | CACTAAAACTCCGGTAGGCA | 71 | 57 |
| 231899 | 2 | 1880 | CCAGCTCTCCAGGATTTGGG | 68 | 58 |
| 231900 | 2 | 1960 | TGGTGGCCCTGAAGAATGGG | 30 | 59 |
| 231901 | 2 | 1991 | GGATCTGCTCCCAGCTCTCC | 85 | 60 |
| 231902 | 2 | 1996 | GTGAAGGATCTGCTCCCAGC | 80 | 61 |
| 231903 | 2 | 2001 | CTCATGTGAAGGATCTGCTC | 69 | 62 |
| 231904 | 2 | 2006 | GGACACTCATGTGAAGGATC | 52 | 63 |
| 231905 | 2 | 2011 | CTGCAGGACACTCATGTGAA | 67 | 64 |
| 231906 | 2 | 2079 | TTCACTGCCTGCACAAACTC | 60 | 65 |
| 231907 | 2 | 2084 | CCTGCTTCACTGCCTGCACA | 72 | 66 |
| 231908 | 2 | 2166 | CTGCTGAGCAGGCTCGAGAA | 51 | 67 |
| 231909 | 2 | 2437 | GTCATCCCCAAAGGGCTTGG | 69 | 68 |
| 231910 | 2 | 2442 | CCCAGGTCATCCCCAAAGGG | 71 | 69 |
| 231911 | 2 | 2469 | GTGAGGGACGAGTACACAAT | 68 | 70 |
| 231912 | 2 | 2497 | TTGCTTCAGGTGGCCACACA | 69 | 71 |
| 231913 | 2 | 2502 | TGGTGTTGCTTCAGGTGGCC | 71 | 72 |
| 231914 | 2 | 2507 | GGCTGTGGTGTTGCTTCAGG | 51 | 73 |
| 231915 | 2 | 2719 | CTGGCTGGGAACAGGAGAGT | 64 | 74 |
| 231916 | 2 | 2788 | AGCAACAACAGCACACTCAC | 78 | 75 |
| 231917 | 2 | 2793 | ACCTCAGCAACAACAGCACA | 82 | 76 |
| 231918 | 2 | 2798 | CACAGACCTCAGCAACAACA | 78 | 77 |
| 231919 | 2 | 2827 | TCCCTGGCTTGGAGGAACCC | 62 | 78 |
| 231920 | 2 | 2859 | CCTGCCAGCTGGGCTGTCTC | 66 | 79 |
| 231921 | 2 | 2869 | TTCTGGGAAACCTGCCAGCT | 73 | 80 |
| 231922 | 2 | 3340 | ACTTTGGGCAATCAAGTTTG | 32 | 81 |
| 231923 | 2 | 3345 | CAGTGACTTTGGGCAATCAA | 64 | 82 |
| 231924 | 2 | 3350 | ACTGGCAGTGACTTTGGGCA | 59 | 83 |
| 231925 | 2 | 3355 | GGGTAACTGGCAGTGACTTT | 56 | 84 |
| 231926 | 2 | 3671 | TAAAGACTTTATTGACATAA | 41 | 85 |
| 231927 | 2 | 3678 | GACAAGATAAAGACTTTATT | 41 | 86 |

All oligonucleotides targeted to the following regions of a GenBank sequence assembled from assembled from M64868.1 and M64879.1 were effective at inhibiting expression of IL-4Rα at least 40% as can be determined by the table above: nucleotides 2506-2525 and 2084-2323. These are validated target segments. All oligonucleotides targeted to the following regions of SEQ ID NO:2 were effective at inhibiting expression of IL-4Rα at least 40% as can be determined by the table above: nucleotides 78-97; 233-263; 330-349; 388-407; 443-462; 611-630; 716-740; 758-777; 918-937; 1014-1033; 1114-1133; 1136-1155; 1385-1414; 1424-1459; 1505-1534; 1575-1594; 1834-1863; 1880-1899; 1991-2030; 2079-2103; 2166-2185; 2437-2461; 2469-2488; 2497-2526; 2719-2738; 2788-2817; 2827-2846; 2859-2888; 3345-3374; and 3671-3697. These are validated target segments, and antisense compounds targeting these segments (or ranges thereof) are contemplated for use in the compositions, methods and kits provided herein.

Antisense Inhibition of Human IL-4Rα by Oligomeric Compounds

A series of oligomeric compounds was designed to target different regions of human IL-4Rα RNA, using published sequences cited in Table 1. The compounds are shown in Tables 4 and 5. All compounds in Tables 4 and 5 are chimeric oligonucleotides (“gapmers”) 20 nucleotides in length, composed of a central “gap” region consisting of 10 2′-deoxynucleotides, which is flanked on both sides (5′ and 3′) by five-nucleotide “wings”. The wings are composed of 2′-O-(2-methoxyethyl) nucleotides, also known as 2′-MOE nucleotides. The internucleoside (backbone) linkages are phosphorothioate throughout the oligonucleotide. All cytidine residues are 5-methylcytidines. The compounds were analyzed for their effect on gene target mRNA levels by quantitative real-time PCR as described in other examples herein, using the human target-specific primers and probes shown in Table 2. Data are averages from two experiments in which A549 cells were treated with 85 nM of the compounds in Table 4, and 70 nM of the compound in Table 5, using Lipofectin™. A reduction in expression is expressed as percent inhibition in Tables 4 and 5. If the target expression level of oligomeric compound-treated cell was higher than control, percent inhibition is expressed as zero inhibition. The target regions to which these oligomeric compounds are inhibitory are herein referred to as “validated target segments.”

TABLE 4
Inhibition of human IL-4Rα mRNA levels by chimeric oligonucleotides
having 2′-MOE wings and deoxy gap

| | Target | | | | |
|---|---|---|---|---|---|
| | SEQ ID | Target | | % | SEQ |
| ISIS # | NO | Site | Sequence (5′ to 3′) | Inhib | ID NO |
| 364941 | 1 | 21 | GTAAATCTTTAATTATCTGC | 9 | 87 |
| 364945 | 1 | 234 | CATGTTCCCAGAGCTTGCCA | 19 | 88 |
| 364946 | 1 | 246 | CTGCAAGACCTTCATGTTCC | 40 | 89 |
| 364947 | 1 | 287 | CAAGTAGAGATGCTCATGTA | 30 | 90 |
| 364948 | 1 | 317 | CAATTGGTGGGACCATTCAT | 36 | 91 |
| 364949 | 1 | 487 | ACAGCAGCTGCTGCCCAGCC | 51 | 92 |
| 364951 | 1 | 741 | AATCCCAGACTTCAGGGTGC | 45 | 93 |
| 364952 | 1 | 777 | GCACTGAGCCCAGGCCCTCA | 56 | 94 |
| 364953 | 1 | 917 | CTGACATAGCACAACAGGCA | 55 | 95 |
| 364954 | 1 | 931 | TAATCTTGGTGATGCTGACA | 48 | 96 |
| 364955 | 1 | 936 | TTTCTTAATCTTGGTGATGC | 25 | 97 |
| 364958 | 1 | 1160 | CCCTGGAAAGGCATCTCTTT | 54 | 98 |
| 364959 | 1 | 1175 | GCTGATTTTCCAGAGCCCTG | 59 | 99 |
| 364960 | 1 | 1182 | GCACCATGCTGATTTTCCAG | 57 | 100 |
| 364962 | 1 | 1492 | CCCAGGGCATGTGAGCACTC | 49 | 101 |
| 364963 | 1 | 1499 | AACTCATCCCAGGGCATGTG | 59 | 102 |
| 364964 | 1 | 1509 | TGCACTTGGGAACTCATCCC | 49 | 103 |
| 364965 | 1 | 1608 | GCAAGTCAGGTTGTCTGGAC | 54 | 104 |
| 364966 | 1 | 1708 | GTGGGTCTGGACCCAGCTCT | 46 | 105 |
| 364967 | 1 | 1716 | GGCCAGCAGTGGGTCTGGAC | 48 | 106 |
| 364968 | 1 | 1845 | TGCCCCATGCTGGAGGACAT | 37 | 107 |
| 364969 | 1 | 1976 | GAGAAGGCCTTGTAACCAGC | 53 | 108 |
| 364970 | 1 | 2000 | ACAGCACTGCTGGCAAGCAG | 35 | 109 |
| 364971 | 1 | 2038 | CCCCACTGCTAGCCCCAAAC | 24 | 110 |
| 364972 | 1 | 2043 | CTCTTCCCCACTGCTAGCCC | 25 | 111 |
| 364973 | 1 | 2058 | GAAAGGCTTATACCCCTCTT | 62 | 112 |
| 364974 | 1 | 2067 | GAGGTCTTGGAAAGGCTTAT | 55 | 113 |
| 364975 | 1 | 2082 | AGGGCAGCCAGGAATGAGGT | 42 | 114 |
| 364976 | 1 | 2087 | TCCCCAGGGCAGCCAGGAAT | 37 | 115 |
| 364977 | 1 | 2230 | GCTTTGGCATGTCCTCTACC | 50 | 116 |
| 364978 | 1 | 2301 | GGCTGAGTAGACAATGCCAC | 26 | 117 |
| 364979 | 1 | 2315 | AGGTGGCAGGTAAGGGCTGA | 39 | 118 |
| 364980 | 1 | 2390 | CCACAGCAAGGACTGGCCAT | 45 | 119 |
| 364981 | 1 | 2469 | CAGTGGAACCCCACCTGGAG | 23 | 120 |
| 364983 | 1 | 2541 | GAAGGATGATGAGGATTTAC | 51 | 121 |
| 364984 | 1 | 2548 | CAGGATGGAAGGATGATGAG | 41 | 122 |
| 364985 | 1 | 2569 | AGCTCTGAGCATTGCCAGGG | 45 | 123 |
| 364986 | 1 | 2626 | CCCTCATGTATGTGGGTCCC | 48 | 124 |
| 364987 | 1 | 2643 | GACATGCACCTAAGAGACCC | 49 | 125 |
| 364988 | 1 | 2674 | TAGTCCTCATCTGCAGACTC | 50 | 126 |
| 364989 | 1 | 2731 | AATCTGCCAGCCTGGCTGCC | 41 | 127 |
| 364991 | 1 | 2751 | GGTTCTTCAAGTCTTTTGGA | 56 | 128 |
| 364993 | 1 | 2772 | GGCCAATCACCTTCATACCA | 47 | 129 |
| 364994 | 1 | 2836 | GAGCCCAGCCCAATGCTGGG | 7 | 130 |
| 364995 | 1 | 2856 | CTACTCTCATGGGATGTGGC | 61 | 131 |
| 364996 | 1 | 2861 | GCCCTCTACTCTCATGGGAT | 58 | 132 |
| 364997 | 1 | 2909 | GGCCTCAGTTTTCCTGCAGG | 30 | 133 |
| 364998 | 1 | 2915 | CCCAAGGGCCTCAGTTTTCC | 55 | 134 |
| 364999 | 1 | 2952 | GAGGGAGCAGCCAACAACTC | 31 | 135 |
| 365000 | 1 | 3048 | AGACAGAGGCAGGTGGGCCT | 35 | 136 |
| 365001 | 1 | 3053 | CAGTGAGACAGAGGCAGGTG | 63 | 137 |
| 365002 | 1 | 3103 | CAAGTCATTCCCTTGATGGC | 48 | 138 |
| 365004 | 1 | 3198 | ATCAACCTAAGGAAGCTCTG | 49 | 139 |
| 365005 | 1 | 3238 | TAACTGAACACCCCTTGACA | 6 | 140 |
| 365006 | 1 | 3290 | AATTGTCCCTGCTTTAGTCA | 16 | 141 |
| 365007 | 1 | 3297 | GGCAGCAAATTGTCCCTGCT | 55 | 142 |
| 365008 | 1 | 3303 | GTGTTTGGCAGCAAATTGTC | 46 | 143 |
| 365009 | 1 | 3420 | GGGTAACTGGTGCCTTATGC | 53 | 144 |
| 365010 | 1 | 3432 | GGCCAACATGCAGGGTAACT | 44 | 145 |
| 365011 | 1 | 3477 | ATTACTCAACCCAAGGTTCC | 20 | 146 |
| 365012 | 1 | 3572 | AAGAAACTTTATTTATACAA | 0 | 147 |
| 365013 | 1 | 3578 | GAGACAAAGAAACTTTATTT | 2 | 148 |
| 365014 | 18636000- | 8231 | CCTAGAATTCAGTCTTCCCT | 41 | 149 |
| | 18639000 | | | | |
| | of NT_01 | | | | |
| | 0393.14 | | | | |
| 365015 | 18636000- | 20215 | GTTTCCATCTAGAGTACTAG | 35 | 150 |
| | 18639000 | | | | |
| | of NT_01 | | | | |
| | 0393.14 | | | | |
| 365016 | 18636000- | 27651 | GCCAAGGCACCTGCAGAGAG | 38 | 151 |
| | 18639000 | | | | |
| | of NT_01 | | | | |
| | 0393.14 | | | | |
| 365017 | 18636000- | 47104 | AGTGAGTGGCAGAGTCAGGA | 48 | 152 |
| | 18639000 | | | | |
| | of NT_01 | | | | |
| | 0393.14 | | | | |
| 365018 | 18636000- | 49717 | CTTCCAGTGTCTGCAAAAGC | 0 | 153 |
| | 18639000 | | | | |
| | of NT_01 | | | | |
| | 0393.14 | | | | |

TABLE 5
Inhibition of human IL-4Rα mRNA levels by chimeric oligonucleotides
having 2′-MOE wings and deoxy gap

| | Target | | | | |
|---|---|---|---|---|---|
| | SEQ | Target | | % | SEQ |
| ISIS # | ID NO | Site | Sequence (5′ to 3′) | Inhib | ID NO |
| 364942 | 1 | 167 | AGCCACCCCATTGGGAGATG | 88 | 154 |
| 364943 | 1 | 173 | GAGCAAAGCCACCCCATTGG | 83 | 155 |
| 369527 | 1 | 176 | CCAGAGCAAAGCCACCCCAT | 51 | 156 |
| 369528 | 1 | 193 | TCACAGGGAACAGGAGCCCA | 48 | 157 |
| 369529 | 1 | 194 | CTCACAGGGAACAGGAGCCC | 62 | 158 |
| 369530 | 1 | 196 | AGCTCACAGGGAACAGGAGC | 44 | 159 |
| 369531 | 1 | 197 | CAGCTCACAGGGAACAGGAG | 54 | 160 |
| 369532 | 1 | 199 | GGCAGCTCACAGGGAACAGG | 69 | 161 |
| 369533 | 1 | 200 | AGGCAGCTCACAGGGAACAG | 64 | 162 |
| 369534 | 1 | 201 | CAGGCAGCTCACAGGGAACA | 64 | 163 |
| 369535 | 1 | 202 | CCAGGCAGCTCACAGGGAAC | 58 | 164 |
| 369536 | 1 | 203 | ACCAGGCAGCTCACAGGGAA | 65 | 165 |
| 369537 | 1 | 205 | GGACCAGGCAGCTCACAGGG | 63 | 166 |
| 369538 | 1 | 206 | AGGACCAGGCAGCTCACAGG | 74 | 167 |
| 369539 | 1 | 207 | CAGGACCAGGCAGCTCACAG | 66 | 168 |
| 369540 | 1 | 208 | GCAGGACCAGGCAGCTCACA | 57 | 169 |
| 369541 | 1 | 209 | AGCAGGACCAGGCAGCTCAC | 48 | 170 |
| 369542 | 1 | 210 | CAGCAGGACCAGGCAGCTCA | 46 | 171 |
| 369543 | 1 | 211 | GCAGCAGGACCAGGCAGCTC | 48 | 172 |
| 369544 | 1 | 212 | TGCAGCAGGACCAGGCAGCT | 39 | 173 |
| 369545 | 1 | 213 | CTGCAGCAGGACCAGGCAGC | 22 | 174 |
| 369546 | 1 | 215 | ACCTGCAGCAGGACCAGGCA | 38 | 175 |
| 369547 | 1 | 217 | CCACCTGCAGCAGGACCAGG | 63 | 176 |
| 369548 | 1 | 219 | TGCCACCTGCAGCAGGACCA | 57 | 177 |
| 369549 | 1 | 220 | TTGCCACCTGCAGCAGGACC | 61 | 178 |
| 369550 | 1 | 221 | CTTGCCACCTGCAGCAGGAC | 60 | 179 |
| 369551 | 1 | 222 | GCTTGCCACCTGCAGCAGGA | 44 | 180 |
| 369552 | 1 | 223 | AGCTTGCCACCTGCAGCAGG | 42 | 181 |
| 364944 | 1 | 224 | GAGCTTGCCACCTGCAGCAG | 56 | 182 |
| 369553 | 1 | 225 | AGAGCTTGCCACCTGCAGCA | 64 | 183 |
| 369554 | 1 | 226 | CAGAGCTTGCCACCTGCAGC | 65 | 184 |
| 369555 | 1 | 227 | CCAGAGCTTGCCACCTGCAG | 66 | 185 |
| 369556 | 1 | 228 | CCCAGAGCTTGCCACCTGCA | 70 | 186 |
| 369557 | 1 | 229 | TCCCAGAGCTTGCCACCTGC | 50 | 187 |
| 369558 | 1 | 284 | GTAGAGATGCTCATGTAGTC | 50 | 188 |
| 369559 | 1 | 353 | AAAACCAGCTGGTACAACAG | 40 | 189 |
| 369560 | 1 | 355 | GAAAAACCAGCTGGTACAAC | 36 | 190 |
| 369561 | 1 | 428 | TCCATGAGCAGGTGGCACAC | 67 | 191 |
| 369562 | 1 | 429 | ATCCATGAGCAGGTGGCACA | 71 | 192 |
| 369563 | 1 | 430 | CATCCATGAGCAGGTGGCAC | 78 | 193 |
| 369564 | 1 | 431 | TCATCCATGAGCAGGTGGCA | 75 | 194 |
| 369565 | 1 | 494 | CCCTTCCACAGCAGCTGCTG | 78 | 195 |
| 369566 | 1 | 496 | AGCCCTTCCACAGCAGCTGC | 86 | 196 |
| 369567 | 1 | 497 | GAGCCCTTCCACAGCAGCTG | 71 | 197 |
| 369568 | 1 | 499 | AGGAGCCCTTCCACAGCAGC | 74 | 198 |
| 369569 | 1 | 500 | AAGGAGCCCTTCCACAGCAG | 76 | 199 |
| 369570 | 1 | 501 | GAAGGAGCCCTTCCACAGCA | 71 | 200 |
| 369571 | 1 | 502 | TGAAGGAGCCCTTCCACAGC | 54 | 201 |
| 369572 | 1 | 503 | TTGAAGGAGCCCTTCCACAG | 35 | 202 |
| 369573 | 1 | 504 | CTTGAAGGAGCCCTTCCACA | 51 | 203 |
| 369574 | 1 | 506 | GGCTTGAAGGAGCCCTTCCA | 40 | 204 |
| 369575 | 1 | 508 | TGGGCTTGAAGGAGCCCTTC | 0 | 205 |
| 369576 | 1 | 509 | CTGGGCTTGAAGGAGCCCTT | 0 | 206 |
| 369577 | 1 | 510 | GCTGGGCTTGAAGGAGCCCT | 3 | 207 |
| 369578 | 1 | 530 | GCCCTGGGTTTCACATGCTC | 64 | 208 |
| 369579 | 1 | 531 | GGCCCTGGGTTTCACATGCT | 62 | 209 |
| 369580 | 1 | 619 | TATACAGGTAATTGTCAGGG | 53 | 210 |
| 369581 | 1 | 620 | TTATACAGGTAATTGTCAGG | 55 | 211 |
| 369582 | 1 | 621 | ATTATACAGGTAATTGTCAG | 40 | 212 |
| 369583 | 1 | 642 | GTTGACTGCATAGGTGAGAT | 70 | 213 |
| 369584 | 1 | 645 | AATGTTGACTGCATAGGTGA | 72 | 214 |
| 369585 | 1 | 647 | CAAATGTTGACTGCATAGGT | 68 | 215 |
| 369586 | 1 | 649 | TCCAAATGTTGACTGCATAG | 61 | 216 |
| 364950 | 1 | 735 | AGACTTCAGGGTGCTGGCTG | 45 | 217 |
| 369587 | 1 | 736 | CAGACTTCAGGGTGCTGGCT | 63 | 218 |
| 369588 | 1 | 737 | CCAGACTTCAGGGTGCTGGC | 63 | 219 |
| 369589 | 1 | 998 | TCCTGGATTATTATAGCCAC | 45 | 220 |
| 364956 | 1 | 999 | ATCCTGGATTATTATAGCCA | 39 | 221 |
| 369590 | 1 | 1000 | CATCCTGGATTATTATAGCC | 43 | 222 |
| 369591 | 1 | 1001 | GCATCCTGGATTATTATAGC | 51 | 223 |
| 369592 | 1 | 1003 | GAGCATCCTGGATTATTATA | 45 | 224 |
| 369593 | 1 | 1004 | TGAGCATCCTGGATTATTAT | 26 | 225 |
| 369594 | 1 | 1005 | CTGAGCATCCTGGATTATTA | 52 | 226 |
| 369595 | 1 | 1006 | CCTGAGCATCCTGGATTATT | 41 | 227 |
| 364957 | 1 | 1053 | GCACTTGGCTGGTTCCTGGC | 77 | 228 |
| 369596 | 1 | 1077 | GGTAAGACAATTCTTCCAGT | 77 | 229 |
| 369597 | 1 | 1078 | TGGTAAGACAATTCTTCCAG | 57 | 230 |
| 369598 | 1 | 1079 | TTGGTAAGACAATTCTTCCA | 66 | 231 |
| 369599 | 1 | 1080 | CTTGGTAAGACAATTCTTCC | 73 | 232 |
| 369600 | 1 | 1082 | AGCTTGGTAAGACAATTCTT | 66 | 233 |
| 369601 | 1 | 1083 | GAGCTTGGTAAGACAATTCT | 61 | 234 |
| 369602 | 1 | 1085 | AAGAGCTTGGTAAGACAATT | 64 | 235 |
| 369603 | 1 | 1087 | GCAAGAGCTTGGTAAGACAA | 64 | 236 |
| 369604 | 1 | 1088 | GGCAAGAGCTTGGTAAGACA | 76 | 237 |
| 369605 | 1 | 1090 | AGGGCAAGAGCTTGGTAAGA | 44 | 238 |
| 369606 | 1 | 1092 | ACAGGGCAAGAGCTTGGTAA | 64 | 239 |
| 369607 | 1 | 1093 | AACAGGGCAAGAGCTTGGTA | 69 | 240 |
| 369608 | 1 | 1094 | AAACAGGGCAAGAGCTTGGT | 77 | 241 |
| 369609 | 1 | 1095 | AAAACAGGGCAAGAGCTTGG | 62 | 242 |
| 369610 | 1 | 1096 | GAAAACAGGGCAAGAGCTTG | 54 | 243 |
| 369611 | 1 | 1098 | CAGAAAACAGGGCAAGAGCT | 62 | 244 |
| 369612 | 1 | 1100 | TCCAGAAAACAGGGCAAGAG | 72 | 245 |
| 369613 | 1 | 1184 | GGGCACCATGCTGATTTTCC | 71 | 246 |
| 369614 | 1 | 1221 | GCTCTCTGGCCAGAGGACTG | 80 | 247 |
| 369615 | 1 | 1223 | ATGCTCTCTGGCCAGAGGAC | 68 | 248 |
| 369616 | 1 | 1224 | GATGCTCTCTGGCCAGAGGA | 58 | 249 |
| 369617 | 1 | 1227 | GCTGATGCTCTCTGGCCAGA | 64 | 250 |
| 369618 | 1 | 1395 | GTCCAGGAACAGGCTCTCTG | 76 | 251 |
| 369619 | 1 | 1397 | AGGTCCAGGAACAGGCTCTC | 68 | 252 |
| 369620 | 1 | 1398 | CAGGTCCAGGAACAGGCTCT | 43 | 253 |
| 369621 | 1 | 1399 | GCAGGTCCAGGAACAGGCTC | 59 | 254 |
| 369622 | 1 | 1400 | AGCAGGTCCAGGAACAGGCT | 45 | 255 |
| 364961 | 1 | 1401 | GAGCAGGTCCAGGAACAGGC | 54 | 256 |
| 369623 | 1 | 1506 | ACTTGGGAACTCATCCCAGG | 58 | 257 |
| 369624 | 1 | 1507 | CACTTGGGAACTCATCCCAG | 58 | 258 |
| 369625 | 1 | 1508 | GCACTTGGGAACTCATCCCA | 66 | 259 |
| 369626 | 1 | 1670 | CTCAGGGAGTTGCTGAAGCT | 63 | 260 |
| 369627 | 1 | 1671 | GCTCAGGGAGTTGCTGAAGC | 62 | 261 |
| 369628 | 1 | 1673 | TGGCTCAGGGAGTTGCTGAA | 28 | 262 |
| 369629 | 1 | 1674 | CTGGCTCAGGGAGTTGCTGA | 47 | 263 |
| 369630 | 1 | 1676 | GACTGGCTCAGGGAGTTGCT | 65 | 264 |
| 369631 | 1 | 1700 | GGACCCAGCTCTCTGGGACA | 57 | 265 |
| 369632 | 1 | 1701 | TGGACCCAGCTCTCTGGGAC | 61 | 266 |
| 369633 | 1 | 1703 | TCTGGACCCAGCTCTCTGGG | 46 | 267 |
| 369634 | 1 | 1705 | GGTCTGGACCCAGCTCTCTG | 70 | 268 |
| 369635 | 1 | 1706 | GGGTCTGGACCCAGCTCTCT | 65 | 269 |
| 369636 | 1 | 1777 | TGGTTGGCTCAGAGAGCTGG | 63 | 270 |
| 369637 | 1 | 1779 | AGTGGTTGGCTCAGAGAGCT | 51 | 271 |
| 369638 | 1 | 1780 | CAGTGGTTGGCTCAGAGAGC | 64 | 272 |
| 369639 | 1 | 1781 | ACAGTGGTTGGCTCAGAGAG | 57 | 273 |
| 369640 | 1 | 1782 | CACAGTGGTTGGCTCAGAGA | 71 | 274 |
| 369641 | 1 | 1997 | GCACTGCTGGCAAGCAGGCT | 52 | 275 |
| 369642 | 1 | 2056 | AAGGCTTATACCCCTCTTCC | 81 | 276 |
| 369643 | 1 | 2057 | AAAGGCTTATACCCCTCTTC | 82 | 277 |
| 364973 | 1 | 2058 | GAAAGGCTTATACCCCTCTT | 60 | 112 |
| 369644 | 1 | 2059 | GGAAAGGCTTATACCCCTCT | 80 | 279 |
| 369645 | 1 | 2060 | TGGAAAGGCTTATACCCCTC | 84 | 280 |
| 369646 | 1 | 2062 | CTTGGAAAGGCTTATACCCC | 68 | 281 |
| 369647 | 1 | 2064 | GTCTTGGAAAGGCTTATACC | 59 | 282 |
| 369648 | 1 | 2065 | GGTCTTGGAAAGGCTTATAC | 58 | 283 |
| 369649 | 1 | 2066 | AGGTCTTGGAAAGGCTTATA | 77 | 284 |
| 364974 | 1 | 2067 | GAGGTCTTGGAAAGGCTTAT | 60 | 113 |
| 369650 | 1 | 2068 | TGAGGTCTTGGAAAGGCTTA | 59 | 286 |
| 369651 | 1 | 2126 | AGTCCAAAGGTGAACAAGGG | 50 | 287 |
| 369652 | 1 | 2128 | CCAGTCCAAAGGTGAACAAG | 55 | 288 |
| 369653 | 1 | 2130 | GTCCAGTCCAAAGGTGAACA | 50 | 289 |
| 369654 | 1 | 2131 | TGTCCAGTCCAAAGGTGAAC | 52 | 290 |
| 369655 | 1 | 2403 | TCCACAGCAGCAGCCACAGC | 57 | 291 |
| 369656 | 1 | 2524 | TACTCTTCTCTGAGATGCCC | 86 | 292 |
| 369657 | 1 | 2526 | TTTACTCTTCTCTGAGATGC | 71 | 293 |
| 369658 | 1 | 2528 | GATTTACTCTTCTCTGAGAT | 57 | 294 |
| 369659 | 1 | 2529 | GGATTTACTCTTCTCTGAGA | 67 | 295 |
| 364982 | 1 | 2530 | AGGATTTACTCTTCTCTGAG | 68 | 296 |
| 369660 | 1 | 2531 | GAGGATTTACTCTTCTCTGA | 87 | 297 |
| 369661 | 1 | 2532 | TGAGGATTTACTCTTCTCTG | 83 | 298 |
| 369662 | 1 | 2578 | TCTGGCTTGAGCTCTGAGCA | 69 | 299 |
| 369663 | 1 | 2579 | GTCTGGCTTGAGCTCTGAGC | 68 | 300 |
| 364990 | 1 | 2743 | AAGTCTTTTGGAAATCTGCC | 69 | 301 |
| 364992 | 1 | 2763 | CCTTCATACCATGGTTCTTC | 81 | 302 |
| 365003 | 1 | 3168 | GAGCACCTCTAGGCAATGAC | 82 | 303 |

Oligonucleotides targeted to the following nucleotides of SEQ ID NO:1 were effective at inhibiting the expression of human IL-4Rα at least about 40% as can be determined by the tables above: nucleotides 167-265; 284-303; 353-372; 428-450; 487-525; 530-550; 619-640; 642-668; 735-760; 777-796; 917-950; 998-1025; 1053-1072; 1077-1121; 1160-1203; 1221-1246; 1395-1420; 1492-1528; 1608-1627; 1670-1695; 1700-1735; 1777-1801; 1976-1995; 1997-2016; 2056-2088; 2056-2101; 2126-2150; 2230-2349; 2390-2422; 2524-2598; 2626-2662; 2674-2693; 2731-2791; 2856-2880; 2915-2934; 3053-3072; 3103-3122; 3168-3187; 3198-3217; 3297-3322; and 3420-3451. These are validated target segments, and antisense compounds targeting these segments (or ranges thereof) are contemplated for use in the compositions, methods and kits provided herein. Although some oligonucleotides within each nucleotide region did not inhibit expression at least 40%, they substantially overlapped (i.e., at least 80% overlapped) oligonucleotides effective at inhibiting expression at least 40%. All oligonucleotides targeted to the following regions of SEQ ID NO:1 were effective at inhibiting expression of IL-4Rα at least 50% as can be determined by the tables above: nucleotides 284-303; 428-450; 494-525; 530-550; 642-668; 1053-1072; 1184-1203; 1221-1246; 1506-1527; 1777-1801; 1976-2016; 2056-2101; 2126-2150; 2230-2349; 2403-2422; 2524-2551; 2578-2598; 2743-2782; 2856-2880; 2915-2934 and 3168-3187. These are validated target segments, and antisense compounds targeting these segments (or ranges thereof) are contemplated for use in the compositions, methods and kits provided herein. All oligonucleotides targeted to the following regions of GenBank nucleotides 18636000-18639000 of NT 010393.14 were effective at inhibiting expression of IL-4Rα at least 40% as can be determined by the table above: nucleotides 8231-8250 and 47104-47123. These are validated target segments.

Screening of Oligonucleotides Containing Nucleotide Mismatches, Dose Response

Based on the screening above, ISIS 231894 (AIR231894) was selected for further study. A series of oligonucleotides were designed based on ISIS 231894 containing 1, 3, 5, and 7 mismatch nucleobases as shown in Table 6 below. It should be noted that the mismatches are interspersed throughout the central portion of the compounds, rather than at the ends. This decreases the affinity of the oligonucleotide for the target mRNA more than mismatch oligonucleotides at the ends. Such concepts are well known and understood by those skilled in the art. The oligonucleotides are 5-10-5 MOE-gapmers, as is ISIS 231894. All cytidine residues are 5-methylcytidines. The mismatch bases are underlined.

TABLE 6
Oligonucleotides targeted to mouse
IL-4Rα containing mismatches

| | Target | | | |
|---|---|---|---|---|
| | SEQ | # mis- | | SEQ |
| ISIS # | ID NO | match | Sequence (5′ to 3′) | ID NO |
| 231894 | 2 | 0 | CCGCTGTTCTCAGGTGACAT | 53 |
| 352489 | 2 | 1 | CCGCTGTTCTCAGGTGACAT | 53 |
| 352490 | 2 | 3 | CCGCTGATCACAGCTGACAT | 304 |
| 352491 | 2 | 5 | CCGCTCATCACTGCTGACAT | 305 |
| 352492 | 2 | 7 | CCACTCATCACTGCTGACTT | 306 |

The compounds were analyzed for their effect on gene target mRNA levels by quantitative real-time PCR as described in other examples herein using the target specific primers shown in Table 2. Data are averages from two experiments in which b.END cells were treated with the concentrations of the compounds listed.

TABLE 7
Inhibition of mouse IL-4Rα by chimeric, mismatch oligonucleotides (%
vehicle control IL-4Rα mRNA expression is shown)

| | Number | | | | | | | SEQ |
|---|---|---|---|---|---|---|---|---|
| Isis No | mismatch | 1 nM | 5 nM | 10 nM | 25 nM | 50 nM | 100 nM | ID NO |
| 231894 | Parent | 100 | 54 | 43 | 31 | 21 | 21 | 53 |
| 352489 | 1 mm | 74 | 55 | 52 | 51 | 44 | 49 | 53 |
| 352490 | 3 mm | 92 | 106 | 98 | 88 | 89 | 88 | 304 |
| 352491 | 5 mm | 104 | 104 | 97 | 102 | 114 | 90 | 305 |
| 352492 | 7 mm | 109 | 118 | 121 | 104 | 88 | 69 | 306 |

Oligonucleotides having at least three mismatched bases interspersed within the central portion of the compound were not able to reduce the expression of the target RNA by at least 40% even at the highest doses of oligonucleotide tested.

Mouse Models of Allergic Inflammation

Asthma is a complex disease with variations in disease severity and duration. In view of this, multiple animal models have been designed to reflect various aspects of the disease. It is understood that the models have some flexibility in regard to days of sensitization and treatment, and that the timelines provided reflect the days used herein. There are several important features common to human asthma and the mouse models of allergic inflammation. One of these is pulmonary inflammation, in which production of Th2 cytokines, e.g., IL-4, IL-5, IL-9, and IL-13 is dominant and increased numbers of total leukocytes and increased numbers or percentages of eosinophils, neutrophils, lymphocytes and macrophages are recruited to the airways. Another is goblet cell metaplasia with increased mucus production. Lastly, airway hyper-responsiveness (AHR) occurs, resulting in increased sensitivity to cholinergic receptor agonists such as acetylcholine or methacholine.

Asthma models and Oligonucleotide Administration

Karras et al. (2007) Am J Respir Crit Care Med 36:276-285 describes the results of administration of an IL-4Rα ASO in an acute ovalbumin (OVA) challenge mouse model and secondary OVA challenge mouse model, in which ISIS 231894 IL-4Rα ASO (a mouse homolog of AIR645 also referred to herein as AIR231894) or mismatch control was administered as part of a prophylactic dosing regimen. The reference also describes data from a chronic model of induced allergic inflammation using a therapeutic treatment regimen, with ASO treatment initiated after the establishment local pulmonary inflammation. The chronic asthma model recapitulates some of the histological features of severe asthma in humans, including collagen deposition and lung tissue remodeling. The chronic OVA asthma model produces a more severe disease than that observed in the acute or rechallenge asthma models.

The results showed that the IL-4Rα ASO, but not mismatch control, specifically inhibits IL-4Rα protein expression in lung eosinophils, macrophages, dendritic cells, and airway epithelium after inhalation in allergen-challenged mice. Inhalation of IL-4Rα ASO attenuated allergen-induced AHR, suppressed airway eosinophilia and neutrophilia, and inhibited production of airway Th2 cytokines and chemokines in previously allergen-primed and -challenged mice. Histologic analysis of lungs from these animals demonstrated reduced goblet cell metaplasia and mucus staining that correlated with inhibition of Muc5AC gene expression in lung tissue. Therapeutic administration of inhaled IL-4Rα ASO in chronically allergen-challenged mice produced a spectrum of anti-inflammatory activity similar to that of systemically administered Dexamethasone with the added benefit of reduced airway neutrophilia.

Airway Hyperreponsiveness in Response to Methacholine

U.S. Pat. No. 7,507,810 describes the assessment of airway responsiveness by inducing airflow obstruction with a methacholine aerosol using a noninvasive method. AIR 231894, but not the mismatch control oligonucleotide, caused a significant (p≦0.05 for both 1 μg/kg and 10 μg/kg vs. vehicle treated controls) dose dependent suppression in methacholine induced AHR in sensitized mice as measured by whole body plethysmography. AIR231894, but not the mismatch control oligonucleotide, also reduced airway resistance and increased lung compliance compared to measurements performed in control animals that inhaled saline only. Methacholine-induced AHR was also suppressed by AIR231894 treatment in chronically allergen-challenged mice, using a therapeutic administration regimen as measured using a noninvasive method.

Mouse Model of Allergic Inflammation, Analysis for Nasal Rhinitis Endpoints

Mouse models of allergen-induced acute and chronic nasal inflammation similar to those above have been used to study allergic rhinitis in mice (Hussain, et al. 2002 Larangyoscope 112: 1819-1826; Iwasaki, et al. 2003 J. Allergy Clin Immunol 112: 134-140; Malm-Erjefaelt, et al. 2001 Am J Respir Cell Mol. Biol. 24:352-352; McCusker, et al., J Allergy Clin Immunol., 110: 891-898; Saito, et al. 2001 Immunology 104:226-234). In all of the models, the mice were sensitized to OVA by injection, as above, followed by intranasal OVA instillation. The most substantial difference in the models is in the endpoints analyzed. Endpoints include, but are not limited to, the amount of sneezing and nasal scratching immediately after administration of allergen challenge (i.e., intranasal OVA), and nasal histology including mucus and eosinophil counts and measurements of cytokines or other inflammatory products in nasal lavage fluid or nasal tissues. Methods for performing such analyses are detailed in the references cited which are incorporated herein by reference. Administration of oligonucleotides targeted to IL-4Rα decrease nasal inflammation, as evidenced by fewer infiltrating eosinophils quantitated by digital imaging, and fewer nasal rubs and sneezes per unit of time in IL-4Rα ASO treated animals as compared to saline treatment.

Rodent Model of Smoking Induced Pulmonary Disease

Smoking is known to cause lung irritation and inflammation which can result in a number of diseases in humans including, but not limited to, emphysema and COPD. A number of smoking animal models are well known to those skilled in the art including those utilizing mice (Churg, et al. 2002 Am. J. Respir. Cell. Mol. Biol. 27:368-347; Churg, et al. 2004. Am. J. Respir. Crit. Care Med. 170:492-498, both incorporated herein by reference), rats (e.g., Sekhon, et al. 1994. Am. J. Physiol. 267:L557-L563, incorporated herein by reference), and guinea pigs (Selman, et al. 1996. Am J. Physiol. 271:L734-L739, incorporated herein by reference). Animals are exposed to whole smoke using a smoking apparatus (e.g., Sekhon, et al. 1994. Am. J. Physiol. 267:L557-L563) well known to those skilled in the art.

Changes in lung physiology are correlated with dose and time of exposure. In short term studies, cell proliferation and inflammation were observed. In one study, exposure of rats to 7 cigarettes for 1, 2, or 7 days resulted in proliferation of pulmonary artery walls at the level of the membranous bronchioles (MB), respiratory bronchioles (RB), and alveolar ducts (AD). Endothelial cell proliferation was only present in vessels associated with AD. In a separate study (Churg, et al. 2002 Am. J. Respir. Cell. Mol. Biol. 27:368-347), mice exposed to whole smoke from four cigarettes were shown to have an increase in neutrophils, desmosine (an indicator of elastin breakdown), and hydroxyproline (an indicator of collagen breakdown) after only 24 hours. In a long term study, an emphysema-like state was induced (Churg, et al. 2004. Am. J. Respir. Crit. Care Med. 170:492-498). Mice exposed to whole smoke from four cigarettes using a standard smoking apparatus, for five days per week for six months were found to have an increase in neutrophils and macrophages in BALF as compared to control mice. Whole lung matrix metalloproteinases (MMP)-2, -9, -12, and -13, and matrix type-1 (MT-1) proteins were increased. An increase in matrix breakdown products was also observed in BALF. These markers correlate with tissue destruction and are observed in human lungs with emphysema.

These models can be used to determine the efficacy of therapeutic interventions for the prevention, amelioration, and/or treatment of the damage and disease caused by cigarette smoke and/or other insults. Administration of oligonucleotide can be performed prior to, concurrent with, and/or after exposure to smoke to provide a prophylactic or therapeutic model. AIR231894 is 100% complimentary to both mouse and rat IL-4Rα; therefore, it can be used in both mouse and rat studies. Dose ranges are determined by the time of oligonucleotide administration relative to smoke inhalation, with lower doses (e.g., 1-100 μg/kg) required for prevention of lung damage. Higher doses (e.g., 100-1000 μg/kg) are required for treatment after, or alternating with, smoke exposure. Positive control (e.g., smoke exposure, no oligonucleotide administration) and negative control (e.g., no smoke exposure, with or without oligonucleotide treatment) animals are also analyzed and validated therapeutic agents such as dexamethasone may be used as controls for comparative efficacy.

Endpoints for analysis include those discussed in the asthma models above. Functional endpoints include AHR, resistance and compliance. Morphological changes include BAL cells, cytokine levels, histological determinations of alveolar destruction (i.e., increase in alveolar space) and airway mucus accumulation, as well as tissue markers of disease, including collagen and elastin. The emphysematous changes specific to this model discussed in this example can also be analyzed to determine the effect of the antisense oligonucleotide.

Mouse Model of Elastase Induced Emphysema

Elastase is an essential mediator in lung damage and inflammation and is released by recruited neutrophils following smoke-induced damage. A rat model of emphysema has been developed to analyze the process of elastase mediated lung damage, and possible therapeutic interventions to prevent, ameliorate, and/or treat the pathologies associated with such damage and resulting disease (Kuraki, et al. 2002 Am J Respir Crit. Care Med 166:496-500, incorporated herein by reference). Intratracheal application of elastase induced emphysematous changes in all lobes of the lung including severe lung hemorrhage as demonstrated by increased hemoglobin in BALF; neutrophil accumulation in BALF; inhibition of hyperinflation and degradation of elastic recoil. Histopathological changes included elastase-induced airspace enlargement and breakdown of alveoli. These changes are similar to those observed in human emphysema.

In the model, rats are treated with human sputum elastase (SE563, Elastin Products, Owensville, Mo.) without further purification. Rats are treated with a sufficient dose of elastase, about 200 to 400 units, by intratracheal administration using a microsprayer. Alternatively, intratracheal administration can be performed as described above in the mouse models. After sufficient time to allow for damage to occur, about eight weeks, functional and morphological changes are analyzed. A similar model can be performed using mice with a lowered dose of elastase relative to weight and/or lung area (e.g., 0.05 U of porcine pancreatic elastase/g body weight).

Administration of oligonucleotide can be performed prior to, concurrent with, and/or after administration of elastase to provide a prophylactic or therapeutic model. AIR231894 is 100% complimentary to both mouse and rat IL-4Rα. Dose ranges are determined by the time of oligonucleotide administration relative to elastase administration with lower doses (e.g., 1-100 μg/kg) required for prevention of lung damage. Higher doses (e.g., 100-1000 μg/kg) are required for treatment after, or alternating with, elastase administration. Positive control (e.g., elastase treatment, no oligonucleotide administration) and negative control (e.g., no elastase, with or without oligonucleotide treatment) animals are also analyzed and validated therapeutic agents such as dexamethasone may be used as controls for comparative efficacy.

Endpoints for analysis include those discussed in the asthma models above. Functional endpoints include AHR, resistance and compliance. Morphological changes include BAL cells, cytokine levels, and mucus accumulation. The emphysematous changes specific to this model discussed in this example can also be analyzed to determine the effect of the antisense oligonucleotide.

### Other Embodiments

All of the features disclosed in this specification can be combined in any combination. Each feature disclosed in this specification can be replaced by an alternative feature serving the same, equivalent, or similar purpose. Thus, unless expressly stated otherwise, each feature disclosed is only an example of a generic series of equivalent or similar features. From the above description and the examples that follow, one skilled in the art can easily ascertain the characteristics of the compositions and methods provided herein, and without departing from the spirit and scope thereof, can make various changes and modifications to adapt to various usages and conditions. For example, the compounds provided herein can be used as research tools (for example, to isolate new targets for performing drug discovery). In other instances, the compounds can be radiolabelled for imaging tissue or organs or be used to form bioconjugates for affinity assays. These and other uses and embodiments of the compounds and compositions provided herein will be apparent to those of ordinary skill in the art.

The disclosure also encompasses all possible permutations of the claim set, as if they were multiple dependent claims.

Various embodiments of the disclosure could also include permutations of the various elements recited in the claims as if each dependent claim was multiple dependent claim incorporating the limitations of each of the preceding dependent claims as well as the independent claims. Such permutations are expressly within the scope of this disclosure.

While the invention has been particularly shown and described with reference to a number of embodiments, it would be understood by those skilled in the art that changes in the form and details can be made to the various embodiments disclosed herein without departing from the spirit and scope of the invention and that the various embodiments disclosed herein are not intended to act as limitations on the scope of the claims.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference and can be employed in the practice of the invention. More generally, documents or references are cited in this text, either in a Reference List before the claims, or in the text itself; and, each of these documents or references (“herein cited references”), as well as each document or reference cited in each of the herein cited references (including any manufacturer's specifications, instructions, etc.), is hereby expressly incorporated herein by reference.

The embodiments of the present invention described above are intended to be merely exemplary, and those skilled in the art will recognize, or be able to ascertain using no more than routine experimentation, numerous equivalents to the specific procedures described herein. All such equivalents are considered to be within the scope of the present invention and are covered by the following claims. Furthermore, as used in this specification and claims, the singular forms “a,” “an” and “the” include plural forms unless the content clearly dictates otherwise. Thus, for example, reference to “an antisense compound” includes a mixture of two or more such antisense compounds, and the like. Additionally, ordinarily skilled artisans will recognize that operational sequences must be set forth in some specific order for the purpose of explanation and claiming, but the present invention contemplates various changes beyond such specific order.

### EQUIVALENTS

Those skilled in the art will recognize, or be able to ascertain using no more than routine experimentation, numerous equivalents to the specific procedures described herein. Such equivalents are considered to be within the scope of the present invention and are covered by the following claims. The contents of all references, patents, and patent applications cited throughout this application are hereby incorporated by reference. The appropriate components, processes, and methods of those patents, applications and other documents can be selected for the present invention and embodiments thereof.

The description of the present invention has been presented for purposes of illustration and description, but is not intended to be exhaustive or limiting of the invention to the form disclosed. The scope of the present invention is limited only by the scope of the following claims. Many modifications and variations will be apparent to those of ordinary skill in the art. The embodiment described and shown in the figures was chosen and described in order to best explain the principles of the invention, the practical application, and to enable others of ordinary skill in the art to understand the invention for various embodiments with various modifications as are suited to the particular use contemplated.

The invention can be further defined by reference to the following examples. It will be apparent to those skilled in the art that many modifications, both to materials and methods, can be practiced without departing from the purpose and interest of this invention. The following examples are set forth to assist in understanding the invention and should not be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

While the invention has been particularly shown and described with reference to a number of embodiments, it would be understood by those skilled in the art that changes in the form and details can be made to the various embodiments disclosed herein without departing from the spirit and scope of the invention and that the various embodiments disclosed herein are not intended to act as limitations on the scope of the claims.

Various embodiments of the disclosure could also include permutations of the various elements recited in the claims as if each dependent claim was multiple dependent claim incorporating the limitations of each of the preceding dependent claims as well as the independent claims. Such permutations are expressly within the scope of this disclosure.

The following examples are offered by way of illustration, and not by way of limitation.

### EXAMPLES

The following examples are provided for illustrative purposes only and are not intended to limit the scope of the invention.

### Example 1

### Evaluation of AIR645Toxicity in Mice and Monkeys

AIR645 is a second generation 2′-O-methoxyethyl-modified ASO targeted to the interleukin-4 receptor alpha (IL-4Rα) chain that can be delivered by nebulization. AIR645 is expected to inhibit expression of IL-4Rα and act as an anti-inflammatory agent with potential for improvement of airflow limitation and, thus, can demonstrate utility in the treatment of asthma, allergic rhinitis and possibly other diseases.

The toxicity of AIR645 was assessed in mice following 6- and 13-week treatment and in monkeys following 4-week treatment. Because it was not possible (or practical) to produce exaggerated systemic exposures by the inhalation route, systemic toxicity was characterized in a 6-week intravenous mouse study.

AIR645 is not complementary to the murine mRNA and is not active in mice. In order to distinguish between toxicities due to IL-4Rα inhibition and toxicities related to chemical class (hybridization-independent), a murine-specific IL-4Rα antisense inhibitor (AIR231894) was studied along with AIR645 in this species.

13-week Mouse Inhalation Toxicity Study of AIR645 with 13-week Recovery

CD-1 mice were given 4 loading doses of inhaled AIR645, followed by once weekly thereafter. The study group size was 10/sex/grp. Recovery was 6/sex/grp (control, high dose, and mouse-specific ASO (AIR231894)). TK satellite group was 3/males/time point. The doses included control (0 mg/kg/wk), low (0.4 mg/kg/wk), mid (2 mg/kg/wk), mid-high (10 mg/kg/wk), high (50 mg/kg/wk), and mouse-specific ASO (50 mg/kg/wk) (10% deposition assumed).

Dose-dependent concentrations of AIR645 were measured in lung and plasma. The plasma exposure was very low. Systemic bioavailability was estimated as 6-8% of calculated inhaled dose. No systemic toxicity was observed. There were no meaningful clinical pathology changes (no changes in serum chemistry; minor increases in WBCs at the 50 mg/kg/wk dose only). No clinical signs manifested (no body or organ weight changes). Any histopathology changes were limited to the site of administration (no systemic target organ changes were observed). Of note, there was no toxicity-associated with inhibition of IL-4R expression.

Thus, inhalation of AIR645 does result in dose-dependent lung exposure. AIR645 is well-tolerated. Histological changes are limited to those associated with uptake and clearance of the ASO.

6-week Intravenous Toxicity Study of AIR645 in Mice

CD-1 mice were given 4 loading doses of AIR645 by intravenous injection, followed by once weekly thereafter. The study group size was 10/sex/grp. The doses included control (0 mg/kg/wk), low (1 mg/kg/wk), mid (5 mg/kg/wk), mid-high (25 mg/kg/wk), high (50 mg/kg/wk), and mouse-specific ASO (50 mg/kg/wk).

Dose-dependent tissue concentrations of AIR645 were documented. No systemic toxicity was observed (no clinical pathology changes (serum chemistry, hematology), no treatment-related clinical signs, no body weight changes, no toxicologically-relevant organ weight changes). Furthermore, there was no toxicity-associated inhibition of IL-4R expression. In terms of histopathology, there were basophilic granules within macrophages. These findings were related to the cellular uptake of the ASO.

4-week Monkey Inhalation Toxicity Study of AIR645 with 13-week Recovery

Cynomolgous macaques were given 4 loading doses of inhaled AIR645, followed by once weekly thereafter. The study group size was 3/sex/grp. Recovery was 2/sex/grp (control & high dose). TK satellite group was 1/sex/timepoint, 5 mg/kg, days 2, 15, 29, and 57. The doses included control (0 mg/kg/wk), low (0.5 mg/kg/wk), mid (1.5 mg/kg/wk), mid-high (5 mg/kg/wk), and high (15 mg/kg/wk) (20% deposition assumed).

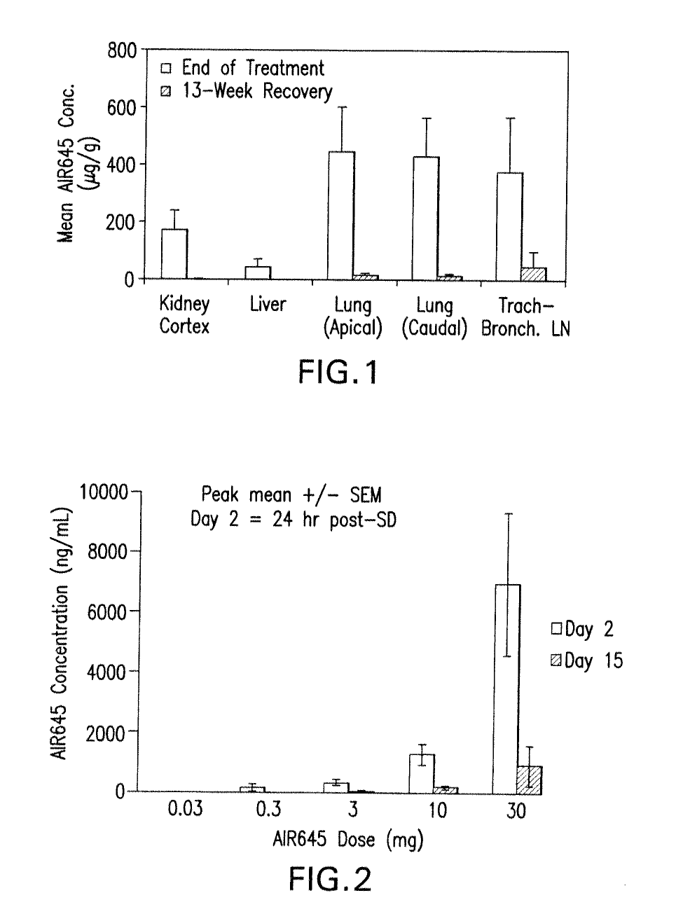

Minimal tissue exposure was measured after 13 weeks of recovery (FIG. 1).

There were dose-dependent concentrations of AIR645 in the lung and plasma (very low plasma exposure). No systemic toxicity was observed (no clinical pathology changes (serum chemistry, hematology, coagulation, or urinalysis), no complement activation, no treatment-related clinical signs, no body or organ weight changes). Histopathology changes were limited to the sites of administration (no systemic target organ changes). As regards histopathology, AIR645 was well-tolerated at inhaled doses up to 15 mg/kg/wk. There were no effects on pulmonary function (no changes in respiration rate, tidal volume, or minute volume).

Thus, the inhalation of AIR645 results in dose-dependent lung exposure with minimal plasma bioavailability and tissue exposure (kidney, liver) (data not shown). AIR645 is well-tolerated. There is very low systemic exposure, i.e., an enhanced safety profile. Finally, histopathological changes are limited to those associated with cellular uptake and clearance of oligonucleotide.

Of note, genetic toxicity studies (in vitro bacterial cell gene mutation, mouse lymphoma gene mutation) were performed were without findings. Safety pharmacology studies were also conducted to evaluate CNS and cardiovascular function, as well as pulmonary and renal function, and showed no effects.

### Example 2

### Evaluation of AIR645Pharmacokinetics in Mice and Monkeys

AIR645 preclinical pharmacokinetics were characterized in mice following both aerosol inhalation and intravenous (i.v.) injection, and in monkeys following aerosol inhalation.

13-week Mouse Inhalation Study

Mice received 0, 0.4, 2, 10, or 50 mg/kg AIR645 via 10-minute aerosol inhalations, q2d for the first week, followed by once per week for the remainder of the 13-week treatment period. Plasma and tissue samples were collected and PK measured following single 2 and 10 mg/kg dose, tissue exposures at the end of the 13-week treatment (day 92), and after 3 months treatment-free recovery period. Plasma samples were analyzed using hybridization ELISA (quantitation range 2-100 ng/mL), and tissue samples were analyzed using HPLC-MS/MS (quantitation range 0.05-10 μg/g).

4-week Monkey Inhalation Study

Monkeys received 0, 0.5, 1.5, 5, and 15 mg/kg AIR645 via 35-minute aerosol inhalations, q2d for the first week, followed by once per week for the remainder of the 4-week treatment period. Plasma and tissue samples were collected and PK measured as follows: plasma PK on days 1 and 29; tissue PK following a single 5 mg/kg dose, tissue exposures at the end of the 4-week treatment (day 31), and after 3 months treatment-free recovery period. Plasma samples were analyzed using hybridization ELISA (quantitation range 2-100 ng/mL), and tissue samples were analyzed using HPLC-MS/MS (quantitation range 0.05-10 μg/g).

Tissue Distribution Following Inhalation of Aerosolized AIR645 in Mice and Monkeys

Following aerosol inhalation in mice and monkeys, the highest concentrations of AIR645 were observed in lungs and tracheobronchial lymph nodes (TBLN) (measured in monkey only), with minimal distribution to nasal epithelium and minimal systemic organ distribution (kidney and liver), and no distribution to brain (Table 8 below). Concentrations of AIR645 in lungs, TBLN, and nasal epithelium were dose-dependent, but increased less than would be predicted based upon dose alone, and suggest saturation in local tissue uptake. Concentrations of AIR645 in liver and kidney were dose-dependent, and increased greater than would be predicted based upon dose alone. The increased concentrations in kidney and liver, together with increased plasma AUC at higher doses can be explained by saturation of local binding sites of the lungs at higher doses.

TABLE 8
Parent Drug (AIR645) Concentrations (μg/g) Measured by Quantitative
LC-MS/MS in Selected Organs or Tissues Collected 24 hours after the Last Dose following 13-
Week Aerosol Inhalations in Mouse or 4-Week Aerosol Inhalations in Monkey

| | Tissue | | | | | | | |
|---|---|---|---|---|---|---|---|---|
| | Mouse | | | | Monkey | | | |
| | Dose, Inhaled (mg/kg/wk) | | | | | | | |
| | 0.4 | 2 | 10 | 50 | 0.5 | 1.5 | 5 | 15 |
| Lung | 11.0 ± 5.3 | 13.5 ± 10.1 | 308 ± 144 | 360 ± 117 | 24.9 ± 15.5 | 97.3 ± 33.1 | 288 ± 192 | 448 ± 160 |
| (Apical)^{a} | | | | | | | | |
| Lung | NM | NM | NM | NM | 28.9 ± 9.1 | 102 ± 72 | 149 ± 89 | 429 ± 137 |
| (Caudal) | | | | | | | | |
| Nasal | NM | NM | NM | NM | 0.441 ± 0.280 | 0.923 ± 0.705 | 2.31 ± 2.07 | 8.09 ± 5.11 |
| Epithelium | | | | | | | | |
| TBLN | NM | NM | NM | NM | 5.21 ± 3.04 | 187 ± 174 | 243 ± 121 | 378 ± 189 |
| Liver | BLQ^{c} | 0.372 ± 0.426 | 2.94 ± 1.75 | 14.2 ± 10.2 | 0.055 ± 0.066 | 0.480 ± 0.251 | 5.92 ± 5.04 | 46.7 ± 25.3 |
| Kidney^{b} | 0.088 ± 0.043 | 1.94 ± 1.57 | 10.0 ± 6.7 | 16.2 ± 11.6 | 0.968 ± 1.056 | 7.01 ± 3.36 | 73.0 ± 18.0 | 175 ± 66 |
| Brain | NM | NM | NM | BLQ^{d} | NM | NM | NM | BLQ^{e} |

Data presented are mean ± standard deviation.
^{a}Whole lung in mouse.
^{b}Whole kidney in mouse; kidney cortex in monkey
^{c}Liver samples in 9 out of 10 animals were below the limit of quantitation (BLQ, <0.05 μg/g), 1 out of 10 had concentration of 0.07 μg/g, possibly resulted from contamination during sample collection or processing.
^{d}Brain samples in 6 out of 10 animals were below the limit of quantitation (BLQ, <0.05 μg/g), 4 out of 10 had concentration ranged from 0.07 to 0.14 μg/g, possibly resulted from contamination during sample collection or processing.
^{e}Brain samples in 5 out of 6 animals were below the limit of quantitation (BLQ, <0.05 μg/g), 1 out of 6 had concentration of 0.05 μg/g, possibly resulted from contamination during sample collection or processing.
TBLN = Tracheobronchial lymph nodes
NM = not measured

Tissue concentrations of AIR645 following 4 weeks of inhalation treatment were generally higher in monkeys than those observed in mice following 13 weeks of inhalation treatment at comparable dose levels (Table 8, above). For example, kidney concentrations of intact AIR645 in monkeys treated with 15 mg/kg/wk were 175±66 μg/g compared to 16.2±11.6 μg/g in mice treated with 50 mg/kg/wk. In tissues of pharmacological interest, i.e., lungs, higher concentrations of AIR645 were measured in monkey when compared to mouse (97.3±33.1 μg/g in monkeys vs. 13.5±10.1 μg/g in mouse, 24 hours after the last dose of 1.5 or 2 mg/kg/wk AIR645, respectively). The distribution within the lung also appeared to be relatively uniform, as concentrations in apical and caudal regions of the lung in monkeys were similar. The observed distribution and accumulation in tissues, favoring lung over other organs, is favorable for the intended clinical application of AIR645.

4-week Non-GLP Mouse Intranasal Study

Balb/c mice received AIR645 via intranasal instillation of a 50 μl drop (25 Onaris) over a four-week period. Administered doses were 0.01, 0.3 and 10 mg/kg (qd5×4 wks) and 0.05, 1.5 and 50 mg/kg (1 qw×4 wks). In the intranasal study, circulating plasma levels of AIR645 were not assessed. Dose-related, but not necessarily dose-proportional, concentrations of full-length AIR645 were detected in all tissues that were evaluated (nasal epithelium, lung, liver and kidney). AIR645 concentrations in the nose were below the limit of quantitation in low-dose groups (0.01 mg/kg qd5×4 wks and 0.05 mg/kg 1 qw×4 wks). Low concentrations of AIR645 were present in the mid-dose groups (0.3 mg/kg qd5×4 wks and 1.5 mg/kg 1 qw×4 wks), while in high-dose groups (10 mg/kg qd5×4 wks and 50 mg/kg 1 qw×4 wks), much higher concentrations were present. Substantially higher concentrations of full length AIR645 were present in the lungs in all dosage groups as compared to those measured in the nose.

The high concentrations of AIR645 in the lungs following intranasal insufflation were attributed to the 50 μl volume of test article given at each dosing, which exceeded the volume of the nasal cavity by more than 50%. The pulmonary exposures resulted in systemic exposure, as evidenced by the significant measurable concentrations of AIR645 in the kidney and liver at all dosages and by both regimens, although the possibility that some systemic exposure occurred via nasal absorption cannot be excluded. Essentially all oligonucleotide detected in the nasal tissue was full-length AIR645, indicating little local metabolism. There was a general trend towards lower percentages of full-length AIR645 as dosage increased, but in no case was the mean percentage of full-length AIR645 less than 75%. This is most likely related to the limitations of the assay as opposed to an increase in metabolism as concentration increases. The metabolites for lower concentrations would have been below the LLOQ or LOD of the assay. A summary of tissue concentrations of full-length AIR645 is presented in Table 9, below:

TABLE 9
Summary of Tissue Concentrations of Full Length AIR645 (μg/g)

| Dosage | | Kidney | Lung | Nose | Liver |
|---|---|---|---|---|---|
| 0.01 mg/kg/dose | Mean | 0.14 | 2.22 | BLQ | 0.53 |
| qd5 × 4 wks | S.D. | 0.27 | 1.58 | BLQ | 0.41 |
| 0.3 mg/kg/dose | Mean | 1.20 | 78.17 | 1.84 | 0.93 |
| qd5 × 4 wks | S.D. | 1.02 | 14.65 | 1.56 | 0.21 |
| 10 mg/kg/dose | Mean | 116.47 | 890.86 | 39.70 | 198.10 |
| qd5 × 4 wks | S.D. | 17.87 | 102.18 | 6.47 | 33.40 |
| 0.05 mg/kg/dose | Mean | 0.47 | 5.56 | BLQ | 0.57 |
| 1qw × 4 wks | S.D. | 0.93 | 2.84 | BLQ | 0.77 |
| 1.5 mg/kg/dose | Mean | 7.63 | 75.63 | 1.42 | 4.23 |
| 1qw × 4 wks | S.D. | 2.10 | 10.01 | 0.51 | 0.95 |
| 50 mg/kg/dose | Mean | 101.71 | 336.88 | 57.20 | 109.33 |
| 1qw × 4 wks | S.D. | 21.38 | 78.37 | 24.92 | 13.46 |

6-week Mouse Intravenous Study

Mice received 0, 1, 5, 25, and 50 mg/kg AIR645 via intravenous injections, q2d for the first week, followed by once per week for the remainder of the 6-week treatment period. Tissue samples were collected and PK measured as follows: tissue exposures at the end of the 6-week treatment (day 44). Tissue samples were analyzed for PK via HPLC-MS/MS (quantitation range 0.05-10 μg/g).

Following intravenous (i.v.) administration in mice at doses that ranged from 1 to 50 mg/kg/wk, tissue concentrations of AIR645 were dose-dependent, increased greater-than-proportional to dose in lungs, were dose-proportional in the liver, and increased less than would be predicted based upon dose alone in the kidneys, indicating saturation in kidney uptake with the dose range studied. Systemic bioavailability was less than 10% of the inhaled dose when compared to i.v. (Table 10, below). Therefore, there was minimal systemic absorption by aerosol inhalation administration, and unwanted effects associated with high systemic organ exposure are not expected.

TABLE 10
Parent Drug (AIR645) Concentrations (μg/g) Measured
by Quantitative LC-MS/MS in Systemic Tissues Collected
24 hours after the Last Dose following 13-Week Aerosol
Inhalations or 6-Week IV Injections in Mouse

| | 10 mg/kg/wk inhaled | 1 mg/kg/wk | % of Absolute Systemic |
|---|---|---|---|
| Tissue | dose | IV dose | Bioavailability^{b} |
| Liver | 2.94 ± 1.75 | 3.90 ± 2.12 | 7.6% |
| Kidney^{a} | 10.0 ± 6.7 | 17.1 ± 18.6 | 5.8% |

Data presented are mean ± standard deviation.
^{a}Whole kidney
^{b}Bioavailability (%) = {(C inhalation/Dose inhalation)/(C i.v./Dose i.v.)} × 100
(C = the respective mean tissue concentration of AIR645 by the end of treatment)

4-week Monkey Inhalation Study

Following a single 5 mg/kg aerosol dose in monkeys, AIR645 cleared slowly from the lungs, with an estimated half-life of 13 to 14 days. Meanwhile, systemic tissue half-lives of AIR645 were 8 to 25 days (Table 11, below). Following 4 weeks of repeated administrations, there was approximately 5-fold accumulation in tissues compared to a single dose, consistent with the frequency of dosing and the tissue elimination half-lives.

TABLE 11
Estimated Elimination Half-Life (In Days) of AIR645 from
Selected Tissues following a Single Aerosol Inhalation

| | |
|---|---|
| Mouse | Monkey |

| | Dose, Inhaled (mg/kg) | | |
|---|---|---|---|
| Tissue | 2 | 10 | 5 |
| Liver | NE | 2 | NE |
| Kidney^{a} | NE | 12 | 25 |
| Lung^{b} (Apical) | 9 | NE | 14 |
| Lung (Caudal) | NM | NM | 13 |
| TBLN | NM | NM | 8.1 |

^{a}Whole kidney in mouse; kidney cortex in monkey
^{b}Whole lung in mouse.
NE = not estimated
NM = not measured

The slow clearance from tissue is the basis for the infrequent treatment interval contemplated herein. The elimination half-life of AIR645 in the lungs in mice and monkeys is 9 and 14 days, respectively. These data support the use of the loading and weekly maintenance dosing regimens employed in the toxicology studies, and demonstrates that continuous tissue exposure was achieved in these studies. Consistent with the half-lives, 87 to 99% of AIR645 was cleared from tissues following 13 weeks of recovery after the last dose for the 15 mg/kg/wk dose group (FIG. 1).

Preclinical Data (Summary)

Thus, in terms of pharmacokinetics and toxicology, tissue exposure of inhaled AIR645 in mice and monkeys was dose-dependent, with the lung and tracheobronchial lymph nodes being the sites of highest concentrations, with minimal systemic organ disposition (kidney and liver) and with no distribution to the brain. Systemic bioavailability was estimated to be 2 to 10%. Following aerosol administration of a single dose of 5 mg/kg to monkeys, AIR645 cleared slowly from the lungs, with an estimated half-life of 13 to 14 days. Drug clearance was continuous, resulting in near complete clearance with 87 to 99% of AIR645 cleared from tissues following 13 weeks of recovery.

The preclinical data indicate that once-weekly administration by inhalation at doses as high as 15 mg/kg in monkeys and 50 mg/kg in mice produced no evidence of clinical signs or organ toxicity and resulted in significant and dose-dependent pulmonary exposure to drug. Two inhalation toxicology studies have been completed, a 13-week study in mice and a 4-week study in monkeys. No significant irritation in lung or the upper respiratory tract was observed in either study.

In mice, the most prominent change resulting from treatment with nebulized oligonucleotide was adaptive in nature and related to cellular uptake of drug into macrophages within alveoli and the tracheobronchial lymph nodes. This uptake resulted in dose-dependent, minimal to mild increases in macrophage size and number. Minimal inflammatory cell infiltrates were present in approximately 25% of mice treated at dose levels >10 mg/kg/wk and were reversible. These findings were made in normal mice; in mouse models of asthma there is an obvious and quantifiable reduction in the numbers of inflammatory cells in the lung following AIR645 treatment, suggesting that the slight increases in cell infiltrates in normal mice are not clinically relevant in the disease state. There were no systemic histological changes in either species, consistent with the limited systemic bioavailability.

No effects on pulmonary function or lymphohistiocytic cell infiltrates were observed in monkeys. The mechanistic basis for the sensitivity of mice to these effects is well understood, and the monkey is considered more predictive of the responses of humans, particularly with respect to pro-inflammatory effects of oligonucleotides (Henry, S. P., et al. 2007 Antisense Drug Technology: Principles, Strategies, and Applications, 2^{nd} edition: “Toxicologic properties of 2′-O-methoxyethyl . . . ”; Kwoh, T. J., et al. 2007 supra “An overview of the clinical safety . . .”).

Furthermore, in allergen-sensitized mice, aerosol administration of a mouse IL-4Rα-specific antisense oligonucleotide (ASO) resulted in dose-dependent pharmacological effects that correlated with antisense inhibition of IL-4Rα. Once-weekly inhaled IL-4Rα ASO treatment suppressed allergen-induced airway hyper-responsiveness (AHR) to methacholine, airway eosinophilia and mucus production. Reduced expression of IL-4Rα following inhaled ASO treatment was associated with reduced levels of Th2 cytokines and chemokines and reduced expression of the epithelial mucin gene, Muc 5AC. Of note, addition of an inhaled IL-4Rα ASO to a suboptimal dose of inhaled budesonide in allergen-challenged mice resulted in greater reductions in allergen-induced airway inflammation and hyper-responsiveness than either agent alone.

Treatment with an inhaled IL-4Rα ASO was also effective in reducing airway eosinophilia, mucus overproduction and hyper-responsiveness in chronically allergen challenged mice. The magnitude and quality of the IL-4Rα ASO effects were similar to those demonstrated by systemically administered Dexamethasone (Dex) in chronically allergen challenged mice, with the added benefit of reduced numbers of airway neutrophils.

Intranasal instillation IL-4Rα ASO in allergen-sensitized and challenged mice resulted in reductions in allergen-induced sneezes, nose rubs and nasal tissue eosinophilia. These pharmacological effects indicate that AIR645 can be an effective therapy for atopic respiratory diseases including asthma and allergic rhinitis. Pharmacodynamic and pharmacological activity of inhaled IL-4Rα ASO has been demonstrated in mice at doses of 30-500 μg/kg/wk, resulting in lung tissue concentrations of >0.05 μg of drug per gram of wet lung tissue. Based on standard species scaling, AIR645 can be effective at 10-250 μg/kg/wk in human subjects.

Of note, the mouse analog of AIR645 has been found to suppress allergen-induced IL-4Rα protein in macrophages, eosinophils, dendritic cells, and epithelial cells (Karras, J., et al. 2007 Am J Respir Cell Mol Biol 36:276-285). It also exhibited a broad activity profile in chronically allergen challenged mice, producing reduction of AHR, percent eosinophils, Th2 cytokines and chemokines in bronchoalveolar lavage fluid, mucus, collagen, and airway wall thickening. Finally, efficacy has consistently been observed at 100 μg/kg in mice at lung concentrations of 150-200 ng/g.

### Example 3

### Assessment of Safety and Tolerability and Pharmacokinetics of Single and Multiple Nebulized Doses of AIR645, Followed by Assessment of Pharmacodynamic Characteristics of AIR645 in Subjects with Controlled Asthma

In order to evaluate the safety and tolerability of AIR645, as well as local exposure and systemic bioavailability of AIR645, the effects of administering the drug single and multiple doses regimen healthy volunteers was investigated and, if well-tolerated, in a group of patients with controlled asthma, as well.

Safety and tolerability were evaluated based on the following parameters: adverse events, physical examination, vital signs, electrocardiograms, continuous cardiac monitoring (telemetry), spirometry, diffusing capacity of Carbon Monoxide, and clinical laboratory values.

Pharmacokinetics were evaluated via measurements of intact (full-length) AIR645 in the plasma and sputum.

Pharmacodynamics were evaluated via detection and measurement of inflammatory biomarkers in induced sputum. This can include, without limitation, assessment of cell counts from induced sputum for percentages of individual types of inflammatory cells, particularly eosinophils. In addition, it can include assessment of quantities of the chemokine, TARC (thymus activated and regulated chemokine), as well as quantities of the arachidonic acid pathway eicosanoid, 15(S)-hydroxy eicosatetrenoic acid, in induced sputum.

Single Dose Cohorts

Cohorts 1 through 5 received 0.03 mg, 0.3 mg, 3 mg, 10 mg, and 30 mg AIR645, respectively. 0.03 mg is 7-fold lower than the human equivalent dose MABEL (minimal anticipated biological effect level). The subjects received a single dose on day 1. AIR645 was diluted with 0.9% sodium chloride (sterile normal saline) in an aseptic environment prior to (less than 24 hrs before) administration via nebulization. Placebo constituted 0.9% sodium chloride alone.

Dose-proportional increases in sputum exposure were observed (FIG. 2). Sputum half-life was about 5 days and independent of dose level. Low levels of AIR645 were only detected in plasma at the 30 mg dose (data not shown).

Multiple Dose Cohorts

Cohorts 6 through 9 and 10 (mild, controlled asthma, not taking ICS—inhaled corticosteroid) received 0.3, 3, 10, 20, and 20 mg AIR645, respectively (subject to safety data evaluation) via nebulization. The top dose was 10-fold higher than the predicted efficacious dose. The subjects received 6 doses as follows: on days 1, 3, 5, 8, 15, and 22.

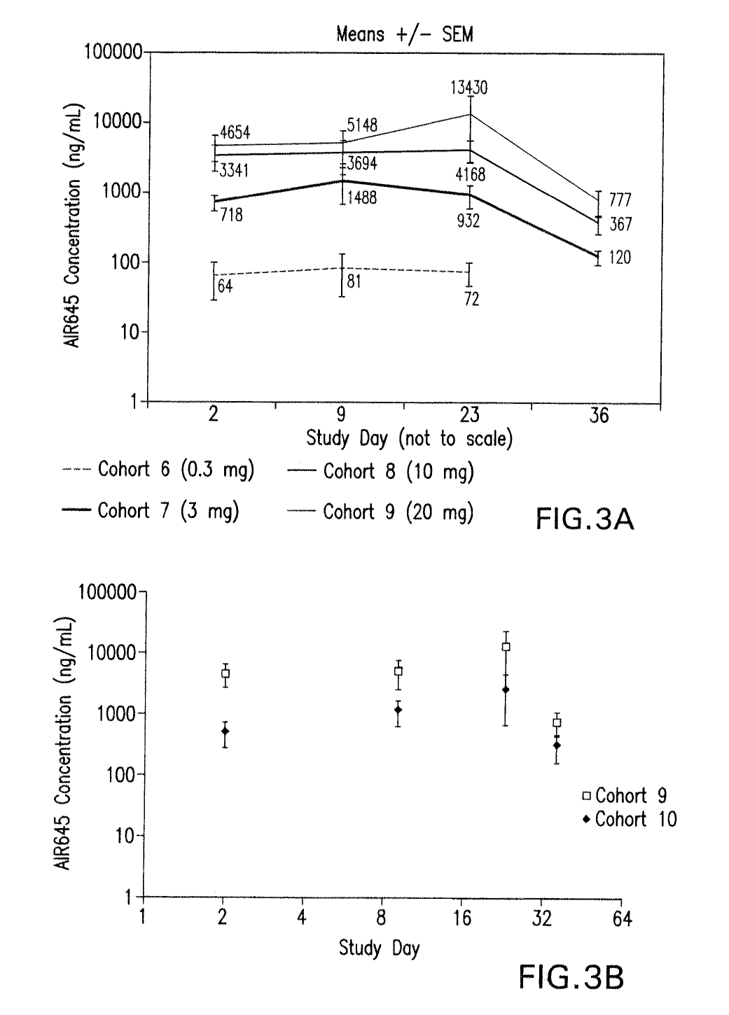

Dose-dependent increases in sputum exposure were observed (FIG. 3). Sputum half-life was about 5 days and independent of dose level. Low levels of AIR645 were only detected in plasma at the 20 mg dose, and there were no signs of “drug” accumulation in the plasma (data not shown). Of note, the asthma cohort exhibited a safety profile and plasma and sputum exposures similar to the healthy volunteers (data not shown).

The dose ranges chosen for the single dose phase (approximately 0.4 to 430 μg/kg body weight in a 70 kg subject) is supported by the non-clinical toxicology studies in which doses of up to 15 and 50 mg/kg/week were administered to monkeys and mice, respectively. In effect, the highest dose proposed in the clinical trials is well below doses that were associated with adverse effects in mice and monkeys. The final dose range for the multiple dose phase is determined based on safety and tolerability data from the single dose phase and is anticipated not to exceed 60 mg/week exposure (−1 mg/kg in a 60 kg subject), such as 30 mg/week exposure (˜500 μg/kg in a 60 kg subject).

The alternate day administration “loading” schedule for the first week of the multiple dose phase is to achieve near steady-state levels by the end of the first week of dosing. Once daily administration also achieves near steady-state levels by the end of the first week of dosing.

Thus, AIR645 exposure in sputum was found to be dose-dependent, and no accumulation of drug was evident. AIR645 half-life in sputum was calculated to be approximately 5 days. AIR645 concentration was >1000-fold higher in sputum than in plasma, indicating a very low systemic bioavailability of the drug.

Adverse events were infrequent, mostly mild, and certainly not severe or serious. No dose-limiting toxicities were observed. AIR645 single dose and multiple dose were both shown to be safe and well-tolerated.

### Example 4

### AIR645-CS1 Mild Asthma Subjects (COHORT 10) Biomarker Report

Inhaled aerosolized AIR645 (human interleukin-4 receptor alpha [IL4Rα] antisense oligonucleotide, formerly ISIS 369645) was administered to subjects with mild asthma to assess safety, tolerability and bioavailability of the drug and to evaluate pharmacodynamic (PD) effects on exploratory inflammatory endpoints, as part of Clinical Trial Protocol AIR645-CS1, “A Phase 1, Single Center, Randomized, Double-Blind, Placebo-Controlled Study of AIR645 in Healthy Subjects and Subjects with Controlled Asthma” (Anapharm, Inc. study number 82043).

Methods

For Cohort 10 of AIR645-CS1, 8 subjects were administered either aerosolized AIR645 or placebo (saline (0.9% sodium chloride), USP pH 5.5 (4.5 to 7.0)) by oral inhalation (6 subjects were randomized to receive AIR645 and 2 to receive placebo). AIR645 dissolved in saline, or saline only (placebo), was nebulized using a PARI LC Sprint nebulizer coupled to a PARI PRONEB ULTRA II compressor. AIR645 concentration in saline was adjusted to aerosolize a dose of 20 mg (inhaled dose).

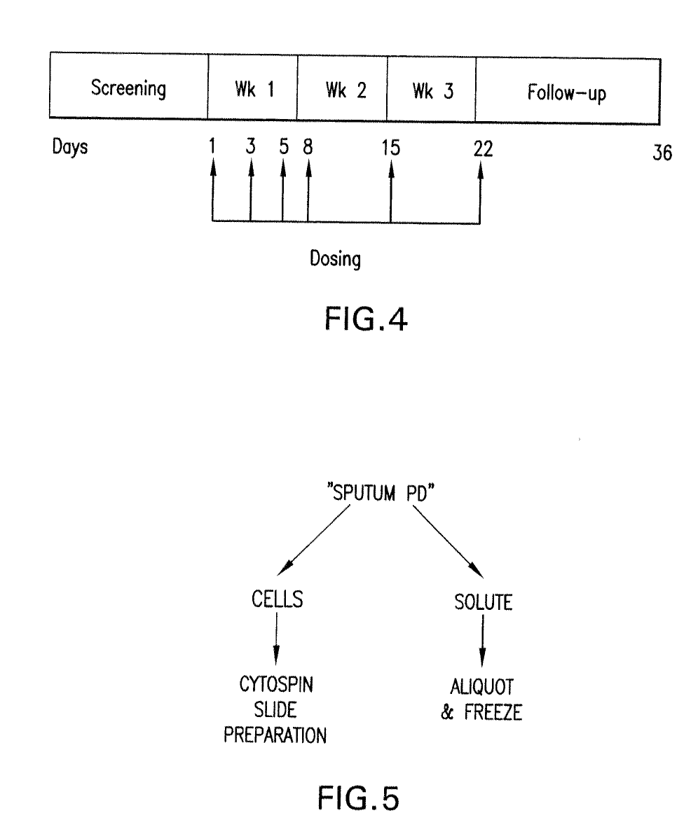

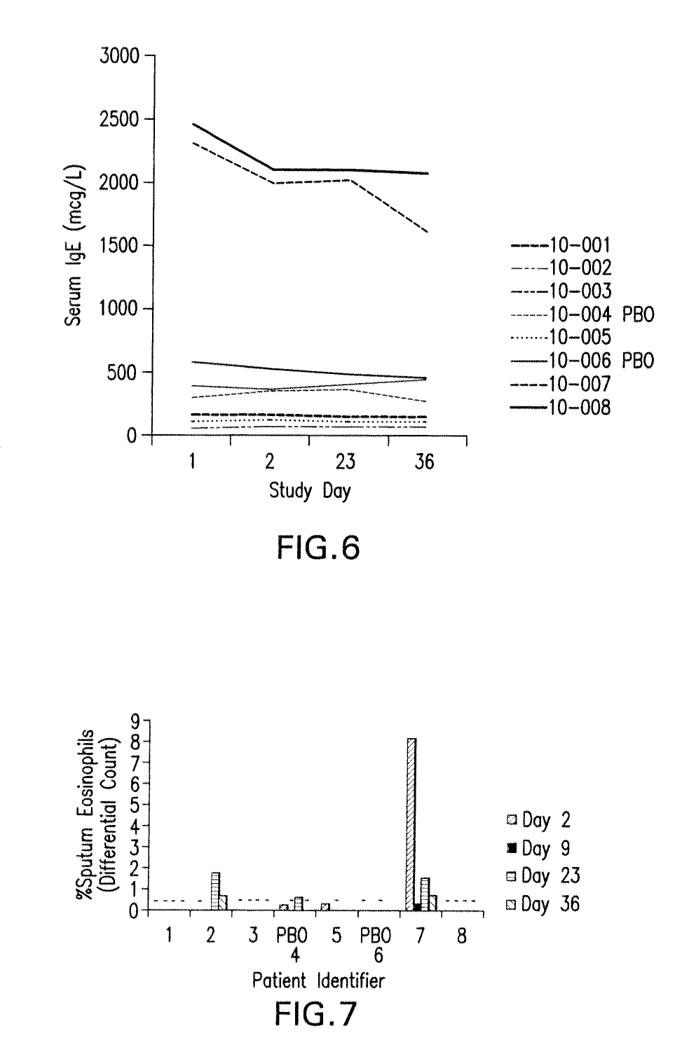

All subjects in Cohort 10 received either AIR645 or saline placebo by inhalation on Days 1, 3, 5, 8, 15 and 22 (FIG. 4).

TABLE 12
Biomarkers of pulmonary inflammation driven
by Th2 response include the following:

| BIOMARKER | IL-4Rα |
|---|---|
| Serum IgE | B cell class switching to IgE production |
| Serum eosinophils | Th2 cytokine/chemokine dependent process |
| Sputum eosinophils | Th2 cytokine/chemokine dependent process |
| Sputum 15-HETE | IL-13 responsive arachidonic acid pathway |
| | mediator |
| Sputum TARC | Th2 chemokine |

Serum Collection and IgE Assay

Venous blood samples were collected for serum measurement of total IgE pre-dose on Day 1 and on Days 2, 23 and 36. Serum was separated and collected following centrifugation of clotted whole blood. The serum fraction of the sample was transferred to labeled cryovials, frozen, and stored at −80° C. until analyzed for total IgE measurement.

Induced Sputum Collection and Processing

Induced sputum was collected following inhalation of increasing concentrations of hypertonic saline solution under the supervision of trained clinical staff. The sputum PD samples were processed for isolation of the cellular and solute fractions (Fahy, J. V., et al. 1994 J Allergy Clin Immunol 93(6):1031). Cellular fractions were diluted with one volume of diluted Sputolysin solution (in mL; 1:10 dilution of Sputolysin stock solution) equal to four times the weight of the sample (in grams; e.g., 100 mg sample, add 400 microliters of diluted Sputolysin solution). Solute fractions were diluted with one volume of diluted Sputolysin solution equal to the weight of the sample (in grams; e.g., 100 mg sample, add 100 microliters of diluted Sputolysin solution). Induced sputum samples were handled and processed as described in FIG. 5. Cells from induced sputum samples were quantified as percentage of total leukocytes. The solute fraction of induced sputum samples was used for quantitation of inflammatory biomarkers that are known to be induced by IL-4 and/or IL-13. Induced sputum was collected for PD endpoint analyses on Days 2, 9, 23 and 36.

Sputum Cell Cytospin Preparation and Differential Cell Counts

A total cell count was performed visually on the cellular fraction of induced sputum samples using a modified Neubauer hemocytometer, and cell viability was determined simultaneously by enumerating the percentage of cells excluding trypan blue to ensure adequate sample quality. If the squamous epithelial cell contamination was <25% of the total number of cells, then the sample was considered of valid quality. Cytospins were prepared by placing 60 microliters of the cell suspension, adjusted with phosphate buffered saline, pH 7, to 0.5×10⁶ non-squamous cells/mL, into a Shandon III cytocentrifuge (Shandon Southern Instruments). Slides were prepared by cyto-centrifugation of cells at 450 rpm for 4 minutes. Two slides were prepared with a Diff-Quick stain and 400 cells were counted. The resulting differential count was expressed as a percentage of the total non-squamous epithelial cell count.

Quantitation of IL-4Rα mRNA in Induced Sputum Cells

Induced sputum cells frozen in Trizol RNA lysis buffer were thawed and RNA extracted using standard chloroform/phenol interphase methodology. RNA was precipitated with ethanol and pelleted by microcentrifugation. IL-4Rα mRNA was detected by quantitative reverse transcriptase polymerase chain reaction (using TaqMan chemistry) and relative level compared with the house-keeping gene, glucuronidase beta (GUS B).

Quantitation of TARC & 15-HETE in Induced Sputum

Frozen aliquots of stored sputum samples (FIG. 5) were thawed at room temperature. Total protein concentration was determined in sputum with a microplate protein assay kit (reducing agent compatible, Pierce). The concentration of T-cell activated and regulated chemokine (TARC) was determined in undiluted sputum utilizing a Quantikine kit (R&D Systems). 15(S)-hydroxy eicosatetraenoic acid (15-HETE) in sputum was first purified using SEP-Pak cartridges (Waters). A 0.6 ml aliquot of sputum sample was diluted with 2 ml of methanol and centrifuged (10,000 rpm, 10 minutes). Supernatant was removed and diluted with 0.1 M phosphate buffer to a final methanol concentration of 20%. Samples were loaded onto SEP-Pak cartridges previously activated with 5 ml of methanol and washed with 5 ml of 20% methanol in phosphate buffer (pH 7). The cartridges were washed with 5 ml of water and then 15-HETE was eluted with 4 ml of 80% methanol. A 1 ml aliquot of each 80% methanol fraction was evaporated to dryness under negative pressure using a speed-vac. Samples were reconstituted with 0.45 ml of assay buffer for immunoassay. This corresponded to a 1:3 dilution of the sample. 15-HETE was quantified by enzyme immunoassay utilizing reagents from Cayman Chemical (Ann Arbor, Mich.).

Results

Serum total IgE concentrations in Cohort 10 subjects 10-007 and 10-008 were determined to exceed 2000 μg/L on Day 1 (FIG. 4). Both subjects 10-007 and 10-008 were treated with AIR645, and total serum IgE levels in these subjects declined ˜15 and ˜30%, respectively, following AIR645 treatment and follow-up (day 36 compared to day 1).

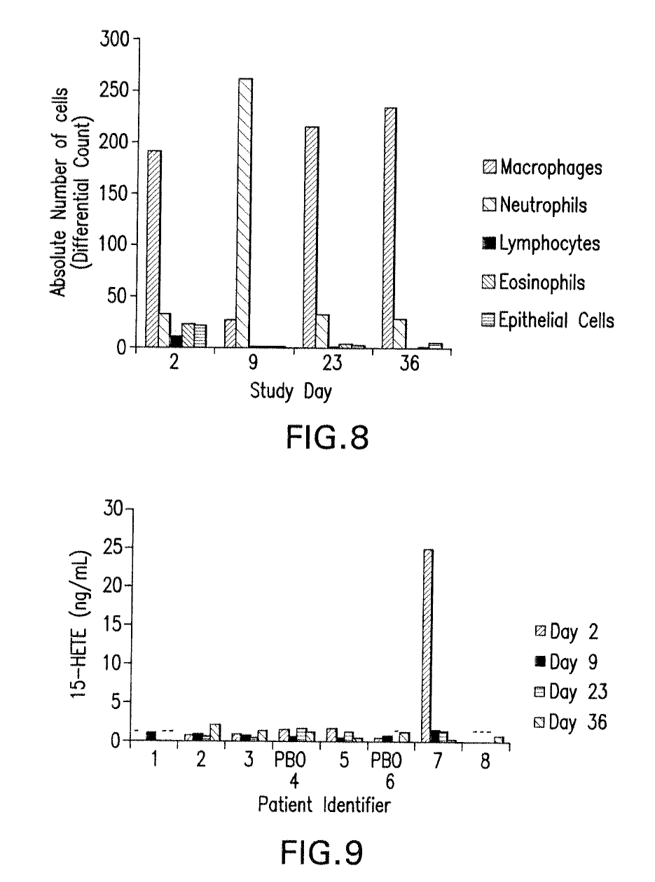

High percent and absolute numbers of eosinophils were detected on Day 2 in induced sputum from one subject (10-007; percent eosinophils shown in FIG. 7). Subjects 1, 3, and 8 either did not produce adequate sputum, and/or their samples did not satisfy acceptability criteria of less than 25% squamous epithelial cell contamination. In fact, the sputum eosinophils in most of the subjects were below 3% at baseline. Induced sputum percent and absolute number of eosinophils were decreased on Days 9, 23 and 36 in subject 10-007 (percent eosinophils shown in FIG. 7; decrease in absolute eosinophils on Days 9, 23, and 36 were similar and are shown in FIG. 8).

Differential cell counts for subject 10-007 showed a transient increase in neutrophils on Day 9 that returned to Day 2 levels by Day 23 and remained at this level on Day 36 (FIG. 8). Subject 10-007 reported symptoms consistent with an upper respiratory tract infection following the first AIR645 dose (Day 1), which can account for the transient change in neutrophils (Gern, J. E., et al. 2000 Am J Respir Crit Care Med 162:2226). However, high variability in the percentage and absolute numbers of neutrophils is commonly observed in induced sputum and can be related to the stress of producing a sample.

TARC levels in induced sputum solute samples from all subjects were mostly below the lower limit of quantitation (data not shown). 15-HETE was detectable in sputum from subject 10-007 on Day 2 and was reduced on Days 9, 23 and 36 (FIG. 9). 15-HETE levels in most subjects were low.

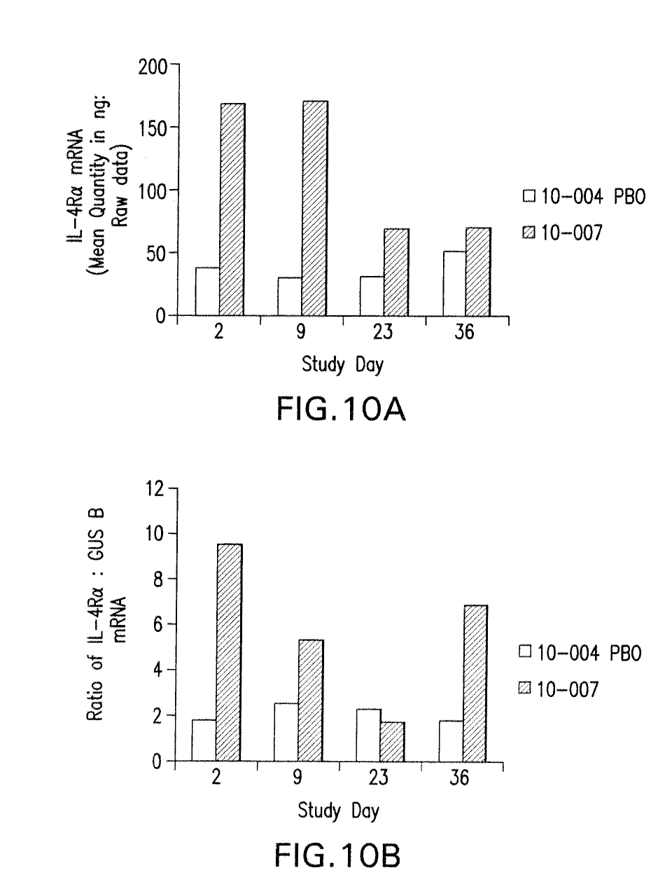

IL-4Rα mRNA levels in induced sputum cell samples were variable but measurable in all subjects studied. IL-4Rα mRNA expression was reduced in subject 10-007 at Days 23 and 36 when expressed as raw data (FIG. 10A) and at Days 9, 23 and 36 when normalized to relative levels of the house-keeping gene, glucuronidase beta (FIG. 10B).

The levels of the measured biomarkers are low in the majority of subjects with controlled mild asthma due to the mild nature of their disease and the tendency for it to be quiescent while they are not exposed to allergens. Furthermore, some of the subjects can not be atopic asthmatics; indeed, non-atopic asthma is also a recognized subset of patients.

The drops observed in serum IgE (15 and 30%) in two subjects with well-controlled mild asthma treated with AIR645 were greater than those observed in patients treated with placebo (5 and 21% drops). Of note, the AIR645-treated subjects with drops of 15 and 30% also had high levels of serum IgE compared to the even typical “high IgE” atopic subjects (2000-2500 μg/L vs. ˜500 μg/L). Total serum IgE concentration above about 100 μg/L serum IgE could be considered above the “normal” range.

Reductions in eosinophils and 15-HETE in sputum and serum IgE in subject 10-007 are consistent with the purported mechanism of action of AIR645, a dual IL-4/IL-13 inhibitor.

In summary, the AIR645Phase I study demonstrated that AIR645 is safe and well-tolerated in healthy subjects and in subjects with controlled asthma. No dose-limiting toxicities or safety signals were detected. In terms of pharmacokinetics, sputum levels of the drug increased in a dose-dependent manner, there was no accumulation, and a half-life of about 5 days was observed. Furthermore, systemic absorption was low.

### Example 5

### AIR-645 CS1 Administration to Mild Asthma Subjects (COHORT 10) Results in Decrease IL-4Rα mRNA in Sputum

Inhaled aerosolized AIR645 (Cohort 10) was administered to 8 subjects with mild asthma as discussed in Example 4. All subjects in Cohort 10 received either AIR645 or saline placebo by inhalation on Days 1, 3, 5, 8, 15 and 22.

Induced sputum was collected following inhalation of increasing concentrations of hypertonic saline solution under the supervision of trained clinical staff. The sputum samples and cells were obtained, processed and prepared as discussed above in Example 4. Induced sputum was collected for endpoint analyses on Days 2, 9, 23 and 36. IL-4Rα mRNA was quantitated as described in Example 4 by quantitative reverse transcriptase polymerase chain reaction (using TaqMan chemistry) and relative level compared with the house-keeping gene, glucuronidase beta (GUS B).

Results

The AIR645-CS1 sputum IL-4Rα mRNA results are shown in Table 13 (raw data; mean quantity IL-4Rα mRNA (ng)) and Table 14 (normalized to housekeeping gene GUSB).

TABLE 13
AIR645-CS1 Sputum mRNA Results (Raw Data)

| | Mean Quantity IL-4Rα mRNA (ng) | | | | |
|---|---|---|---|---|---|
| Patient No. | Day 0 | Day 2 | Day 9 | Day 23 | Day 36 |
| 10-001 | N/A | N/A | N/A | N/A | N/A |
| 10-002 | 128.6 | 44.5 | 29.6 | 26.6 | 103 |
| 10-003 | 38.8 | 53.6 | 33.5 | 14.6 | 36.8 |
| 10-004 | 50.4 | 34.1 | 28.1 | 25.7 | 48.2 |
| 10-005 | 130.9 | 120.0 | 51.8 | 63.9 | 112 |
| 10-006 | 92.5 | 7.04 | 10.2 | N/A | 18.5 |
| 10-007 | 43.24 | 118 | 108 | 49.3 | 52 |
| 10-008 | N/A | N/A | N/A | N/A | N/A |

TABLE 14
AIR645-CS1 Sputum mRNA Results (Normalized to GUSB)

| | Mean Quantity IL-4Rα mRNA (ng)/GUSB mRNA (ng) | | | | |
|---|---|---|---|---|---|
| Patient No. | Day 0 | Day 2 | Day 9 | Day 23 | Day 36 |
| 10-001 | N/A | N/A | N/A | N/A | N/A |
| 10-002 | 3.00 | 1.54 | 1.08 | 2.74 | 5.67 |
| 10-003 | 4.08 | 1.34 | 1.13 | 1.27 | 1.75 |
| 10-004 | 3.02 | 1.66 | 2.13 | 1.84 | 1.72 |
| 10-005 | 4.22 | 13.1 | 10.6 | 3.1 | 5.51 |
| 10-006 | 1.89 | 1.86 | 1.80 | N/A | 1.97 |
| 10-007 | 3.09 | 7.32 | 4.02 | 1.45 | 6.00 |
| 10-008 | N/A | N/A | N/A | N/A | N/A |

A reduction of IL-4Rα mRNA was seen over the drug treatment phase of the study for subjects on AIR645 who also expressed high levels of IL-4Rα mRNA on day 2, but not those on placebo treatment (compare data for AIR645 treated subjects 10-005 and 10-007 day 2 to day 36 vs. that for placebo treated subjects 10-004 and 10-006 day 2 to day 36). Subjects 10-001 and 10-008 either did not produce adequate sputum, and/or their samples did not satisfy acceptability criteria of less than 25% squamous epithelial cell contamination.

These results closely match the initial mRNA analysis discussed in Example 4 that included subjects 10-003, 10-004, 10-005 and 10-007, indicating that the results are reproducible in separate runs on separate days. This is also true of the raw data representations shown in Table 13.

A 75-80% maximal reduction of IL-4Rα mRNA in sputum cells was observed in subjects who have high basal levels of IL-4Rα mRNA expression at day 2 (e.g., subjects 10-005 and 10-007). Overall, subjects 10-002 and 10-003 had minor reductions at day 9 and low expression of basal IL-4Rα mRNA. Subject 10-006 did not provide a sputum sample at day 23 but the day 36 data point is in line with the day 2 and day 9 readings.

Overall, these data provide confirmation of the target mRNA reduction effects of AIR645 the initial analysis indicates that the drug is reducing IL-4Rα mRNA in mild asthmatics, even in the absence of demonstrable airway inflammation. Subject 10-005 did not have elevated sputum eosinophils and 15-HETE (data not shown).

### Example 6

### Evaluation of AIR231894 in a Murine Model of Allergic Rhinitis

IL-4Rα is a key regulator of atopy and allergy mediated by aberrant T helper type 2 (Th2) cytokine expression. Allergic rhinitis develops as a result of a dysregulated response to environmental allergens and is characterized by a Th2 cytokine-mediated eosinophilic inflammation of the nasal submucosa and epithelium accompanied by nasal congestion, sneezing, coughing, itching, tearing, and swelling.

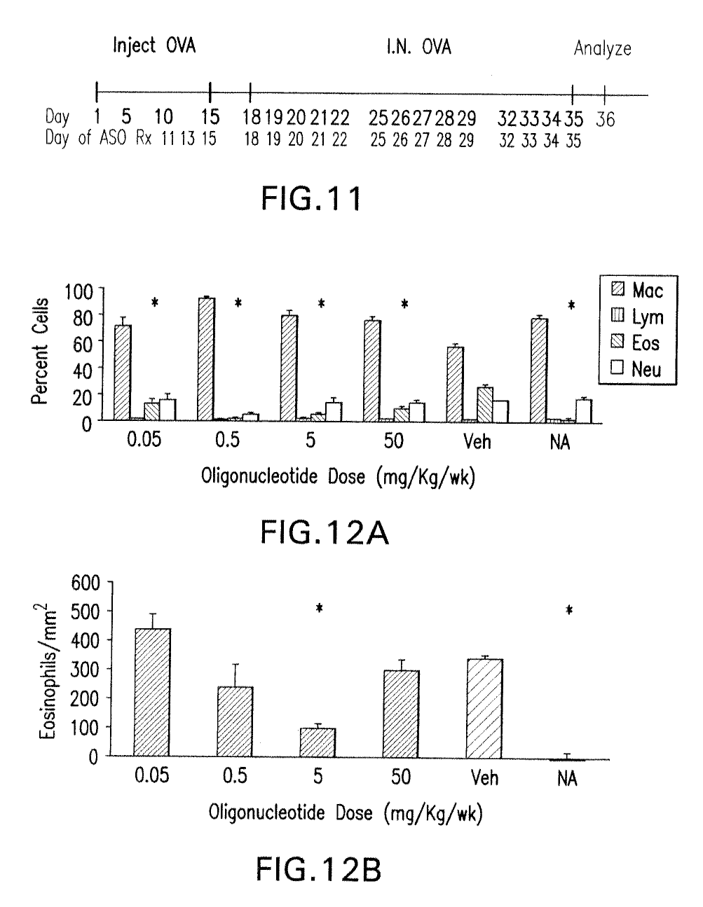

In mice, upper airway inflammation can be modeled by systemic allergen sensitization followed by repeated intranasal allergen challenge (see, e.g., FIG. 11). In these models, nasal eosinophil influx can be demonstrated along with the occurrence of allergic symptoms (Sato, et al., 1999 Int Arch Allergy Immunol 119(3):197-204). Systemic administration of corticosteroids has been shown to reduce nasal tissue eosinophilia in mice (Yu, et al., 1997 Int Arch Allergy Immunol 112(1):73-82; Rhee, et al., Immunology 2004 113(1):106-113). This 36-day study was conducted to assess potential pharmacological effects of an intranasally administered IL-4Rα ASO over a range of dosages in a mouse model of allergic rhinitis.

Materials and Methods

Eleven groups of 10 male BALB/c mice, approximately 6-8 weeks of age, were obtained from Charles River Laboratories, Inc., Wilmington, Mass. for this study.

AIR231894 (“Murine IL-4Rα ASO”), a mouse homolog of AIR645, was dissolved in 0.9% sodium chloride injection (sterile saline), USP pH 5.5 (4.5 to 7.0)) for administration. Concentrations of the AIR231894 were varied to attain the desired dosages (see Table 15).

TABLE 15
IL-4Rα Dose Level and Treatment Groups

| | Treatment | |
|---|---|---|
| | IL4Rα | Administered Dose of IL4Rα |
| Groups | Antisense | Antisense (mg/kg) |
| 1. (N = 10) | AIR231894 | 0.01 |
| 2. (N = 10) | AIR231894 | 0.1 |
| 3. (N = 10) | AIR231894 | 1 |
| 4. (N = 10) | AIR231894 | 10 |
| 5. (N = 10) | Vehicle | saline |
| 6. (N = 10) | Naive | none |

All groups of mice except the naïve group were sensitized by intraperitoneal injection of ovalbumin (OVA) allergen and challenged with OVA by intranasal instillation, as described in FIG. 11. AIR231894 was administered to mice in groups 1-4 by intranasal instillation at a constant volume of 50 μL/dose (25 μL/naris), as described in FIG. 11. Additional groups of mice were treated with budesonide (35 mcg/kg in 5% DMSO/PBS) or the combination of AIR231894 and budesonide via the intranasal route. Groups of vehicle control for oligonucleotide and budesonide treatment and a naive control group of the same size were also included.

All endpoints were evaluated twenty-four hours after the last dose administration. Symptom assessment (nasal rubs and sneezes) were assessed by visual observation. Following evaluation of nasal nose rubs and sneezing, mice were sacrificed and nasal lavage was performed for differential cell count analysis, using standard cytospin slide preparation and Diff-Quick staining procedures (fixation followed by eosin and Wright-Giemsa stains). Nasal tissue was collected and prepared for nasal histology to quantitate eosinophils.

Results

For this study, only data from mice treated with AIR231894 alone, with vehicle (saline), or left untreated (naïve) are presented (six groups of mice). Results from groups treated with budesonide alone, or with the combination of budesonide and AIR231894 were similar to groups treated with AIR231894 alone. Groups 1-4 were dosed 17 times over a period of 25 days and Group 5 was dosed similarly, only with vehicle.

AIR231894 delivered by intranasal instillation (0.05 to 50 mg/kg/wk in normal saline seventeen times over 25 days; FIG. 11 and Table 15), beginning just prior to the first intranasal ovalbumin (OVA) challenge, suppressed both the percentage of nasal lavage eosinophils relative to total cells and the absolute number of nasal tissue eosinophils, detected by histopathological analysis (FIG. 12). The suppressive activity of the IL-4Rα ASO was decreased at the high dose (50 mg/kg/wk). No changes in the percentages of neutrophils or macrophages were observed in nasal lavage fluid (FIG. 12). There were no pro-inflammatory effects detected in histopathologic analyses (numbers of hematopoietic cells) after inhalation of nebulized AIR231894 at doses up to 1 mg/kg/wk.

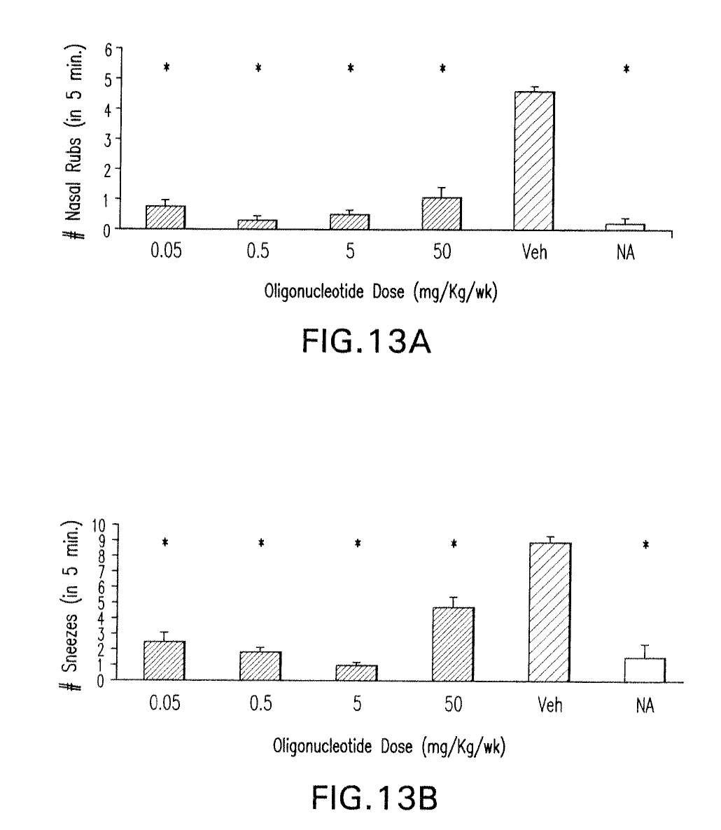

Observational analyses of mice following intranasal IL-4Rα ASO treatment and intranasal OVA challenge showed inhibition of behavioral responses, quantified as frequencies of sneezing and nasal rubbing, consistent with manifestation of allergic symptoms (FIG. 13).

These data demonstrate suppression of allergen-induced nasal inflammation and surrogates of clinical symptoms in mice treated with intranasal administration of IL-4Rα ASO. Intranasal application of IL-4Rα ASO was not associated with any pro-inflammatory changes in the nasal tissue, as assessed by examination of nasal lavage cell differential cell counts and nasal tissue histopathology. Reduction of nasal lavage and tissue eosinophils indicates repression of the local Th2 cytokine-mediated allergic inflammatory response.

### Example 7

### IL-4Rα Antisense Oligonucleotide Therapy Prevents Secondary Virus-mediated Disease in Mice

Mice

BALB/c mice are purchased as breeders from Harlan Laboratories. All mice are housed in a vivarium and maintained in ventilated micro-isolator cages housed in a specific pathogen-free animal facility. Sentinel mice within this animal colony are negative for antibodies to specific viral and other known mouse pathogens. Breeders are time-mated and two day old pups are used for experiments. All animal protocols are prepared in accordance with the Guide for the Care and Use of Laboratory Animals (National Research Council, 1996).

Oligonucleotides—Preparation and Dosage

IL-4Rα ASO and mismatch oligonucleotides (MM) are provided by Isis Pharmaceuticals (Carlsbad, Calif.) or a commercial manufacturer. The oligonucleotides are synthesized and purified as previously described in Karras et al. (2007) Am J Respir Cell Mol Biol 36:276-285. Both oligonucleotides are designed to avoid any murine immune stimulatory motifs and are about 20 bases in length with 2′-O-methoxyethylribose modification on bases 1 to 5 and 16 to 20 (underlined). The sequences of the ASO and MM are shown below; the mismatched bases are presented in lower case.

| | |
|---|---|
| ASO: | 5′-CCGCTGTICTCAGGTGACAT-3′ |
| MM: | 5′-CCaCTcaTCaCtGcTGACtT-3′. |

The oligonucleotides are suspended in sterile saline and administered intranasally (i.n.) to mouse pups at a dose of 100 or 500 μg/kg body weight. Control pups (i.e., SHAM, SV (sham plus virus), and SVR (sham plus virus, reinfected) receive sterile saline.

Virus Infection and Pulmonary Viral Load Determination

A respiratory virus (e.g., RSV, influenza, rhinovirus, or coronavirus) is purchased as a sucrose gradient-purified virus obtained from a commercial source. The virus preparation is determined to be free of bacteria, yeast, and fungi. Seven day old mice or 4-6 week old mice (protocol day 0) are anesthetized with 5% isofluorane and infected intranasally (i.n.) with optimized lethal or sublethal dose of virus in 10 μl serum-free media (VP-SFM; Invitrogen) per gram body weight or media alone as previously described (see, e.g., Becnel et al. (2005) Respir Res 6:122; You et al. (2006) Respir Res 7:107). Thirty-nine days later, (protocol day 39) the mice are similarly infected with the same optimized lethal or sublethal dose of virus in 10 μl of the same media.

To determine lung viral load, lungs are isolated at 6 days post-infection (dpi), quick-frozen in liquid nitrogen, and stored at −80° C. until processing. RNA is extracted from the lungs with TRlzol Reagent (Invitrogen) and purified with RNeasy Mini Kit (Qiagen) and DNase treated (Ambion). The RNA is then reverse transcribed into cDNA with a virus specific primer or oligo dT for GAPDH using the Superscript Ill-RT kit (Invitrogen) using standard, optimized conditions, for example, 65° C. for 5 min, 4° C. for 1 min, 42° C. for 50 min, and 85° C. for 5 min. The samples are placed at 4° C. for 1 min, RNase H is added, and the samples are incubated for 20 min. Real-time PCR is performed using specific forward and reverse primers for the given virus. After a 2 min denaturation at 95° C., PCR cycling conditions are 40 cycles of 95° C. for 10 sec, 60° C. for 30 sec, and 72° C. for 30 sec. This is followed by a melt-curve analysis: 1 min denaturation at 95° C., 1 min anneal at 55° C., and a 55-90° C. melt-curve (±0.5° C. Icycle; 30 sec) in a BioRad iQ5 machine. Selected PCR products are cloned into a TA cloning vector (pGEM-T, Promega), and the sequence is determined to confirm the identity of the virus detected by the PCR reaction. GAPDH internal control (Invitrogen Mouse/Rat GAPDH-Certified LUX™ Primer Set) is used to confirm equal quantities of input cDNA. Virus copy number is determined from standard curves of a plasmid vector containing a fragment of the virus gene.

Experimental Design

IL-4Rα ASO, MM, or saline is administered intranasally (i.n.) to mice on protocol days −5, −3, −1, and 1. On protocol day 0 (7 d of age or 4-6 weeks of age), mice are infected with the virus or sham infected. Mice receiving ASO/saline and infected with the virus are referred to as AV and SV, respectively. The mice receiving saline and sham infected are referred to as SHAM. For all secondary infections, mice are reinfected with the virus (AVR (antisense plus virus, reinfected), SVR (virus, reinfected)) or vehicle (SHAM) on protocol day 35. Various endpoints are measured including IL-4Rα levels, T cell populations in the lung, pulmonary function, bronchoalveolar lavage numbers and cytokine profile, pulmonary viral copy number, lung histology, and virus-specific antibody levels in serum at the indicated time points.

Isotyping and Quantification of Virus-specific Antibody

Serum is isolated from of mice following primary infection (7 and 12 dpi) using serum separator tubes (BO) and stored at −80° C. until use. Microtiter plates (Nunc-Immuno Maxisorp) are coated with 50 μl of an optimized amount of virus (e.g., 5×10⁴ pfu/ml) overnight at 4° C. in PBS. The plates are then blocked with 1× Blocker BSA (Pierce) for 5 min, 25 of serum is then added to each well and allowed to incubate for 2 h at room temperature. Bound virus-specific antibody is then isotyped and quantified using peroxidase-conjugated goat antibodies specific for mouse IgA, IgG1, IgG2a, and IgM (Southern Biotech) and 3,3′,5,5′-tetramethylbenzidine (TMB) (Pierce) as substrate. Color development is stopped with 2M H₂SO₄ and the optical density is read at 450 nm. Virus-specific antibody levels are determined by subtracting the absorbance value of the blank wells (media only) from the absorbance of each serum sample, then normalized to sham infected serum samples using the following equation: [(valueSHAM)/SHAM].

Determination of Surface Expression of IL-4Rα and Assessment of Pulmonary T Cell Populations

A single-cell suspension of lung cells is prepared using a standardized protocol (You et al. (2008) J Immunol 181:3486-3494). Lungs are perfused, excised, cut into small pieces, and incubated at for 1 h in RPMI 1640 media (HyClone) supplemented by 5% heat-inactivated FBS (HyClone), 100 U/ml penicillin, 100 mg/ml streptomycin (HyClone), 1 mg/ml collagenase I (Invitrogen), and 150 μg/ml DNase I (Sigma-Aldrich). After incubation, single cells are obtained by mashing the lung pieces through a cell strainer (BO Biosciences). Red blood cells are lysed using RBC lysis buffer (eBioscience) and cells are stained with combinations of the following antibodies purchased from BO and eBioscience: Pacific Blue-CD3e (17A2), PerCP-CD4 (RM4-5), FITC-CD8a (53-6.7), Biotin-CD124 (mIL4R-M1), APC-CD11b (M1/70), and PE Cy7-CD11c (N418), and E-cadherin (36/E-Cadherin).

For determining T cell subsets, lung cells (single-cell suspensions prepared as above and after RBC lysis) are stimulated for 5 h with 5 ng/ml phorbol-12-myristate-13acetate (PMA) and 500 ng/ml ionomycin (Sigma-Aldrich) in the presence of a protein transport inhibitor (10⁶ cells; GolgiPlug, BO Biosciences). After stimulation, cells are harvested, stained for surface markers (i.e., CD3, CD4, CD8), fixed, permeabilized (Fixation and Permeabilization Buffer; eBioscience), and stained with PE-IFN-γ (XMG1.2) and PE-Cy7-IL-4 (BVO4-24G2). Cell staining is determined with a FACSCanto II (BO Biosciences) flow cytometer after gating on specific live cell populations as determined by forward and side scatter properties and on CD3+ cells for T cell population analyses. Flow data are analyzed and plotted using FlowJo software (version 7.2.2 for Windows, Tree Star).

Assessment of Pulmonary Function

Six days after secondary infection, lung resistance and compliance to increasing doses of methacholine (MeCh, Sigma; 0, 12.5, 25, and 50 mg/ml in isotonic saline) are assessed using the forced oscillation technique. Animals are anesthetized with ketamine/xylazine (180/10 mg/kg) and mechanically ventilated with a tidal volume of 10 ml/kg and a frequency of 2.5 Hz using a computer controlled piston ventilator (FlexiVent Ver. 5.2R02, SCIREQ). Resistance and compliance data are calculated using the single compartment model. For comparison among the groups, all data are normalized to their individual baseline resistance values ((value-baseline)/baseline) and plotted as normalized resistance. Baseline values are not statistically different among the groups.

Determination of Bronchoalveolar Lavage Fluid Numbers and Cytokine Measurement

Bronchoalveolar lavage fluid (BALF) is isolated in 0.9 ml of PBS containing 2% BSA. Total BAL number is determined with the use of a hemocytometer. Cells (20,000) are centrifuged onto slides and are fixed and stained using the Hema-3 staining kit (Fisher Scientific). Two unbiased observers count 200-300 cells per slide using standard morphological criteria to classify individual leukocyte populations. Cytokine levels are measured from 50 μl of cell-free BALF using a high-throughput multiplex cytokine assay system (x-Plex Mouse Assay; Bio-Rad) according to the manufacturer's instructions. Three to four BALF samples per group are analyzed in duplicate on the Bio-Plex 200 system (Bio-Rad). Standards ranging from 0.2 to 6,296 pg/ml (depending on the analyte) are used to quantitate a dynamic range of cytokine concentrations. The concentrations of analytes in the samples are quantified using a standard curve, and a five-parameter logistic regression is performed to derive an equation that is then used to predict the concentration of the unknown samples. The following cytokines are assayed: IL-4, IL-5, IL-12(p40), IL12(p70), IL-13, and IFN-γ.

Assessment of Pulmonary Histopathology

Lungs are perfused with PBS containing 20 U/ml heparin, inflated gently to total lung capacity, and fixed in HistoChoice Tissue Fixative (Amresco, Inc) for 24 hours at 4° C. These tissues are then embedded in paraffin, cut in 4 frontal sections and stained with hematoxylin and eosin (H&E) or periodic acid-Schiff (PAS) to show inflammation and mucus hyperproduction in airway goblet cells, respectively. To evaluate the level of inflammation associated with secondary virus infection, two independent observers quantify the total number of airways in each lung section and then score each of these airways for inflammation (0: no inflammation or 1: inflammation consisting of cells thick). Data are reported as percentage of inflamed airways/total number of airways per lung section.

### Example 8

### Evaluation of Immune Response Following RSV Infection in Adult Mice Following Local Administration of IL-4Rα ASO

The present experiment was performed to determine whether inhibition of IL-4Rα using local delivery of antisense oligonucleotides alters the immune response to a viral infection.

Adult BALB/c mice 4-6 weeks of age were treated with IL-4-a ASO, IL-4Rα ASO-MM, or negative control diluent on protocol days 0, 2, 4, 6 and 8.

TABLE 16
Experimental Design

| | | | # An- | |
|---|---|---|---|---|
| | Expo- | | imals/ | Experimental |
| Group | sure | Treatment | Group | Endpoints |
| A | Sham | — | 12 | 4: BALs, Histology |
| | | | | 8: Viral titer |
| B | Sham | IL4-Rα ASO | 12 | 4: BALs, Histology |
| | | (100 μg/kg dose) | | 8: Viral titer |
| C | RSV | — | 12 | 4: BALs, Histology |
| | | | | 8: Viral titer |
| D | RSV | IL4-Rα ASO | 12 | 4: BALs, Histology |
| | | (1 μg/kg dose) | | 8: Viral titer |
| E | RSV | IL4-Rα ASO | 12 | 4: BALs, Histology |
| | | (100 μg/kg dose) | | 8: Viral titer |
| F | RSV | IL4-Rα ASO-MM | 12 | 4: BALs, Histology |
| | | (100 μg/kg dose) | | 8: Viral titer |

On protocol day 7, groups C-F were infected i.n. with RSV (10⁴ TCID₅₀/g body weight), and groups A-B received diluent. On protocol day 11 (peak viral titer), eight mice were euthanized and samples processed as outlined below, using the methods essentially as provided in Example 6.

Four animals—BALs were isolated with PBS+2% FBS and stored at −80° C. until use. Lungs for pathology were fixed in 10% neutral buffered formalin, 0/N, and transferred to EtOH until use.

Four animals—Lung homogenates were prepared, and viral titer was assessed using the TCID₅₀ method.

On protocol day 15, four mice were euthanized to assay for possible changes in viral clearance. The samples were processed as outlined below, using the methods essentially as provided in Example 6.

Four animals—Lung homogenates were prepared, and viral titer was assessed using the TCID₅₀ method.

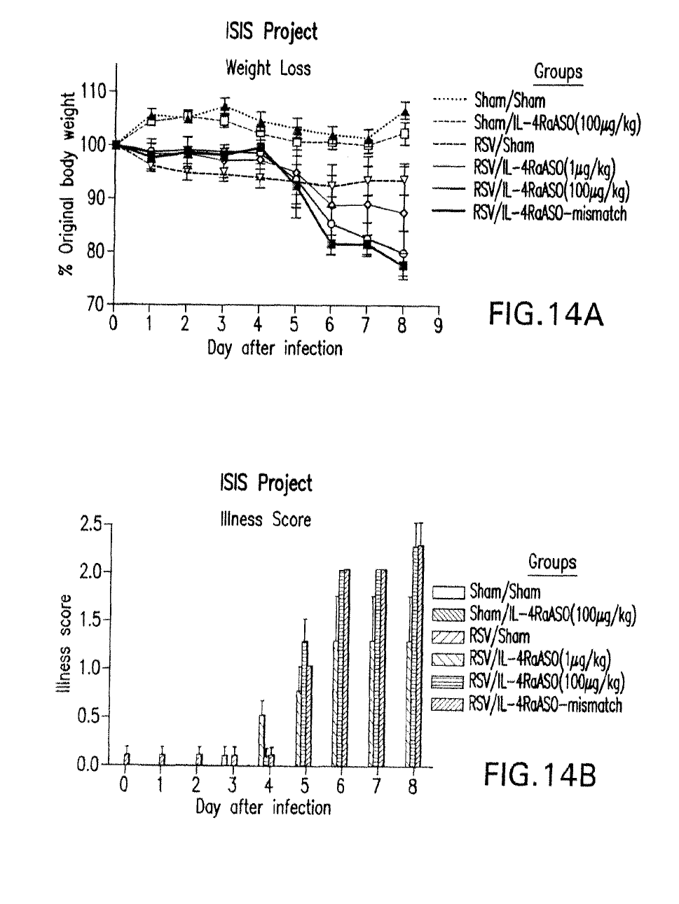

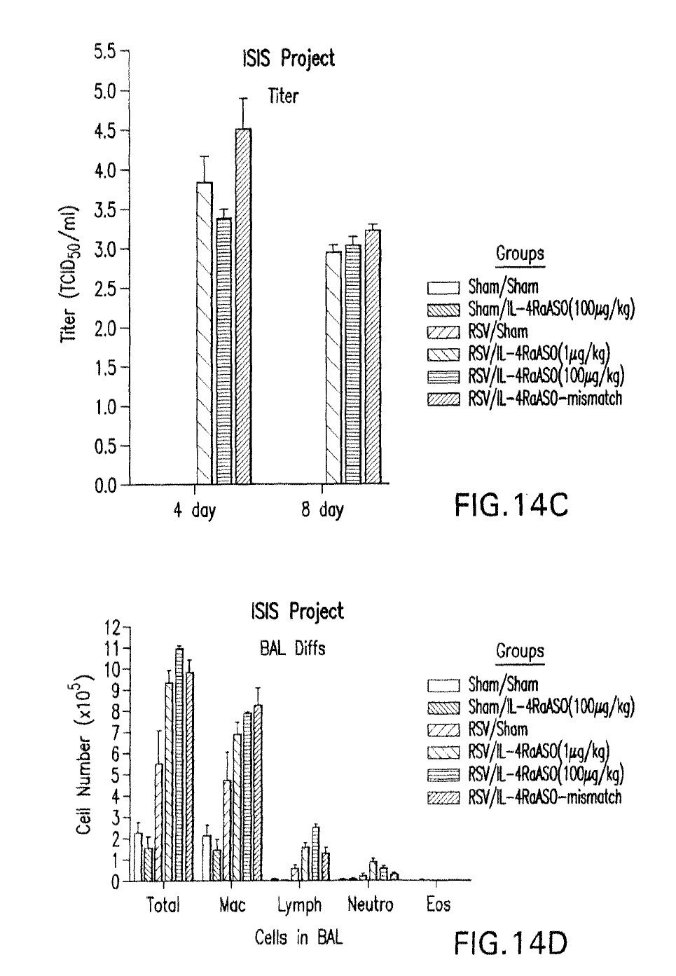

Various endpoints were evaluated, including BAL cytokines, pulmonary pathology, pulmonary viral titer, and pulmonary viral clearance using methods essentially as described in Example 6. As shown in FIG. 14, there was no significant difference in viral titers between sham treated mice, and mice treated with the IL-4Rα ASOs (FIG. 14C). Similarly, there was no significant differences seen between sham and ASO-treated mice in weight loss (FIG. 11A), illness score (FIG. 14B), and/or the numbers of macrophages, lymphocytes, neutrophils or eosinophils (or total) cell counts in the BAL (FIG. 14D).

Overall, these data show that administration of IL-4Rα ASOs does not exacerbate or cause deleterious Th1-based immune responses. These data also show that administration of IL-4Rα ASOs does not exacerbate viral infections.

### Sequence Listing

The present application is being filed with a computer readable form (CRF) copy of the Sequence Listing. The CRF entitled 12792-016-228_SEQLIST.txt, which was created on Mar. 31, 2010 and is 63,502 bytes in size, is identical to the paper copy of the Sequence Listing and is herein incorporated by reference in its entirety.

Other embodiments are within the following claims.

1-237. (canceled)

238. A method for modulating an immune response to a viral infection in a child or adult subject, comprising administering to the subject an effective amount of an antisense compound 12 to 35 nucleobases in length targeted to a nucleic acid molecule encoding human IL-4 receptor alpha (IL-4Rα) (SEQ ID NO:1), wherein said antisense compound inhibits expression of human IL-4Rα, thereby modulating the immune response to the viral infection in the child or adult subject.

239. The method of claim 238, wherein the viral infection is a primary viral infection.

240. The method of claim 238, wherein the viral infection is a secondary viral infection.

241. The method of claim 238, wherein the modulating comprises decreasing a Th2 response and/or increasing a Th1 response in the subject.

242. The method of claim 238, wherein the modulating comprises: (a) decreasing CD4+Th2+ T cell production in the subject; (b) increasing CD4+IFNγ+ T cell production in the subject; (c) decreasing CD8+IL-4+ T cell production in the subject; (d) increasing CD8+Th1+ T cell production in a subject; (e) increasing CD8+IFNγ T cell production in a subject; (f) decreasing airway hyperreactivity in the subject; (g) decreasing pulmonary inflammation in the subject; (h) maintaining or improving lung function in the subject; (i) decreasing airway resistance in the subject; (j) maintaining or increasing airway compliance in the subject; (k) decreasing absolute numbers of airway eosinophils or neutorphils in the subject; (l) decreasing an airway Th2 cytokine in the subject; (m) increasing serum IgG2a levels in the subject; (n) decreasing serum IgE levels in the subject; (o) decreasing eosinophils in the sputum or BAL of the subject; (p)
decreasing neutrophils in the sputum or BAL of the subject; (q) decreasing the level of a chemokine in the subject; (r) decreasing anti-viral antibody titers in the subject; or (s) any combination (a) to (r) thereof.

243. The method of claim 238, wherein the subject is a human child, adult or elderly adult.

244. The method of claim 238, wherein the subject isimmunocompromised and/or immunosuppressed.

245. The method of claim 238, wherein said modulating results in the management, treatment and/or amelioration of pulmonary inflammation, airway hyperreactivity and/or loss of lung function, or a symptom thereof, in the subject during the course of or resulting from the viral infection.

246. The method of claim 238, wherein said modulating results in the prevention or delay in the onset of pulmonary inflammation, airway hyperreactivity and/or loss of lung function, or a symptom thereof, in the subject during the course of or resulting from the viral infection.

247. The method of claim 238, wherein the viral infection is a rhinovirus, influenza virus or coronavirus infection.

248. The method of claim 238, wherein the viral infection is a respiratory syncytial virus infection.

249. The method of claim 238. wherein the antisense compound is administered to the upper and/or lower respiratory tract of the subject.

250. The method of claim 238, wherein the antisense compound is targeted to nucleotides 167-265, 487-525, 2056-2101, 2524-2598, 2731-2791, 3053-3072, or 3168-3187 of SEQ ID NO: 1.

251. A method for modulating an immune response to a rhinovirus, influenza virus or coronavirus infection in an infant subject, comprising administering to the subject an effective amount of an antisense compound 12 to 35 nucleobases in length targeted to a nucleic acid molecule encoding IL-4Rα (SEQ ID NO:1), wherein said antisense compound inhibits expression of human IL-4Rα.

252. The method of claim 251, wherein the modulating comprises decreasing a Th2 response and/or increasing a Th1 response in the subject.

253. The method of claim 251, wherein the modulating comprises: (a) decreasing CD4+Th2+ T cell production in the subject; (b) increasing CD4+IFNγ+ T cell production in the subject; (c) decreasing CD8+IL-4+ T cell production in the subject; (d) increasing CD8+Th1+ T cell production in a subject; (e) increasing CD8+IFNγ T cell production in a subject; (f) decreasing airway hyperreactivity in the subject; (g) decreasing pulmonary inflammation in the subject; (h) maintaining or improving lung function in the subject; (i) decreasing airway resistance in the subject; (j) maintaining or increasing airway compliance in the subject; (k) decreasing absolute numbers of airway eosinophils or neutorphils in the subject; (l) decreasing an airway Th2 cytokine in the subject; (m) increasing serum IgG2a levels in the subject; (n) decreasing serum IgE levels in the subject; (o) decreasing eosinophils in the sputum or BAL of the subject; (p) decreasing neutrophils in the sputum or BAL of the subject; (q) decreasing the level of a chemokine in the subject; (r) decreasing anti-viral antibody titers in the subject; or (s) any combination (a) to (r) thereof.

254. The method of claim 251, wherein the subject is a human infant.

255. The method of claim 251, wherein said modulating results in the management, treatment and/or amelioration of pulmonary inflammation, airway hyperreactivity and/or loss of lung function, or a symptom thereof, in the subject during the course of or resulting from the viral infection.

256. The method of claim 251, wherein said modulating results in the prevention or delay in the onset of pulmonary inflammation, airway hyperreactivity and/or loss of lung function, or a symptom thereof, in the subject during the course of or resulting from the viral infection.

257. The method of claim 251, wherein the antisense compound is administered to the upper and/or lower respiratory tract of the subject.

258. The method of claim 251, wherein the antisense compound is targeted to nucleotides 167-265, 487-525, 2056-2101, 2524-2598, 2731-2791, 3053-3072, or 3168-3187 of SEQ ID NO:1.


SEQUENCE LISTING (306 sequences)

SEQ ID NO: 1 - DNA, 3597 bp, Homo sapiens
ggcgaatggagcaggggcgcgcagataattaaagatttacacacagctggaagaaatcatagagaagccgggcgtggtggctcatgcctataatcccagcacttttggaggctgaggcgggcagatcacttgagatcaggagttcgagaccagcctggtgccttggcatctcccaatggggtggctttgctctgggctcctgttccctgtgagctgcctggtcctgctgcaggtggcaagctctgggaacatgaaggtcttgcaggagcccacctgcgtctccgactacatgagcatctctacttgcgagtggaagatgaatggtcccaccaattgcagcaccgagctccgcctgttgtaccagctggtttttctgctctccgaagcccacacgtgtatccctgagaacaacggaggcgcggggtgcgtgtgccacctgctcatggatgacgtggtcagtgcggataactatacactggacctgtgggctgggcagcagctgctgtggaagggctccttcaagcccagcgagcatgtgaaacccagggccccaggaaacctgacagttcacaccaatgtctccgacactctgctgctgacctggagcaacccgtatccccctgacaattacctgtataatcatctcacctatgcagtcaacatttggagtgaaaacgacccggcagatttcagaatctataacgtgacctacctagaaccctccctccgcatcgcagccagcaccctgaagtctgggatttcctacagggcacgggtgagggcctgggctcagtgctataacaccacctggagtgagtggagccccagcaccaagtggcacaactcctacagggagcccttcgagcagcacctcctgctgggcgtcagcgtttcctgcattgtcatcctggccgtctgcctgttgtgctatgtcagcatcaccaagattaagaaagaatggtgggatcagattcccaacccagcccgcagccgcctcgtggctataataatccaggatgctcaggggtcacagtgggagaagcggtcccgaggccaggaaccagccaagtgcccacactggaagaattgtcttaccaagctcttgccctgttttctggagcacaacatgaaaagggatgaagatcctcacaaggctgccaaagagatgcctttccagggctctggaaaatcagcatggtgcccagtggagatcagcaagacagtcctctggccagagagcatcagcgtggtgcgatgtgtggagttgtttgaggccccggtggagtgtgaggaggaggaggaggtagaggaagaaaaagggagcttctgtgcatcgcctgagagcagcagggatgacttccaggagggaagggagggcattgtggcccggctaacagagagcctgttcctggacctgctcggagaggagaatgggggcttttgccagcaggacatgggggagtcatgccttcttccaccttcgggaagtacgagtgctcacatgccctgggatgagttcccaagtgcagggcccaaggaggcacctccctggggcaaggagcagcctctccacctggagccaagtcctcctgccagcccgacccagagtccagacaacctgacttgcacagagacgcccctcgtcatcgcaggcaaccctgcttaccgcagcttcagcaactccctgagccagtcaccgtgtcccagagagctgggtccagacccactgctggccagacacctggaggaagtagaacccgagatgccctgtgtcccccagctctctgagccaaccactgtgccccaacctgagccagaaacctgggagcagatcctccgccgaaatgtcctccagcatggggcagctgcagcccccgtctcggcccccaccagtggctatcaggagtttgtacatgcggtggagcagggtggcacccaggccagtgcggtggtgggcttgggtcccccaggagaggctggttacaaggccttctcaagcctgcttgccagcagtgctgtgtccccagagaaatgtgggtttggggctagcagtggggaagaggggtataagcctttccaagacctcattcctggctgccctggggaccctgccccagtccctgtccccttgttcacctttggactggacagggagccacctcgcagtccgcagagctcacatctcccaagcagctccccagagcacctgggtctggagccgggggaaaaggtagaggacatgccaaagcccccacttccccaggagcaggccacagacccccttgtggacagcctgggcagtggcattgtctactcagcccttacctgccacctgtgcggccacctgaaacagtgtcatggccaggaggatggtggccagacccctgtcatggccagtccttgctgtggctgctgctgtggagacaggtcctcgccccctacaacccccctgagggccccagacccctctccaggtggggttccactggaggccagtctgtgtccggcctccctggcaccctcgggcatctcagagaagagtaaatcctcatcatccttccatcctgcccctggcaatgctcagagctcaagccagacccccaaaatcgtgaactttgtctccgtgggacccacatacatgagggtctcttaggtgcatgtcctcttgttgctgagtctgcagatgaggactagggcttatccatgcctgggaaatgccacctcctggaaggcagccaggctggcagatttccaaaagacttgaagaaccatggtatgaaggtgattggccccactgacgttggcctaacactgggctgcagagactggaccccgcccagcattgggctgggctcgccacatcccatgagagtagagggcactgggtcgccgtgccccacggcaggcccctgcaggaaaactgaggcccttgggcacctcgacttgtgaacgagttgttggctgctccctccacagcttctgcagcagactgtccctgttgtaactgcccaaggcatgttttgcccaccagatcatggcccacgtggaggcccacctgcctctgtctcactgaactagaagccgagcctagaaactaacacagccatcaagggaatgacttgggcggccttgggaaatcgatgagaaattgaacttcagggagggtggtcattgcctagaggtgctcattcatttaacagagcttccttaggttgatgctggaggcagaatcccggctgtcaaggggtgttcagttaaggggagcaacagaggacatgaaaaattgctatgactaaagcagggacaatttgctgccaaacacccatgcccagctgtatggctgggggctcctcgtatgcatggaacccccagaataaatatgctcagccaccctgtgggccgggcaatccagacagcaggcataaggcaccagttaccctgcatgttggcccagacctcaggtgctagggaaggcgggaaccttgggttgagtaatgctcgtctgtgtgttttagtttcatcacctgttatctgtgtttgctgaggagagtggaacagaaggggtggagttttgtataaataaagtttctttgtctc

SEQ ID NO: 2 - DNA, 3697 bp, Mus musculus
cgcaggaaagccccgcgcggcgcgtggagcctgaactcgcaggttctggctggacttctcgaagctgaggagaagcagagggacctggcttctgattttggatctgcgtgcttgctggttctggcgcctgctggtcttgttcctgtaacctaggactcggggcttgcacatgctttttttttgaagttgctggagagggagcccaggaccttgtgcaggcaccttttgtgtccccaatggggcggctttgcaccaagttcctgacctctgtgggctgtctgattttgctgttggtgactggatctgggagcatcaaggtcctgggtgagcccacctgcttctctgactacatccgcacttccacgtgtgagtggttcctggatagcgctgtggactgcagttctcagctctgcctacactacaggctgatgttcttcgagttctctgaaaacctcacatgcatcccgaggaacagtgccagcactgtgtgtgtgtgccacatggaaatgaataggccggtccaatcagacagataccagatggaactgtgggctgagcacagacagctgtggcagggctccttcagccccagtggtaatgtgaagcccctagctccagacaacctcacactccacaccaatgtgtccgacgaatggctgctgacctggaataacctgtacccatcgaacaacttactgtacaaagacctcatctccatggtcaacatctccagagaggacaaccctgcagaattcatagtctataatgtgacctacaaggaacccaggctgagcttcccgatcaacatcctgatgtcaggggtctactatacggcgcgtgtgagggtcagatcccagatactcactggcacctggagtgagtggagtcctagcatcacgtggtacaacccaagtaatgaaaatctgtgactgagtgaccttgggggctgcggtggtgaggagagctcacgggaatcctggagcagtgtagctggcgtgtcaaaagcagaaacgcaggagatggacttccagctgcccctgatacagcgccttccactgggggtcaccatctcctgcctctgcatcccgttgttttgcctgttctgttacttcagcattaccaagattaagaagatatggtgggaccagattcccaccccagcacgcagtcccttggtggccatcatcattcaggatgcacaggtgcccctctgggataagcagacccgaagccaggagtcaaccaagtacccgcactggaaaacttgtctagacaagctgctgccttgcttgctgaagcacagagtaaagaagaagacagacttcccgaaggctgccccaaccaagtctctccagagtcctggaaaggcaggctggtgtcccatggaggtcagcaggaccgtcctctggccagagaatgttagtgtcagtgtggtgcgctgtatggagctgtttgaggccccagtacagaatgtggaggaggaagaagatgagatagtcaaagaggacctgagcatgtcacctgagaacagcggaggctgcggcttccaggagagccaggcagacatcatggctcggctcactgagaacctgttttccgacttgttggaggctgagaatgggggccttggccagtcagccttggcagagtcatgctcccctctgccttcaggaagtgggcaggcttctgtatcctgggcctgcctccccatggggcccagtgaggaggccacatgccaggtcacagagcagccttcacacccaggccctctttcaggcagcccagcccagagtgcacctactctggcttgcacgcaggtcccacttgtccttgcagacaatcctgcctaccggagttttagtgactgctgtagcccggccccaaatcctggagagctggctccagagcagcagcaggctgatcatctggaagaagaggagcctccaagcccggctgacccccattcttcagggccaccaatgcagccagtggagagctgggagcagatccttcacatgagtgtcctgcagcatggggcagctgctggctccaccccagcccctgccggtggctaccaggagtttgtgcaggcagtgaagcagggtgccgcccaggatcctggggtgcctggtgtcaggccttctggagaccccggttacaaggccttctcgagcctgctcagcagcaatggcatccgcggggacacagcagcagcggggactgacgatgggcatggaggctacaagcccttccagaatcctgttcctaaccagtcccctagctccgtgcccttatttactttcggactagacacggagctgtcacccagtcctctgaactcagacccacccaaaagccccccagaatgccttggtctggagctggggctcaaaggaggtgactgggtgaaggcccctcctcctgcagatcaggtgcccaagccctttggggatgacctgggctttggtattgtgtactcgtccctcacttgccacttgtgtggccacctgaagcaacaccacagccaggaggaaggtggccagagccccatcgttgctagccctggctgtggctgctgctacgatgacagatcaccatccctggggagcctctcgggggccttggaaagctgtcctgagggaataccaccagaagccaacctcatgtcagcacccaagacaccctcaaacttgtcaggggagggcaagggccctggtcactctcctgttcccagccagacgaccgaggtgcctgtgggcgccctgggcattgctgtttcttaggtgagtgagtgtgctgttgttgctgaggtctgtgctgaggccagggttcctccaagccagggaagtacttcctgggagacagcccagctggcaggtttcccagaaatccagagaatggtgaattgaagatgtaaacttggcctgaccctggacgctcggagcctggctgtctcctcttccactggcctgggctctcctccctcccaagggatacaggggctcactgtgcttggtcccacagcagtgctgacgttcctaagtcctgggctttcctagctgatgttgtcctacctactcagtcccattttgtccaccgaatagacctgtcactcaaggctctcagcggtcctgccatagctgctggacgctcccagctggaagctgggcctagaaactcacagatggcctggcagtggcatgggaggccctaaaaattagtggaaattttgagagaggacaggtattgccccacagaggccattcattgaacagccaggactgggactagaggcagagcctgctgtcctccgctcagttgtagaaagcaacaaggacacaaacttgattgcccaaagtcactgccagttacccacatatgaccagaagccagggctcctgggatgtggaagataaacaaacacagttgccgggtggcagggccccagcgggcacgataactggcagtcaaggcgatacctcgagggaactgtggggctggtcctggttggtggtcaggtggtagggatagcagatggcagactttggtgagtgagtgagtctgactgtgttctggaagatgggaccgggctcagcactgtctgctcacgtccccactgttgcaacacctagtctgtttgcaaggaggacaggacaggtcacatggagctttatgtcaataaagtctttatcttgtc

SEQ ID NO: 3 - DNA, 19 bp, Artificial Sequence
  Antisense Compound
aatggtcccaccaattgca

SEQ ID NO: 4 - DNA, 23 bp, Artificial Sequence
  Antisense Compound
ctccgttgttctcagggatacac

SEQ ID NO: 5 - DNA, 21 bp, Artificial Sequence
  Antisense Compound
tttttctgctctccgaagccc

SEQ ID NO: 6 - DNA, 21 bp, Artificial Sequence
  Antisense Compound
tcccattttgtccaccgaata

SEQ ID NO: 7 - DNA, 20 bp, Artificial Sequence
  Antisense Compound
gtttctaggcccagcttcca

SEQ ID NO: 8 - DNA, 25 bp, Artificial Sequence
  Antisense Compound
tgtcactcaaggctctcagcggtcc

SEQ ID NO: 9 - DNA, 20 bp, Artificial Sequence
  Antisense Compound
acccgcacaaggtcctgggc

SEQ ID NO: 10 - DNA, 20 bp, Artificial Sequence
  Antisense Compound
caggtcttaccattaccact

SEQ ID NO: 11 - DNA, 20 bp, Artificial Sequence
  Antisense Compound
gcccactcacttctgcaggg

SEQ ID NO: 12 - DNA, 20 bp, Artificial Sequence
  Antisense Compound
cggttgtaccacgtgatgct

SEQ ID NO: 13 - DNA, 20 bp, Artificial Sequence
  Antisense Compound
tgatactcacggttgtacca

SEQ ID NO: 14 - DNA, 20 bp, Artificial Sequence
  Antisense Compound
aggaactcacttggtaatgc

SEQ ID NO: 15 - DNA, 20 bp, Artificial Sequence
  Antisense Compound
tgtaccctcttacctgtgca

SEQ ID NO: 16 - DNA, 20 bp, Artificial Sequence
  Antisense Compound
caaaaggtgcctgcgagttc

SEQ ID NO: 17 - DNA, 20 bp, Artificial Sequence
  Antisense Compound
ggctgggttacaggaacaag

SEQ ID NO: 18 - DNA, 20 bp, Artificial Sequence
  Antisense Compound
agctggaagtggttgtacca

SEQ ID NO: 19 - DNA, 20 bp, Artificial Sequence
  Antisense Compound
aatcagaagccaggtccctc

SEQ ID NO: 20 - DNA, 20 bp, Artificial Sequence
  Antisense Compound
caaaaggtgcctgcacaagg

SEQ ID NO: 21 - DNA, 20 bp, Artificial Sequence
  Antisense Compound
tgcaaagccgccccattggg

SEQ ID NO: 22 - DNA, 20 bp, Artificial Sequence
  Antisense Compound
caggaacttggtgcaaagcc

SEQ ID NO: 23 - DNA, 20 bp, Artificial Sequence
  Antisense Compound
tagtcagagaagcaggtggg

SEQ ID NO: 24 - DNA, 20 bp, Artificial Sequence
  Antisense Compound
agtgcggatgtagtcagaga

SEQ ID NO: 25 - DNA, 20 bp, Artificial Sequence
  Antisense Compound
ctgagaactgcagtccacag

SEQ ID NO: 26 - DNA, 20 bp, Artificial Sequence
  Antisense Compound
gtgaggttttcagagaactc

SEQ ID NO: 27 - DNA, 20 bp, Artificial Sequence
  Antisense Compound
tgcatgtgaggttttcagag

SEQ ID NO: 28 - DNA, 20 bp, Artificial Sequence
  Antisense Compound
gtgtgaggttgtctggagct

SEQ ID NO: 29 - DNA, 20 bp, Artificial Sequence
  Antisense Compound
acattggtgtggagtgtgag

SEQ ID NO: 30 - DNA, 20 bp, Artificial Sequence
  Antisense Compound
ctctggagatgttgaccatg

SEQ ID NO: 31 - DNA, 20 bp, Artificial Sequence
  Antisense Compound
gtcctctctggagatgttga

SEQ ID NO: 32 - DNA, 20 bp, Artificial Sequence
  Antisense Compound
gggttgtcctctctggagat

SEQ ID NO: 33 - DNA, 20 bp, Artificial Sequence
  Antisense Compound
tgtaggtcacattatagact

SEQ ID NO: 34 - DNA, 20 bp, Artificial Sequence
  Antisense Compound
gggttgtaccacgtgatgct

SEQ ID NO: 35 - DNA, 20 bp, Artificial Sequence
  Antisense Compound
tcactcagtcacagattttc

SEQ ID NO: 36 - DNA, 20 bp, Artificial Sequence
  Antisense Compound
agctggaagtccatctcctg

SEQ ID NO: 37 - DNA, 20 bp, Artificial Sequence
  Antisense Compound
cttcttaatcttggtaatgc

SEQ ID NO: 38 - DNA, 20 bp, Artificial Sequence
  Antisense Compound
accatatcttcttaatcttg

SEQ ID NO: 39 - DNA, 20 bp, Artificial Sequence
  Antisense Compound
gtcccaccatatcttcttaa

SEQ ID NO: 40 - DNA, 20 bp, Artificial Sequence
  Antisense Compound
atctggtcccaccatatctt

SEQ ID NO: 41 - DNA, 20 bp, Artificial Sequence
  Antisense Compound
tgggaatctggtcccaccat

SEQ ID NO: 42 - DNA, 20 bp, Artificial Sequence
  Antisense Compound
ggttgactcctggcttcggg

SEQ ID NO: 43 - DNA, 20 bp, Artificial Sequence
  Antisense Compound
ggacggtcctgctgacctcc

SEQ ID NO: 44 - DNA, 20 bp, Artificial Sequence
  Antisense Compound
ccagaggacggtcctgctga

SEQ ID NO: 45 - DNA, 20 bp, Artificial Sequence
  Antisense Compound
tctggccagaggacggtcct

SEQ ID NO: 46 - DNA, 20 bp, Artificial Sequence
  Antisense Compound
tacagcgcaccacactgaca

SEQ ID NO: 47 - DNA, 20 bp, Artificial Sequence
  Antisense Compound
gctccatacagcgcaccaca

SEQ ID NO: 48 - DNA, 20 bp, Artificial Sequence
  Antisense Compound
aaacagctccatacagcgca

SEQ ID NO: 49 - DNA, 20 bp, Artificial Sequence
  Antisense Compound
gcctcaaacagctccataca

SEQ ID NO: 50 - DNA, 20 bp, Artificial Sequence
  Antisense Compound
cctccacattctgtactggg

SEQ ID NO: 51 - DNA, 20 bp, Artificial Sequence
  Antisense Compound
caggtgacatgctcaggtcc

SEQ ID NO: 52 - DNA, 20 bp, Artificial Sequence
  Antisense Compound
gttctcaggtgacatgctca

SEQ ID NO: 53 - DNA, 20 bp, Artificial Sequence
  Antisense Compound
ccgctgttctcaggtgacat

SEQ ID NO: 54 - DNA, 20 bp, Artificial Sequence
  Antisense Compound
aacaggttctcagtgagccg

SEQ ID NO: 55 - DNA, 20 bp, Artificial Sequence
  Antisense Compound
ccggtaggcaggattgtctg

SEQ ID NO: 56 - DNA, 20 bp, Artificial Sequence
  Antisense Compound
aaactccggtaggcaggatt

SEQ ID NO: 57 - DNA, 20 bp, Artificial Sequence
  Antisense Compound
cactaaaactccggtaggca

SEQ ID NO: 58 - DNA, 20 bp, Artificial Sequence
  Antisense Compound
ccagctctccaggatttggg

SEQ ID NO: 59 - DNA, 20 bp, Artificial Sequence
  Antisense Compound
tggtggccctgaagaatggg

SEQ ID NO: 60 - DNA, 20 bp, Artificial Sequence
  Antisense Compound
ggatctgctcccagctctcc

SEQ ID NO: 61 - DNA, 20 bp, Artificial Sequence
  Antisense Compound
gtgaaggatctgctcccagc

SEQ ID NO: 62 - DNA, 20 bp, Artificial Sequence
  Antisense Compound
ctcatgtgaaggatctgctc

SEQ ID NO: 63 - DNA, 20 bp, Artificial Sequence
  Antisense Compound
ggacactcatgtgaaggatc

SEQ ID NO: 64 - DNA, 20 bp, Artificial Sequence
  Antisense Compound
ctgcaggacactcatgtgaa

SEQ ID NO: 65 - DNA, 20 bp, Artificial Sequence
  Antisense Compound
ttcactgcctgcacaaactc

SEQ ID NO: 66 - DNA, 20 bp, Artificial Sequence
  Antisense Compound
cctgcttcactgcctgcaca

SEQ ID NO: 67 - DNA, 20 bp, Artificial Sequence
  Antisense Compound
ctgctgagcaggctcgagaa

SEQ ID NO: 68 - DNA, 20 bp, Artificial Sequence
  Antisense Compound
gtcatccccaaagggcttgg

SEQ ID NO: 69 - DNA, 20 bp, Artificial Sequence
  Antisense Compound
cccaggtcatccccaaaggg

SEQ ID NO: 70 - DNA, 20 bp, Artificial Sequence
  Antisense Compound
gtgagggacgagtacacaat

SEQ ID NO: 71 - DNA, 20 bp, Artificial Sequence
  Antisense Compound
ttgcttcaggtggccacaca

SEQ ID NO: 72 - DNA, 20 bp, Artificial Sequence
  Antisense Compound
tggtgttgcttcaggtggcc

SEQ ID NO: 73 - DNA, 20 bp, Artificial Sequence
  Antisense Compound
ggctgtggtgttgcttcagg

SEQ ID NO: 74 - DNA, 20 bp, Artificial Sequence
  Antisense Compound
ctggctgggaacaggagagt

SEQ ID NO: 75 - DNA, 20 bp, Artificial Sequence
  Antisense Compound
agcaacaacagcacactcac

SEQ ID NO: 76 - DNA, 20 bp, Artificial Sequence
  Antisense Compound
acctcagcaacaacagcaca

SEQ ID NO: 77 - DNA, 20 bp, Artificial Sequence
  Antisense Compound
cacagacctcagcaacaaca

SEQ ID NO: 78 - DNA, 20 bp, Artificial Sequence
  Antisense Compound
tccctggcttggaggaaccc

SEQ ID NO: 79 - DNA, 20 bp, Artificial Sequence
  Antisense Compound
cctgccagctgggctgtctc

SEQ ID NO: 80 - DNA, 20 bp, Artificial Sequence
  Antisense Compound
ttctgggaaacctgccagct

SEQ ID NO: 81 - DNA, 20 bp, Artificial Sequence
  Antisense Compound
actttgggcaatcaagtttg

SEQ ID NO: 82 - DNA, 20 bp, Artificial Sequence
  Antisense Compound
cagtgactttgggcaatcaa

SEQ ID NO: 83 - DNA, 20 bp, Artificial Sequence
  Antisense Compound
actggcagtgactttgggca

SEQ ID NO: 84 - DNA, 20 bp, Artificial Sequence
  Antisense Compound
gggtaactggcagtgacttt

SEQ ID NO: 85 - DNA, 20 bp, Artificial Sequence
  Antisense Compound
taaagactttattgacataa

SEQ ID NO: 86 - DNA, 20 bp, Artificial Sequence
  Antisense Compound
gacaagataaagactttatt

SEQ ID NO: 87 - DNA, 20 bp, Artificial Sequence
  Antisense Compound
gtaaatctttaattatctgc

SEQ ID NO: 88 - DNA, 20 bp, Artificial Sequence
  Antisense Compound
catgttcccagagcttgcca

SEQ ID NO: 89 - DNA, 20 bp, Artificial Sequence
  Antisense Compound
ctgcaagaccttcatgttcc

SEQ ID NO: 90 - DNA, 20 bp, Artificial Sequence
  Antisense Compound
caagtagagatgctcatgta

SEQ ID NO: 91 - DNA, 20 bp, Artificial Sequence
  Antisense Compound
caattggtgggaccattcat

SEQ ID NO: 92 - DNA, 20 bp, Artificial Sequence
  Antisense Compound
acagcagctgctgcccagcc

SEQ ID NO: 93 - DNA, 20 bp, Artificial Sequence
  Antisense Compound
aatcccagacttcagggtgc

SEQ ID NO: 94 - DNA, 20 bp, Artificial Sequence
  Antisense Compound
gcactgagcccaggccctca

SEQ ID NO: 95 - DNA, 20 bp, Artificial Sequence
  Antisense Compound
ctgacatagcacaacaggca

SEQ ID NO: 96 - DNA, 20 bp, Artificial Sequence
  Antisense Compound
taatcttggtgatgctgaca

SEQ ID NO: 97 - DNA, 20 bp, Artificial Sequence
  Antisense Compound
tttcttaatcttggtgatgc

SEQ ID NO: 98 - DNA, 20 bp, Artificial Sequence
  Antisense Compound
ccctggaaaggcatctcttt

SEQ ID NO: 99 - DNA, 20 bp, Artificial Sequence
  Antisense Compound
gctgattttccagagccctg

SEQ ID NO: 100 - DNA, 20 bp, Artificial Sequence
  Antisense Compound
gcaccatgctgattttccag

SEQ ID NO: 101 - DNA, 20 bp, Artificial Sequence
  Antisense Compound
cccagggcatgtgagcactc

SEQ ID NO: 102 - DNA, 20 bp, Artificial Sequence
  Antisense Compound
aactcatcccagggcatgtg

SEQ ID NO: 103 - DNA, 20 bp, Artificial Sequence
  Antisense Compound
tgcacttgggaactcatccc

SEQ ID NO: 104 - DNA, 20 bp, Artificial Sequence
  Antisense Compound
gcaagtcaggttgtctggac

SEQ ID NO: 105 - DNA, 20 bp, Artificial Sequence
  Antisense Compound
gtgggtctggacccagctct

SEQ ID NO: 106 - DNA, 20 bp, Artificial Sequence
  Antisense Compound
ggccagcagtgggtctggac

SEQ ID NO: 107 - DNA, 20 bp, Artificial Sequence
  Antisense Compound
tgccccatgctggaggacat

SEQ ID NO: 108 - DNA, 20 bp, Artificial Sequence
  Antisense Compound
gagaaggccttgtaaccagc

SEQ ID NO: 109 - DNA, 20 bp, Artificial Sequence
  Antisense Compound
acagcactgctggcaagcag

SEQ ID NO: 110 - DNA, 20 bp, Artificial Sequence
  Antisense Compound
ccccactgctagccccaaac

SEQ ID NO: 111 - DNA, 20 bp, Artificial Sequence
  Antisense Compound
ctcttccccactgctagccc

SEQ ID NO: 112 - DNA, 20 bp, Artificial Sequence
  Antisense Compound
gaaaggcttatacccctctt

SEQ ID NO: 113 - DNA, 20 bp, Artificial Sequence
  Antisense Compound
gaggtcttggaaaggcttat

SEQ ID NO: 114 - DNA, 20 bp, Artificial Sequence
  Antisense Compound
agggcagccaggaatgaggt

SEQ ID NO: 115 - DNA, 20 bp, Artificial Sequence
  Antisense Compound
tccccagggcagccaggaat

SEQ ID NO: 116 - DNA, 20 bp, Artificial Sequence
  Antisense Compound
gctttggcatgtcctctacc

SEQ ID NO: 117 - DNA, 20 bp, Artificial Sequence
  Antisense Compound
ggctgagtagacaatgccac

SEQ ID NO: 118 - DNA, 20 bp, Artificial Sequence
  Antisense Compound
aggtggcaggtaagggctga

SEQ ID NO: 119 - DNA, 20 bp, Artificial Sequence
  Antisense Compound
ccacagcaaggactggccat

SEQ ID NO: 120 - DNA, 20 bp, Artificial Sequence
  Antisense Compound
cagtggaaccccacctggag

SEQ ID NO: 121 - DNA, 20 bp, Artificial Sequence
  Antisense Compound
gaaggatgatgaggatttac

SEQ ID NO: 122 - DNA, 20 bp, Artificial Sequence
  Antisense Compound
caggatggaaggatgatgag

SEQ ID NO: 123 - DNA, 20 bp, Artificial Sequence
  Antisense Compound
agctctgagcattgccaggg

SEQ ID NO: 124 - DNA, 20 bp, Artificial Sequence
  Antisense Compound
ccctcatgtatgtgggtccc

SEQ ID NO: 125 - DNA, 20 bp, Artificial Sequence
  Antisense Compound
gacatgcacctaagagaccc

SEQ ID NO: 126 - DNA, 20 bp, Artificial Sequence
  Antisense Compound
tagtcctcatctgcagactc

SEQ ID NO: 127 - DNA, 20 bp, Artificial Sequence
  Antisense Compound
aatctgccagcctggctgcc

SEQ ID NO: 128 - DNA, 20 bp, Artificial Sequence
  Antisense Compound
ggttcttcaagtcttttgga

SEQ ID NO: 129 - DNA, 20 bp, Artificial Sequence
  Antisense Compound
ggccaatcaccttcatacca

SEQ ID NO: 130 - DNA, 20 bp, Artificial Sequence
  Antisense Compound
gagcccagcccaatgctggg

SEQ ID NO: 131 - DNA, 20 bp, Artificial Sequence
  Antisense Compound
ctactctcatgggatgtggc

SEQ ID NO: 132 - DNA, 20 bp, Artificial Sequence
  Antisense Compound
gccctctactctcatgggat

SEQ ID NO: 133 - DNA, 20 bp, Artificial Sequence
  Antisense Compound
ggcctcagttttcctgcagg

SEQ ID NO: 134 - DNA, 20 bp, Artificial Sequence
  Antisense Compound
cccaagggcctcagttttcc

SEQ ID NO: 135 - DNA, 20 bp, Artificial Sequence
  Antisense Compound
gagggagcagccaacaactc

SEQ ID NO: 136 - DNA, 20 bp, Artificial Sequence
  Antisense Compound
agacagaggcaggtgggcct

SEQ ID NO: 137 - DNA, 20 bp, Artificial Sequence
  Antisense Compound
cagtgagacagaggcaggtg

SEQ ID NO: 138 - DNA, 20 bp, Artificial Sequence
  Antisense Compound
caagtcattcccttgatggc

SEQ ID NO: 139 - DNA, 20 bp, Artificial Sequence
  Antisense Compound
atcaacctaaggaagctctg

SEQ ID NO: 140 - DNA, 20 bp, Artificial Sequence
  Antisense Compound
taactgaacaccccttgaca

SEQ ID NO: 141 - DNA, 20 bp, Artificial Sequence
  Antisense Compound
aattgtccctgctttagtca

SEQ ID NO: 142 - DNA, 20 bp, Artificial Sequence
  Antisense Compound
ggcagcaaattgtccctgct

SEQ ID NO: 143 - DNA, 20 bp, Artificial Sequence
  Antisense Compound
gtgtttggcagcaaattgtc

SEQ ID NO: 144 - DNA, 20 bp, Artificial Sequence
  Antisense Compound
gggtaactggtgccttatgc

SEQ ID NO: 145 - DNA, 20 bp, Artificial Sequence
  Antisense Compound
ggccaacatgcagggtaact

SEQ ID NO: 146 - DNA, 20 bp, Artificial Sequence
  Antisense Compound
attactcaacccaaggttcc

SEQ ID NO: 147 - DNA, 20 bp, Artificial Sequence
  Antisense Compound
aagaaactttatttatacaa

SEQ ID NO: 148 - DNA, 20 bp, Artificial Sequence
  Antisense Compound
gagacaaagaaactttattt

SEQ ID NO: 149 - DNA, 20 bp, Artificial Sequence
  Antisense Compound
cctagaattcagtcttccct

SEQ ID NO: 150 - DNA, 20 bp, Artificial Sequence
  Antisense Compound
gtttccatctagagtactag

SEQ ID NO: 151 - DNA, 20 bp, Artificial Sequence
  Antisense Compound
gccaaggcacctgcagagag

SEQ ID NO: 152 - DNA, 20 bp, Artificial Sequence
  Antisense Compound
agtgagtggcagagtcagga

SEQ ID NO: 153 - DNA, 20 bp, Artificial Sequence
  Antisense Compound
cttccagtgtctgcaaaagc

SEQ ID NO: 154 - DNA, 20 bp, Artificial Sequence
  Antisense Compound
agccaccccattgggagatg

SEQ ID NO: 155 - DNA, 20 bp, Artificial Sequence
  Antisense Compound
gagcaaagccaccccattgg

SEQ ID NO: 156 - DNA, 20 bp, Artificial Sequence
  Antisense Compound
ccagagcaaagccaccccat

SEQ ID NO: 157 - DNA, 20 bp, Artificial Sequence
  Antisense Compound
tcacagggaacaggagccca

SEQ ID NO: 158 - DNA, 20 bp, Artificial Sequence
  Antisense Compound
ctcacagggaacaggagccc

SEQ ID NO: 159 - DNA, 20 bp, Artificial Sequence
  Antisense Compound
agctcacagggaacaggagc

SEQ ID NO: 160 - DNA, 20 bp, Artificial Sequence
  Antisense Compound
cagctcacagggaacaggag

SEQ ID NO: 161 - DNA, 20 bp, Artificial Sequence
  Antisense Compound
ggcagctcacagggaacagg

SEQ ID NO: 162 - DNA, 20 bp, Artificial Sequence
  Antisense Compound
aggcagctcacagggaacag

SEQ ID NO: 163 - DNA, 20 bp, Artificial Sequence
  Antisense Compound
caggcagctcacagggaaca

SEQ ID NO: 164 - DNA, 20 bp, Artificial Sequence
  Antisense Compound
ccaggcagctcacagggaac

SEQ ID NO: 165 - DNA, 20 bp, Artificial Sequence
  Antisense Compound
accaggcagctcacagggaa

SEQ ID NO: 166 - DNA, 20 bp, Artificial Sequence
  Antisense Compound
ggaccaggcagctcacaggg

SEQ ID NO: 167 - DNA, 20 bp, Artificial Sequence
  Antisense Compound
aggaccaggcagctcacagg

SEQ ID NO: 168 - DNA, 20 bp, Artificial Sequence
  Antisense Compound
caggaccaggcagctcacag

SEQ ID NO: 169 - DNA, 20 bp, Artificial Sequence
  Antisense Compound
gcaggaccaggcagctcaca

SEQ ID NO: 170 - DNA, 20 bp, Artificial Sequence
  Antisense Compound
agcaggaccaggcagctcac

SEQ ID NO: 171 - DNA, 20 bp, Artificial Sequence
  Antisense Compound
cagcaggaccaggcagctca

SEQ ID NO: 172 - DNA, 20 bp, Artificial Sequence
  Antisense Compound
gcagcaggaccaggcagctc

SEQ ID NO: 173 - DNA, 20 bp, Artificial Sequence
  Antisense Compound
tgcagcaggaccaggcagct

SEQ ID NO: 174 - DNA, 20 bp, Artificial Sequence
  Antisense Compound
ctgcagcaggaccaggcagc

SEQ ID NO: 175 - DNA, 20 bp, Artificial Sequence
  Antisense Compound
acctgcagcaggaccaggca

SEQ ID NO: 176 - DNA, 20 bp, Artificial Sequence
  Antisense Compound
ccacctgcagcaggaccagg

SEQ ID NO: 177 - DNA, 20 bp, Artificial Sequence
  Antisense Compound
tgccacctgcagcaggacca

SEQ ID NO: 178 - DNA, 20 bp, Artificial Sequence
  Antisense Compound
ttgccacctgcagcaggacc

SEQ ID NO: 179 - DNA, 20 bp, Artificial Sequence
  Antisense Compound
cttgccacctgcagcaggac

SEQ ID NO: 180 - DNA, 20 bp, Artificial Sequence
  Antisense Compound
gcttgccacctgcagcagga

SEQ ID NO: 181 - DNA, 20 bp, Artificial Sequence
  Antisense Compound
agcttgccacctgcagcagg

SEQ ID NO: 182 - DNA, 20 bp, Artificial Sequence
  Antisense Compound
gagcttgccacctgcagcag

SEQ ID NO: 183 - DNA, 20 bp, Artificial Sequence
  Antisense Compound
agagcttgccacctgcagca

SEQ ID NO: 184 - DNA, 20 bp, Artificial Sequence
  Antisense Compound
cagagcttgccacctgcagc

SEQ ID NO: 185 - DNA, 20 bp, Artificial Sequence
  Antisense Compound
ccagagcttgccacctgcag

SEQ ID NO: 186 - DNA, 20 bp, Artificial Sequence
  Antisense Compound
cccagagcttgccacctgca

SEQ ID NO: 187 - DNA, 20 bp, Artificial Sequence
  Antisense Compound
tcccagagcttgccacctgc

SEQ ID NO: 188 - DNA, 20 bp, Artificial Sequence
  Antisense Compound
gtagagatgctcatgtagtc

SEQ ID NO: 189 - DNA, 20 bp, Artificial Sequence
  Antisense Compound
aaaaccagctggtacaacag

SEQ ID NO: 190 - DNA, 20 bp, Artificial Sequence
  Antisense Compound
gaaaaaccagctggtacaac

SEQ ID NO: 191 - DNA, 20 bp, Artificial Sequence
  Antisense Compound
tccatgagcaggtggcacac

SEQ ID NO: 192 - DNA, 20 bp, Artificial Sequence
  Antisense Compound
atccatgagcaggtggcaca

SEQ ID NO: 193 - DNA, 20 bp, Artificial Sequence
  Antisense Compound
catccatgagcaggtggcac

SEQ ID NO: 194 - DNA, 20 bp, Artificial Sequence
  Antisense Compound
tcatccatgagcaggtggca

SEQ ID NO: 195 - DNA, 20 bp, Artificial Sequence
  Antisense Compound
cccttccacagcagctgctg

SEQ ID NO: 196 - DNA, 20 bp, Artificial Sequence
  Antisense Compound
agcccttccacagcagctgc

SEQ ID NO: 197 - DNA, 20 bp, Artificial Sequence
  Antisense Compound
gagcccttccacagcagctg

SEQ ID NO: 198 - DNA, 20 bp, Artificial Sequence
  Antisense Compound
aggagcccttccacagcagc

SEQ ID NO: 199 - DNA, 20 bp, Artificial Sequence
  Antisense Compound
aaggagcccttccacagcag

SEQ ID NO: 200 - DNA, 20 bp, Artificial Sequence
  Antisense Compound
gaaggagcccttccacagca

SEQ ID NO: 201 - DNA, 20 bp, Artificial Sequence
  Antisense Compound
tgaaggagcccttccacagc

SEQ ID NO: 202 - DNA, 20 bp, Artificial Sequence
  Antisense Compound
ttgaaggagcccttccacag

SEQ ID NO: 203 - DNA, 20 bp, Artificial Sequence
  Antisense Compound
cttgaaggagcccttccaca

SEQ ID NO: 204 - DNA, 20 bp, Artificial Sequence
  Antisense Compound
ggcttgaaggagcccttcca

SEQ ID NO: 205 - DNA, 20 bp, Artificial Sequence
  Antisense Compound
tgggcttgaaggagcccttc

SEQ ID NO: 206 - DNA, 20 bp, Artificial Sequence
  Antisense Compound
ctgggcttgaaggagccctt

SEQ ID NO: 207 - DNA, 20 bp, Artificial Sequence
  Antisense Compound
gctgggcttgaaggagccct

SEQ ID NO: 208 - DNA, 20 bp, Artificial Sequence
  Antisense Compound
gccctgggtttcacatgctc

SEQ ID NO: 209 - DNA, 20 bp, Artificial Sequence
  Antisense Compound
ggccctgggtttcacatgct

SEQ ID NO: 210 - DNA, 20 bp, Artificial Sequence
  Antisense Compound
tatacaggtaattgtcaggg

SEQ ID NO: 211 - DNA, 20 bp, Artificial Sequence
  Antisense Compound
ttatacaggtaattgtcagg

SEQ ID NO: 212 - DNA, 20 bp, Artificial Sequence
  Antisense Compound
attatacaggtaattgtcag

SEQ ID NO: 213 - DNA, 20 bp, Artificial Sequence
  Antisense Compound
gttgactgcataggtgagat

SEQ ID NO: 214 - DNA, 20 bp, Artificial Sequence
  Antisense Compound
aatgttgactgcataggtga

SEQ ID NO: 215 - DNA, 20 bp, Artificial Sequence
  Antisense Compound
caaatgttgactgcataggt

SEQ ID NO: 216 - DNA, 20 bp, Artificial Sequence
  Antisense Compound
tccaaatgttgactgcatag

SEQ ID NO: 217 - DNA, 20 bp, Artificial Sequence
  Antisense Compound
agacttcagggtgctggctg

SEQ ID NO: 218 - DNA, 20 bp, Artificial Sequence
  Antisense Compound
cagacttcagggtgctggct

SEQ ID NO: 219 - DNA, 20 bp, Artificial Sequence
  Antisense Compound
ccagacttcagggtgctggc

SEQ ID NO: 220 - DNA, 20 bp, Artificial Sequence
  Antisense Compound
tcctggattattatagccac

SEQ ID NO: 221 - DNA, 20 bp, Artificial Sequence
  Antisense Compound
atcctggattattatagcca

SEQ ID NO: 222 - DNA, 20 bp, Artificial Sequence
  Antisense Compound
catcctggattattatagcc

SEQ ID NO: 223 - DNA, 20 bp, Artificial Sequence
  Antisense Compound
gcatcctggattattatagc

SEQ ID NO: 224 - DNA, 20 bp, Artificial Sequence
  Antisense Compound
gagcatcctggattattata

SEQ ID NO: 225 - DNA, 20 bp, Artificial Sequence
  Antisense Compound
tgagcatcctggattattat

SEQ ID NO: 226 - DNA, 20 bp, Artificial Sequence
  Antisense Compound
ctgagcatcctggattatta

SEQ ID NO: 227 - DNA, 20 bp, Artificial Sequence
  Antisense Compound
cctgagcatcctggattatt

SEQ ID NO: 228 - DNA, 20 bp, Artificial Sequence
  Antisense Compound
gcacttggctggttcctggc

SEQ ID NO: 229 - DNA, 20 bp, Artificial Sequence
  Antisense Compound
ggtaagacaattcttccagt

SEQ ID NO: 230 - DNA, 20 bp, Artificial Sequence
  Antisense Compound
tggtaagacaattcttccag

SEQ ID NO: 231 - DNA, 20 bp, Artificial Sequence
  Antisense Compound
ttggtaagacaattcttcca

SEQ ID NO: 232 - DNA, 20 bp, Artificial Sequence
  Antisense Compound
cttggtaagacaattcttcc

SEQ ID NO: 233 - DNA, 20 bp, Artificial Sequence
  Antisense Compound
agcttggtaagacaattctt

SEQ ID NO: 234 - DNA, 20 bp, Artificial Sequence
  Antisense Compound
gagcttggtaagacaattct

SEQ ID NO: 235 - DNA, 20 bp, Artificial Sequence
  Antisense Compound
aagagcttggtaagacaatt

SEQ ID NO: 236 - DNA, 20 bp, Artificial Sequence
  Antisense Compound
gcaagagcttggtaagacaa

SEQ ID NO: 237 - DNA, 20 bp, Artificial Sequence
  Antisense Compound
ggcaagagcttggtaagaca

SEQ ID NO: 238 - DNA, 20 bp, Artificial Sequence
  Antisense Compound
agggcaagagcttggtaaga

SEQ ID NO: 239 - DNA, 20 bp, Artificial Sequence
  Antisense Compound
acagggcaagagcttggtaa

SEQ ID NO: 240 - DNA, 20 bp, Artificial Sequence
  Antisense Compound
aacagggcaagagcttggta

SEQ ID NO: 241 - DNA, 20 bp, Artificial Sequence
  Antisense Compound
aaacagggcaagagcttggt

SEQ ID NO: 242 - DNA, 20 bp, Artificial Sequence
  Antisense Compound
aaaacagggcaagagcttgg

SEQ ID NO: 243 - DNA, 20 bp, Artificial Sequence
  Antisense Compound
gaaaacagggcaagagcttg

SEQ ID NO: 244 - DNA, 20 bp, Artificial Sequence
  Antisense Compound
cagaaaacagggcaagagct

SEQ ID NO: 245 - DNA, 20 bp, Artificial Sequence
  Antisense Compound
tccagaaaacagggcaagag

SEQ ID NO: 246 - DNA, 20 bp, Artificial Sequence
  Antisense Compound
gggcaccatgctgattttcc

SEQ ID NO: 247 - DNA, 20 bp, Artificial Sequence
  Antisense Compound
gctctctggccagaggactg

SEQ ID NO: 248 - DNA, 20 bp, Artificial Sequence
  Antisense Compound
atgctctctggccagaggac

SEQ ID NO: 249 - DNA, 20 bp, Artificial Sequence
  Antisense Compound
gatgctctctggccagagga

SEQ ID NO: 250 - DNA, 20 bp, Artificial Sequence
  Antisense Compound
gctgatgctctctggccaga

SEQ ID NO: 251 - DNA, 20 bp, Artificial Sequence
  Antisense Compound
gtccaggaacaggctctctg

SEQ ID NO: 252 - DNA, 20 bp, Artificial Sequence
  Antisense Compound
aggtccaggaacaggctctc

SEQ ID NO: 253 - DNA, 20 bp, Artificial Sequence
  Antisense Compound
caggtccaggaacaggctct

SEQ ID NO: 254 - DNA, 20 bp, Artificial Sequence
  Antisense Compound
gcaggtccaggaacaggctc

SEQ ID NO: 255 - DNA, 20 bp, Artificial Sequence
  Antisense Compound
agcaggtccaggaacaggct

SEQ ID NO: 256 - DNA, 20 bp, Artificial Sequence
  Antisense Compound
gagcaggtccaggaacaggc

SEQ ID NO: 257 - DNA, 20 bp, Artificial Sequence
  Antisense Compound
acttgggaactcatcccagg

SEQ ID NO: 258 - DNA, 20 bp, Artificial Sequence
  Antisense Compound
cacttgggaactcatcccag

SEQ ID NO: 259 - DNA, 20 bp, Artificial Sequence
  Antisense Compound
gcacttgggaactcatccca

SEQ ID NO: 260 - DNA, 20 bp, Artificial Sequence
  Antisense Compound
ctcagggagttgctgaagct

SEQ ID NO: 261 - DNA, 20 bp, Artificial Sequence
  Antisense Compound
gctcagggagttgctgaagc

SEQ ID NO: 262 - DNA, 20 bp, Artificial Sequence
  Antisense Compound
tggctcagggagttgctgaa

SEQ ID NO: 263 - DNA, 20 bp, Artificial Sequence
  Antisense Compound
ctggctcagggagttgctga

SEQ ID NO: 264 - DNA, 20 bp, Artificial Sequence
  Antisense Compound
gactggctcagggagttgct

SEQ ID NO: 265 - DNA, 20 bp, Artificial Sequence
  Antisense Compound
ggacccagctctctgggaca

SEQ ID NO: 266 - DNA, 20 bp, Artificial Sequence
  Antisense Compound
tggacccagctctctgggac

SEQ ID NO: 267 - DNA, 20 bp, Artificial Sequence
  Antisense Compound
tctggacccagctctctggg

SEQ ID NO: 268 - DNA, 20 bp, Artificial Sequence
  Antisense Compound
ggtctggacccagctctctg

SEQ ID NO: 269 - DNA, 20 bp, Artificial Sequence
  Antisense Compound
gggtctggacccagctctct

SEQ ID NO: 270 - DNA, 20 bp, Artificial Sequence
  Antisense Compound
tggttggctcagagagctgg

SEQ ID NO: 271 - DNA, 20 bp, Artificial Sequence
  Antisense Compound
agtggttggctcagagagct

SEQ ID NO: 272 - DNA, 20 bp, Artificial Sequence
  Antisense Compound
cagtggttggctcagagagc

SEQ ID NO: 273 - DNA, 20 bp, Artificial Sequence
  Antisense Compound
acagtggttggctcagagag

SEQ ID NO: 274 - DNA, 20 bp, Artificial Sequence
  Antisense Compound
cacagtggttggctcagaga

SEQ ID NO: 275 - DNA, 20 bp, Artificial Sequence
  Antisense Compound
gcactgctggcaagcaggct

SEQ ID NO: 276 - DNA, 20 bp, Artificial Sequence
  Antisense Compound
aaggcttatacccctcttcc

SEQ ID NO: 277 - DNA, 20 bp, Artificial Sequence
  Antisense Compound
aaaggcttatacccctcttc

SEQ ID NO: 278 - skipped

SEQ ID NO: 279 - DNA, 20 bp, Artificial Sequence
  Antisense Compound
ggaaaggcttatacccctct

SEQ ID NO: 280 - DNA, 20 bp, Artificial Sequence
  Antisense Compound
tggaaaggcttatacccctc

SEQ ID NO: 281 - DNA, 20 bp, Artificial Sequence
  Antisense Compound
cttggaaaggcttatacccc

SEQ ID NO: 282 - DNA, 20 bp, Artificial Sequence
  Antisense Compound
gtcttggaaaggcttatacc

SEQ ID NO: 283 - DNA, 20 bp, Artificial Sequence
  Antisense Compound
ggtcttggaaaggcttatac

SEQ ID NO: 284 - DNA, 20 bp, Artificial Sequence
  Antisense Compound
aggtcttggaaaggcttata

SEQ ID NO: 285 - skipped

SEQ ID NO: 286 - DNA, 20 bp, Artificial Sequence
  Antisense Compound
tgaggtcttggaaaggctta

SEQ ID NO: 287 - DNA, 20 bp, Artificial Sequence
  Antisense Compound
agtccaaaggtgaacaaggg

SEQ ID NO: 288 - DNA, 20 bp, Artificial Sequence
  Antisense Compound
ccagtccaaaggtgaacaag

SEQ ID NO: 289 - DNA, 20 bp, Artificial Sequence
  Antisense Compound
gtccagtccaaaggtgaaca

SEQ ID NO: 290 - DNA, 20 bp, Artificial Sequence
  Antisense Compound
tgtccagtccaaaggtgaac

SEQ ID NO: 291 - DNA, 20 bp, Artificial Sequence
  Antisense Compound
tccacagcagcagccacagc

SEQ ID NO: 292 - DNA, 20 bp, Artificial Sequence
  Antisense Compound
tactcttctctgagatgccc

SEQ ID NO: 293 - DNA, 20 bp, Artificial Sequence
  Antisense Compound
tttactcttctctgagatgc

SEQ ID NO: 294 - DNA, 20 bp, Artificial Sequence
  Antisense Compound
gatttactcttctctgagat

SEQ ID NO: 295 - DNA, 20 bp, Artificial Sequence
  Antisense Compound
ggatttactcttctctgaga

SEQ ID NO: 296 - DNA, 20 bp, Artificial Sequence
  Antisense Compound
aggatttactcttctctgag

SEQ ID NO: 297 - DNA, 20 bp, Artificial Sequence
  Antisense Compound
gaggatttactcttctctga

SEQ ID NO: 298 - DNA, 20 bp, Artificial Sequence
  Antisense Compound
tgaggatttactcttctctg

SEQ ID NO: 299 - DNA, 20 bp, Artificial Sequence
  Antisense Compound
tctggcttgagctctgagca

SEQ ID NO: 300 - DNA, 20 bp, Artificial Sequence
  Antisense Compound
gtctggcttgagctctgagc

SEQ ID NO: 301 - DNA, 20 bp, Artificial Sequence
  Antisense Compound
aagtcttttggaaatctgcc

SEQ ID NO: 302 - DNA, 20 bp, Artificial Sequence
  Antisense Compound
ccttcataccatggttcttc

SEQ ID NO: 303 - DNA, 20 bp, Artificial Sequence
  Antisense Compound
gagcacctctaggcaatgac

SEQ ID NO: 304 - DNA, 20 bp, Artificial Sequence
  Antisense Compound
ccgctgatcacagctgacat

SEQ ID NO: 305 - DNA, 20 bp, Artificial Sequence
  Antisense Compound
ccgctcatcactgctgacat

SEQ ID NO: 306 - DNA, 20 bp, Artificial Sequence
  Antisense Compound
ccactcatcactgctgactt